(19) United States
(12) Patent Application Publication
(10) Pub. No.: US 2009/0170847 A1
(43) Pub. Date: Jul. 2, 2009

(54) Imidazopyridine Derivatives Inhibiting Protein Kinase Activity, Method for the Preparation Thereof and Pharmaceutical Composition Containing Same

(76) Inventors: Seung Chul Lee, Seoul (KR); Jin Seok Choi, Seoul (KR); Jung Hoon Oh, Seoul (KR); Boonsaeng Park, Seoul (KR); Yong Eun Kim, Seoul (KR); Jun Hee Lee, Seoul (KR); Dongkyu Shin, Seoul (KR); Cheol Min Kim, Gyeonggi-do (KR); Young-Lan Hyun, Seoul (KR); Cheol Soon Lee, Gyeonggi-do (KR); Joong-Myung Cho, Seoul (KR); Seonggu Ro, Gyeonggi-do (KR)

Correspondence Address: BAKER BOTTS L.L.P., 30 ROCKEFELLER PLAZA, 44TH FLOOR, NEW YORK, NY, 10112-4498, US

(21) Appl. No.: 12/161,916

(22) PCT Filed: Jan. 23, 2007
(86) PCT No.: PCT/KR2007/000393

§ 371 Date: Aug. 14, 2008

Related U.S. Application Data
(60) Provisional application No. 60/846,411, filed on Sep. 21, 2006.

(30) Foreign Application Priority Data
Jan. 23, 2006 (KR) 10-2006-0006834

(51) Int. Cl. A61K 31/5377; A61K 31/53; A61K 31/506; A61K 31/496; A61K 31/4745; C07D 471/02

(52) U.S. Cl. 514/234.5; 514/241; 514/256; 514/303; 544/212; 544/333; 514/253.04; 544/128; 544/362; 546/118

(57) ABSTRACT

The inventive imidazopyridine derivative can be used in a pharmaceutical composition for preventing or treating diseases such as diabetes, obesity, dementia, cancer, and inflammation, since it can efficiently inhibit the activities of several protein kinases including glycogen synthase kinase-3 (GSK-3), aurora kinase, extracellular signal-regulated kinase (ERK), protein kinase B (AKT), and the likes, to control signal transductions thereof.

### FIELD OF THE INVENTION

The present invention relates to a novel compound which inhibits protein kinase activity, a method for the preparation thereof, and a pharmaceutical composition comprising the same as an active ingredient.

### BACKGROUND OF THE INVENTION

Protein kinases are enzymes mediating intracellular signal transduction by delivering the phosphoryl group derived from nucleoside triphosphate (NTP) to specific proteins to phosphorylate them. Many protein kinases have been reported to be involved in several signal pathways which control cellular functions including cell proliferation, differentiation and death (Schlessinger et al., Neuron, 9, 383, 1992).

Accordingly, abnormal activation of protein kinases may cause diverse diseases, e.g., disorders of central nervous system, such as Alzheimer's disease (Mandelkow, E. M. et al., FEBS Lett., 314, 315, 1992; Sengupta, A. et al., Mol. Cell. Biochem., 167, 99, 1997), inflammatory disorders (Badger, J. Pharm. Exp. Ther., 279, 1453, 1996), psoriasis (Dvir et al., J. Cell Biol., 113, 857, 1991), bone disorders such as osteoporosis (Tanaka et al., Nature, 383, 528, 1996), cancers (Hunter et al., Cell, 79, 573, 1994), arteriosclerosis (Hajjar et al., FASEB J., 6, 2933, 1992), thrombosis (Salari, FEBS, 263, 104, 1990), metabolic disorders such as diabetes (Borthwick, A. C. et al., Biochem. Biophys. Res. Commun., 210, 738, 1995), vascular proliferative disorders such as angiogenesis (Strawn et al., Cancer Res., 56, 3540, 1996; Jackson et al., J. Pharm. Exp. Ther., 284, 687, 1998), stent restenosis (Buchdunger et al., Proc. Nat. Acad. Sci. USA, 92, 2258, 1991), autoimmune diseases such as transplantation rejection (Bolen et al., Ann. Rev. Immunol., 15, 371, 1997), infectious diseases such as fungus infection (International Patent Publication No. WO9805335), chronic renal failure (Liu, I. et al., Int. J. Cardiology, 69, 77-82, 1999) and chronic obstructive pulmonary disease (Nguyen, L. T. et al., Clinical Nutr., 18, 255-257, 1999; Solar, N. et al., Eur. Respir. J, 14, 1015-1022, 1997).

Aurora kinase is a Ser/Thr protein kinase involved in mitosis, and has been demonstrated to be a putative oncoprotein overexpressed in several cancer cells of breast, colon, pancreas and ovarian (Carvajal R D et al., Clin. Cancer Res., 12(23), 6869-75, 2006), and recently, there has been a report that an aurora kinase inhibitor developed by Vertex (USA) represses tumor in a nude mouse (Elizabeth A Harrington et al., Nature Medicine, 10, 262-267, 2004).

p38 mitogen-activated protein kinase (MAPK) is a proline-directed Ser/Thr kinase such as c-jun-N-terminal kinase (JNK) and extracelluar signal-regulated kinase (ERK), and it has been known to be activated by bacterial lipopolysaccharides, physico-chemical stresses, pro-inflammatory cytokines including tumor necrosis factor (TNF-α) and interleukin-1 (IL-1), to mediate a signal pathway inducing the expression of inflammatory cytokines such as TNF-α, IL-8, IL-1 and cyclooxygenase-2.

Among such inflammatory cytokines expressed by p38 MAPK activation, TNF-α has been know to be involved in viral infections such as human immunodeficiency virus (HIV), influenza virus and herpes virus infection, as well as inflammatory disorders such as rheumatoid inflammation, multiple sclerosis and asthma (Newton R et al., BioDrugs, 17(2), 113-129, 2003). Further, IL-8 is expressed in monocytes, fibroblasts, endothelial cells and keratinocytes to participate in inflammatory disorders, and IL-1 is expressed by activated monocytes and macrophases to take part in inflammations accompanying rheumatoid, fever and reduction of bone resorption (Bryan Coburn et al., British Journal of Cancer, 95, 1568-1575, 2006).

C-jun-N-terminal kinase (JNK) has been demonstrated to be activated by extracellular stimuli, e.g., Fas/FasL interaction, cytokines including IL-1 and TNF-α, UV, and alteration in potassium homeostasis and osmotic pressure, to mediate a signal pathway inducing the activation of AP1 transcription factor, and participate in apoptosis and inflammatory diseases (Samadder, P. et al., J. Med. Chem., 47(10), 2710-2713, 2004).

Extracellular signal-regulated kinase (ERK) can activate other protein kinases such as Rsk90 (Bjorbaek et al., J. Biol. Chem., 270, 18848, 1995) and MAPKAP2 (Rouse et al., Cell, 78, 1027, 1994), as well as transcription factors such as ATF2 (Raingeaud et al., Mol. Cell Biol., 16, 1247, 1996), Elk-1 (Raingeaud et al., Mol. Cell. Biol., 16(3), 1247-55, 1996), c-Fos (Chen et al., Proc. Natl. Acad. Sci. USA, 90, 10952, 1993) and c-Myc (Oliver et al., Proc. Soc. Exp. Biol. Med., 210, 162, 1995) to mediate the expression of several oncoprotein. Further, ERK has been reported to be overexpressed in human breast cancer cells (Sivaraman et al., J. Clin. Invest., 99, 1478, 1997), regulating the negative growth thereof (Frey et al., Cancer Res., 57, 628, 1997), and it is also reported to be involved in asthma (Whelchel et al., Am. J. Respir. Cell Mol. Biol., 16, 589, 1997).

Cycline-dependent kinase (CDK) is known to play a prominent role in G1/S transition and G2/M transition in the cell cycle (Kim Nasmyth, Science, 274, 1643-1677, 1996) to regulate the cell growth. In particular, there have been found mutations of genes encoding CDK or CDK regulator in cancer cells in the exponential growth phage (Webster, Exp. Opin. Invest. Drugs, 7, 865-887, 1998).

Protein kinase B (PKB or AKT) is activated through the phosphatidyl inositol 3 kinase (PI3K) activation induced by platelet-derived growth factor (PDGF), nerve growth factor (NGF) or insulin-like growth factor-1 (IGF-1) (Kulik et al., Mol. Cell Biol., 17, 1595-1606, 1997; and Hemmings, B. A., Science, 275, 628-630, 1997) to mediate insulin metabolism (Calera, M. R. et al., J. Biol. Chem., 273, 7201-7204, 1998), cell differentiation, and/or cell proliferation, as well as stress response of protein synthesis (Alessi, D. R. et al., Curr. Opin. Genet. Dev., 8, 55-62, 1998).

Further, AKT is reported to be overexpressed in several cancers (Khwaja, A., Nature, 401, 33-34, 1999; Yuan, Z. Q. et al., Oncogene, 19, 2324-2330, 2000; and Namikawa, K., et al., J. Neurosci., 20, 2875-2886, 2000), particularly in ovarian cancer cells (Cheng, J. Q. et al., Proc. Natl. Acad. Sci. USA, 89, 9267-9271, 1992) and pancreas cancer (Cheng, J. Q. et al., Proc. Natl. Acad. Sci. USA, 93, 3636-3641, 1996).

Glycogen synthase kinase 3 (GSK-3) known as one of the target proteins for treating diabetes and dementia is an enzyme that phosphorylates glycogen synthase (GS) to suppress its activity. There have been reports that the activity of GSK-3 in obese diabetic mice is about twice as high as that in control (H. Eldar-Finkelman, Diabetes, 48, 1662-1666, 1999), and the activity and expression of GSK-3 in patients with type 2 diabetes is significantly higher relatively to that in normal persons (S. E. Nikoulina et al., Diabetes, 49, 263-171, 2000).

Accordingly, the present inventors have endeavored to develop a compound which is effective in inhibiting the activity of several protein kinases, and have found that an imidazopyridine derivative can efficiently inhibit the activity of protein kinases including glycogen synthase kinase-3 (GSK-3), aurora kinase, extracellular signal-regulated kinase (ERK), protein kinase B (AKT), cyclin-dependent kinase (CDK), p38 (protein 38) mitogen-activated protein kinase (MAPK), kinase insert domain protein receptor (KDR) or vascular endothelial growth factor receptor-2 (VEGFR-2), c-Jun N-terminal kinase (JNK) and pyruvate dehydrogenase kinase (PDK).

### SUMMARY OF THE INVENTION

Accordingly, it is an object of the present invention to provide a novel compound a pharmaceutically acceptable salt, hydrate, solvate or isomer thereof, that can efficiently inhibit the activity of protein kinases.

It is another object of the present invention to provide a method for preparing such compound.

It is a further object of the present invention to provide a pharmaceutical composition comprising said compound, a pharmaceutically acceptable salt, hydrate, solvate, or isomer thereof.

In accordance with one aspect of the present invention, there is provided an imidazopyridine derivative of formula 1, and a pharmaceutically acceptable salt, hydrate, solvate and isomer thereof:

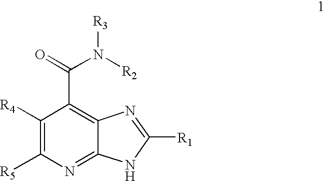

wherein,

R₁ is hydroxy, halogen, C₁₋₆alkyloxy, C₁₋₆alkyl, amino, C₁₋₆alkylamino, carboxyl, nitro, sulfonylamide, C₁₋₆alkylsulfonyl, amide, aryl or heteroaryl optionally substituted with halogen, —CN, NO₂, C₁₋₆alkyl, C₁₋₆alkylpiperazinyl, C₁₋₆alkylsulfinyl C₁₋₆alkyl, piperidinyl, morpholinyl, pyrrolidinyl, morpholinyl C₁₋₆ alkylamino, pyrrolidinyl C₁₋₆ alkylamino, —OR′, —C(O)OR′, —OC(O)R′, —NR′R″, —NHC(O)R′, —C(O)NR′R″, —NHC(S)R′, —C(S)NR′R″, —SR′, —S(O)R′, —SO₂R′, —NHSO₂R′, —SO₂NR′R″, —OSO₂R′, —SO₂₀R′, aryl, heteroaryl, aryl-C₁₋₄alkyl, formyl or trifluoromethyl, R′ or R″ being each independently hydrogen; or C₁₋₄alkyl, C₃₋₇cycloalkyl, aryl or heteroaryl optionally substituted with C₁₋₄alkyl, C₁₋₄ alkoxy, CN, NO₂, NH₂, (C₁₋₄alkyl)amino, OH, COOH, COO(C₁₋₄alkyl), —CONH₂, formyl or trifluoromethyl; the aryl being phenyl, indanyl or naphthyl; and heteroaryl being 5-10 membered-ring aryl, or mono- or bicyclic heterocycle comprising one or more nitrogen, sulfur or oxygen atom in its ring structure;

R₂ is hydrogen; unsubstituted or substituted C₁₋₈alkyl; or unsubstituted or substituted C₁₋₇alkyl comprising nitrogen, sulfur or oxygen in its chain structure, the substituent of the alkyl being hydroxy, halogen, C₁₋₆alkyloxy, alkyl, amino, C₁₋₆alkylamino, carboxyl, nitro, sulfonylamide, alkylsulfonyl or amide; aryl or heteroaryl optionally substituted with C₁₋₄alkyl, hydroxy, halogen, C₁₋₆alkyloxy, amino, C₁₋₆alkylamino, aminoC₁₋₆alkyl, acetylamino, carboxyl, amide, dioxoindole, —CN, NO₂, —OR′, —C(O)OR′, —OC(O)R′, —NR′R″, —NHC(O)R′, —NHC(O)OR′, —C(O)NR′R″, —NHC(S)R′, —C(S)NR′R″, —SR′, —S(O)R′, —SO₂R′, —NHSO₂R′, —SO₂NR′R″, —OSO₂R′, —SO₂OR′, aryl, heteroaryl, aryl-C₁₋₄alkyl, formyl or trifluoromethyl, R′ or R″ being each independently hydrogen; or C₁₋₄alkyl, C₃₋₇cycloalkyl, aryl or heteroaryl optionally substituted with halogen, C₁₋₄alkyl, C₁₋₄alkoxy, CN, NO₂, NH₂, C₁₋₄alkylamino, aminoC₁₋₄alkyl, OH, COOH, —COOC₁₋₄alkyl, —CONH₂, formyl, C₁₋₆alkylpiperazinyl, morpholinyl or trifluoromethyl; the aryl being phenyl, indanyl or naphthyl; and heteroaryl being 5-10 membered-ring aryl, pyridone or mono or bicyclic heterocycle comprising one to four nitrogen, sulfur or oxygen atom in its ring structure; or

unsubstituted or substituted aryl; or unsubstituted or substituted aryl comprising one or more nitrogen, sulfur or oxygen in its ring structure, the substituent of the aryl being hydroxy; halogen; C₁₋₆alkyloxy; C₁₋₆alkyl; amino; C₁₋₆alkylamino; carboxyl; nitro; sulfonylamide; C₁₋₆alkylsulfonyl; amide; unsubstituted or substituted C₁₋₆alkyl; or cyclicC₁₋₆alkyl comprising one or more nitrogen, sulfur or oxygen atome in its ring structure, the substituent of the alkyl being hydroxy; halogen; C₁₋₆alkyloxy; C₁₋₆alkyl; amino; C₁₋₆alkylamino; carboxyl; nitro; sulfonylamide; C₁₋₆alkylsulfonyl; amide; aryl optionally substituted with hydroxy, halogen, C₁₋₆alkyloxy, C₁₋₆alkyl, amino, C₁₋₆alkylamino, carboxyl, nitro, amide or dioxoisoindole; sulfonylaminoaryl having an aryl group substituted with hydroxy, halogen, C₁₋₆alkyloxy, C₁₋₆alkyl, amino, C₁₋₆alkylamino, carboxyl, nitro, sulfonylamide, C₁₋₆alkylsulfonyl or amide; aryl comprising one or more nitrogen, sulfur or oxygen atoms in its ring structure which is represented by pyrrole, pyrazole, imidazole, 1,2,3-triazole, 1,2,4-triazole, isooxazole, oxazole, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, oxypyridien, pyrimidine or triazine optionally substituted with hydroxy, halogen, C₁₋₆alkyloxy, C₁₋₆alkyl, amino, C₁₋₆alkylamino, carboxyl, nitro, sulfonylamide, C₁₋₆alkylsulfonyl or amide; or C₃₋₈cycloalkyl optionally substituted with hydroxy, halogen, C₁₋₆alkyloxy, C₁₋₆alkyl, amino, C₁₋₆alkylamino, carboxyl, nitro or amide;

R₃ is hydrogen; or C₁₋₄alkyl or C₃₋₇cycloalkyl optionally substituted with one or more substituent selected from the group consisting of halogen, C₁₋₄alkyl, C₁₋₄alkoxy, CN, NO₂, NH₂, (C₁₋₄alkyl)-amino, amino-(C₁₋₄alkyl), OH, COOH, —COO(C₁₋₄alkyl), and —CONH₂, having an optional substituent selected from the group consisting of hydroxy; halogen; alkyloxy; alkyl; amino; alkylamino; carboxyl; nitro; sulfonylamide; alkylsulfonyl; or amide; or

R₂ and R₃ are fused together with the nitrogen to which they are attached to form a ring, and

R₄ and R₅ are each independently hydrogen; or C₁₋₄alkyl or C₃₋₇cycloalkyl substituted with an optional substituent selected from the group consisting of halogen, C₁₋₄alkyl, C₁₋₄alkoxy, CN, NO₂, NH₂, C₁₋₄alkylamino, aminoC₁₋₄alkyl, OH, COOH, COOC₁₋₄alkyl and —CONH₂, each of which having an optional substituent, be selected from the group consisting of hydroxy, halogen, alkyloxy, alkyl, amino, alkylamino, carboxyl, nitro, sulfonylamide, alkylsulfonyl and amide.

### DETAILED DESCRIPTION OF THE INVENTION

Among the compound of the formula 1, preferred are those wherein:

R₁ is phenyl, pyrrolidinylphenyl, dichlorophenyl, chlorophenyl, fluorophenyl, difluorophenyl, furanyl, thiophene, cyclopropyl, C₁₋₂alkylpiperazinylphenyl, C₁₋₂alkylpiperazinylC₁₋₃alkylphenyl, C₁₋₂alkylpiperazinylC₁₋₃alkylaminophenyl, methanesulfinylphenyl, diC₁₋₂alkylaminophenyl, morpholinylphenyl, piperidinylphenyl, morpholinylC₁₋₃alkylaminophenyl, pyrrolidinylC₁₋₃alkylaminophenyl, dimethylaminoC₁₋₄alkylaminophenyl, diC₁₋₂alkylaminoethylmethylaminophenyl, piperazinylaminophenyl, piperazinylC₁₋₂alkylaminophenyl, thiomorpholinylphenyl, piperidinylaminophenyl, piperidinylC₁₋₂alkylaminophenyl, methoxyphenyl, diC₁₋₃alkylaminopyrrolidinylphenyl or pyridinyl;

R₂ is C₁₋₅alkyl optionally substituted with sulfonylphenyl, C₁₋₂alkylpyridinyl, diC₁₋₂alkyl, triC₁₋₂alkyl, tetraC₁₋₂alkyl, pyridinyl, oxypyridinyl, chloropyridinyl, morpholinyl, aminoC₁₋₂alkylpyridinyl, acetylaminophenyl, imidazole, dichloroimidazole, C₁₋₂alkylimidazole, diC₁₋₂alkylaminosulfonylaminophenyl, trifluoroC₁₋₂alkylphenyl, benzyloxyoxopyridinyl, hydroxyoxopyridinyl, C₁₋₂alkanesulfonylaminophenyl, diC₁₋₂alkylaminoacetylaminophenyl, trifluoromethanesulfonylaminophenyl, fluoropyridinyl, fluorohydroxyphenyl, C₁₋₂alkylpiperazinylacetylaminophenyl, chlorooxypyridinyl, thiophenyl, C₁₋₂alkyloxypyridinyl, aminophenyl, hydroxyphenyl, C₁₋₂alkylpiperazincarbonylaminophenyl, morpholinylC₁₋₃alkoxyphenyl, benzyl, hydroxyl diC₁₋₂alkyl or diC₁₋₂alkylaminoC₁₋₂alkyl; cyclo C₃₋₇alkyl optionally substituted with triC₁₋₂alkyl, amino or hydroxy; pyridinyl optionally substituted with C₁₋₂alkyl, diC₁₋₂alkyl, chloroC₁₋₂alkoxy, C₁₋₂alkylamino, aminoC₁₋₂alkyl, C₁₋₂alkoxy, C₁₋₂alkoxyC₁₋₂alkyl, C₁₋₂alkylsulfanyl, chloroC₁₋₂alkyl, isobutoxy, cyclopropylmethoxy, diC₁₋₂alkylaminoC₁₋₂alkoxy, morpholinylC₁₋₂alkoxy, halogen, acetylamino or C₁₋₂alkylsulfanylC₁₋₂alkyl; phenyl substituted with benzoylamino, piperidinyl, hydroxy, C₁₋₂alkoxy, C₁₋₂alkyl, diC₁₋₂alkyl, diisopropyl, isopropyl, diC₁₋₂alkylaminoacetylamino, fluoro C₁₋₂alkyl, fluorohydroxy, trifluoroC₁₋₂alkoxy, diC₁₋₂alkoxy, acetylamino, cyano, benzyloxy, trifluoromethanesulfonylamino or C₁₋₂alkanesulfonyl; benzothiazoyl, indazolyl, C₁₋₂alkylindolyl, indolyl, naphthalenyl, quinolinyl, C₁₋₂alkylpyrazolyl, phenylthiazolyl, tolyl, benzodioxolyl, C₁₋₂alkylphenylacetamide, C₁₋₂alkylphenoxyacetyl, ethanesulfonylC₁₋₂alkylphenylamide, C₁₋₂alkylphenoxyacetic acid tert-butylester, C₁₋₂alkylphenylmethanesulfonamide, C₁₋₂alkylpiperazinyl, C₁₋₂alkoxyphenylamide, piperidinyl, benzyl piperidinyl, C₁₋₂alkylphenoxyacetyl, triC₁₋₂alkylbicycloheptinyl, adamantanyl, aminobicycloheptanecarboxyl, azabicyclooctyl, bicycloheptinyl, tert-butylamide or C₁₋₂alkylpyridinyl C₁₋₂alkylcarbamic acid tert-butylester; and

R₃ is H, or R₂ and R₃ are fused together with the nitrogen to which they are attached to form a ring; R₄ is H or halogen; and R₅ is H.

Representative examples of the inventive compound are shown in Table 1.

TABLE 1

| Ex. | Compound | Structure | ¹H-NMR | MW |
|---|---|---|---|---|
| 1 | 2-phenyl-3H- imidazo[4,5-b]pyridine- 7-carboxylic acid[2-(4- acetylamino-phenyl)- ethyl]-amide | 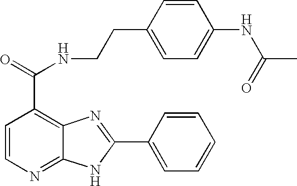 | 2.07 (s, 3H), 2.99 (t, 2H), 3.89 (t, 2H), 7.29 (d, 2H), 7.57~7.49 (m, 5H), 8.06 (m, 2H), 8.55 (d, 1H) | 399.5 |
| 2 | (4-{2-[(2-phenyl-3H- imidazo[4,5-b]pyridine- 7-carbonyl)-amino]- ethyl}phenoxy)-acetic acid | 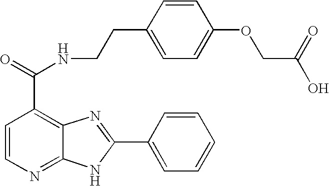 | 2.97 (t, 2H), 3.84 (t, 2H), 4.38 (s, 2H), 6.82 (d, 1H), 6.85 (s, 1H), 6.84 (d, 1H), 7.11 (t, 1H), 7.54 (t, 3H), 7.70 (d, 2H), 7.99 (d, 2H), 8.29 (d, 1H) | 416.4 |
| 3 | 2-phenyl-3H- imidazo[4,5-b]pyridine- 7-carboxylic acid[2-(2- hydroxy-phenyl)-ethyl]- amide | 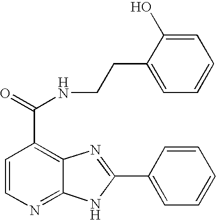 | 2.98 (t, 2H), 3.88 (t, 2H), 6.67 (m, 1H), 6.79 (m, 2H), 7.56 (m, 3H), 7.79 (d, 1H), 8.06 (m, 2H), 8.43 (d, 1H) | 358.4 |
| 4 | (3-{2-[(2-phenyl-3H- imidazo[4,5-b]pyridine- 7-carbonyl)-amino]- ethyl}-phenoxy)-acetic acid | 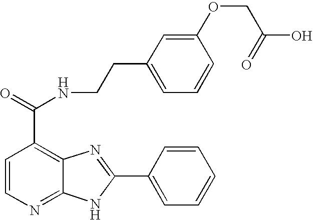 | 2.95 (t, 2H), 3.85 (t, 2H), 4.38 (s, 2H), 6.86 (d, 1H), 6.78 (s, 1H), 6.94 (d, 1H), 7.21 (t, 1H), 7.54 (t, 3H), 7.75 (d, 2H), 8.01 (d, 2H), 8.39 (d, 1H) | 416.4 |
| 5 | 2-phenyl-3H- imidazo[4,5-b]pyridine- 7-carboxylic acid[2-(4- ethanesulfonylamino- phenyl)-ethyl]-amide | 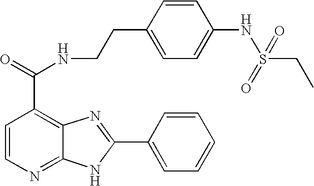 | 1.19 (t, 3H), 2.93 (t, 2H), 2.98 (t, 2H), 3.86 (t, 2H), 7.19 (d, 2H), 7.32 (d, 2H), 7.60 (m, 3H), 7.78 (d, 1H), 8.11 (m, 2H), 8.44 (d, 1H) | 449.5 |
| 6 | (3-{2-[(2-phenyl-3H- imidazo[4,5-b]pyridine- 7-carbonyl)-amino]- ethyl}-phenoxy)-acetic acid tert-butylester | 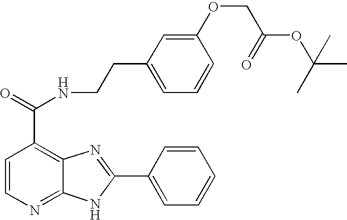 | 1.43 (s, 9H), 2.97 (t, 2H), 3.85 (t, 2H), 4.38 (s, 2H), 6.86 (d, 1H), 6.87 (s, 1H), 6.94 (d, 1H), 7.21 (t, 1H), 7.54 (t, 3H), 7.75 (d, 2H), 8.01 (d, 2H), 8.39 (d, 1H) | 472.5 |
| 7 | (4-{2-[(2-phenyl-3H- imidazo[4,5-b]pyridine- 7-carbonyl)-amino]- ethyl}-phenoxy)-acetic acid tert-butylester | 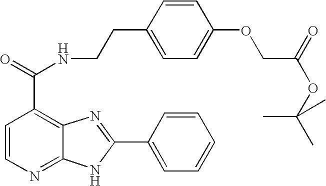 | 1.28 (s, 9H), 2.97 (t, 2H), 3.85 (t, 2H), 4.49 (s, 2H), 6.83 (d, 2H), 7.27 (d, 2H), 7.57 (m, 3H), 7.78 (d, 1H), 8.05 (m, 2H), 8.42 (d, 1H) | 472.5 |
| 8 | 2-phenyl-3H- imidazo[4,5-b]pyridine- 7-carboxylic acid [2-(4- fluoro-3-hydroxy- phenyl)-ethyl]-amide | 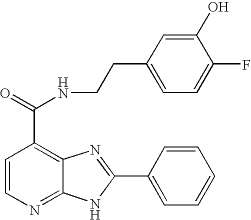 | 2.90 (t, 2H), 3.86 (t, 2H), 6.77 (m, 1H), 6.99~6.78 (m, 2H), 7.57 (m, 3H), 7.79 (d, 1H), 8.05 (m, 2H), 8.44 (d, 1H) | 376.4 |
| 9 | 2-phenyl-3H- imidazo[4,5-b]pyridine- 7-carboxylic acid[2-(3- hydroxy-phenyl)-ethyl]- amide | 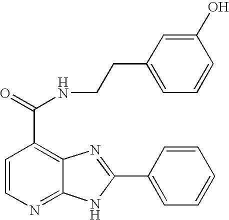 | 2.95 (d, 2H), 3.86 (t, 2H), 6.66 (t, 2H), 6.80 (m, 2H), 7.12 (t, 1H), 7.59 (m, 3H), 7.80 (d, 1H), 8.00 (m, 2H), 8.47 (d, 1H) | 358.4 |
| 10 | 2-phenyl-3H- imidazo[4,5-b]pyridine- 7-carboxylic acid[3- (4,5-dichloro-3H- imidazo-1-yl)-propyl]- amide | 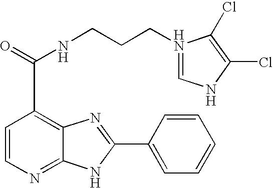 | 2.25 (m, 2H), 3.64 (t, 2H), 4.22 (t, 2H), 7.62 (m, 3H), 7.79 (m, 2H), 8.28 (m, 2H), 8.49 (d, 1H), 7.90 (m, 1H), 8.34 (m, 2H), 8.44 (d, 1H) | 417.3 |
| 11 | 2-phenyl-3H- imidazo[4,5-b]pyridine- 7-carboxylic acid[2-(4- methanesulfonylamino- phenyl)-ethyl]-amide | 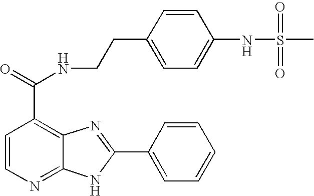 | 2.80 (s, 3H), 3.03 (t, 2H), 3.85 (t, 2H), 7.20 (d, 2H), 7.35 (d, 1H), 7.79 (m, 3H), 8.11 (m, 2H), 8.44 (d, 1H) | 435.5 |
| 12 | 2-phenyl-3H- imidazo[4,5-b]pyridine- 7-carboxylic acid(2- pyridin-2-yl-ethyl)- amide | 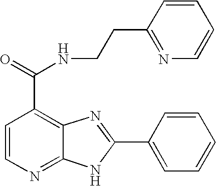 | 3.3 (t, 1H), 4.12 (t, 2H), 7.63 (m, 3H), 7.72 (d, 1H), 7.96 (t, 1H), 8.11 (d, 1H), 8.23 (m, 2H), 8.51 (m, 2H), 8.72 (d, 1H) | 343.4 |
| 13 | 2-phenyl-3H- imidazo[4,5-b]pyridine- 7-carboxylic acid(2- pyridin-3-yl-ethyl)- amide | 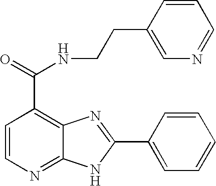 | 3.30 (t, 2H), 4.02 (t, 2H), 7.59 (m, 3H), 7.78 (d, 1H), 7.91 (m, 1H), 8.15 (m, 2H), 8.43 (d, 1H), 8.67 (m, 1H), 8.83 (s, 1H) | 343.4 |
| 14 | 2-phenyl-3H- imidazo[4,5-b]pyridine- 7-carboxylic acid(2- pyridin-4-yl-ethyl)- amide | 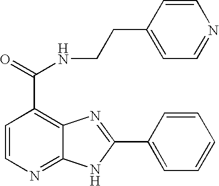 | 3.36 (t, 2H), 40.5 (t, 2H), 7.65 (m, 2H), 7.79 (d, 1H), 8.14 (d, 2H), 8.23 (m, 2H), 8.47 (d, 1H), 8.73 (m, 2H) | 343.4 |
| 15 | 2-phenyl-3H- imidazo[4,5-b]pyridine- 7-carboxylic acid(3- morpholin-4-yl-propyl)- amide | 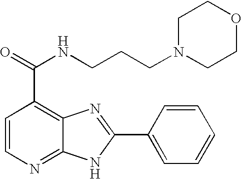 | 2.22 (q, 2H), 3.60 (t, 2H), 4.20 (t, 2H), 3.30~5.00 (br, 8H), 7.79 (m, 2H), 8.28 (m, 2H), 8.49 (d, 1H), 7.90 (m, 1H), 8.34 (m, 2H), 8.44 (d, 1H) | 365.4 |
| 16 | 2-phenyl-3H- imidazo[4,5-b]pyridine- 7-carboxylic acid[2-(1- oxy-pyridin-2-yl)-ethyl]- amide | 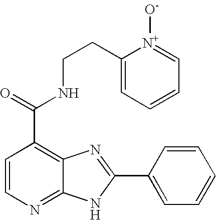 | 3.10 (t, 2H), 3.96 (t, 2H), 7.68~7.85 (m, 4H), 8.15 (d, 2H), 8.30~8.55 (m, 3H), 8.70~8.88 (m, 2H) | 359.4 |
| 17 | 2-phenyl-3H- imidazo[4,5-b]pyridine- 7-carboxylic acid[2-(1- oxy-pyridin-3-yl)-ethyl]- amide | 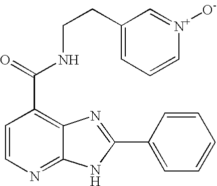 | 3.11 (t, 2H), 3.94 (t, 2H), 7.68~7.85 (m, 3H), 8.00 (m, 1H), 8.15 (d, 2H), 8.35~8.65 (m, 3H), 8.70 (m, 1H), 8.92 (s, 1H) | 359.4 |
| 18 | 2-phenyl-3H- imidazo[4,5-b]pyridine- 7-carboxylic acid[2-(1- oxy-pyridin-4-yl)-ethyl]- amide | 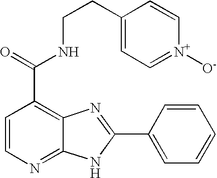 | 3.14 (t, 2H), 4.00 (t, 2H), 7.68~7.85 (m, 3H), 8.00 (m, 1H), 8.24 (m, 2H), 8.35~8.65 (m, 4H), 8.70 (m, 1H) | 359.4 |
| 19 | 2-phenyl-3H- imidazo[4,5-b]pyridine- 7-carboxylic acid[2-(2- chloro-pyridin-3-yl)- ethyl]-amide | 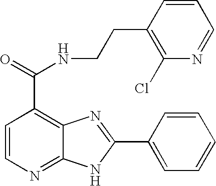 | 3.06 (t, 2H), 3.86 (t, 2H), 7.39 (d, 1H), 7.63 (m, 3H), 7.84~7.78 (m, 2H), 8.14 (m, 2H), 8.33 (s, 1H), 8.53 (m, 1H) | 377.8 |
| 20 | 2-phenyl-3H- imidazo[4,5-b]pyridine- 7-carboxylic acid[2-(2- chloro-pyridin-4-yl)- ethyl]-amide | 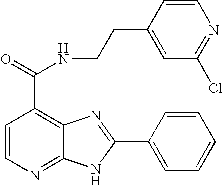 | 3.07 (t, 2H), 3.92 (t, 2H), 7.37 (s, 1H), 7.50 (s, 1H), 7.62 (s, 3H), 7.80 (s, 1H), 8.12 (s, 2H), 8.26 (d, 1H), 8.51 (br, 1H) | 377.8 |
| 21 | 2-phenyl-3H- imidazo[4,5-b]pyridine- 7-carboxylic acid[2-(6- chloro-pyridln-3-yl)- ethyl]-amide | 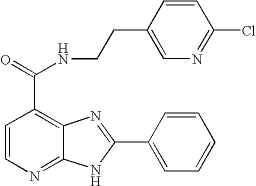 | 3.20 (t, 2H), 3.92 (t, 2H), 7.65~7.80 (m, 3H), 7.88 (d, 1H), 8.05~8.14 (m, 2H), 8.44~8.60 (m, 2H), 8.70 (m, 1H), 8.90 (s, 1H) | 377.8 |
| 22 | 2-phenyl-3H- imidazo[4,5-b]pyridine- 7-carboxylic acid[2-(2- aminomethyl-pyridin-3- yl)-ethyl]-amide | 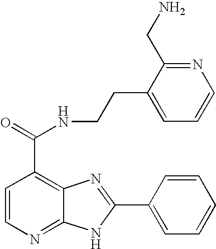 | 3.31 (t, 2H), 3.97 (t, 2H), 4.17 (s, 1H), 5.70 (s, 1H), 7.36 (t, 2H), 7.78 (d, 1H), 7.91 (m, 1H), 8.31 (m, 2H), 8.49 (m, 2H), 8.82 (d, 1H) | 372.4 |
| 23 | 2-phenyl-3H- imidazo[4,5-b]pyridine- 7-carboxylic acid[2-(2- aminomethyl-pyridin-4- yl)-ethyl]-amide | 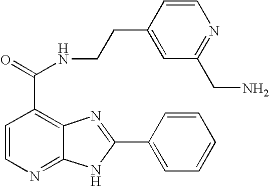 | 3.40 (t, 2H), 4.02 (t, 2H), 4.46 (s, 2H), 7.62 (m, 3H), 7.75 (d, 1H), 7.97 (d, 1H), 8.08 (s, 1H), 8.26 (m, 1H), 8.45 (s, 1H), 8.78 (d, 1H) | 372.4 |
| 24 | 2-phenyl-3H- imidazo[4,5-b]pyridine- 7-carboxylic acid(4- benzoylamino-phenyl)- amide | 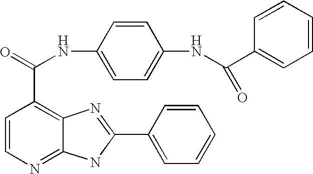 | 7.10 (d, 1H), 7.31~7.62 (m, 7H), 7.66 (s, 1H), 7.69 (m, 1H), 7.81-7.92 (m, 3H), 8.23 (d, 1H), 8.58 (d, 1H), 8.83 (s, 1H) | 433.5 |
| 25 | 2-phenyl-3H- imidazo[4,5-b]pyridine- 7-carboxylic acid(4- piperidin-1-yl-phenyl)- amide | 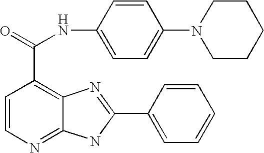 | 8.26 (m, 2H), 8.13~7.92 (m, 3H), 7.82~7.38 (m, 2H), 7.44~7.56 (m, 2H), 7.11 (d, 2H), 3.39 (m, 2H), 2.55 (s, 2H), 1.71 (m, 6H) | 397.5 |
| 26 | 6-bromo-2-phenyl-3H- imidazo[4,5-b]pyridine- 7-carboxylic acid(4- benzoylamino-phenyl)- amide | 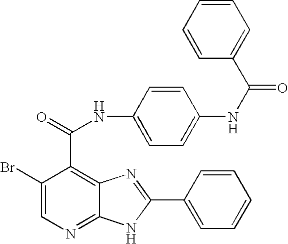 | 7.10 (d, 1H), 7.31-7.62 (m, 6H), 7.66 (s, 1H), 7.67 (m, 1H), 7.81-7.92 (m, 3H), 8.33 (d, 1H), 8.56 (d, 1H), 8.87 | 512.4 |
| 27 | 6-bromo-2-phenyl-3H- imidazo[4,5-b]pyridine- 7-carboxylic acid[2-(2- chloro-pyridin-4-yl)- ethyl]-amide | 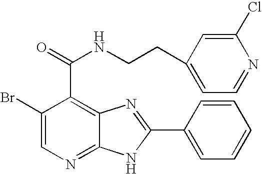 | 3.22 (t, 2H), 3.93 (t, 2H), 7.63~7.80 (m, 3H), 7.88 (d, 1H), 8.05~8.14 (m, 2H), 8.44~8.60 (m, 1H), 8.70 (m, 1H), 8.90 (s, 1H) | 456.7 |
| 28 | 6-bromo-2-phenyl-3H- imidazo[4,5-b]pyridine- 7-carboxylic acid(2- thiophen-2-yl-ethyl)- amide | 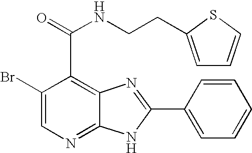 | 3.22 (t, 2H), 3.93 (t, 2H), 7.63~8.10 (m, 2H), 7.68 (d, 1H), 8.05~8.24 (m, 2H), 8.44~8.60 (m, 2H), 8.70 (m, 1H), 8.90 (br, 1H) | 427.3 |
| 29 | 6-chloro-2-phenyl-3H- imidazo[4,5-b]pyridine- 7-carboxylic acid[3- (4,5-dichloro-imidazo- 1-yl)-propyl]-amide | 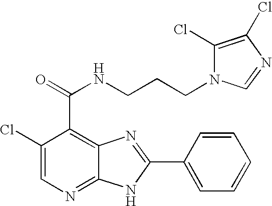 | 2.26 (m, 2H), 3.66 (m, 2H), 4.20 (t, 2H), 7.57 (t, 1H), 7.74 (d, 1H), 7.81 (s, 1H), 8.21 (m, 1H), 8.45 (m, 2H), 8.82 (s, 1H) | 449.7 |
| 30 | 2-phenyl-3H- imidazo[4,5-b]pyridine- 7-carboxylic acid phenylamide | 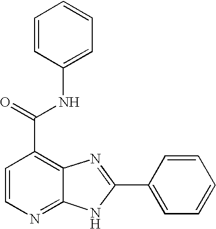 | 7.21-7.29 (m, 7H), 7.38 (d, 1H), 7.46 (d, 1H), 7.88-7.90 (d, 1H), 8.29-8.35 (m, 2H) | 314.3 |
| 31 | 2-phenyl-3H- imidazo[4,5-b]pyridine- 7-carboxylic acid cyclohexylamide | 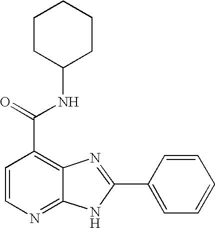 | 1.36-1.66 (m, 5H), 1.62- 1.79 (m, 5H), 4.01-4.18 (m, 1H), 7.18 (d, 2H), 7.76 (d, 1H), 7.90~8.12 (m, 3H), 8.41 (d, 1H) | 320.4 |
| 32 | 2-(2,4-dichloro-phenyl)- 3H-imidazo[4,5- b]pyridine-7-carboxylic acid[2-(4-acetylamino- phenyl)-ethyl]-amide | 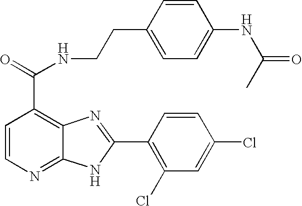 | 2.07 (s, 3H), 2.99 (t, 2H), 3.89 (t, 2H), 7.29 (d, 2H), 7.57~7.49 (m, 5H), 8.06 (m, 2H), 8.55 (d, 1H) | 468.3 |
| 33 | 2-(2,4-dichloro-phenyl)- 3H-imidazo[4,5- b]pyridine-7-carboxylic acid[2-(3-hydroxy- phenyl)-ethyl]-amide | 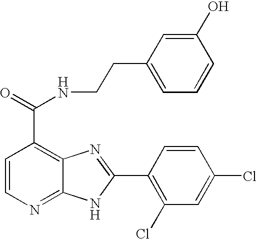 | .91 (t, 2H), 3.80 (t, 2H), 6.62 (d, 1H), 6.73 (m, 2H), 7.05 (t, 1H), 7.56 (m, 1H), 7.72 (s, 1H), 7.82 (m, 2H) 8.52 (d, 1H) | 427.3 |
| 34 | 2-(2,4-dichloro-phenyl)- 3H-imidazo[4,5-b] pyridine-7-carboxylic acid[2-(2-hydroxy- phenyl)-ethyl]-amide | 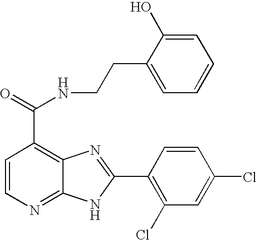 | 2.95 (t, 2H), 3.80 (t, 2H), 6.65 (t, 1H), 6.74 (d, 1H), 7.12 (d, 1H), 7.56 (d, 1H), 7.74 (s, 1H), 7.82 (m, 2H), 8.53 (d, 1H) | 427.3 |
| 35 | [4-(2-{[2-(2,4- dichloro-phenyl)-3H- imidazo[4,5-b]pyridine- 7-carbonyl]-amino}- ethyl)-phenoxy]-acetic acid | 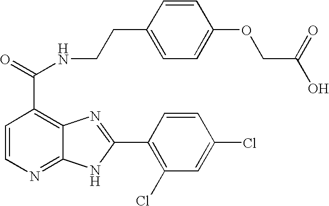 | 2.96 (t, 2H), 3.95 (t, 2H), 4.56 (s, 2H), 6.77 (d, 2H), 7.20 (d, 2H), 7.62 (d, 1H), 7.81 (m, 3H), 8.53 (d, 1H) | 485.3 |
| 36 | [3-(2-{[2-(2,4- dichloro-phenyl)-3H- imidazo[4,5-b]pyridine- 7-carbonyl]-amino}- ethyl)-phenoxy]-acetic acid | 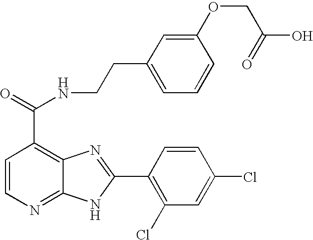 | 2.93 (t, 2H), 3.79 (t, 2H), 4.56 (s, 2H), 6.81 (d, 1H), 7.23 (s, 1H), 7.22 (d, 1H), 7.82 (m, 3H), 8.56 (d, 1H) | 485.3 |
| 37 | [4-(2-{[2-(2,4- dichloro-phenyl)-3H- imidazo[4,5-b]pyridine- 7-carbonyl]-amino}- ethyl)-phenoxy]-acetic acid tert-butylester | 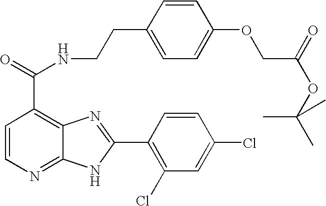 | 1.45 (s, 9H), 2.92 (t, 2H), 3.78 (t, 2H), 4.48 (s, 2H), 6.77 (d, 2H), 7.21 (d, 2H), 7.60 (d, 1H), 7.79 (m, 3H), 8.52 (d, 1H) | 541.4 |
| 38 | [3-(2-{[2-(2,4- dichloro-phenyl)-3H- imidazo[4,5-b]pyridine- 7-carbonyl]-amino}- ethyl)-phenoxy]-acetic acid tert-butylester | 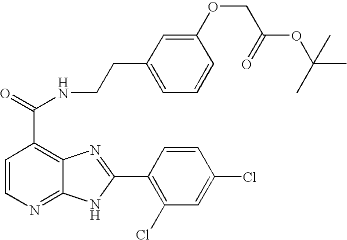 | 1.43 (s, 9H), 2.97 (t, 2H), 3.85 (t, 2H), 4.38 (s, 2H), 6.86 (d, 1H), 6.87 (s, 1H), 6.94 (d, 1H), 7.21 (t, 1H), 7.54 (t, 3H), 7.75 (d, 2H), 8.01 (d, 2H), 8.39 (d, 1H) | 541.4 |
| 39 | 2-(2,4-dichloro-phenyl)- 3H-imidazo[4,5-b] pyridine-7-carboxylic acid[3-(3H-imidazo-1- yl)-propyl]-amide | 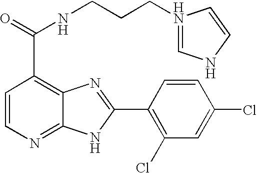 | 2.31 (t, 2H), 3.62 (t, 2H), 4.40 (t, 2H), 7.47 (s, 1H), 7.57 (d, 1H), 7.75 (d, 2H), 7.84 (d, 1H), 7.97 (d, 1H), 8.56 (d, 2H), 8.99 (s, 1H) | 417.3 |
| 40 | 2-(2,4-dichloro-phenyl)- 3H-imidazo[4,5-b] pyridine-7-carboxylic acid{2-[4-(2- dimethylamino- acetylamino)-phenyl]- ethyl}-amide | 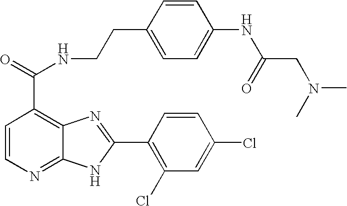 | 3.97 (m, 8H), 3.80 (t, 2H), 4.08 (s, 2H), 7.29 (d, 2H), 7.49 (d, 2H), 7.59 (d, 1H), 7.74 (s, 1H), 7.83 (m, 2H), 8.53 (d, 1H) | 511.4 |
| 41 | 2-(2,4-dichloro-phenyl)- 3H-imidazo[4,5-b] pyridine-7-carboxylic acidacid[2-(4- sulfonylphenyl)-ethyl]- amide | 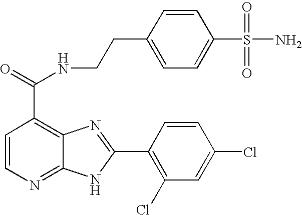 | 3.97 (m, 8H), 3.80 (t, 2H), 4.08 (s, 2H), 7.29 (d, 2H), 7.49 (d, 2H), 7.59 (d, 1H), 7.72 (s, 1H), 8.03 (m, 2H), 8.73 (d, 1H) | 490.4 |
| 42 | [2-(2-{[2-(2,4- dichloro-phenyl)-3H- imidazo[4,5-b]pyridine- 7-carbonyl]-amino}- ethyl)-phenoxy]-acetic acid | 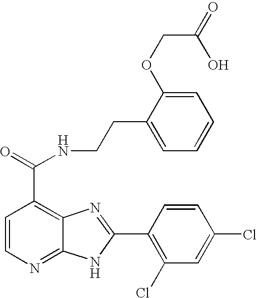 | 3.07 (t, 3H), 3.86 (t, 2H), 4.64 (s, 2H), 6.80 (m, 3H), 7.15 (m, 3H), 7.54 (d, 1H), 7.74 (s, 1H), 7.81 (m, 2H), 8.52 (d, 1H) | 485.3 |
| 43 | 2-(4-chloro-phenyl)- 3H-imidazo[4,5- b]pyridine-7-carboxylic acid[2-(4- ethanesulfonylamino- phenyl)-ethyl]-amide | 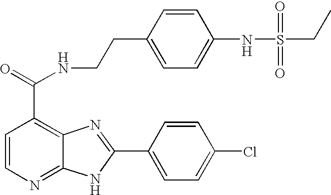 | 1.19 (t, 3H), 2.99 (q, 2H), 3.02 (t, 2H), 3.85 (t, 2H), 7.18 (d, 2H), 7.32 (d, 4H), 7.79 (d, 1H), 8.06 (d, 2H), 8.30 (d, 1H) | 484.0 |
| 44 | 2-(4-chloro-phenyl)- 3H-imidazo[4,5- b]pyridine-7-carboxylic acid[2-(4-acetylamino- phenyl)-ethyl]-amide | 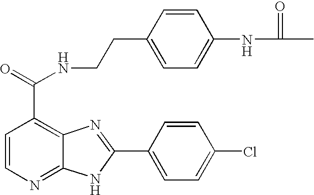 | 2.11 (s, 3H), 2.98 (t, 2H), 3.89 (t, 2H), 7.27 (d, 2H), 7.53 (m, 4H), 7.80 (m, 2H), 7.94 (d, 2H), 8.44 (d, 1H) | 433.9 |
| 45 | 2-(4-chloro-phenyl)- 3H-imidazo[4,5- b]pyridine-7-carboxylic acid[2-(3-hydroxy- phenyl)-ethyl]-amide | 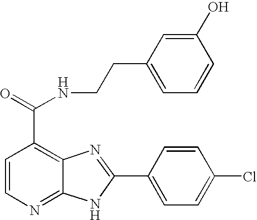 | 2.96 (t, 2H), 3.90 (t, 2H), 6.68 (d, 1H), 6.80 (d, 2H), 7.13 (t, 1H), 7.54 (d, 2H), 7.80 (d, 1H), 8.00 (d, 2H), 8.42 (d, 1H) | 392.8 |
| 46 | 2-(4-chloro-phenyl)- 3H-imidazo[4,5- b]pyridine-7-carboxylic acid{2-[4-(2- dimethylamino- acetylamino)-phenyl]- ethyl}-amide | 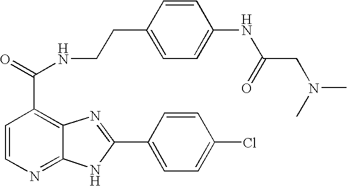 | 2.86 (s, 6H), 3.02 (t, 2H), 3.88 (t, 2H), 4.08 (s, 2H), 7.30 (d, 2H), 7.54 (d, 4H), 7.79 (d, 1H), 8.03 (d, 2H), 8.44 (d, 1H) | 477.0 |
| 47 | 2-(4-chloro-phenyl)- 3H-imidazo[4,5- b]pyridine-7-carboxylic acid(3-imidazo-1-yl- propyl)-amide | 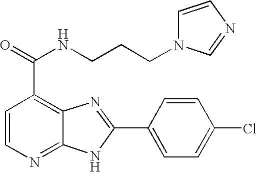 | 2.36 (m, 2H), 3.65 (t, 2H), 4.44 (t, 2H), 7.54 (m, 1H), 7.60 (d, 2H), 7.75 (m, 1H), 7.81 (d, 1H), 8.25 (d, 2H), 8.48 (d, 2H), 9.03 (s, 1H) | 380.8 |
| 48 | 2-(4-chloro-phenyl)- 3H-imidazo[4,5- b]pyridine-7-carboxylic acid[3-(4,5-dichloro- imidazo-1-yl)-propyl]- amide | 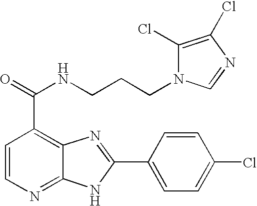 | 2.26 (m, 2H), 3.66 (m, 2H), 4.20 (t, 2H), 7.57 (t, 2H), 7.74 (d, 1H), 7.81 (s, 1H), 8.21 (m, 2H), 8.45 (d, 1H) | 449.7 |
| 49 | 2-(4-chloro-phenyl)- 3H-imidazo[4,5- b]pyridine-7-carboxylic acid[3-(4-methyl- imidazo-1-yl)-propyl]- amide | 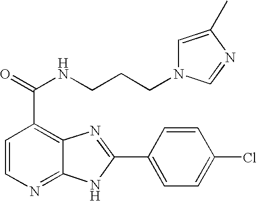 | 2.22 (d, 3H), 2.34 (m, 2H), 3.68 (t, 2H), 4.35 (t, 2H), 7.26 (d, 1H), 7.60 (d, 2H), 7.80 (d, 1H), 8.25 (d, 2H), 8.48 (d, 1H), 8.85 (d, 1H) | 394.9 |
| 50 | 2-(4-chloro-phenyl)- 3H-imidazo[4,5- b]pyridine-7-carboxylic acid[2-(4- methanesulfonylamino- phenyl)-ethyl]-amide | 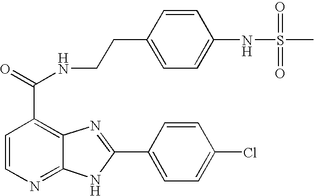 | 2.84 (s, 3H), 3.03 (t, 2H), 3.88 (t, 2H), 7.11 (d, 2H), 7.32 (d, 2H), 7.56 (d, 2H), 7.80 (d, 1H), 8.04 (d, 2H), 8.44 (d, 1H) | 470.0 |
| 51 | 2-(4-chloro-phenyl)- 3H-imidazo[4,5- b]pyridine-7-carboxylic acid[2-(4- dimethyaminosulfonylamino- phenyl)-ethyl]-amide | 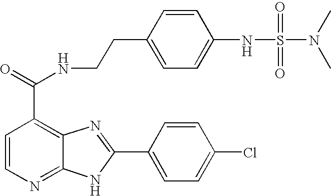 | 2.69 (s, 3H), 3.01 (t, 2H), 3.89 (t, 2H), 7.16 (d, 2H), 7.29 (d, 2H), 7.62 (d, 2H), 7.79 (d, 1H), 8.06 (d, 2H), 8.45 (d, 1H) | 499.0 |
| 52 | 2-(4-chloro-phenyl)- 3H-imidazo[4,5- b]pyridine-7-carboxylic acid[2-(2-hydroxy- phenyl)-ethyl]-amide | 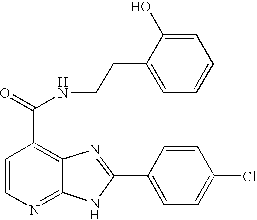 | 3.02 (t, 2H), 3.89 (t, 2H), 6.73 (t, 1H), 6.80 (d, 1H), 7.07 (t, 1H), 7.15 (d, 1H), 7.52 (d, 2H), 7.80 (d, 1H), 8.01 (d, 2H), 8.44 (d, 1H) | 392.8 |
| 53 | 2-(4-chloro-phenyl)- 3H-imidazo[4,5- b]pyridine-7-carboxylic acid[2-(3- trifluoromethyl-phenyl)- ethyl]-amide | 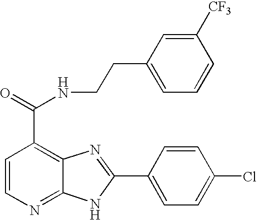 | 3.10 (t, 2H), 3.94 (t, 2H), 7.32-7.63 (m, 6H), 7.81 (d, 1H), 7.97 (d, 2H), 8.43 (d, 1H) | 444.8 |
| 54 | 2-(4-chloro-phenyl)- 3H-imidazo[4,5- b]pyridine-7-carboxylic acid[3-(4-benzyloxy-2- oxo-2H-pyridin-1-yl)- propyl]-amide | 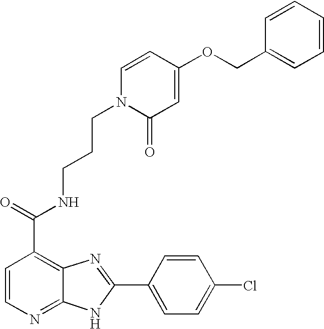 | 2.20 (2H, m), 4.00 (2H, t, J = 6.4 Hz), 4.20 (2H, t, J = 6.4 Hz), 5.05 (2H, s), 6.13 (2H, d, J = 5.7 Hz), 6.34 (1H, s), 7.31~7.42 (8H, m), 7.99 (1H, bs), 8.18 (2H, d, J = 8.4 Hz), 8.35 (1H, bs), 9.70 (1H, bs) | 514.0 |
| 55 | 2-(4-chloro-phenyl)- 3H-imidazo[4,5- b]pyridine-7-carboxylic acid[2-(4-benzyloxy-2- oxo-2H-pyridin-1-yl)- ethyl]-amide | 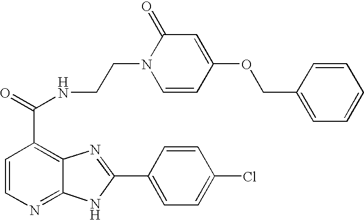 | 3.97 (2H, t, J = 6.0 Hz), 4.23 (2H, t, J = 6.0 Hz), 5.02 (2H, s), 5.97 (1H, s), 6.07 (1H, s), 7.31~7.39 (6H, m), 7.52 (2H, J = 8.4 Hz), 7.87 (2H, d, J = 6.0 Hz), 8.12 (2H, d, J = 8.4 Hz), 8.44 (1H, bs), 9.82 (1H, bs) | 500.0 |
| 56 | 2-(4-chloro-phenyl)- 3H-imidazo[4,5- b]pyridine-7-carboxylic acid[2-(4-hydroxy-2- oxo-2H-pyridin-1-yl)- ethyl]-amide | 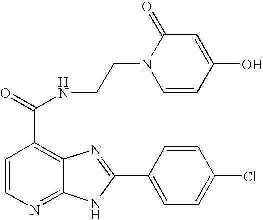 | 3.89 (2H, t, J = 5.7 Hz), 4.14 (2H, t, J = 5.7 Hz), 5.83 (1H, bs), 7.13 (1H, d, J = 7.5 Hz), 7.50~7.60 (2H, m), 7.74~7.79 (2H, m), 8.19~8.22 (2H, m), 8.39 (1H, d, J = 6.0 Hz) | 409.8 |
| 57 | 2-(4-chloro-phenyl)- 3H-imidazo[4,5- b]pyridine-7-carboxylic acid(2-pyridin-4-yl- ethyl)-amide | 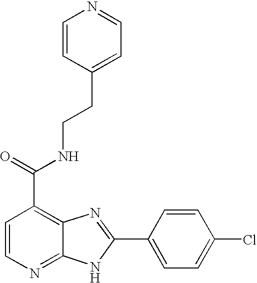 | 3.10 (t, 2H), 3.94 (t, 2H), 7.42-7.63 (m, 5H), 7.91 (d, 1H), 8.10 (d, 1H), 8.24 (m, 1H), 8.43 (d, 1H) | 377.8 |
| 58 | 2-(4-chloro-phenyl)- 3H-imidazo[4,5- b]pyridine-7-carboxylic acid(4-methyl-pyridin- 3-yl)-amide | 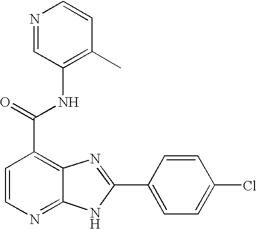 | 11.60 (s, 1H), 9.58 (s, 1H), 8.56 (m, 2H), 8.33 (m, 3H), 7.89 (m, 2H), 7.68 (m, 2H), 2.75 (s, 3H) | 363.8 |
| 59 | 2-(4-chloro-phenyl)- 3H-imidazo[4,5- b]pyridine-7-carboxylic acid(4-ethoxy-pyridin- 3-yl)-amide | 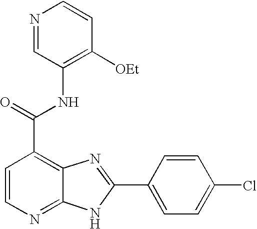 | 11.92 (s, 1H), 9.70 (s, 1H), 8.60 (m, 2H), 8.40 (d, 2H), 7.90 (d, 1H), 7.79 (d, 2H), 7.69 (d, 1H), 4.64 (q, 2H), 1.45 (t, 3H) | 393.8 |
| 60 | 2-(4-chloro-phenyl)- 3H-imidazo[4,5- b]pyridine-7-carboxylic acid(6-methyl-pyridin- 3-yl)-amide | 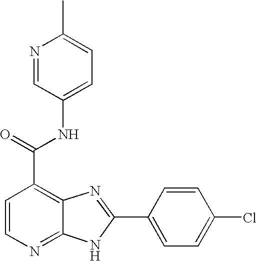 | 11.75 (s, 1H), 9.24 (s, 1H), 8.60 (m, 2H), 8.46 (d, 2H), 7.82 (d, 2H), 7.71 (d, 2H), 7.63 (m, 1H), 2.70 (s, 3H) | 363.8 |
| 61 | 2-(4-chloro-phenyl)- 3H-imidazo[4,5- b]pyridine-7-carboxylic acid(4-ethylsulfanyl- pyridin-3-yl)-amide | 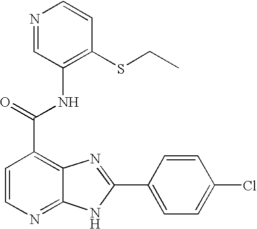 | 11.58 (S, 1H), 9.26 (s, 1H), 8.55 (d, 1H), 8.49 (d, 2H), 8.33 (m, 1H), 7.85 (d, 1H), 7.69 (d, 2H), 7.51 (d, 1H), 3.26 (q, 2H), 1.37 (t, 3H) | 409.9 |
| 62 | 2-furan-2-yl-3H- imidazo[4,5-b]pyridine- 7-carboxylic acid[3- (4,5-dichloro-3H- imidazo-1-yl)-propyl]- amide | 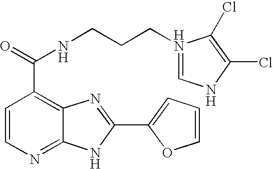 | 2.25 (q, 2H), 3.61 (t, 2H), 4.24 (t, 2H), 6.75 (d, 1H), 7.45 (d, 1H), 7.76 (m, 1H), 7.84 (m, 2H), 8.45 (d, 1H) | 407.3 |
| 63 | 2-furan-2-yl-3H- imidazo[4,5-b]pyridine- 7-carboxylic acid[2-(4- methanesulfonylamino- phenyl)-ethyl]-amide | 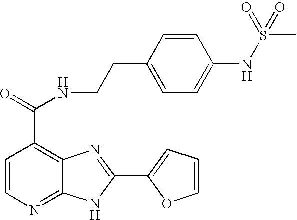 | 2.88 (s, 3H), 3.00 (t, 2H), 3.80 (t, 2H), 6.79 (s, 1H), 7.20 (d, 2H), 7.33 (d, 2H), 7.77 (d, 2H), 7.92 (s, 1H), 8.50 (d, 1H) | 425.5 |
| 64 | 2-furan-2-yl-3H- imidazo[4,5-b]pyridine- 7-carboxylic acid[2-(3- hydroxy-phenyl)-ethyl]- amide | 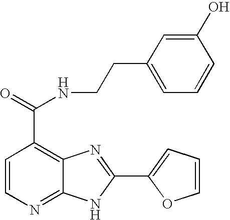 | 2.94 (t, 2H), 3.79 (t, 2H), 6.66 (m, 1H), 6.76 (m, 3H), 7.11 (t, 1H), 7.43 (m, 1H), 7.76 (d, 1H), 7.90 (d, 1H), 8.47 (d, 1H) | 348.4 |
| 65 | 2-furan-2-yl-3H- imidazo[4,5-b]pyridine- 7-carboxylic acid[2-(4- ethanesulfonylamino- phenyl)-ethyl]-amide | 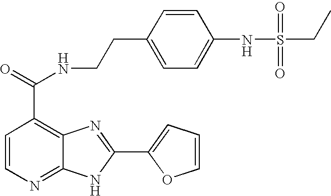 | 1.24 (t, 3H), 2.96 (q, 2H), 3.03 (t, 2H), 3.80 (t, 2H), 6.63 (m, 1H), 7.16 (m, 3H), 7.30 (d, 2H), 7.63 (d, 1H), 7.68 (d, 1H), 8.25 (d, 1H) | 439.5 |
| 66 | 2-furan-2-yl-3H- imidazo[4,5-b]pyridine- 7-carboxylic acid{2-[4- (2-dimethylamino- acetylamino)-phenyl]- ethyl}-amide | 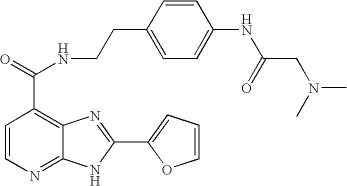 | 3.02 (6H), 3.19 (t, 2H), 3.79 (t, 2H), 4.09 (s, 2H), 6.74 (m, 1H), 7.32 (m, 3H), 7.52 (d, 2H), 7.73 (d, 1H), 7.87 (d, 1H), 8.41 (d, 1H) | 432.5 |
| 67 | 2-furan-2-yl-3H- imidazo[4,5-b]pyridine- 7-carboxylic acid[3-(4- methyl-imidazo-1-yl)- propyl]-amide | 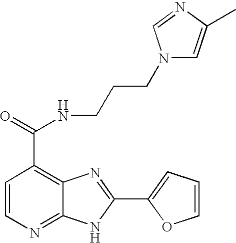 | 2.24 (d, 3H), 2.31 (m, 2H), 3.63 (t, 2H), 4.35 (t, 2H), 6.77 (m, 1H), 7.27 (d, 1H), 7.48 (s, 1H), 7.77 (d, 1H), 7.89 (s, 1H), 8.46 (d, 1H), 8.88 (d, 1H) | 350.4 |
| 68 | 2-furan-2-yl-3H- imidazo[4,5-b]pyridine- 7-carboxylic acid[2-(2- hydroxy-phenyl)-ethyl]- amide | 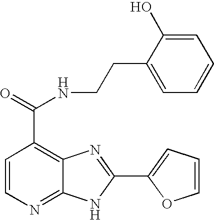 | .13 (t, 2H), 3.80 (t, 2H), 6.60 (m, 1H), 6.76 (m, 2H), 7.01 (m, 1H), 7.18 (m, 2H), 7.60 (d, 1H), 7.71 (m, 2H), 8.24 (d, 1H) | 348.4 |
| 69 | 2-furan-2-yl-3H- imidazo[4,5-b]pyridine- 7-carboxylic acid[2-(4- dimethylaminosulfonyl- amino-phenyl)-ethyl]-amide | 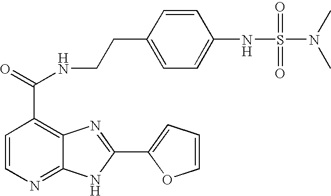 | 2.69 (s, 6H), 2.99 (t, 2H), 3.79 (t, 2H), 6.66 (m, 1H), 7.14 (d, 2H), 7.21 (d, 1H), 7.26 (d, 2H), 7.69 (d, 1H), 7.73 (s, 1H), 8.30 (d, 1H) | 454.5 |
| 70 | 2-furan-2-yl-3H- imidazo[4,5-b]pyridine- 7-carboxylic acid(2- pyridin-2-yl-ethyl)- amide | 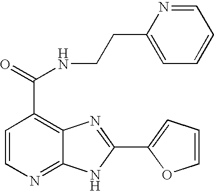 | 3.41 (t, 2H), 4.05 (t, 2H), 6.80 (m, 1H), 7.40 (d, 1H), 7.71 (d, 1H), 7.89 (m, 2H), 8.07 (d, 1H), 8.49 (m, 2H), 8.75 (d, 1H) | 333.4 |
| 71 | 2-furan-2-yl-3H- imidazo[4,5-b]pyridine- 7-carboxylic acid(2- pyridin-3-yl-ethyl)- amide | 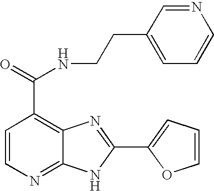 | 3.38 (t, 2H), 4.00 (t, 2H), 6.80 (m, 1H), 7.40 (d, 1H), 7.71 (d, 1H), 7.89 (m, 2H), 8.02 (m, 1H), 8.75 (m, 2H), 8.89 (s, 1H) | 333.4 |
| 72 | 2-furan-2-yl-3H- imidazo[4,5-b]pyridine- 7-carboxylic acid(2- pyridin-4-yl-ethyl)- amide | 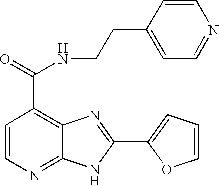 | 3.36 (t, 2H), 4.05 (t, 2H), 6.77 (d, 1H), 7.41 (d, 1H), 7.72 (d, 1H), 7.90 (d, 1H), 8.09 (d, 2H), 8.43 (d, 1H), 8.73 (d, 2H) | 333.4 |
| 73 | 2-furan-2-yl-3H- imidazo[4,5-b]pyridine- 7-carboxylic acid[2-(4- fluoro-3-hydroxy- phenyl)-ethyl]-amide | 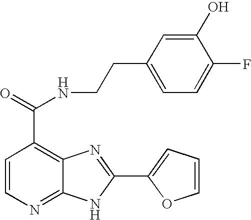 | 2.93 (t, 2H), 3.81 (t, 2H), 6.76 (m, 2H), 6.91 (m, 1H), 6.97 (m, 1H), 7.32 (d, 1H), 7.78 (d, 1H), 7.88 (8, 1H), 8.45 (d, 1H) | 366.4 |
| 74 | 2-furan-2-yl-3H- imidazo[4,5-b]pyridine- 7-carboxylic acid(3- imidazo-1-yl-propyl)- amide | 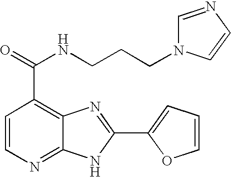 | 2.36 (q, 2H), 3.66 (t, 2H), 4.46 (t, 2H), 6.78 (s, 1H), 7.47 (d, 1H), 7.58 (s, 1H), 7.79 (m, 2H), 7.90 (d, 1H), 8.47 (d, 1H), 9.09 (s, 1H) | 336.4 |
| 75 | 2-furan-2-yl-3H- imidazo[4,5-b]pyridine- 7-carboxylic acid[2-(1- oxy-pyridin-2-yl)-ethyl]- amide | 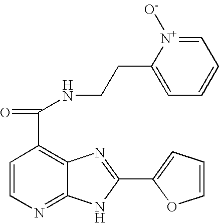 | 3.46 (t, 2H), 4.01 (t, 2H), 6.84 (m, 1H), 7.50 (m, 1H), 7.64 (m, 2H), 7.71 (m, 2H), 7.99 (s, 1H), 8.48 (d, 1H), 8.52 (d, 1H) | 349.4 |
| 76 | 2-furan-2-yl-3H- imidazo[4,5-b]pyridine- 7-carboxylic acid[2-(1- oxy-pyridin-3-yl)-ethyl]- amide | 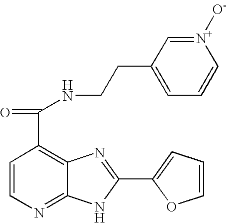 | 3.1 (t, 2H), 3.89 (t, 2H), 6.78 (m, 1H), 7.45 (d, 1H), 7.55 (t, 1H), 7.75 (d, 2H), 7.91 (s, 1H), 8.28 (d, 1H), 8.47 (m, 2H) | 349.4 |
| 77 | 2-furan-2-yl-3H- imidazo[4,5-b]pyridine- 7-carboxylic acid[2-(1- oxy-pyridin-4-yl)-ethyl]- amide | 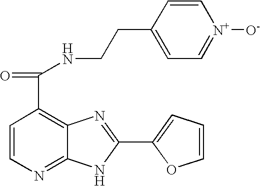 | 3.16 (t, 2H), 3.92 (t, 2H), 6.78 (m, 1H), 7.40 (d, 1H), 7.64 (d, 2H), 7.76 (d, 1H), 7.82 (m, 2H), 7.92 (s, 1H), 8.33 (d, 2H), 8.46 (d, 1H) | 349.4 |
| 78 | 2-furan-2-yl-3H- imidazo[4,5-b]pyridine- 7-carboxylic acid[2-(2- chloro-pyridin-4-yl)- ethyl]-amide | 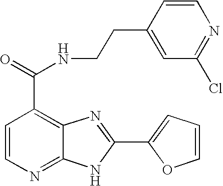 | 3.08 (t, 2H), 3.87 (t, 2H), 6.76 (s, 1H), 7.37 (m, 2H), 7.48 (s, 1H), 7.75 (d, 1H), 7.87 (s, 1H), 8.25 (d, 1H), 8.45 (br, 1H) | 367.8 |
| 79 | 2-furan-2-yl-3H- imidazo[4,5-b]pyridine- 7-carboxylic acid[2-(6- chloro-pyridin-3-yl)- ethyl]-amide | 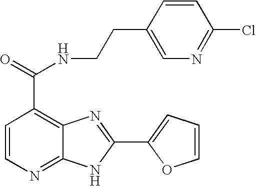 | 3.37 (t, 2H), 3.96 (t, 2H), 7.69 (m, 2H), 7.83 (d, 1H), 7.93 (d, 1H), 8.41 (s, 1H), 8.47 (d, 1H), 8.51 (br, 1H), 8.80 (br, 1H) | 367.8 |
| 80 | 2-furan-2-yl-3H- imidazo[4,5-b]pyridine- 7-carboxylic acid[2-(2- aminomethyl-pyridin-3- yl)-ethyl]-amide | 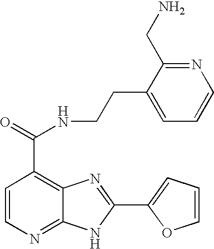 | 3.31 (t, 2H), 3.94 (t, 2H), 4.17 (s, 1H), 5.71 (s, 1H), 6.78 (s, 1H), 7.45 (s, 1H), 7.70 (m, 1H), 7.86 (m, 2H), 8.43~8.53 (m, 2H), 8.81 (m, 1H) | 362.4 |
| 81 | 2-furan-2-yl-3H- imidazo[4,5-b]pyridine- 7-carboxylic acid(3- pyridin-3-yl-propyl)- amide | 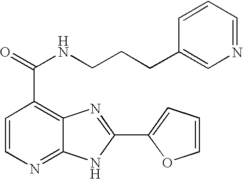 | 2.18 (t, 2H), 3.08 (t, 2H), 3.65 (t, 2H), 6.76 (s, 1H), 7.43 (s, 1H), 7.77 (d, 1H), 7.87 (s, 1H), 7.99 (m, 1H), 8.46 (s, 1H), 8.63 (m, 2H), 8.83 (s, 1H) | 347.4 |
| 82 | 2-furan-2-yl-3H- imidazo[4,5-b]pyridine- 7-carboxylic acid[2-(4- trifluoromethane sulfonylamino-phenyl)- ethyl]-amide | 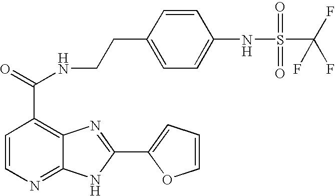 | 3.03 (t, 2H), 3.82 (t, 2H), 6.73 (s, 1H), 7.21 (d, 2H), 7.29 (d, 1H), 7.38 (d, 2H), 7.75 (d, 1H), 7.85 (d, 1H), 8.42 (d, 1H) | 479.4 |
| 83 | 2-furan-2-yl-3H- imidazo[4,5-b]pyridine- 7-carboxylic acid[3-(6- chloro-pyridin-3-yl)- propyl]-amide | 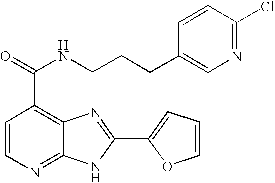 | 2.08 (t, 2H), 2.86 (t, 2H), 3.62 (t, 2H), 7.16 (s, 1H), 7.31 (d, 1H), 7.73 (s, 2H), 7.85 (d, 1H), 8.21 (d, 1H), 8.27 (s, 1H), 8.44 (s, 1H) | 381.8 |
| 84 | 2-furan-2-yl-3H- imidazo[4,5-b]pyridine- 7-carboxylic acid(3- pyridin-4-yl-propyl)- amide | 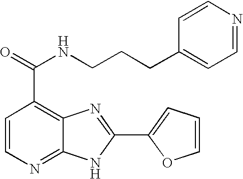 | 2.22 (m, 2H), 3.18 (t, 2H), 3.66 (t, 2H), 6.76 (s, 1H), 7.44 (d, 1H), 7.77 (d, 1H), 7.88 (s, 1H), 8.05 (d, 2H), 8.46 (d, 1H), 8.68 (d, 2H) | 347.4 |
| 85 | 2-furan-2-yl-3H- imidazo[4,5-b]pyridine- 7-carboxylic acid[3-(2- fluoro-pyridin-3-yl)- propyl]-amide | 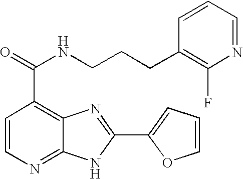 | 2.12 (t, 2H), 2.90 (t, 2H), 3.63 (t, 2H), 7.01 (s, 1H), 7.23 (d, 1H), 7.68 (s, 1H), 7.77 (m, 1H), 7.83 (m, 1H), 8.02 (d, 1H), 8.43 (m, 2H) | 365.4 |
| 86 | 2-thiophen-2-yl-3H- imidazo[4,5-b]pyridine- 7-carboxylic acid[2-(4- ethanesulfonylamino- phenyl)-ethyl]-amide | 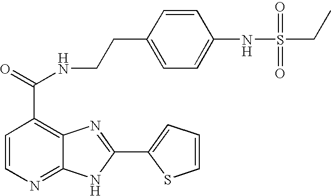 | 1.21 (t, 3H), 2.98 (m, 4H), 3.83 (t, 2H), 7.18 (d, 2H), 7.27 (m, 1H), 7.33 (d, 2H), 7.76 (m, 2H), 7.91 (m, 1H), 8.41 (d, 1H) | 455.6 |
| 87 | 2-thiophen-2-yl-3H- imidazo[4,5-b]pyridine- 7-carboxylic acid[3-(5- methyl-3H-imidazo-1- yl)-propyl]-amide | 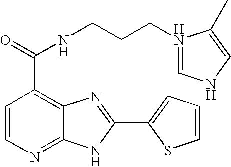 | 2.22 (d, 3H), 2.34 (m, 2H), 3.68 (t, 2H), 4.35 (t, 2H), 7.29 (m, 1H), 7.74 (m, 2H), 7.89 (s. 1H), 7.91 (d, 2H), 8.41 (d, 1H), 8.88 (d, 1H) | 368.5 |
| 88 | 2-thiophen-2-yl-3H- imidazo[4,5-b]pyridine- 7-carboxylic acid(2- pyridin-2-yl-ethyl)- amide | 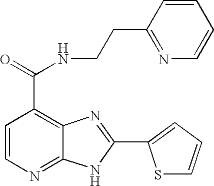 | 3.50 (t, 2H), 4.09 (t, 2H), 7.30 (t, 1H), 7.65 (d, 1H), 7.80 (d, 1H), 7.98 (m, 2H), 8.18 (d, 1H), 8.45 (d, 1H), 8.45 (t, 1H), 8.75 (d, 1H) | 349.4 |
| 89 | 2-thiophen-2-yl-3H- imidazo[4,5-b]pyridine- 7-carboxylic acid(2- pyridin-3-yl-ethyl)- amide | 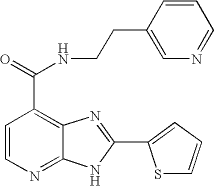 | 3.31 (t, 2H), 3.95 (t, 2H), 7.28 (t, 1H), 7.73 (d, 1H), 7.85 (d, 1H), 7.95 (d, 1H), 8.05 (m, 1H), 8.41 (d, 1H), 8.73 (m, 1H), 8.89 (s, 1H) | 349.4 |
| 90 | 2-thiophen-2-yl-3H- imidazo[4,5-b]pyridine- 7-carboxylic acid(2- pyridin-4-yl-ethyl)- amide | 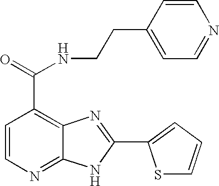 | 3.41 (t, 2H), 4.05 (t, 2H), 7.31 (m, 1H), 7.75 (d, 1H), 7.80 (d, 1H), 7.99 (d, 1H), 8.18 (d, 2H), 8.45 (d, 1H), 8.80 (d, 2H) | 349.4 |
| 91 | 2-thiophen-2-yl-3H- imidazo[4,5-b]pyridine- 7-carboxylic acid[2-(2- hydroxy-phenyl)-ethyl]- amide | 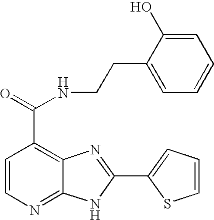 | 3.15 (t, 2H), 3.90 (t, 3H), 6.80 (m, 2H), 7.10 (d, 1H), 7.29 (m, 2H), 7.80 (m, 2H), 7.91 (m, 1H), 8.41 (d, 1H) | 364.4 |
| 92 | 2-thiophen-2-yl-3H- imidazo[4,5-b]pyridine- 7-carboxylic acid[2-(3- hydroxy-phenyl)-ethyl]- amide | 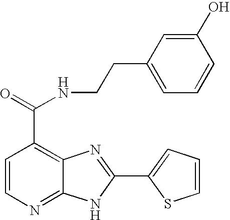 | 2.95 (t, 2H), 3.95 (t, 3H), 6.62 (m, 1H), 6.81 (m, 2H), 7.15 (d, 1H), 7.29 (m, 1H), 7.79 (m, 2H), 7.99 (m, 1H), 8.45 (d, 1H) | 364.4 |
| 93 | 2-thiophen-2-yl-3H- imidazo[4,5-b]pyridine- 7-carboxylic acid{2-[4- (2-dimethylamino- acetylamino)-phenyl]- ethyl}-amide | 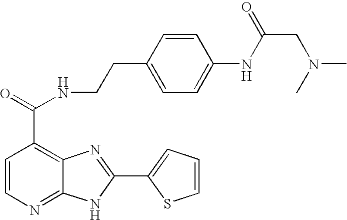 | 3.00 (s, 3H), 3.05 (t, 2H), 4.09 (s, 2H), 7.28 (m, 1H), 7.39 (d, 2H), 7.58 (d, 2H), 7.95 (d, 1H), 8.42 (d, 1H) | 448.6 |
| 94 | 2-thiophen-2-yl-3H- imidazo[4,5-b]pyridine- 7-carboxylic acid[2-(4- dimethylaminosulfonyl- amino-phenyl)-ethyl]-amide | 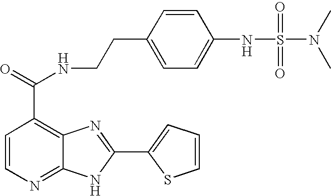 | 2.68 (s, 6H), 3.31 (t, 2H), 3.85 (t, 2H), 7.19 (m, 2H), 7.33 (m, 3H), 7.79 (m, 2H), 7.91 (m, 1H), 8.43 (m, 1H) | 470.6 |
| 95 | 2-thiophen-2-yl-3H- imidazo[4,5-b]pyridine- 7-carboxylic acid[2-(4- methanesulfonylamino- phenyl)-ethyl]- amide | 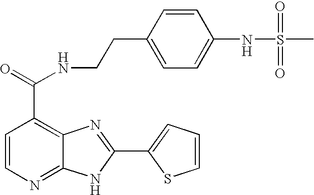 | 2.86 (s, 3H), 3.01 (t, 2H), 3.86 (t, 2H), 7.21~7.40 (m, 5H), 7.77 (d, 2m), 7.91 (d, 1H), 8.42 (d, 1H) | 441.5 |
| 96 | 2-thiophen-2-yl-3H- imidazo[4,5-b]pyridine- 7-carboxylic acid[2-(4- fluoro-3-hydroxy- phenyl)-ethyl]- amide | 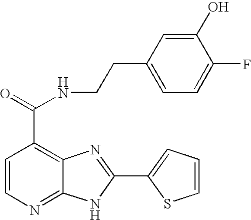 | 2.94 (t, 2H), 3.83 (t, 2H), 6.79 (s. 1H), 6.90~6.98 (m, 2H), 7.27 (t, 1H), 7.77 (t, 2H), 7.92 (d, 1H), 8.43 (m, 1H) | 382.4 |
| 97 | 2-thiophen-2-yl-3H- imidazo[4,5-b]pyridine- 7-carboxylic acid(3- imidazo-1-yl-propyl)- amide | 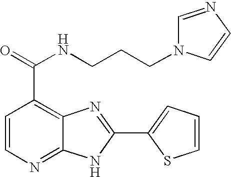 | 2.37 (t, 2H), 3.67 (t, 2H), 4.48 (t, 2H), 7.30 (t, 1H), 7.58 (s, 1H), 7.78 (m, 3H), 8.00 (d, 1H), 8.45 (d, 1H), 9.07 (s, 1H) | 352.4 |
| 98 | 2-thiophen-2-yl-3H- imidazo[4,5-b]pyridine- 7-carboxylic acid[2-(1- oxy-pyridin-2-yl)-ethyl]- amide | 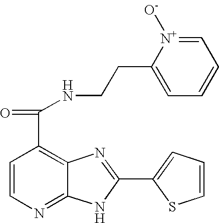 | 3.14 (t, 2H), 3.90 (t, 2H), 7.56 (t, 1H), 7.72 (m, 1H), 7.76 (m, 2H), 7.83 (d, 1H), 8.30 (d, 1H), 8.49 (m, 2H), 8.51 (d, 1H) | 365.4 |
| 99 | 2-thiophen-2-yl-3H- imidazo[4,5-b]pyridine- 7-carboxylic acid[2-(1- oxy-pyridin-3-yl)-ethyl]- amide | 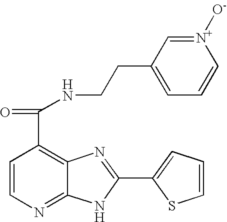 | 3.15 (t, 2H), 3.92 (t, 2H), 7.31 (t, 2H), 7.59 (t, 1H), 7.76 (d, 1H), 7.82 (m, 2H), 8.04 (d, 1H), 8.33 (d, 1H), 8.47 (d, 1H), 8.51 (s, 1H) | 365.4 |
| 100 | 2-thiophen-2-yl-3H- imidazo[4,5-b]pyridine- 7-carboxylic acid[2-(1- oxy-pyridin-4-yl)-ethyl]- amide | 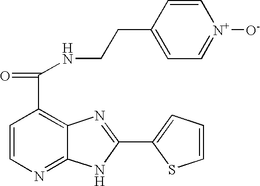 | 3.16 (t, 2H), 3.98 (t, 2H), 7.41 (t, 2H), 7.63 (t, 1H), 7.79 (d, 1H), 8.01 (m, 2H), 8.04 (d, 1H), 8.22 (m, 1H), 8.47 (m, 2H) | 365.4 |
| 101 | 2-thiophen-2-yl-3H- imidazo[4,5-b]pyridine- 7-carboxylic acid[2-(2- chloro-pyridin-4-yl)- ethyl]-amide | 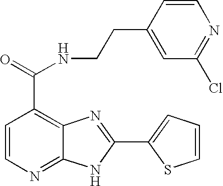 | 3.09 (t, 2H), 7.28 (m, 1H), 7.38 (d, 1H), 7.49 (s, 1H), 7.76 (d, 1H), 7.80 (d, 1H), 8.00 (d, 1H), 8.26 (d, 1H), 8.43 (m, 1H) | 383.9 |
| 102 | 2-thiophen-2-yl-3H- imidazo[4,5-b]pyridine- 7-carboxylic acid[2-(6- chloro-pyridin-3-yl)- ethyl]-amide | 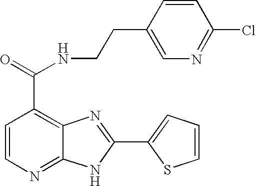 | 3.31 (t, 2H), 3.98 (t, 2H), 7.33 (m, 2H), 7.73 (m, 1H), 7.92 (s, 1H), 8.21 (m, 2H), 8.50 (m, 2H), 8.75 (br, 1H) | 383.9 |
| 103 | 2-thiophen-2-yl-3H- imidazo[4,5-b]pyridine- 7-carboxylic acid[2-(2- aminomethyl-pyridin-3- yl)-ethyl]-amide | 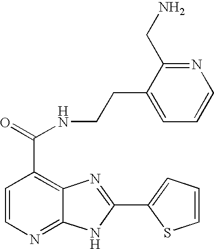 | 3.31 (t, 2H), 3.96 (t, 2H), 4.14 (s, 2H), 7.29 (m, 1H), 7.63 (d, 1H), 7.80 (d, 1H), 7.90 (m, 1H), 7.97 (d, 1H), 8.40 (m, 1H), 8.52 (d, 1H), 8.80 (d, 1H) | 378.5 |
| 104 | (3-{2-[(2-thiophen-2- yl-3H-imidazo[4,5- b]pyridine-7-carbonyl)- amino]-ethyl}-pyridin-2- ylmethyl)-carbamic acid tert-butylester | 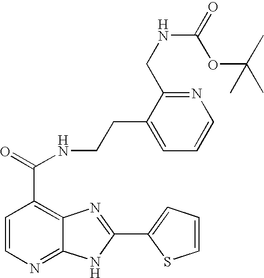 | 1.39 (s, 9H), 3.31 (t, 2H), 3.97 (t, 2H), 4.75 (s, 2H), 7.28 (m, 1H), 7.69 (m, 2H), 7.80 (m, 1H), 7.90 (s, 1H), 8.05 (s, 1H), 8.40 (m, 1H), 8.55 (d, 1H) | 478.6 |
| 105 | 2-thiophen-2-yl-3H- imidazo[4,5-b]pyridine- 7-carboxylic acid[2-(2- aminomethyl-pyridin-4- yl)-ethyl]-amide | 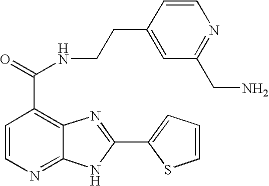 | 3.38 (t, 2H), 4.01 (t, 2H), 4.64 (s, 2H), 7.29 (m, 1H), 7.72 (d, 1H), 7.79 (d, 1H), 7.97 (m, 2H), 8.09 (s, 1H), 8.42 (d, 1H), 8.80 (d, 1H) | 378.5 |
| 106 | 2-thiophen-2-yl-3H- imidazo[4,5-b]pyridine- 7-carboxylic acid(3- pyridin-3-yl-propyl)- amide | 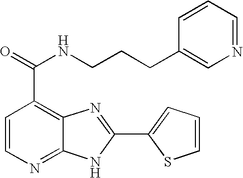 | 2.19 (q, 2H), 3.11 (t, 2H), 3.66 (t, 2H), 7.28 (t, 1H), 7.73 (m, 2H), 7.98 (m, 2H), 8.46 (s, 1H), 8.70 (m, 2H), 8.62 (s, 1H) | 363.4 |
| 107 | 2-thiophen-2-yl-3H- imidazo[4,5-b]pyridine- 7-carboxylic acid[2-(4- trifluoromethanesulfonyl- amino-phenyl)-ethyl]-amide | 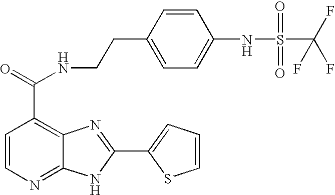 | 3.03 (t, 2H), 3.83 (t, 2H), 7.19~7.26 (m, 4H), 7.39 (d, 2H), 7.75 (d, 2H), 7.91 (d, 1H), 8.40 (m, 1H) | 495.5 |
| 108 | 2-thiophen-2-yl-3H- imidazo[4,5-b]pyridine- 7-carboxylic acid[3-(6- chloro-pyridin-3-yl)- propyl]-amide | 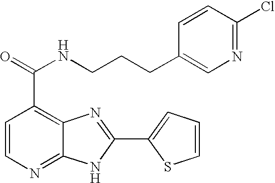 | 2.09 (t, 2H), 2.85 (t, 2H), 3.64 (t, 2H), 7.27 (d, 1H), 7.73 (s, 1H), 7.83 (m, 2H), 7.85 (m, 2H), 8.18~8.44 (m, 2H) | 397.9 |
| 109 | 2-thiophen-2-yl-3H- imidazo[4,5-b]pyridine- 7-carboxylic acid(3- pyridin-4-yl-propyl)- amide | 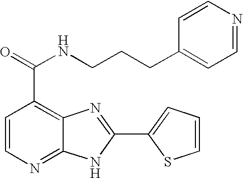 | 2.22 (m, 2H), 3.18 (t, 2H), 3.67 (t, 2H), 7.27 (m, 1H), 7.75 (m, 2H), 7.96 (m, 1H), 8.03 (m, 1H), 8.49 (br, 1H), 8.67 (br, 1H) | 363.4 |
| 110 | 2-thiophen-2-yl-3H- imidazo[4,5-b]pyridine- 7-carboxylic acid[3-(2- fluoro-pyridin-3-yl)- propyl]-amide | 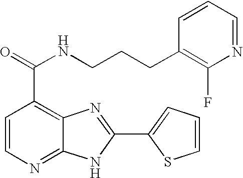 | 2.13 (t, 2H), 2.95 (t, 2H), 3.63 (t, 2H), 7.02 (s, 1H), 7.26 (m, 2H), 7.75 (m, 2H), 7.95 (s, 1H), 8.03 (d, 1H), 8.41 (m, 1H) | 381.4 |
| 111 | 2-thiophen-2-yl-3H- imidazo[4,5-b]pyridine- 7-carboxylic acid[3-(2- chloro-pyridin-4-yl)- propyl]-amide | 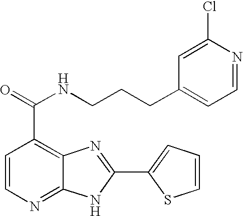 | 2.12 (t, 2H), 2.93 (t, 2H), 3.63 (t, 2H), 7.26 (m, 1H), 7.29 (d, 1H), 7.75 (m, 2H), 7.96 (s, 1H), 8.15 (d, 1H), 8.49 (br, 1H) | 397.9 |
| 112 | 2-thiophen-2-yl-3H- imidazo[4,5-b]pyridine- 7-carboxylic acid[2-(2- methyl-pyridin-3-yl)- ethyl]-amide | 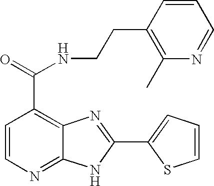 | 2.87 (s, 3H), 3.31 (t, 2H), 3.93 (t, 2H), 6.76 (m, 1H), 7.40 (d, 1H), 7.73 (d, 1H), 7.76~7.80 (m, 1H), 7.88 (s, 1H), 8.42 (d, 1H), 8.40~8.55 (m, 3H) | 363.4 |
| 113 | 2-thiophen-2-yl-3H- imidazo[4,5-b]pyridine- 7-carboxylic acid[3-(6- fluoro-pyridin-3-yl)- propyl]-amide | 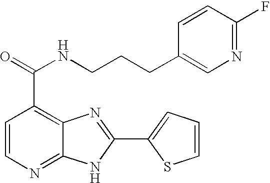 | 2.10 (m, 2H), 2.90 (t, 2H), 3.60 (t, 2H), 6.97 (d, 1H), 7.27 (d, 1H), 7.77 (m, 2H), 7.90 (t, 1H), 7.98 (s, 1H), 8.12 (s, 1H), 8.44 (s, 1H) | 381.4 |
| 114 | 2-furan-3-yl-3H- imidazo[4,5-b]pyridine- 7-carboxylic acid[2-(4- methanesulfonylamino- phenyl)-ethyl]-amide | 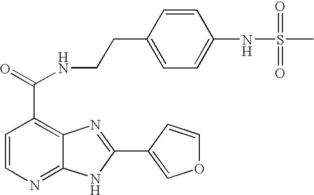 | 2.87 (s, 3H), 3.00 (t, 2H), 3.82 (t, 2H), 7.04 (s, 1H), 7.18 (d, 2H), 7.32 (d, 2H), 7.82 (m, 2H), 8.34 (s, 1H), 8.43 (br, 1H) | 425.5 |
| 115 | 2-furan-3-yl-3H- imidazo[4,5-b]pyridine- 7-carboxylic acid[2-(4- ethanesulfonylamino- phenyl)-ethyl]-amide | 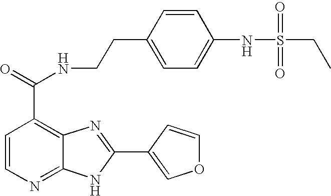 | 2.88 (s, 3H), 3.00 (t, 2H), 3.80 (t, 2H), 6.79 (s, 1H), 7.20 (d, 2H), 7.33 (d, 2H), 7.77 (d, 2H), 7.92 (s, 1H), 8.50 (d, 1H) | 439.5 |
| 116 | 2-furan-3-yl-3H- imidazo[4,5-b]pyridine- 7-carboxylic acid[2-(4- dimethylaminosulfonyl- amino-phenyl)-ethyl]-amide | 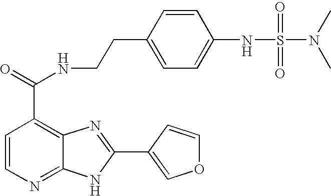 | 2.28 (s, 6H), 2.98 (t, 2H), 3.80 (t, 3H), 7.07 (s, 1H), 7.17 (d, 2H), 7.27 (d, 2H), 7.75 (m, 2H), 8.38 (s, 1H), 8.44 (d, 1H) | 454.5 |
| 117 | 2-furan-3-yl-3H- imidazo[4,5-b]pyridine- 7-carboxylic acid[3- (4,5- dichloro-3H- imidazo-1-yl)-propyl]- amide | 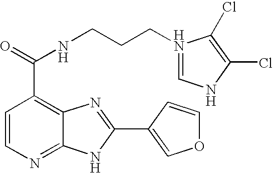 | 2.21 (m, 2H), 3.61 (t, 2H), 4.20 (t, 2H), 7.19 (s, 1H), 7.77 (m, 3H), 8.47 (m, 2H) | 407.3 |
| 118 | 2-furan-3-yl-3H- imidazo[4,5-b]pyridine- 7-carboxylic acid[3-(5- methyl-3H-imidazo-1- yl)-propyl]-amide | 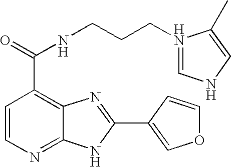 | 2.38~2.12 (m, 5H), 3.66 (t, 2H), 4.34 (t, 2H), 7.19 (s, 2H), 7.41~7.27 (d, 1H), 7.77 (s, 2H), 8.45 (m, 2H), 8.97~8.86 (d, 1H) | 352.4 |
| 119 | 2-furan-3-yl-3H- imidazo[4,5-b]pyridine- 7-carboxylic acid[2-(3- hydroxy-phenyl)-ethyl]- amide | 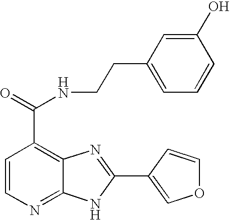 | 2.94 (t, 2H), 3.82 (t, 2H), 6.65 (d, 1H), 6.76 (m, 2H), 7.03 (s, 1H), 7.11 (m, 1H), 7.74 (m, 1H), 8.30 (s, 1H), 8.42 (d, 1H) | 348.4 |
| 120 | 2-furan-3-yl-3H- imidazo[4,5-b]pyridine- 7-carboxylic acid[2-(2- hydroxy-phenyl)-ethyl]- amide | 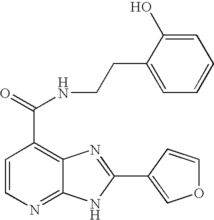 | 3.00 (t, 2H), 3.80 (t, 2H), 6.74 (m, 2H), 6.94 (m, 2H), 7.15 (m, 1H), 7.75 (s, 2H), 8.37 (s, 1H), 8.45 (d, 1H) | 348.4 |
| 121 | 2-furan-3-yl-3H- imidazo[4,5-b]pyridine- 7-carboxylic acid[2-(4- acetylamino-phenyl)- ethyl]-amide | 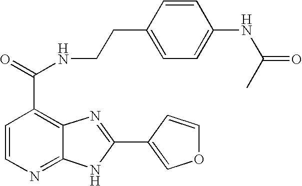 | 2.10 (s, 3H), 2.97 (t, 2H), 3.78 (t, 2H), 7.11 (s, 1H), 7.25 (d, 2H), 7.48 (d, 1H), 7.77 (d, 2H), 8.44 (s, 1H), 8.51 (d, 1H) | 389.4 |
| 122 | 2-furan-3-yl-3H- imidazo[4,5-b]pyridine- 7-carboxylic acid(2-{4- [2-(4-ethyl-piperazin-1- yl)-acetylamino]- phenyl}-ethyl)-amide | 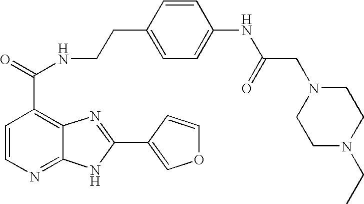 | 1.35 (t, 3H), 3.01~2.97 (m, 6H), 3.19 (q, 2H), 3.45~3.18 (m, 6H), 3.78 (t, 2H), 7.08 (s, 1H), 7.31 (d, 2H), 7.53 (d, 2H), 7.75 (s, 2H), 8.36 (s, 1H), 8.45 (d, 1H) | 501.6 |
| 123 | 2-furan-3-yl-3H- imidazo[4,5-b]pyridine- 7-carboxylic acid(2- pyridin-2-yl-ethyl)- amide | 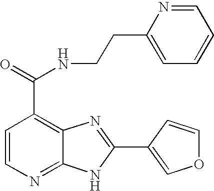 | 3.40 (t, 2H), 3.90 (t, 2H), 6.71 (d, 1H), 7.40 (d, 1H), 7.67 (d, 1H), 7.83~7.95 (m, 2H), 8.11 (d, 1H), 8.55 (m, 1H), 8.76 (d, 1H) | 333.4 |
| 124 | 2-furan-3-yl-3H- imidazo[4,5-b]pyridine- 7-carboxylic acid(2- pyridin-3-yl-ethyl)- amide | 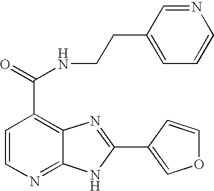 | 3.42 (t, 2H), 3.88 (t, 2H), 6.72 (d, 1H), 7.42 (d, 1H), 7.67 (d, 1H), 7.83 (d, 1H), 8.02 (d, 1H), 8.42 (d, 1H), 8.70 (m, 2H), 8.88 (s, 1H) | 333.4 |
| 125 | 2-furan-3-yl-3H- imidazo[4,5-b]pyridine- 7-carboxylic acid(2- pyridin-4-yl-ethyl)- amide | 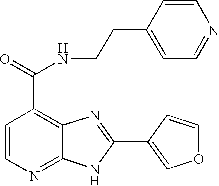 | 3.43 (t, 2H), 4.02 (t, 2H), 7.14 (s, 1H), 7.72 (d, 1H), 7.78 (s, 1H), 8.08 (d, 1H), 8.43 (m, 1H), 8.73 (d, 1H) | 333.4 |
| 126 | 2-furan-3-yl-3H- imidazo[4,5-b]pyridine- 7-carboxylic acid[2-(4- fluoro-3-hydroxy- phenyl)-ethyl]-amide | 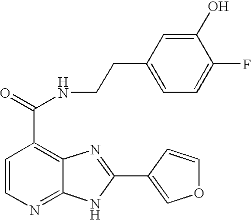 | 2.93 (t, 2H), 3.79 (t, 2H), 6.76 (m, 1H), 6.89 (m, 1H), 6.97 (m, 1H), 7.77 (m, 2H), 8.42 (s, 1H), 8.48 (d, 1H) | 366.4 |
| 127 | 2-furan-3-yl-3H- imidazo[4,5-b]pyridine- 7-carboxylic acid(3- imidazo-1-yl-propyl)- amide | 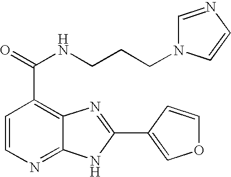 | 2.36 (q, 2H), 3.66 (t, 2H), 4.44 (t, 2H), 7.21 (s, 1H), 7.57 (s, 1H), 7.78 (m, 3H), 8.47 (m, 2H), 9.06 (s, 1H) | 336.4 |
| 128 | 2-furan-3-yl-3H- imidazo[4,5-b]pyridine- 7-carboxylic acid[2-(1- oxy-pyridin-2-yl)-ethyl]- amide | 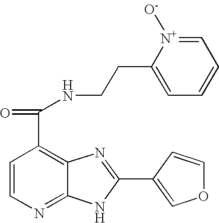 | 3.44 (t, 2H), 4.01 (t, 2H), 7.24 (d, 1H), 7.45 (m, 1H), 7.65 (m, 1H), 7.71 (d, 1H), 7.81 (s, 1H), 8.41 (d, 1H), 8.50 (d, 1H), 8.58 (s, 1H) | 349.4 |
| 129 | 2-furan-3-yl-3H- imidazo[4,5-b]pyridine- 7-carboxylic acid[2-(1- oxy-pyridin-3-yl)-ethyl]- amide | 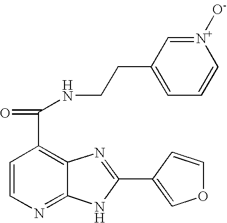 | 3.31 (t, 2H), 3.88 (t, 2H), 7.80 (s, 1H), 8.31 (d, 1H), 8.48~8.53 (m, 3H) | 349.4 |
| 130 | 2-furan-3-yl-3H- imidazo[4,5-b]pyridine- 7-carboxylic acid[2-(1- oxy-pyridin-4-yl)-ethyl]- amide | 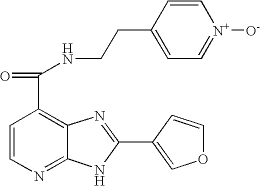 | 3.19 (t, 2H), 3.91 (t, 2H), 7.17 (d, 1H), 7.67 (d, 2H), 7.76 (d, 1H), 7.80 (m, 1H), 8.39 (d, 2H), 8.51 (m, 2H) | 349.4 |
| 131 | 2-furan-3-yl-3H- imidazo[4,5-b]pyridine- 7-carboxylic acid[2-(2- chloro-pyridin-3-yl)- ethyl]-amide | 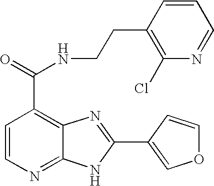 | 3.06 (t, 2H), 3.84 (t, 2H), 7.09 (s, 1H), 7.39 (d, 1H), 7.76~7.80 (m, 3H), 8.32 (s, 1H), 8.45 (m, 2H) | 367.8 |
| 132 | 2-furan-3-yl-3H- imidazo[4,5-b]pyridine- 7-carboxylic acid[2-(2- chloro-pyridin-4-yl)- ethyl]-amide | 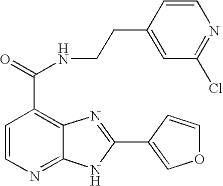 | 3.08 (t, 2H), 3.87 (t, 2H), 7.07 (s, 1H), 7.35 (d, 1H), 7.46 (s, 1H), 7.75 (d, 2H), 8.25 (d, 1H), 8.39 (s, 1H), 8.48 (m, 1H) | 367.8 |
| 133 | 2-furan-3-yl-3H- imidazo[4,5-b]pyridine- 7-carboxylic acid[2-(6- chloro-pyridin-3-yl)- ethyl]-amide | 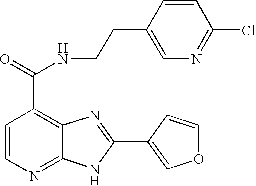 | 3.41 (t, 2H), 3.90 (t, 2H), 6, 71 (d, 1H), 7.42 (d, 1H), 7.67 (d, 1H), 7.83 (d, 1H), 8.10 (d, 1H), 8.50 (d, 1H), 8.70 (m, 1H), 8.90 (s, 1H) | 367.8 |
| 134 | 2-furan-3-yl-3H- imidazo[4,5-b]pyridine- 7-carboxylic acid[2-(2- aminomethyl-pyridin-3- yl)-ethyl]-amide | 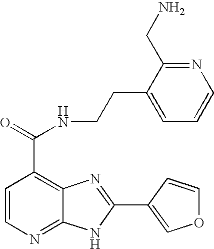 | 3.09 (t, 2H), 3.82 (t, 2H), 4.47 (s, 2H), 7.13 (s, 1H), 7.33 (m, 1H), 7.73~7.82 (m, 3H), 8.44~8.51 (m, 3H) | 362.4 |
| 135 | 2-furan-3-yl-3H- imidazo[4,5-b]pyridine- 7-carboxylic acid[2-(2- aminomethyl-pyridin-4- yl)-ethyl]-amide | 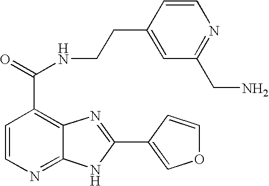 | 3.37 (t, 2H), 3.98 (t, 2H), 4.21 (s, 2H), 7.16 (s, 1H), 7.71 (d, 1H), 7.78 (s, 1H), 7.95 (d, 1H), 7.75 (d, 1H), 7.88 (d, 1H), 8.27 (m, 2H), 8.49 (br, 1H), 8.61 (s, 1H) | 362.4 |
| 136 | 2-furan-3-yl-3H- imidazo[4,5-b]pyridine- 7-carboxylic acid(3- pyridin-3-yl-propyl)- amide | 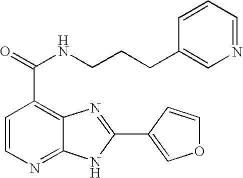 | 2.18 (q, 2H), 3.07 (t, 2H), 3.65 (t, 2H), 6.75 (m, 1H), 7.43 (d, 1H), 7.77 (d, 1H), 7.87 (s, 1H), 7.98 (m, 1H), 8.45 (d, 1H), 8.61 (d, 1H), 8.66 (m, 1H), 8.83 (br, 1H) | 347.4 |
| 137 | 2-furan-3-yl-3H- imidazo[4,5-b]pyridine- 7-carboxylic acid[2-(4- trifluoromethane sulfonylamino-phenyl)- ethyl]-amide | 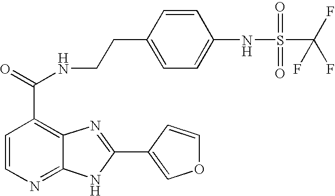 | 3.02 (t, 2H), 3.82 (t, 2H), 7.04 (s, 1H), 7.19 (d, 2H), 7.35 (d, 2H), 7.73 (m, 2H), 8.34 (s, 1H), 8.41 (br, 1H), | 479.4 |
| 138 | 2-furan-3-yl-3H- imidazo[4,5-b]pyridine- 7-carboxylic acid[3-(6- chloro-pyridin-3-yl)- propyl]-amide | 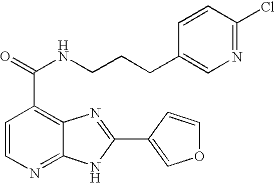 | 2.08 (t, 2H), 2.89 (t, 2H), 3.62 (t, 2H), 7.26 (m, 1H), 7.30 (d, 1H), 7.78 (m, 2H), 7.86 (d, 1H), 7.94 (m, 1H), 8.17 (m, 1H), 8.28 (s, 1H), 8.42 (s, 1H) | 381.8 |
| 139 | 2-furan-3-yl-3H- imidazo[4,5-b]pyridine- 7-carboxylic acid(3- pyridin-4-yl-propyl]- amide | 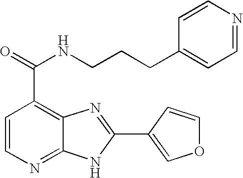 | 2.21 (t, 1H), 3.14 (t, 2H), 3.66 (t, 2H), 7.18 (s, 1H), 7.77 (s, 1H), 8.03 (d, 1H), 8.47 (s, 1H), 8.67 (br, 1H) | 347.4 |
| 140 | 6-bromo-2-furan-3-yl- 3H-imidazo[4,5- b]pyridine-7-carboxylic acid[3-(4,5-dichloro- imidazo-1-yl)-propyl]- amide | 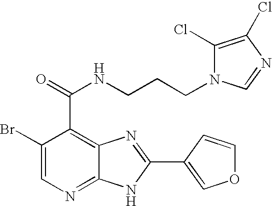 | DMSO: 1.88 (d, J = 2.1 Hz, 2H), 3.41-3.52 (m, 2H), 4.08 (d, J = 2.1 Hz, 2H), 7.21 (s, 1H), 7.61 (s, 1H), 7.78 (d, J = 2.6 Hz, 2H), 8.21 (d, J = 2.1 Hz, 1H), 8.48 (d, J = 2.6 Hz, 1H) | 484.1 |
| 141 | 2-furan-3-yl-3H- imidazo[4,5-b]pyridine- 7-carboxylic acid(4- benzoylamino-phenyl)- amide | 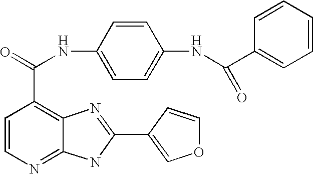 | DMSO: 6.58 (d, J = 2.6 Hz, 1H), 7.31-7.62 (m, 7H), 7.66 (s, 1H), 7.69 (d, J = 2.3 Hz, 2H), 7.81-7.92 (m, 3H), 8.23 (d, J = 2.1 Hz, 1H), 8.31 (t, J = 1.3 Hz, 2H), 8.58 (d, J = 2.1 Hz, 1H), 8.83 (s, 1H), 10.20 (s, 1H) | 423.4 |
| 142 | 2-(2,4-difluoro-phenyl)- 3H-imidazo[4,5- b]pyridine-7-carboxylic acid[2-(4- ethanesulfonylamino- phenyl)-ethyl]-amide | 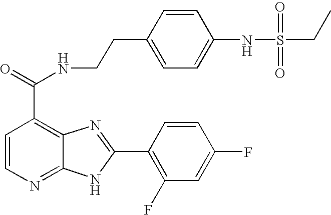 | 1.21 (t, 3H), 2.96 (q, 2H), 3.01 (t, 2H), 3.85 (t, 2H), 7.16 (d, 2H), 7.32 (m, 2H), 7.79 (m, 1H), 8.13 (m, 1H), 8.43 (d, 1H) | 485.5 |
| 143 | 2-(2,4-difluoro-phenyl)- 3H-imidazo[4,5- b]pyridine-7-carboxylic acid[2-(4- methanesulfonylamino- phenyl)-ethyl]-amide | 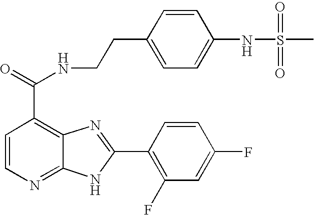 | 2.86 (s, 3H), 3.03 (t, 2H), 3.85 (t, 2H), 7.17 (m, 2H), 7.35 (m, 2H), 7.31 (m, 2H), 7.77 (m, 1H), 8.07 (m, 1H), 8.48 (d, 1H) | 471.5 |
| 144 | 2-(2,4-difluoro-phenyl)- 3H-imidazo[4,5- b]pyridine-7-carboxylic acid[3-(4,5-dichloro- imidazo-1-yl)-propyl]- amide | 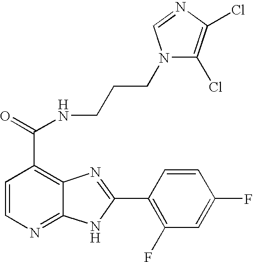 | 2.23 (m, 2H), 3.64 (t, 2H), 4.20 (t, 2H), 7.20 (m, 2H), ), 7.77 (s, 1H), 7.82 (m, 1H), 8.34 (m, 1H), 8.50 (d, 1H) | 451.3 |
| 145 | 2-cyclopropyl-3H- imidazo[4,5-b]pyridine- 7-carboxylic acid[2-(4- methanesulfonylamino- phenyl)-ethyl]-amide | 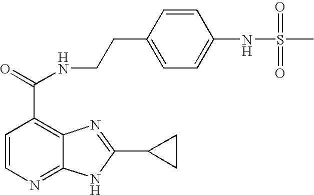 | 1.25 (m, 2H), 1.31 (m, 2H), 2.31 (m, 1H), 2.91 (t, 2H), 3.75 (t, 2H), 7.00 (d, 2H), 7.25 (d, 2H), 7.70 (d, 1H), 8.41 (d, 1H) | 399.5 |
| 146 | 2-cyclopropyl-3H- imidazo[4,5-b]pyridine- 7-carboxylic acid[2-(4- ethanesulfonylamino- phenyl)-ethyl]-amide | 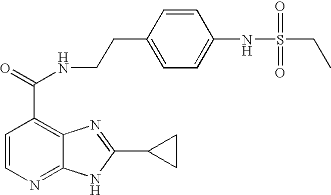 | 1.26 (m, 7H), 2.31 (m, 1H), 2.99 (m, 4H), 3.72 (t, 2H), 7.02 (m, 4H), 7.69 (d, 1H), 8.40 (d, 1H) | 413.5 |
| 147 | 2-cyclopropyl-3H- imidazo[4,5-b]pyridine- 7-carboxylic acid[2-(4- dimethylaminosulfonyl-amino-phenyl)-ethyl]-amide | 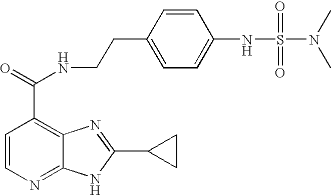 | 1.36 (m, 4H), 2.37 (m, 1H), 2.73 (s, 6H), 2.94 (t, 2H), 3.73 (t, 2H), 7.16 (d, 2H), 7.23 (d, 2H), 7.70 (d, 1H), 8.44 (d, 1H) | 428.5 |
| 148 | 2-cyclopropyl-3H- imidazo[4,5-b]pyridine- 7-carboxylic acid[3- (4,5-dichloro-3H- imidazo-1-yl)-propyl]- amide | 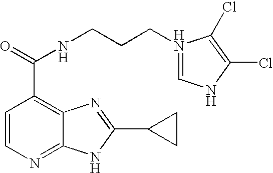 | 1.48 (m, 4H), 2.15 (m, 2H), 2.50 (s, 1H), 3.54 (t, 2H), 4.17 (t, 2H), 7.76 (d, 1H), 7.82 (s, 1H), 8.56 (d, 1H) | 381.3 |
| 149 | 2-cyclopropyl-3H- imidazo[4,5-b]pyridine- 7-carboxylic acid[3-(5- methyl-3H-imidazo-1- yl)-propyl]-amide | 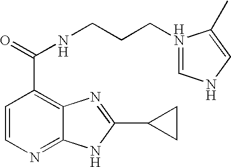 | 1.38 (m, 4H), 2.25 (m, 2H), 2.39 (s, 3H), 2.41 (m, 1H), 3.58 (t, 2H), 4.31 (t, 2H), 7.49~7.31 (d, 1H), 7.76 (d, 1H), 8.52 (d, 1H), 8.96~8.81 (d, 1H) | 326.4 |
| 150 | 2-cyclopropyl-3H- imidazo[4,5-b]pyridine- 7-carboxylic acid[2-(3- hydroxy-phenyl)-ethyl]- amide | 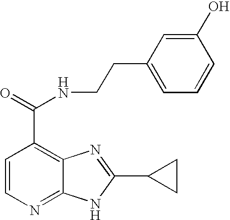 | 1.41 (m, 4H), 2.44 (m, 1H), 2.90 (t, 2H), 3.70 (t, 2H), 6.72 (m, 3H), 7.10 (t, 1H), 7.73 (d, 1H), 8.51 (d, 1H) | 322.4 |
| 151 | 2-thiophen-3-yl-3H- imidazo[4,5-b]pyridine- 7-carboxylic acid[2-(3- hydroxy-phenyl)-ethyl]- amide | 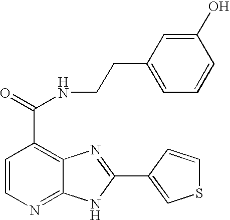 | 2.95 (t, 2H), 3.86 (t, 2H), 6.65 (m, 1H), 6.80 (m, 2H), 7.13 (t, 1H), 7.60 (m, 1H), 7.69 (d, 1H), 7.76 (d, 1H), 8.19 (s, 1H), 8.40 (d, 1H) | 364.4 |
| 152 | 2-thiophen-3-yl-3H- imidazo[4,5-b]pyridine- 7-carboxylic acid[2-(4- acetylamino-phenyl)- ethyl]-amide | 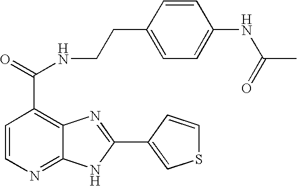 | 2.17 (s, 3H), 2.98 (t, 2H), 3.84 (t, 2H), 7.29 (d, 2H), 7.47 (d, 2H), 7.62 (s, 1H), 7.68 (s, 1H), 7.77 (d, 1H), 8.16 (s, 1H), 8.43 (d, 1H) | 405.5 |
| 153 | 2-thiophen-3-yl-3H- imidazo[4,5-b]pyridine- 7-carboxylic acid[2-(2- hydroxy-phenyl)-ethyl]- amide | 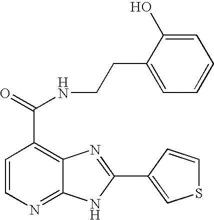 | 3.02 (t, 2H), 3.87 (t, 2H), 6.75 (m, 2H), 7.05 (t, 1H), 7.18 (d, 1H), 7.62 (m, 1H), 7.69 (d, 1H), 7.78 (d, 1H), 8.17 (s, 1H), 8.41 (d, 1H) | 364.4 |
| 154 | 2-thiophen-3-yl-3H- imidazo[4,5-b]pyridine- 7-carboxylic acid[2-(4- ethanesulfonylamino- phenyl)-ethyl]-amide | 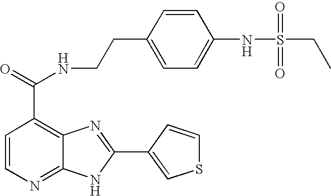 | 1.22 (t, 3H), 2.96 (t, 2H), 3.86 (t, 2H), 7.19 (d, 2H), 7.32 (d, 2H), 7.66 (m, 2H), 7.78 (s, 1H), 8.40 (d, 1H) | 455.6 |
| 155 | 2-(1-oxo-thiophen-3- yl)-3H-imidazo[4,5- b]pyridine-7-carboxylic acid[2-(4- ethanesulfonylamino- phenyl)-ethyl]-amide | 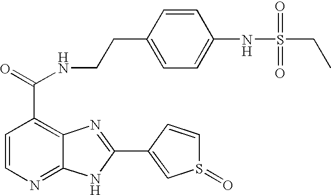 | 1.24 (t, 3H), 2.99 (m, 4H), 365 (t, 2H), 7.18 (d, 2H), 7.28 (d, 2H), 7.75 (m, 2H), 7.84 (m, 1H), 8.28 (d, 1H), 8.45 (br, 1H) | 471.6 |
| 156 | 2-thiophen-3-yl-3H- imidazo[4,5-b]pyridine- 7-carboxylic acid(2- pyridin-2-yl-ethyl)- amide | 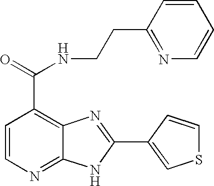 | 3.45 (t, 2H), 4.05 (t, 2H), 7.68 (d, 2H), 7.85~8.10 (m, 3H), 8.40-8.55 (m, 3H), 8.75 (d, 1H), 8.81 (d, 1H) | 349.4 |
| 157 | 2-thiophen-3-yl-3H- imidazo[4,5-b]pyridine- 7-carboxylic acid(2- pyridin-3-yl-ethyl)- amide | 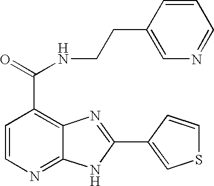 | 3.30 (t, 2H), 3.97 (t, 2H), 7.69 (m, 1H), 7.72 (d, 1H), 7.81 (d, 1H), 7.90 (m, 1H), 8.35 (m, 1H), 8.43 (d, 1H), 8.53 (d, 1H), 8.65 (d, 1H), 8.82 (s, 1H) | 349.4 |
| 158 | 2-thiophen-3-yl-3H- imidazo[4,5-b]pyridine- 7-carboxylic acid(2- pyridin-4-yl-ethyl)- amide | 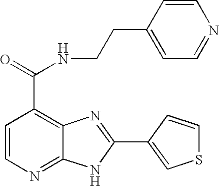 | 3.35 (t, 2H), 4.01 (t, 2H), 7.67 (m, 2H), 7.83 (d, 1H), 8.05 (m, 1H), 8.45 (m, 2H), 8.70 (m, 2H), 8.89 (s, 1H) | 349.4 |
| 159 | 2-thiophen-3-yl-3H- imidazo[4,5-b]pyridine- 7-carboxylic acid[2-(4- methanesulfonylamino- phenyl)-ethyl]-amide | 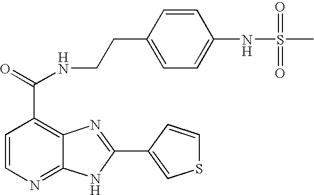 | 2.86 (s, 3H), 3.07 (t, 2H), 3.84 (t, 2H), 7.25 (d, 2H), 7.38 (d, 2H), 7.65~7.82 (m, 3H), 8.28 (m, 1H), 8.45 (d, 1H) | 441.5 |
| 160 | 2-thiophen-3-yl-3H- imidazo[4,5-b]pyridine- 7-carboxylic acid[2-(4- dimethylaminosulfonyl- amino-phenyl)-ethyl]-amide | 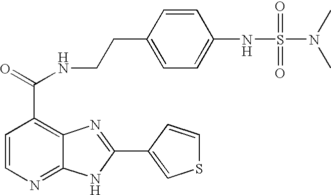 | 2.69 (s, 6H), 3.05 (t, 2H), 4.88 (t, 2H), 7.18 (d, 2H), 7.35 (d, 2H), 7.65~7.80 (m, 2H), 8.25 (m, 1H), 8.42 (d, 1H) | 470.6 |
| 161 | 2-thiophen-3-yl-3H- imidazo[4,5-b]pyridine- 7-carboxylic acid[2-(4- fluoro-3-hydroxy- phenyl)-ethyl]-amide | 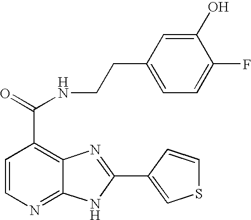 | 2.94 (q, 2H), 3.85 (t, 2H), 6.68 (m, 1H), 6.95 (m, 2H), 7.63~7.80 (m, 3H), 8.27 (m, 1H), 8.45 (d, 1H) | 382.4 |
| 162 | 2-thiophen-3-yl-3H- imidazo[4,5-b]pyridine- 7-carboxylic acid[3-(4- methyl-imidazo-1-yl)- propyl]-amide | 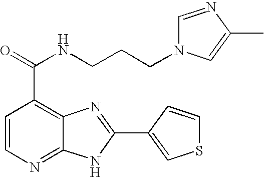 | 2.24 (s, 3H), 2.33 (m, 2H), 3.67 (t, 2H), 4.37 (t, 2H), 7.43 (s, 1H), 7.70 (m, 1H), 7.80 (m, 1H), 7.93 (d, 1H), 8.46 (m, 2H), 8.88 (s, 1H) | 366.4 |
| 163 | 2-thiophen-3-yl-3H- imidazo[4,5-b]pyridine- 7-carboxylic acid(3- imidazo-1-yl-propyl)- amide | 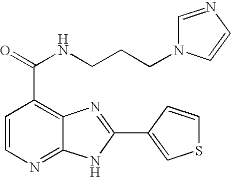 | 2.37 (t, 2H), 3.68 (t, 2H), 4.45 (t, 2H), 7.55 (s, 1H), 7.68 (m, 1H), 7.76 (s, 1H), 7.79 (d, 1H), 7.93 (d, 1H), 8.42 (m, 1H), 8.45 (d, 1H), 9.02 (s, 1H) | 352.4 |
| 164 | 2-thiophen-3-yl-3H- imidazo[4,5-b]pyridine- 7-carboxylic acid[2-(1- oxy-pyridin-2-yl)-ethyl]- amide | 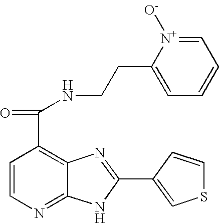 | 3.46 (t, 2H), 4.01 (t, 2H), 7.47 (m, 1H), 7.62 (t, 1H), 7.70-7.75 (m, 2H), 7.78 (m, 1H), 8.01 (m, 1H), 8.43 (d, 1H), 8.57 (d, 1H), 8.73 (m, 1H) | 365.4 |
| 165 | 2-thiophen-3-yl-3H- imidazo[4,5-b]pyridine- 7-carboxylic acid[2-(1- oxy-pyridin-3-yl)-ethyl]- amide | 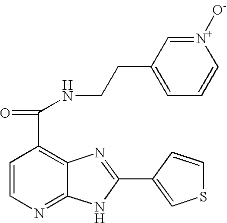 | 3.35 (t, 2H), 4.01 (t, 2H), 7.67 (m, 1H), 7.71 (d, 1H), 7.80 (d, 1H), 7.99 (m, 2H), 8.34 (s, 1H), 8.40 (d, 1H), 8.67 (m, 1H) | 365.4 |
| 166 | 2-thiophen-3-yl-3H- imidazo[4,5-b]pyridine- 7-carboxylic acid[2-(1- oxy-pyridin-4-yl)-ethyl]- amide | 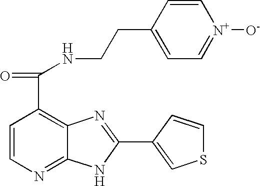 | 3.20 (t, 2H), 3.93 (t, 2H), 7.71 (d, 2H), 7.74 (m, 1H), 7.78 (d, 1H), 7.88 (d, 1H), 8.43 (d, 2H), 8.51 (m, 1H), 8.53 (d, 1H) | 365.4 |
| 167 | 2-thiophen-3-yl-3H- imidazo[4,5-b]pyridine- 7-carboxylic acid[2-(2- chloro-pyridin-4-yl)- ethyl]-amide | 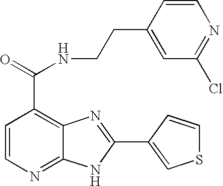 | 3.09 (t, 2H), 3.88 (t, 2H), 7.36 (d, 1H), 7.48 (s, 1H), 7.69 (s, 1H), 7.77 (m, 2H), 8.25 (d, 1H), 8.41 (s, 1H), 8.41 (s, 1H), 8.50 (br, 1H) | 383.9 |
| 168 | 2-thiophen-3-yl-3H- imidazo[4,5-b]pyridine- 7-carboxylic acid[2-(2- aminomethyl-pyridin-3- yl)-ethyl]-amide | 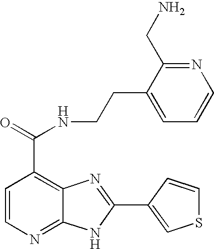 | 3.31 (t, 2H), 3.95 (t, 2H), 4.15 (s, 2H), 7.70 (m, 2H), 7.87 (m, 2H), 8.41 (m, 2H), 8.50 (d, 1H), 8.79 (d, 1H) | 378.5 |
| 169 | 2-thiophen-3-yl-3H- imidazo[4,5-b]pyridine- 7-carboxylic acid[2-(6- chloro-1-oxy-pyridin-3- yl)-ethyl]-amide | 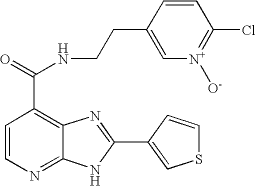 | 3.22 (t, 2H), 3.93 (t, 2H), 7.26 (m, 1H), 7.59 (d, 1H), 7.76 (m, 2H), 7.78 (s, 1H), 8.14 (d, 1H), 8.41 (s, 2H) | 399.9 |
| 170 | 2-thiophen-3-yl-3H- imidazo[4,5-b]pyridine- 7-carboxylic acid[2-(2- fluoro-pyridin-4-yl)- ethyl]-amide | 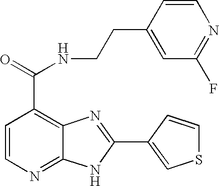 | 3.14 (t, 2H), 3.91 (t, 2H), 7.10 (s, 1H), 7.31 (d, 1H), 7.68 (s, 1H), 7.79 (s, 1H), 8.10 (d, 1H), 8.35 (s, 1H), 8.45 (br, 1H) | 367.4 |
| 171 | 2-thiophen-3-yl-3H- imidazo[4,5-b]pyridine- 7-carboxylic acid[2-(2- methyl amino-pyridin-4- yl)-ethyl]-amide | 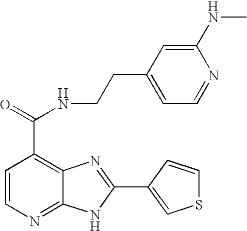 | 2.83 (s, 3H), 3.06 (t, 2H), 3.93 (t, 2H), 6.86 (s, 1H), 6.94 (d, 1H), 7.28 (m, 1H), 7.76 (m, 3H), 7.92 (d, 2H), 8.42 (d, 1H) | 378.5 |
| 172 | 2-thiophen-3-yl-3H- imidazo[4,5-b]pyridine- 7-carboxylic acid(3- pyridin-3-yl-propyl)- amide | 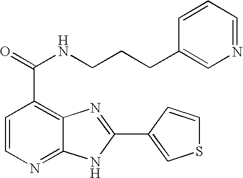 | 2.18 (q, 2H), 3.07 (t, 2H), 3.66 (t, 2H), 7.66 (s, 1H), 7.77 (d, 1H), 7.88 (d, 1H), 7.97 (t, 1H), 8.44 (m, 2H), 8.59 (d, 1H), 8.65 (br, 1H), 8.82 (br, 1H) | 363.4 |
| 173 | 2-thiophen-3-yl-3H- imidazo[4,5-b]pyridine- 7-carboxylic acid[2-(4- trifluoromethanesulfonyl- amino-phenyl)-ethyl]- amide | 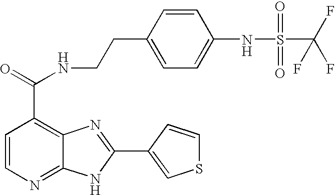 | 3.04 (t, 2H), 3.84 (t, 2H), 7.20 (d, 2H), 7.36 (d, 2H), 7.65 (m, 1H), 7.75 (d, 2H), 8.28 (s, 1H), 8.43 (d, 1H) | 495.5 |
| 174 | 2-thiophen-3-yl-3H- imidazo[4,5-b]pyridine- 7-carboxylic acid[3-(6- chloro-pyridin-3-yl)- propyl]-amide | 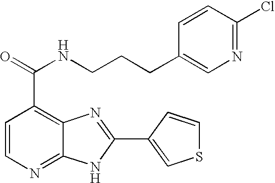 | 2.08 (t, 2H), 2.85 (t, 2H), 3.62 (t, 2H), 6.74 (s, 1H), 7.31 (d, 1H), 7.51 (s, 1H), 7.78 (m, 1H), 8.17 (m, 1H), 8.30 (s, 1H), 8.44 (s, 1H) | 397.9 |
| 175 | 2-thiophen-3-yl-3H- imidazo[4,5-b]pyridine- 7-carboxylic acid(3- pyridin-4-yl-propyl)- amide | 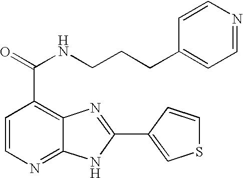 | 2.27 (q, 2H), 3.15 (t, 2H), 3.67 (t, 2H), 7.68 (q, 1H), 7.77 (d, 1H), 7.90 (d, 2H), 8.43 (m, 2H), 8.67 (d, 2H) | 363.4 |
| 176 | 2-thiophen-3-yl-3H- imidazo[4,5-b]pyridine- 7-carboxylic acid[3-(2- chloro-pyridin-4-yl)- propyl]-amide | 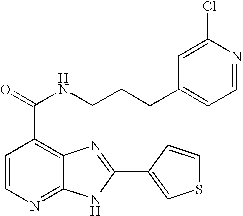 | 2.14 (t, 2H), 2.87 (t, 2H), 3.65 (t, 2H), 7.28~7.38 (m, 3H), 7.78 (d, 1H), 8.14 (d, 1H), 8.29 (m, 2H), 8.46 (br, 1H) | 397.9 |
| 177 | 2-thiophen-3-yl-3H- imidazo[4,5-b]pyridine- 7-carboxylic acid[2-(2- methyl-pyridin-3-yl)- ethyl]-amide | 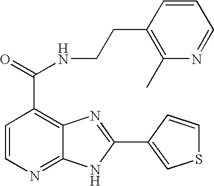 | 2.87 (s, 3H), 3.31 (t, 2H), 3.95 (t, 2H), 7.69 (m, 3H), 7.79 (m, 1H), 7.87 (d, 1H), 8.40~8.55 (m, 3H) | 363.4 |
| 178 | 2-thiophen-3-yl-3H- imidazo[4,5-b]pyridine- 7-carboxylic acid[3-(6- fluoro-pyridin-3-yl)- propyl]-amide | 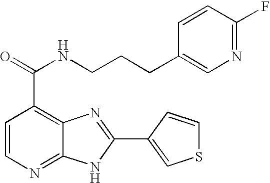 | 2.08 (m, 2H), 2.86 (t, 2H), 3.61 (t, 2H), , 6.96 (d, 1H), 7.66 (s, 1H), 7.76 (d, 1H), 7.87 (m, 2H), 8.09 (s, 1H), 8.35~8.50 (m, 2H) | 381.4 |
| 179 | 2-(1-oxo-thiophen-3- yl)-3H-imidazo[4,5- b]pyridine-7-carboxylic acid[3-(1-oxy-pyridin- 3-yl)-propyl]-amide | 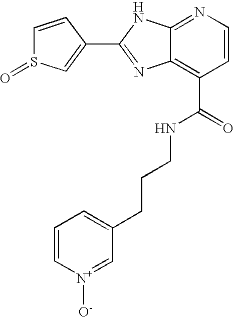 | 2.06 (t, 2H), 2.84 (t, 2H), 3.55 (t, 2H), 7.53 (t, 1H), 7.65~7.73 (m, 3H), 7.94 (s, 1H), 8.24 (d, 1H), 8.33 (d, 1H), 8.39 (s, 1H), 8.56 (s, 1H) | 395.4 |
| 180 | 2-(1-oxo-1H-thiophen- 3-yl)-3H-imidazo[4,5- b]pyridine-7-carboxylic acid(3-pyridin-3-yl- propyl)-amide | 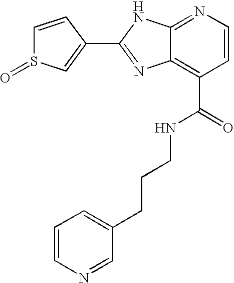 | 2.12 (m, 2H), 2.87 (m, 2H), 3.62 (m, 2H), 7.45~7.75 (m, 4H), 7.82~8.05 (m, 2H), 8.20 (m, 1H), 8.37 (m, 1H), 8.53 (m, 1H) | 379.4 |
| 181 | 2-thiophen-3-yl-3H- imidazo[4,5-b]pyridine- 7-carboxylic acid pyridin-3-yl-amide | 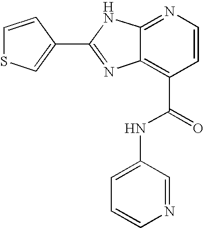 | 7.71 (m, 1H), 7.92 (d, 1H), 7.96 (m, 1H), 7.98 (m, 1H), 8.00~8.52 (m, 4H), 8.82 (d, 1H) | 321.4 |
| 182 | 2-thiophen-3-yl-3H- imidazo[4,5-b]pyridine- 7-carboxylic acid pyridin-4-yl-amide | 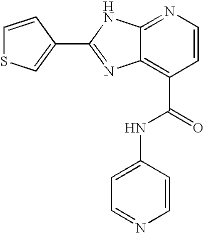 | 7.52 (s, 1H), 7.76 (m, 2H), 7.92 (m, 1H), 8.11 (m, 2H), 8.50 (s, 1H) | 321.4 |
| 183 | 2-thiophen-3-yl-3H- imidazo[4,5-b]pyridine- 7-carboxylic acid(6- methyloxy-pyridin-3-yl)- amide | 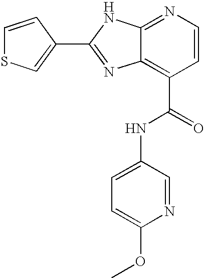 | 3.96 (s, 3H), 8.93 (d, 1H), 7.7 (s, 1H), 7.89~7.94 (m, 2H), 8.19 (d, 1H), 8.45~8.65 (m, 2H), 8.67 (s, 1H) | 351.4 |
| 184 | 2-thiophen-3-yl-3H- imidazo[4,5-b]pyridine- 7-carboxylic acid[2-(4- amino-phenyl)-ethyl]- amide | 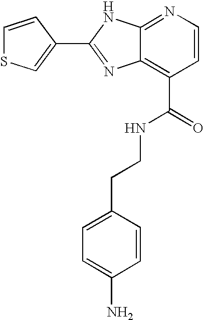 | 3.11 (t, 2H), 3.85 (t, 2H), 7.29 (d, 2H), 7.52 (d, 2H), 7.67~7.82 (m, 4H), 8.32~8.45 (m, 2H) | 363.4 |
| 185 | 2-thiophen-3-yl-3H- imidazo[4,5-b]pyridine- 7-carboxylic acid(6- methyl-pyridin-3-yl)- amide | 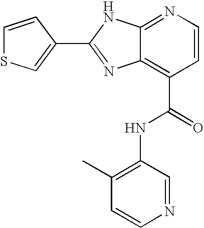 | 2.86 (s, 3H), 7.72 (m, 1H), 7.98~8.20 (m, 4H), 8.38 (d, 1H), 8.55 (m, 2H) | 335.4 |
| 186 | 2-thiophen-3-yl-3H- imidazo[4,5-b]pyridine- 7-carboxylic acid cycloheptyl amide | 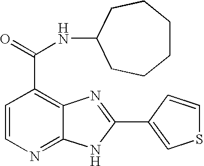 | 1.838~1.72 (m, 10H), 2.05 (m, 2H), 4.28 (m, 1H), 7.67 (s, 1H), 7.78 (s, 1H), 7.83 (d, 1H), 8.31 (s, 1H), 8.43 (d, 1H) | 340.5 |
| 187 | 2-thiophen-3-yl-3H- imidazo[4,5-b]pyridine- 7-carboxylic acid(5- methyl-2H-pyrazol-3- yl)-amide | 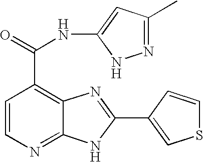 | 2.69 (s, 3H), 7.72~7.67 (m, 2H), 7.86 (d, 1H), 8.46 (s, 1H), 8.65 (br, 1H) | 324.4 |
| 188 | 2-thiophen-3-yl-3H- imidazo[4,5-b]pyridine- 7-carboxylic acid(6- methyl amino-pyridin-3- yl)-amide | 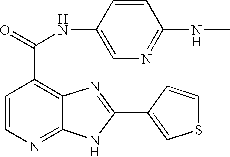 | 3.06 (s, 3H), 7.10 (d, 1H), 7.66 (m, 1H), 7.76 (d, 1H), 7.88 (d, 1H), 8.02 (m, 1H), 8.42 (s, 2H), 8.76 (s, 1H) | 350.4 |
| 189 | 2-thiophen-3-yl-1H- pyrrolo[2,3-b]pyridine- 4-carboxylic acid(4- benzolamino-phenyl)-amide | 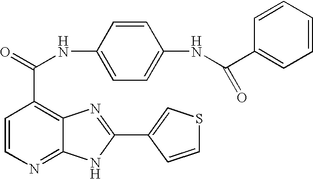 | DMSO: 6.68 (d, J = 2.3 Hz, 1H), 7.41-7.52 (m, 7H), 7.61 (s, 1H), 7.66 (d, J = 2.3 Hz, 2H), 7.91-7.92 (m, 3H), 8.11 (d, J = 2.1 Hz, 1H), 8.21 (t, J = 1.3 Hz, 2H), 8.48 (d, J = 2.1 Hz, 1H), 8.84 (s, 1H), 10.22 (s, 1H) | 439.5 |
| 190 | 2-thiophen-3-yl-3H- imidazo[4,5-b]pyridine- 7-carboxylic acid[3-(6- methanesulfonylamino- pyridin-3-yl)-propyl]- amide | 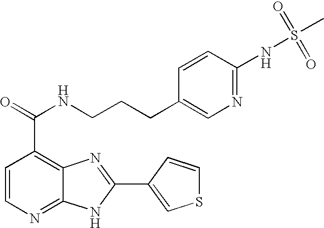 | 2.80 (s, 3H), 3.03 (t, 2H), 3.50 (s, 3H), 3.85 (t, 2H), 7.20 (d, 1H), 7.35 (d, 1H), 7.79 (d, 1H), 8.11 (d, 2H), 8.44 (s, 1H) | 456.5 |
| 191 | 6-bromo-2-thiophen-3- yl-3H-imidazo[4,5- b]pyridine- 7-carboxylic acid[2-(2-chloro- pyridin-4-yl)-ethyl]-amide | 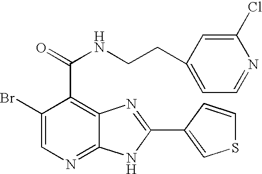 | 3.09 (t, 2H), 3.88 (t, 2H), 7.48 (s, 1H), 7.69 (s, 1H), 7.77 (m, 2H), 8.25 (d, 1H), 8.41 (s, 1H), 8.41 (s, 1H), 8.50 (br, 1H) | 462.8 |
| 192 | 2-thiophen-3-yl-3H- imidazo[4,5-b]pyridine- 7-carboxylic acid(6- methyl-pyridin-3-yl)- amide | 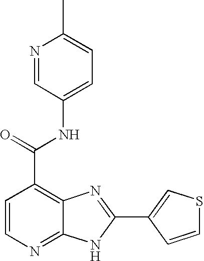 | 3.26 (s, 3H), 7.32 (s, 1H), 7.77-7.83 (m, 2H), 7.99 (s, 1H), 8.21 (d, 1H), 8.50 (d, 1H), 8.64 (s, 1H), 8.92 (s, 1H), 11.65 (s, 1H) | 335.4 |
| 193 | 2-thiophen-3-yl-3H- imidazo[4,5-b]pyridine- 7-carboxylic acid(4- ethoxy-pyridin-3-yl)- amide | 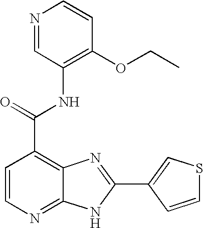 | 1.38 (t, 3H), 4.55 (q, 2H), 7.37 (s, 1H), 7.85 (t, 2H), 7.93 (d, 2H), 8.36 (s, 1H), 8.51 (t, 2H), 9.55 (s, 1H), 11.72 (s, 1H) | 365.4 |
| 194 | 2-thiophen-3-yl-3H- imidazo[4,5-b]pyridine- 7-carboxylic acid(4- ethylsulfanyl-pyridin-3- yl)-amide | 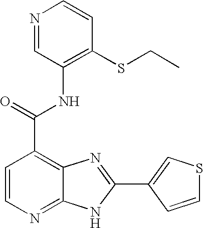 | 1.20-1.29 (m, 3H), 2.88- 3.00 (m, 2H), 6.40 (d, 1H), 7.34 (s, 1H), 7.73-7.81 (m, 2H), 7.88 (d, 1H), 8.48 (t, 2H), 10.88 (s, 1H), 11.72 (s, 1H) | 381.5 |
| 195 | 2-thiophen-3-yl-3H- imidazo[4,5-b]pyridine- 7-carboxylic acid(4- ethoxy-6-methyl-pyridin- 3-yl)-amide | 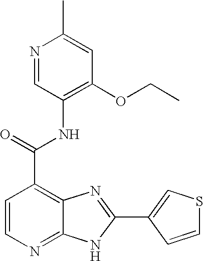 | 1.36 (t, 3H), 2.48 (t, 3H), 4.36 (q, 2H), 7.10 (s, 1H), 7.83 (q, 2H), 7.92 (d, 1H), 8.49 (q, 2H), 9.35 (s, 1H), 11.52 (s, 1H) | 379.4 |
| 196 | 2-(4-fluoro-phenyl)-3H- imidazo[4,5-b]pyridine- 7-carboxylic acid[2-(4- acetylamino-phenyl)- ethyl]-amide | 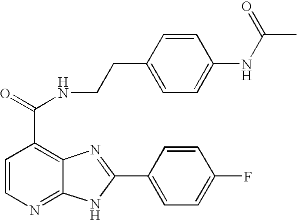 | 2.09 (s, 3H), 2.99 (t, 2H), 3.89 (t, 2H), 7.28 (m, 4H), 7.50 (m, 2H), 7.80 (m, 1H), 8.05 (m, 2H), 8.42 (d, 1H) | 417.4 |
| 197 | 2-(4-fluoro-phenyl)-3H- imidazo[4,5-b]pyridine- 7-carboxylic acid[2-(2- hydroxy-phenyl)-ethyl]- amide | 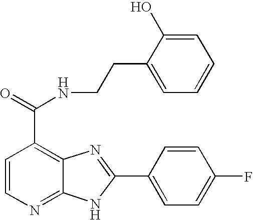 | .02 (t, 2H), 3.86 (t, 2H), 6.75 (m, 2H), 7.07 (m, 1H), 7.16 (m, 1H), 7.26 (m, 2H), 7.79 (m, 1H), 8.10 (m, 2H), 8.43 (d, 1H) | 376.4 |
| 198 | 2-(4-fluoro-phenyl)-3H- imidazo[4,5-b]pyridine- 7-carboxylic acid[2-(4- ethanesulfonylamino- phenyl)-ethyl]-amide | 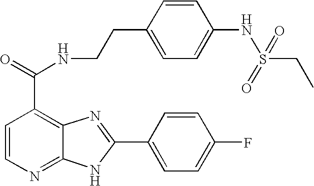 | 1.19 (t, 3H), 2.99 (q, 2H), 3.02 (t, 2H), 3.85 (t, 2H), 7.19 (d, 2H), 7.32 (m, 4H), 7.79 (m, 1H), 8.13 (m, 2H), 8.43 (d, 1H) | 467.5 |
| 199 | 2-(4-fluoro-phenyl)-3H- imidazo[4,5-b]pyridine- 7-carboxylic acid[3-(3H- imidazo-1-yl)-propyl]- amide | 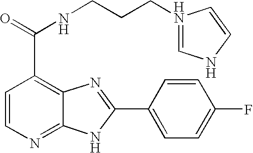 | 2.35 (m, 2H), 3.64 (t, 2H), 4.24 (t, 2H), 7.17 (m, 1H), 7.23 (m, 1H), 7.35 (t, 2H), 7.81 (m, 1H), 7.90 (m, 1H), 8.34 (m, 2H), 8.44 (d, 1H) | 366.4 |
| 200 | 2-(4-fluoro-phenyl)-3H- imidazo[4,5-b]pyridine- 7-carboxylic acid{2-[4- (2-dimethylamino- acetylamino)-phenyl}- ethyl}-amide | 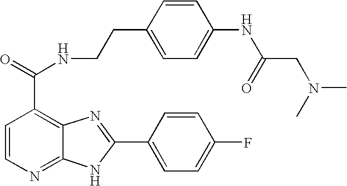 | 2.98 (m, 8H), 3.85 (t, 2H), 4.08 (s, 2H), 7.35~7.25 (m, 4H), 7.55 (d, 2H), 7.76 (d, 2H), 6.06 (m, 2H), 8.41 (d, 1H) | 460.5 |
| 201 | 2-(4-fluoro-phenyl)-3H- imidazo[4,5-b]pyridine- 7-carboxylic acid[2-(3- hydroxy-phenyl)-ethyl]- amide | 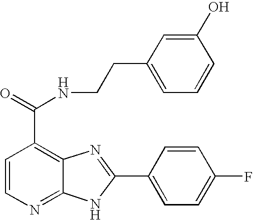 | 2.96 (t, 2H), 3.90 (t, 2H), 6.67 (d, 1H), 6.80 (d, 2H), 7.13 (t, 1H), 7.30 (t, 2H), 7.79 (m, 1H), 8.05 (m, 2H), 8.42 (d, 1H) | 376.4 |
| 202 | 2-(4-fluoro-phenyl)-3H- imidazo[4,5-b]pyridine- 7-carboxylic acid[3- (4,5-dichloro-imidazo- 1-yl)-propyl]-amide | 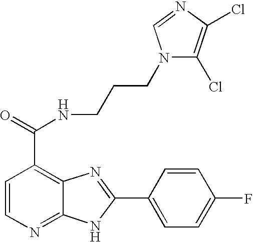 | 2.26 (m, 2H), 3.66 (t, 2H), 4.22 (t, 2H), 7.32 (t, 2H), 7.74 (d, 1H), 7.80 (s, 1H), 8.32 (m, 2H), 8.40 (d, 1H) | 433.3 |
| 203 | 2-(4-fluoro-phenyl)-3H- imidazo[4,5-b]pyridine- 7-carboxylic acid[2-(3- chloro-phenyl)-ethyl]- amide | 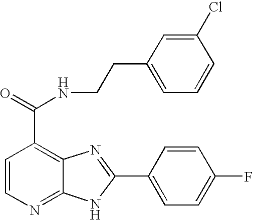 | 3.02 (t, 2H), 3.90 (t, 2H), 7.33 (m, 6H), 7.81 (d, 1H), 8.07 (m, 2H), 8.44 (d, 2H) | 394.8 |
| 204 | 2-(4-fluoro-phenyl)-3H- imidazo[4,5-b]pyridine- 7-carboxylic acid(2-{4- [(4-methyl-piperazine-1- carbonyl)-amino]-phenyl}- ethyl)-amide | 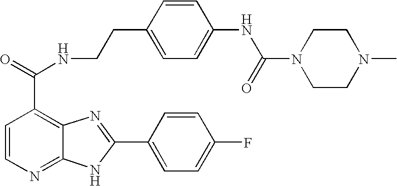 | 2.82 (s, 3H), 3.00 (t, 2H), 3.85~3.23 (m, 8H), 3.86 (t, 2H), 7.34 (m, 4H), 7.56 (d, 2H), 7.80 (d, 1H), 8.11 (m, 2H), 8.46 (d, 1H) | 501.6 |
| 205 | 2-(4-fluoro-phenyl)-3H- imidazo[4,5-b]pyridine- 7-carboxylic acid[3-(4- methyl-imidazo-1-yl)- propyl]-amide | 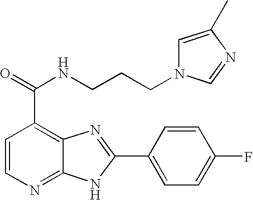 | 2.22 (d, 3H), 2.34 (m, 2H), 3.68 (t, 2H), 4.35 (t, 2H), 7.33 (d, 1H), 7.39 (m, 2H), 7.81 (d, 1H), 8.32 (m, 2H), 8.47 (d, 1H), 8.86 (d, 1H) | 378.4 |
| 206 | 2-(4-fluoro-phenyl)-3H- imidazo[4,5-b]pyridine- 7-carboxylic acid[2-(4- methanesulfonylamino- phenyl)-ethyl]-amide | 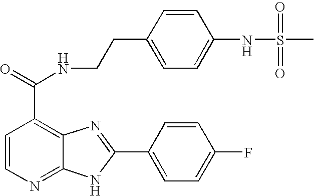 | 2.81 (s, 3H), 3.02 (t, 2H), 3.87 (t, 2H), 7.19 (d, 2H), 7.35 (d, 1H), 7.44 (d, 1H), 7.77 (m, 2H), 8.12 (m, 2H), 8.43 (d, 1H) | 453.5 |
| 207 | 2-(4-fluoro-phenyl)-3H- imidazo[4,5-b]pyridine- 7-carboxylic acid[2-(4- dimethylamino- sulfonylamino-phenyl)- ethyl]-amide | 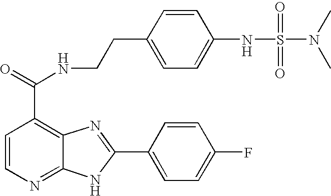 | 2.69 (s, 3H), 3.00 (t, 2H), 3.86 (t, 2H), 7.16 (d, 2H), 7.30 (m, 4H), 7.80 (m, 1H), 8.10 (m, 2H), 8.42 (d, 1H) | 482.5 |
| 208 | 2-(4-fluoro-phenyl)-3H- imidazo[4,5-b]pyridine- 7-carboxylic acid[2-(3- trifluoro methyl-phenyl)- ethyl]-amide | 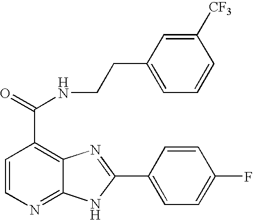 | 3.07 (t, 2H), 3.91 (t, 2H), 7.25 (m, 2H), 7.45 (m, 2H), 7.57 (m, 2H), 7.80 (d, 1H), 8.04 (m, 2H), 8.41 (d, 1H) | 428.4 |
| 209 | 2-(4-fluoro-phenyl)-3H- imidazo[4,5-b]pyridine- 7-carboxylic acid{2-[3- (2-morpholin-4-yl- ethoxy)-phenyl]-ethyl}- amide | 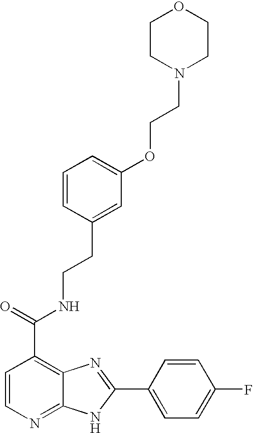 | 3.02 (t, 2H), 3.10-3.40 (m, 2H), 3.40-3.60 (m, 2H), 3.53 (t, 2H), 3.61-3.82 (m, 2H), 3.88 (t, 2H), 3.82- 4.10 (m, 2H), 4.28 (t, 2H), 6.88-6.92 (m, 1H), 6.98 (s, 1H), 7.05 (d, 1H), 7.21- 7.38 (m, 3H), 7.77 (d, 1H), 8.02-8.16 (m, 1H), 8.43 (d, 1H) | 489.6 |
| 210 | 2-(4-fluoro-phenyl)-3H- imidazo[4,5-b]pyridine- 7-carboxylic acid{2-[3- (3-morpholin-4-yl- propoxy)-phenyl]- ethyl}-amide | 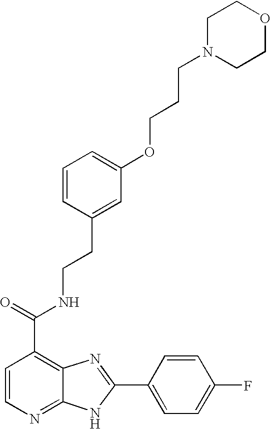 | 1.82-1.87 (m, 2H), 2.09- 2.20 (m, 2H), 3.01 (t, 2H), 3.11-3.20 (m, 2H), 3.21- 3.31 (m, 2H), 3.40-3.61 (m, 2H), 3.62-3.82 (m, 2H), 3.85-3.92 (m, 2H), 4.05 (t, 2H), 6.81-6.89 (m, 1H), 6.91 (s, 1H), 6.99 (s, 1H), 7.20-7.40 (m, 3H), 7.79 (d, 1H), 8.05-8.15 (m, 2H), 8.28-8.32 (m, 1H), 8.44 (d, 1H) | 503.6 |
| 211 | 2-(4-fluoro-phenyl)-3H- imidazo[4,5-b]pyridine- 7-carboxylic acid{2-[3- (2-dimethylamino- ethoxy)-phenyl]-ethyl}- amide | 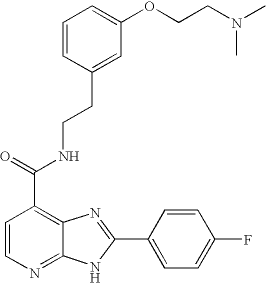 | 2.90 (s, 6H), 3.03 (t, 2H), 3.50 (t, 2H), 3.88 (t, 2H), 4.23 (t, 2H), 6.85-6.92 (m, 1H), 7.0 (s, 1H), 7.05 (d, 1H), 7.25-7.40 (m, 3H), 7.78 (s, 1H), 8.05-8.15 (m, 2H), 8.43 (d, 1H) | 447.5 |
| 212 | 2-(4-fluoro-phenyl)-3H- imidazo[4,5-b]pyridine- 7-carboxylic acid(2- pyridin-2-yl-ethyl)- amide | 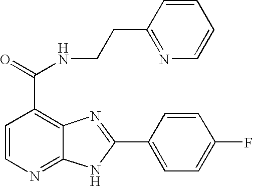 | 3.59 (t, 2H), 4.09 (t, 2H), 7.36 (t, 2H), 7.88 (d, 1H), 7.95 (m, 1H), 8.10 (d, 1H), 8.28)(m, 1H), 8.45~8.55 (m, 2H), 8.76 (d, 1H) | 361.4 |
| 213 | 2-(4-fluoro-phenyl)-3H- imidazo[4,5-b]pyridine- 7-carboxylic acid(2- pyridin-3-yl-ethyl)- amide | 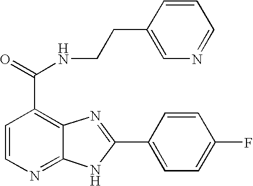 | 3.31 (t, 2H), 4.00 (t, 2H), 7.38 (t, 2H), 7.75 (d, 1H), 8.02 (m, 1H), 8.31 (m, 2H), 8.45 (d, 1H), 8.70 (m, 2H), 8.88 (s, 1H) | 361.4 |
| 214 | 2-(4-fluoro-phenyl)-3H- imidazo[4,5-b]pyridine- 7-carboxylic acid(2- pyridin-4-yl-ethyl)- amide | 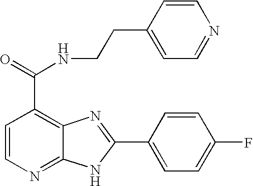 | 3.38 (t, 2H), 4.04 (t, 2H), 7.36 (m, 1H), 7.75 (d, 1H), 8.02 (d, 1H), 8.24 (m, 1H), 8.45 (d, 1H), 8.70 (d, 1H) | 361.4 |
| 215 | 2-(4-fluoro-phenyl)-3H- imidazo[4,5-b]pyridine- 7-carboxylic acid[2-(4- fluoro-3-hydroxy- phenyl)-ethyl]-amide | 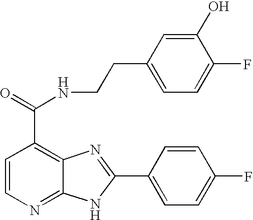 | 2.95 (t, 2H), 3.89 (t, 2H), 6.78 (m, 1H), 6.84 (m, 1H), 6.99 (m, 1H), 7.33 (t, 2H), 7.81 (d, 1H), 8.08 (m, 2H), 8.45 (d, 1H) | 394.4 |
| 216 | 2-(4-fluoro-phenyl)-3H- imidazo[4,5-b]pyridine- 7-carboxylic acid[2-(1- oxy-pyridin-4-yl)-ethyl]- amide | 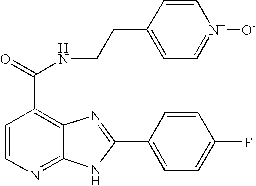 | 3.22 (t, 2H), 3.96 (t, 2H), 7.40 (t, 2H), 7.72 (d, 1H), 7.79 (d, 1H), 8.25 (m, 2H), 8.43 (d, 2H), 8.50 (d, 1H) | 377.4 |
| 217 | 2-(4-fluoro-phenyl)-3H- imidazo[4,5-b]pyridine- 7-carboxylic acid[2-(2- chloro-pyridin-3-yl)- ethyl]-amide | 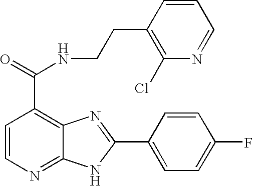 | 3.06 (t, 2H), 3.87 (t, 2H), 7.33~7.41 (m, 2H), 7.79~7.82 (m, 1H), 8.18 (m, 1H), 8.33 (d, 1H), 8.50 (d, 1H) | 395.8 |
| 218 | 2-(4-fluoro-phenyl)-3H- imidazo[4,5-b]pyridine- 7-carboxylic acid[2-(2- chloro-pyridin-4-yl)- ethyl]-amide | 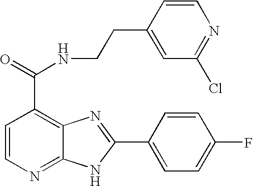 | 3.09 (t, 2H), 3.93 (t, 2H), 7.31~7.39 (m, 2H), 7.50 (s, 1H), 7.79 (d, 1H), 8.15 (m, 2H), 8.26 (d, 1H), 8.48 (m, 1H) | 395.8 |
| 219 | 2-(4-fluoro-phenyl)-3H- imidazo[4,5-b]pyridine- 7-carboxylic acid[2-(6- chloro-pyridin-3-yl)- ethyl]-amide | 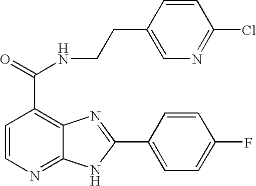 | 3.20 (t, 2H), 3.92 (t, 2H), 7.38~7.42 (m, 3H), 7.68 (d, 1H), 7.80~8.14 (m, 3H), 8.28 (m, 1H), 8.90 (s, 1H) | 395.8 |
| 220 | 2-(4-fluoro-phenyl)-3H- imidazo[4,5-b]pyridine- 7-carboxylic acid[2-(2- aminomethyl-pyridin-3- yl)-ethyl]-amide | 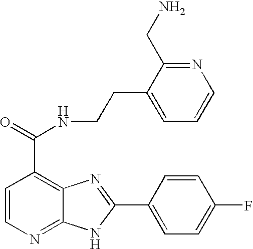 | 3.11 (t, 2H), 3.86 (t, 2H), 4.46 (s, 2H), 7.38 (m, 1H), 7.60 (m, 2H), 7.78~7.94 (m, 2H), 8.18 (m, 2H), 8.50 (d, 2H) | 390.4 |
| 221 | 2-(4-fluoro-phenyl)-3H- imidazo[4,5-b]pyridine- 7-carboxylic acid[2-(2- aminomethyl-pyridin-4- yl)-ethyl]-amide | 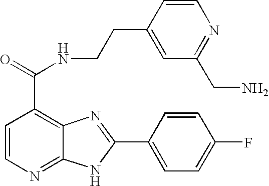 | 3.13 (t, 2H), 3.91 (t, 2H), 4.20 (s, 2H), 7.35 (t, 2H), 7.46 (m, 2H), 7.75 (d, 1H), 8.21 (m, 2H), 8.46 (m, 1H), 8, 54 (d, 1H) | 390.4 |
| 222 | 2-(4-fluoro-phenyl)-3H- imidazo[4,5-b]pyridine- 7-carboxylic acid[2-(6- aminomethyl-pyridin-3- yl)-ethyl]-amide | 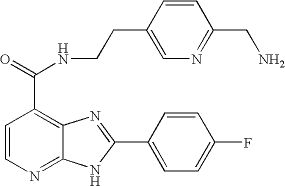 | 3.13 (t, 2H), 3.88 (t, 2H), 4.18 (s, 2H), 7.36 (m, 3H), 7.75 (d, 1H), 7.88 (d, 1H), 8.27 (m, 2H), 8.49 (br, 1H), 8.61 (s, 1H) | 390.4 |
| 223 | 2-(4-fluoro-phenyl)-3H- imidazo[4,5-b]pyridine- 7-carboxylic acid[2-(2- fluoro-pyridin-4-yl)- ethyl]-amide | 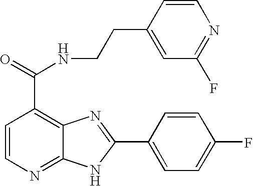 | 3.14 (t, 2H), 3.95 (t, 2H), 7.11 (s, 1H), 7.32 (m, 3H), 7.80 (s, 1H), 8.11 (m, 3H), 8.50 (br, 1H) | 379.4 |
| 224 | 2-(4-fluoro-phenyl)-3H- imidazo[4,5-b]pyridine- 7-carboxylic acid[2-(2- methylamino-pyridin-4- yl)-ethyl]-amide | 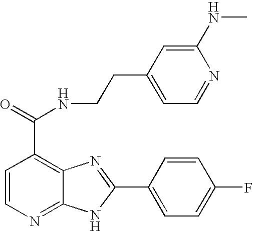 | 2.82 (s, 3H), 3.09 (t, 2H), 3.94 (t, 2H), 6.87 (s, 1H), 6.95 (d, 1H), 7.35 (t, 2H), 7.77 (m, 2H), 8.21 (m, 2H), 8.45 (d, 1H) | 390.4 |
| 225 | 2-(4-fluoro-phenyl)-3H- imidazo[4,5-b]pyridine- 7-carboxylic acid[2-(4- trifluoromethane sulfonylamino-phenyl)- ethyl]-amide | 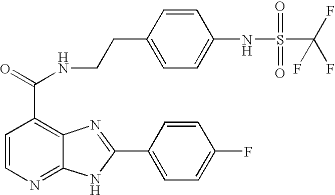 | 3.05 (t, 2H), 3.05 (t, 2H), 3.87 (t, 2H), 7.20 (m, 3H), 7.29~7.39 (m, 3H), 7.80 (s, 1H), 8.15 (m, 2H), 8.44 (d, 1H) | 507.5 |
| 226 | 2-(4-fluoro-phenyl)-3H- imidazo[4,5-b]pyridine- 7-carboxylic acid(3- pyridin-3-yl-propyl)-amide | 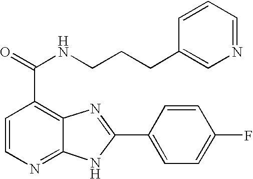 | 2.20 (q, 2H), 3.08 (t, 2H), 3.68 (t, 2H), 7.34 (t, 2H), 7.79 (d, 1H), 7.98 (t, 1H), 8.30 (m, 2H), 8.48 (s, 1H), 8.59 (d, 1H), 8.65 (s, 1H), 8.82 (s, 1H) | 375.4 |
| 227 | 2-(4-fluoro-phenyl)-3H- imidazo[4,5-b]pyridine- 7-carboxylic acid(3- pyridin-4-yl-propyl)-amide | 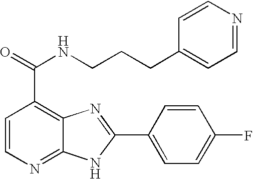 | 2.23 (q, 2H), 3.13 (t, 2H), 3.69 (t, 2H), 7.35 (t, 2H), 7.78 (d, 1H), 7.98 (d, 1H), 8.31 (m, 2H), 8.46 (d, 1H), 8.63 (br, 1H) | 375.4 |
| 228 | 2-(4-fluoro-phenyl)-3H- imidazo[4,5-b]pyridine- 7-carboxylic acid[3-(2- chloro-pyridin-4-yl)- propyl]-amide | 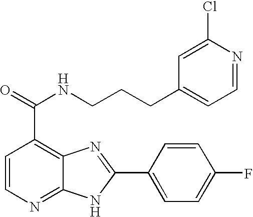 | 2.11 (t, 2H), 2.86 (t, 2H), 3.62 (t, 2H), 7.29 (d, 1H), 7.38 (s, 1H), 7.69 (s, 1H), 7.77 (d, 1H), 7.83 (s, 1H), 8.16 (d, 1H), 8.45 (m, 2H) | 409.9 |
| 229 | 2-(4-fluoro-phenyl)-3H- imidazo[4,5-b]pyridine- 7-carboxylic acid[2-(2- methyl-pyridin-3-yl)- ethyl]-amide | 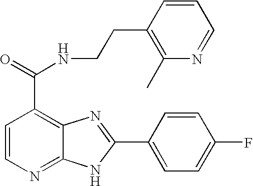 | 2.87 (s, 3H), 3.31 (t, 2H), 3.95 (t, 3H), 7.35 (t, 2H), 7.71 (d, 1H), 7.78 (m, 1H), 8.28 (m, 2H), 8.44~8.54 (m, 3H) | 375.4 |
| 230 | 2-(4-fluoro-phenyl)-3H- imidazo[4,5-b]pyridine- 7-carboxylic acid[3-(6- fluoro-pyridin-3-yl)- propyl]-amide | 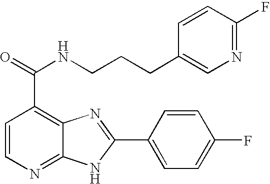 | 2.10 (t, 2H), 2.85 (t, 2H), 3.63 (t, 3H), 6.98 (d, 1H), 7.33 (m, 2H), 7.78~7.90 (m, 2H), 8.00 (s, 1H), 8.25~8.55 (m, 2H) | 393.4 |
| 231 | 2-(4-fluoro-phenyl)-3H- imidazo[4,5-b]pyridine- 7-carboxylic acid pyridin-2-ylamide | 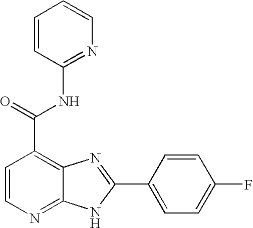 | 7.24 (m, 1H), 7.31 (t, 2H), 7.88~7.98 (m, 2H), 8.42~8.51 (m, 5H) | 333.3 |
| 232 | 2-(4-fluoro-phenyl)-3H- imidazo[4,5-b]pyridine- 7-carboxylic acid pyridin-3-ylamide | 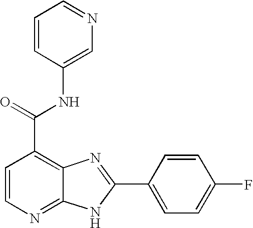 | 7.38 (t, 2H), 7.92 (d, 2H), 8.39 (m, 2H), 8.48~8.68 (m, 4H), 9.65 (br, 1H) | 333.3 |
| 233 | 2-(4-fluoro-phenyl)-3H- imidazo[4,5-b]pyridine- 7-carboxylic acid pyridin-4-ylamide | 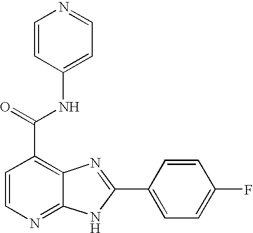 | 7.35~7.41 (t, 2H), 7.93 (d, 1H), 8.36~8.52 (m, 5H), 8.54 (m, 1H), 8.55 (br, 1H) | 333.3 |
| 234 | [2-(4-fluoro-phenyl)-3H- imidazo[4,5-b]pyridin- 7-yl]-piperidin-1-yl- methanone | 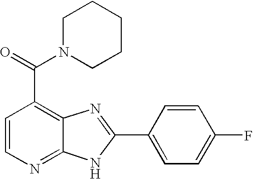 | 1.58 (s, 2H), 1.76 (s, 4H), 3.35 (d, 2H), 3.84 (d, 2H), 7.37 (t, 2H), 7.43 (d, 1H), 8.26~8.30 (m, 2H), 8.52 (s, 1H) | 324.4 |
| 235 | [2-(4-fluoro-phenyl)-3H- imidazo[4,5-b]pyridin- 7-yl]-(4-methyl-piperidin- 1-yl)-methanone | 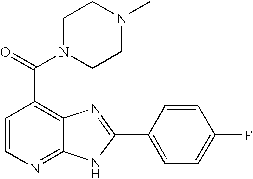 | 2, 99 (s, 3H), 3.250~3.80 (br, 8H), 7.32~7.39 (m, 3H), 8.22~8.27 (m, 2H), 8.47 (d, 1H) | 339.4 |
| 236 | 2-(4-fluoro-phenyl)-3H- imidazo[4,5-b]pyridine- 7-carboxylic acid[2-(4- amino-phenyl)-ethyl]- amide | 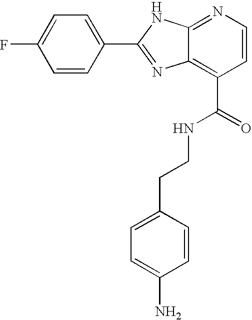 | 3.11 (t, 2H), 3.87 (t, 2H), 7.25 (d, 2H), 7.38 (t, 2H), 7.49 (d, 2H), 7.76 (d, 1H), 8.23 (m, 2H), 8.44 (m, 1H) | 375.4 |
| 237 | 2-(4-fluoro-phenyl)-3H- imidazo[4,5-b]pyridine- 7-carboxylic acid(6- methyloxy-pyridin-3-yl)- amide | 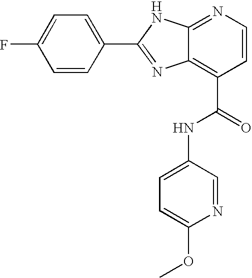 | 4.00 (s, 3H), 7.00 (d, 1H), 7.39 (t, 2H), 7.93 (d, 1H), 8.23 (d, 1H), 8.38 (m, 2H), 8.62 (m, 1H), 8.77 (m, 1H) | 363.4 |
| 238 | 22-(4-fluoro-phenyl)-3H- imidazo[4,5-b]pyridine- 7-carboxylic acid[3-(6- chloro- pyridin-3-yl)- propyl]-amide | 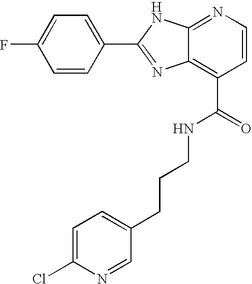 | 2.10 (m, 2H), 2.82~2.98 (m, 2H), 3.62 (m, 2H), 7.28~7.38 (m, 3H), 7.73~7.85 (m, 2H), 8.19 (d, 1H), 8.18 (m, 2H), 8.32 (m, 1H) | 409.9 |
| 239 | 2-(4-fluoro-phenyl)-3H- imidazo[4,5-b]pyridine- 7-carboxylic acid(2- methyloxy-pyridin-3-yl)- amide | 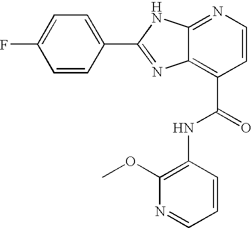 | 4.23 (s, 3H), 7.05 (m, 2H), 7.42 (t, 2H), 7.95 (m, 2H), 8.48 (m, 2H), 8.98 (d, 1H) | 363.4 |
| 240 | 2-(4-fluoro-phenyl)-3H- imidazo[4,5-b]pyridine- 7-carboxylic acid cycloheptylamide | 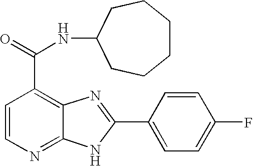 | 1.84~1.72 (m, 10H), 2.07 (m, 2H), 4.28 (m, 1H), 7.35 (t, 2H), 7.81 (d, 1H), 8.29~8.25 (m, 2H), 8.44 (d, 2H) | 352.4 |
| 241 | 2-(4-fluoro-phenyl)-3H- imidazo[4,5-b]pyridine- 7-carboxylic acid(4- methyl-pyridin-3-yl)- amide | 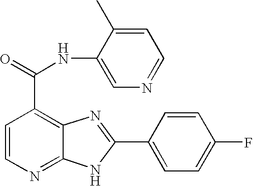 | 2.69 (s, 3H), 7.35 (t, 2H), 7.43 (d, 1H), 7.94 (d, 1H), 8.35~8.27 (m, 2H), 8.51 (d, 1H), 9.48 (br, 1H) | 347.4 |
| 242 | 2-(4-fluoro-phenyl)-3H- imidazo[4,5-b]pyridine- 7-carboxylic acid(6- chloro-pyridin-3-yl)- amide | 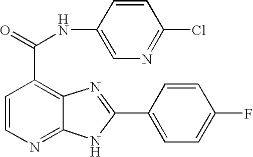 | 7.36 (t, 2H), 7.52 (d, 1H), 7.88 (d, 1H), 8.39~8.34 (m, 3H), 8.50 (d, 1H), 8.91 (br, 1H) | 367.8 |
| 243 | 2-(4-fluoro-phenyl)-3H- imidazo[4,5-b]pyridine- 7-carboxylic acid(5- methyl-2H-pyrazol-3- yl)-amide | 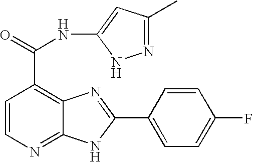 | 2.38 (s, 3H), 7.38~7.33 (m, 3H), 7.82 (s, 1H), 8.31~8.60 (m, 3H) | 336.3 |
| 244 | 2-(4-fluoro-phenyl)-3H- imidazo[4,5-b]pyridine- 7-carboxylic acid(5- methyl-pyridin-2-yl)- amide | 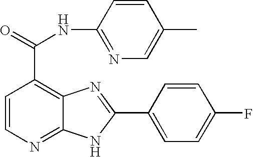 | 2.55 (s, 3H), 6.97 (d, 1H), 7.33 (m, 2H), 7.83 (d, 1H), 8.18 (m, 1H), 8.38 (m, 2H), 8.55 (d, 1H), 8.62 (d, 1H) | 347.4 |
| 245 | 2-(4-fluoro-phenyl)-3H- imidazo[4,5-b]pyridine- 7-carboxylic acid(2- chloro-pyridin-3-yl)- amide | 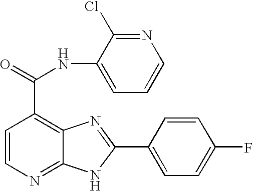 | 7.50 (m, 3H), 7.84 (s, 1H), 8.22 (s, 1H), 8.38 (m, 2H), 8.53 (d, 1H), 8.90 (d, 1H) | 367.8 |
| 246 | 2-(4-fluoro-phenyl)-3H- imidazo[4,5-b]pyridine- 7-carboxylic acid(4- methyloxy-pyridin-3-yl)- amide | 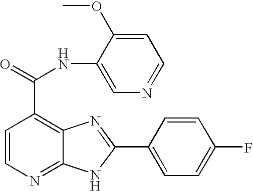 | 4.32 (s, 3H), 7.41 (t, 3H), 7.91 (d, 2H), 8.38 (m, 3H), 8.50 (s, 1H) | 363.4 |
| 247 | 2-(4-fluoro-phenyl)-3H- imidazo[4,5-b]pyridine- 7-carboxylic acid(4- chloro-pyridin-3-yl)- amide | 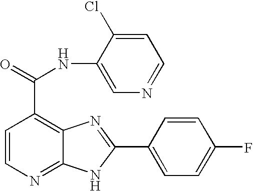 | 7.38 (m, 2H), 7.89 (s, 1H), 8.02 (s, 1H), 8.35 (m, 3H), 7.59 (d, 1H), 8.66 (d, 1H) | 367.8 |
| 248 | 2-(4-fluoro-phenyl)-3- methyl-3H-imidazo[4,5- b]pyridine-7-carboxylic acid [2-(4- ethanesulfonylamino- phenyl)-ethyl]-amide | 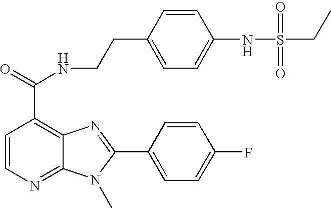 | 1.19 (t, 3H), 2.99 (q, 2H), 3.02 (t, 2H), 3.63 (s, 3H), 3.85 (t, 2H), 7.19 (d, 2H), 7.32 (m, 4H), 7.79 (m, 1H), 8.13 (m, 2H), 8.43 (d, 1H) | 481.6 |
| 249 | 2-(4-fluoro-phenyl)-3H- imidazo[4,5- b]pyridine-7-carboxylic acid(4- piperidin-1-yl-phenyl)- amide | 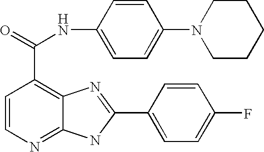 | DMSO: 1.58~1.72 (m, 6H), 3.98~4.02 (m , 4H), 7.25 (t, J = 2.9 Hz, 2H), 7.31 (d, J = 3.1 Hz, 1H), 7.4l (d, J = 2.1 Hz, 2H), 7.64 (d, J = 2.1 Hz, 2H), 7.95 (t, J = 2.9 Hz, 2H), 8.15 (d, J = 2.1 Hz, 1H) | 415.5 |
| 250 | 2-(4-fluoro-phenyl)-3H- imidazo[4,5- b]pyridine-7-carboxylic acid(4- morpholin-4-yl-phenyl)- amide | 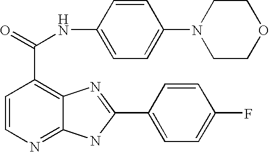 | DMSO: 3.98 (d, J = 2.6 Hz, 4H), 4.08 (d, J = 2.6 Hz, 4H), 7.25 (t, J = 2.9 Hz, 2H), 7.31 (d, J = 3.1 Hz, 1H), 7.41 (d, J = 2.1 Hz, 2H), 7.64 (d, J = 2.1 Hz, 2H), 7.95 (t, J = 2.9 Hz, 2H), 8.15 (d, J = 2.1 Hz, 1H) | 417.4 |
| 251 | 6-bromo-2-(4-fluoro- phenyl)-3H-imidazo[4,5- b]pyridine-7-carboxylic acid[2-(2-chloro- pyridin-4-yl)-ethyl]- amide | 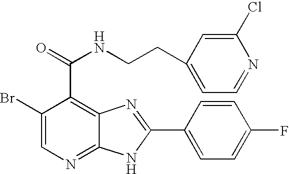 | 3.09 (t, 2H), 3.93 (t, 2H), 7.31~7.39 (m, 2H), 7.50 (s, 1H), 8.15 (m, 2H), 8.26 (d, 1H), 8.48 (m, 1H) | 474.7 |
| 252 | 6-chloro-2-(4-fluoro- phenyl)-3H-imidazo[4,5- b]pyridine-7-carboxylic acid[2-(2-chloro- pyridin-4-yl)-ethyl]- amide | 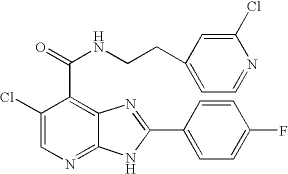 | DMSO: 3.98 (d, J = 2.6 Hz, 2H, 4.08 (d, J = 2.6 Hz, 2H), 7.25 (t, J = 2.9 Hz, 2H), 7.45 (s, 1H), 7.65 (d, J = 3.2 Hz, 1H), 7.85 (d, J = 3.2 Hz, 1H), 7.89 (s, 1H), 7.91 (t, J = 2.9 Hz, 2H) | 430.3 |
| 253 | 2-(4-fluoro-phenyl)-3H- imidazo[4,5-b]pyridine- 7-carboxylic acid phenylamide | 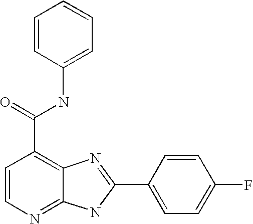 | CD3Cl: 7.21-7.29 (m, 7H), 7.38 (d, J = 2.1 Hz, 1H), 7.46 (d, J = 2.1 Hz, 1H), 7.88-7.90 (d, J = 2.1 Hz, 1H), 8.29-8.35(m, 1H) | 332.3 |
| 254 | 2-(4-fluoro-phenyl)-3H- imidazo[4,5-b]pyridine- 7-carboxylic acid(4- hydroxy-cyclohexyl)-amide | 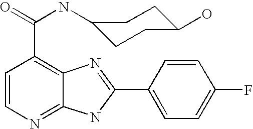 | CD₃OD: 1.53-1.59 (m, 4H), 2.09-2.23 (m, 4H), 3.82- 3.88 (m, 1H), 3.79-4.12 (m, 2H), 6.92 (d, J = 3.4 Hz, 2H), 7.79 (d, J = 2.4 Hz, 1H), 8.11 (d, J = 3.4 Hz, 2H), 8.43 (d, J = 2.4 Hz, 1H) | 354.4 |
| 255 | 2-(4-fluoro-phenyl)-3H- imidazo[4,5-b]pyridine- 7-carboxylic acid(1- benzyl-piperidin-4-yl)- amide | 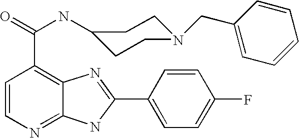 | CD₃OD: 1.33-1.43 (m, 4H), 2.22-2.45 (m, 4H), 4.02- 4.12 (m, 1H), 5.21 (s, 2H), 7.22-7.37 (m, 3H), 7.49- 7.52 (m, 5H), 8.21 (d, J = 3.4 Hz, 2H) | 429.5 |
| 256 | 2-(4-fluoro-phenyl)-3H- imidazo[4,5-b]pyridine- 7-carboxylic acid piperidin-4-ylamide | 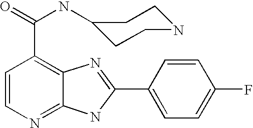 | CD3OD: 1.88-1.95 (m, 4H), 2.52-2.61 (m, 4H), 4.12- 4.22 (m, 1H), 7.25-7.40 (m, 3H), 8.25 (d, J = 3.1 Hz, 2H) | 339.4 |
| 257 | 2-(4-fluoro-phenyl)-3H- imidazo[4,5-b]pyridine- 7-carboxylic acid (2,6- diethyl-phenyl)-amide | 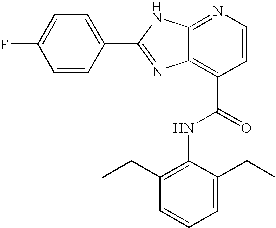 | CDCl₃: 1.25 (t, 2.7 Hz, 6H), 2.76 (q, 2.9 Hz, 6H), 7.19- 7.32 (m, 5H), 8.25-8.30 (m, 3H), 8.52-8.55 (b, 1H), 11.11 (s, 1H) | 388.4 |
| 258 | 2-(4-fluoro-phenyl)-3H- imidazo[4,5-b]pyridine- 7-carboxylic acid (2,6- diisopropyl-phenyl)- amide | 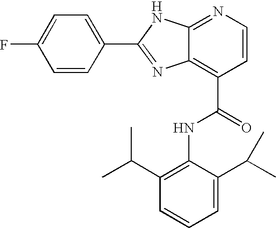 | CDCl3: 1.25 (s, 6H), 1.28 (s, 6H), 3.21-3.38 (m, 2H), 7.25-7.40 (m, 5H), 8.28- 8.32 (m, 3H), 8.45-8.52 (b, 1H), 10.99 (s, 1H) | 416.5 |
| 259 | 2-(4-fluoro-phenyl)-3H- imidazo[4,5-b]pyridine- 7-carboxylic acid benzyl- ethyl-amide | 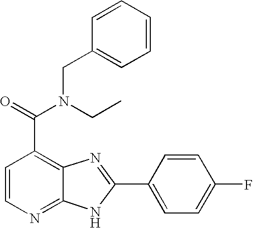 | CDCl3: 1.43 (t, 2.5 Hz, 3H), 2.96 (q, 2.1 Hz, 2H), 5.20 (s, 2H), 7.19-7.32 (m, 6H), 8.15-8.23 (m, 4H), 8.52- 8.55 (b, 1H) | 374.4 |
| 260 | 2-(4-fluoro-phenyl)-3H- imidazo[4,5-b]pyridine- 7-carboxylic acid(1- benzyl-pyrrolidin-3-yl)- amide | 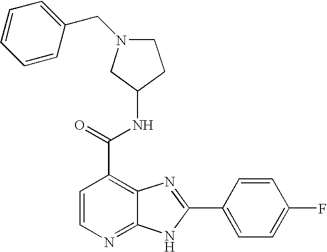 | CDCl₃: 2.01-2.32 (m, 6H), 5.20 (s, 2H), 4.23-4.44 (m, 1H) 7.21-7.25 (m, 3H), 7.33-7.35 (m, 3H), 8.21- 8.28 (m, 4H), 8.42-8.50 (b, 1H) | 415.5 |
| 261 | 2-(4-fluoro-phenyl)-3H- imidazo[4,5-b]pyridine- 7-carboxylic acid isopropyl-phenyl-amide | 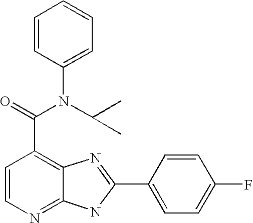 | CDCl₃: 1.25 (d, 2.7 Hz, 6H), 3.96-3.99 (m, 1H), 7.29- 7.37 (m, 6H), 8.25-8.33 (m, 4H), 8.22-8.31 (b, 1H) | 374.4 |
| 262 | 2-(4-fluoro-phenyl)-3H- imidazo[4,5-b]pyridine- 7-carboxylic acid (1,7,7- trimethyl- bicyclo[2.2.1]heptin-2-yl)-amide | 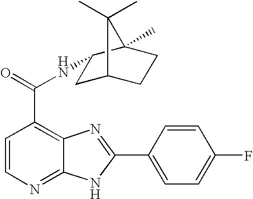 | CD₃OD: 0.96 (s, 3H), 0.99 (s, 3H), 1.13 (s, 3H), 1.17- 2.02 (m, 1H), 1.41-1.63 (m, 3H), 1.78-2.02 (m, 3H), 4.48-4.54 (m, 1H), 6.82 (d, J = 3.6 Hz, 2H), 7.79 (d, J = 2.4 Hz, 1H), 8.12 (d, J = 3.6 Hz, 2H), 8.40 (d, J = 2.4 Hz, 1H) | 392.5 |
| 263 | 2-(4-fluoro-phenyl)-3H- imidazo[4,5-b]pyridine- 7-carboxylic acid adamantan-1-ylamide | 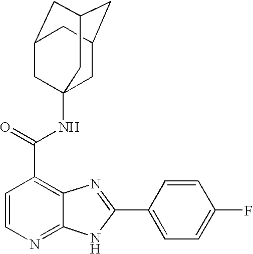 | CDCl₃: 1.25-2.01 (m, 15H), 6.89-7.79 (m, 5H), 8.22- 8.28 (m, 3H), 8.48-8.51 (b, 1H) | 390.5 |
| 264 | 2-(4-fluoro-phenyl)-3H- imidazo[4,5-b]pyridine- 7-carboxylic acid adamantan-2-ylamide | 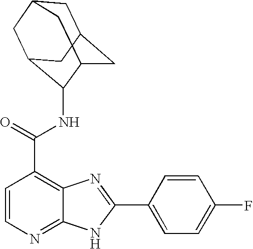 | CDCl₃: 1.25-2.01 (m, 15H), 6.99-7.81 (m, 5H), 8.25- 8.29 (m, 3H), 8.49-8.53 (b, 1H) | 390.5 |
| 265 | 2-{[2-(4-fluoro- phenyl)-3H-imidazo[4,5- b]pyridine-7-carbonyl]- amino}- bicyclo[2.2.1]heptane- 2-carboxylic acid | 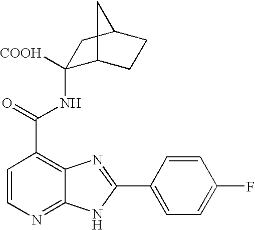 | CD₃OD: 2.28-2.33 (m, 3H), 2.54-2.53 (m, 2H), 3.50- 3.71 (m, 3H), 3.90-3.97 (m, 1H), 7.21 (t, J = 3.1 Hz, 2H), 7.80 (d, J = 2.5 Hz, 1H), 8.20 (d, J = 3.1 Hz, 2H), 8.42 (d, J = 2.5 Hz, 1H) | 394.4 |
| 266 | 2-(4-fluoro-phenyl)-3H- imidazo[4,5-b]pyridine- 7-carboxylic acid(1- azabicyclo[2.2.2]octyn- 3-yl)-amide | 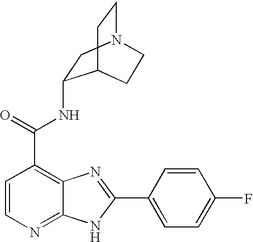 | CD₃OD: 2.18-2.24 (m, 3H), 2.40-2.53 (m, 2H), 3.20- 3.64 (m, 3H), 3.92-3.99 (m, 1H), 4.02-4.22 (m, 1H), 4.61-4.31 (m, 1H), 7.23 (t, J = 3.4 Hz, 2H), 7.78 (d, J = 2.4 Hz, 1H), 8.18 (d, J = 3.4 Hz, 2H), 8.41 (d, J = 2.4 Hz, 1H) | 365.4 |
| 267 | 2-(4-fluoro-phenyl)-3H- imidazo[4,5-b]pyridine- 7-carboxylic acid cyclohexylamide | 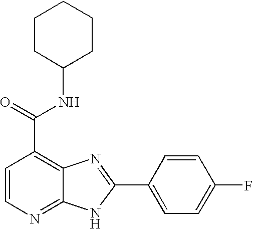 | CD₃OD: 1.36-1.66 (m, 5H), 1.62-1.79 (m, 5H), 4.01- 4.18 (m, 1H), 7.18 (d, J = 3.4 Hz, 2H), 7.76 (d, J = 2.4 Hz, 1H), 8.14 (d, J = 3.4 Hz, 2H), 8.41 (d, J = 2.4 Hz, 1H) | 338.4 |
| 268 | 2-(4-fluoro-phenyl)-3H- imidazo[4,5-b]pyridine- 7-carboxylic acid bicyclo[2.2.1]heptin-2- ylamide | 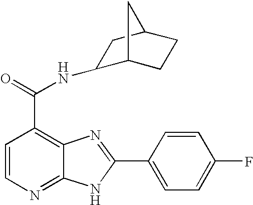 | CD₃OD: 1.36-1.66 (m, 4H), 1.62-1.79 (m, 3H), 2.01- 2.13 (m, 3H), 4.01-4.18 (m, 1H), 7.18 (d, J = 3.4 Hz, 2H), 7.76 (d, J = 2.4 Hz, 1H), 8.14 (d, J = 3.4 Hz, 2H), 8.41 (d, J = 2.4 Hz, 1H) | 350.4 |
| 269 | 2-(4-fluoro-phenyl)-3H- imidazo[4,5-b]pyridine- 7-carboxylic acid (2,6,6- trimethyl- bicyclo[3.1.1]heptin-3- yl)-amide | 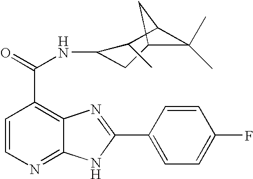 | CD₃OD: 1.17 (m, 3H), 1.23 (d, 2.6 Hz, 3H), 1.27- 1.38 (m, 1H), 1.37 (s, 3H), 1.79-1.83 (m, 1H), 1.92- 1.94 (m, 1H), 2.01-2.10 (m, 1H), 2.12-2.21 (m, 1H), 2.56-2.61 (m, 1H), 2.69- 2.81 (m, 1H), 4.62-4.65 (m, 1H), 6.93 (d, J = 3.4 Hz, 2H), 7.78 (d, J = 2.4 Hz, 1H), 8.10 (d, J = 3.4 Hz, 2H), 8.42 (d, J = 2.4 Hz, 1H) | 392.5 |
| 270 | 2-(4-fluoro-phenyl)-3H- imidazo[4,5-b]pyridine- 7-carboxylic acid (2,6,6- trimethyl- bicyclo[3.1.1]heptin-3- yl)-amide | 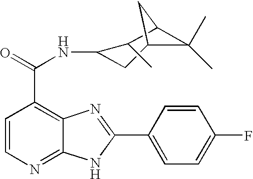 | CD₃OD: 1.17 (m, 3H), 1.23 (d, 2.6 Hz, 3H), 1.27- 1.38 (m, 1H), 1.37 (s, 3H), 1.79-1.83 (m, 1H), 1.92- 1.94 (m, 1H), 2.01-2.10 (m, 1H), 2.12-2.21 (m, 1H), 2.56-2.61 (m, 1H), 2.69- 2.81 (m, 1H), 4.62-4.65 (m, 1H), 6.93 (d, J = 3.4 Hz, 2H), 7.78 (d, J = 2.4 Hz, 1H), 8.10 (d, J = 3.4 Hz, 2H), 8.42 (d, J = 2.4 Hz, 1H) | 392.5 |
| 271 | 2-(4-fluoro-phenyl)-3H- imidazo[4,5-b]pyridine- 7-carboxylic acid (2,6- dimethyl-phenyl)-amide | 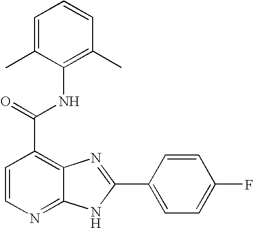 | CD₃OD: 2.60 (s, 6H), 6.94 (d, J = 3.6 Hz, 2H), 7.95 (d, J = 2.4 Hz, 1H), 8.19 (d, J = 3.6 Hz, 2H), 8.48 (d, J = 2.4 Hz, 1H) | 360.4 |
| 272 | 2-(4-fluoro-phenyl)-3H- imidazo[4,5-b]pyridine- 7-carboxylic acid(3- amino-cyclohexyl)-amide | 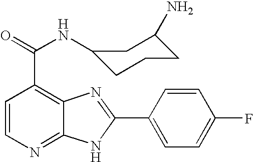 | CD₃OD: 1.21-1.64 (m, 4H), 1.77-1.79 (m, 1H), 1.82- 2.19 (m, 2H), 2.43-2.63 (m, 2H), 4.42-4.47 (m, 1H), 6.72 (d, J = 3.4 Hz, 2H), 7.79 (d, J = 2.4 Hz, 1H), 8.01 (d, J = 3.4 Hz, 2H), 8.31 (d, J = 2.4 Hz, 1H) | 353.4 |
| 273 | 2-(4-fluoro-phenyl)-3H- imidazo[4,5-b]pyridine- 7-carboxylic acid cyclopentylamide | 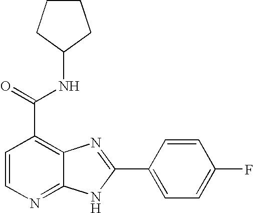 | CD₃OD: 1.60-1.78 (b, 4H), 1.80-1.90 (b, 2H), 2.07- 2.09 (b, 2H), 4.48-4.54 (m, 1H), 6.85 (d, J = 3.4 Hz, 2H), 7.27 (d, J = 2.4 Hz, 1H), 8.23 (d, J = 3.4 Hz, 2H), 8.42 (d, J = 2.4 Hz, 1H) | 324.4 |
| 274 | 2-(4-fluoro-phenyl)-3H- imidazo[4,5-b]pyridine- 7-carboxylic acid bicyclo[2.2.1]heptin-2- ylamide | 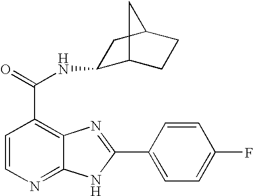 | CD₃OD + CDCl₃: 1.05- 1.07 (m, 2H), 1.25-1.30 (m, 4H), 1.78-1.83 (m, 1H), 2.39 (s, 2H), 3.25-3.30 (m, 1H), 3.92-3.98 (m, 1H), 7.14 (t, J = 3.4 Hz, 1H), 7.81 (d, J = 2.4 Hz, 1H) 8.17 (t, J = 3.4 Hz, 2H), 8.26 (d, J = 2.4 Hz, 1H) | 350.4 |
| 275 | 2-(4-fluoro-phenyl)-3H- imidazo[4,5-b]pyridine- 7-carboxylic acid(2- hydroxy-1,1-dimethyl- ethyl)-amide | 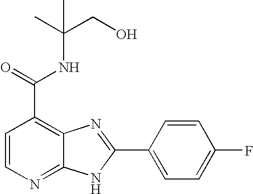 | CD₃OD + CDCl₃: 1.41 (s, 6H), 3.61 (s, 2H), 6.93 (d, J = 4.4 Hz, 1H), 7.18 (t, J = 3.6 Hz, 2H), 7.64 (d, J = 2.4 Hz, 1H), 8.21 (t, J = 3.4 Hz, 2H), 8.25 (d, J = 2.4 Hz, 1H) | 328.3 |
| 276 | 2-(4-fluoro-phenyl)-3H- imidazo[4,5-b]pyridine- 7-carboxylic acid(6- methoxy-4-methyl- pyridine-3-yl)-amide | 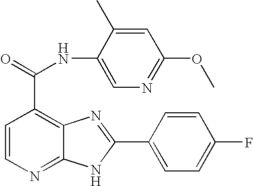 | 10.97 (s, 1H), 8.48 (d, 1H), 8.41 (s, 1H), 8.24 (d, 2H), 7.77 (d, 1H), 7.18 (d, 2H), 6.40 (s, 1H), 3.87 (s, 3H), 2.37 (s, 3H) | 377.4 |
| 277 | 2-(4-fluoro-phenyl)-3H- imidazo[4,5-b]pyridine- 7-carboxylic acid tert- butylamide | 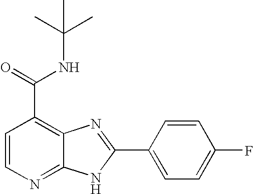 | CD₃OD: 0.93 (s, 9H), 6.76 (d, J = 3.6 Hz, 2H), 7.69 (d, J = 2.4 Hz, 1H), 8.11 (d, J = 3.6 Hz, 2H), 8.29 (d, J = 2.4 Hz, 1H) | 312.3 |
| 278 | 2-(4-fluoro-phenyl)-3H- imidazo[4,5-b]pyridine- 7-carboxylic acid(2- dimethylamino-1-methyl- ethyl)-amide | 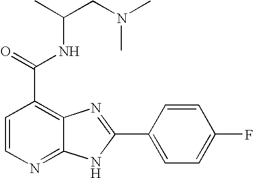 | CD₃OD: 1.45 (d, J = 3.6 Hz, 3H), 2.99 (s, 6H), 3.04- 3.69 (m, 3H), 6.88 (d, J = 3.4 Hz, 2H), 7.79 (d, J = 2.4 Hz, 1H), 8.14 (d, J = 3.4 Hz, 2H), 8.21 (d, J = 2.4 Hz, 1H) | 341.4 |
| 279 | 2-(4-fluoro-phenyl)-3H- imidazo[4,5-b]pyridine- 7-carboxylic acid(2- ethoxy-phenyl)-amide | 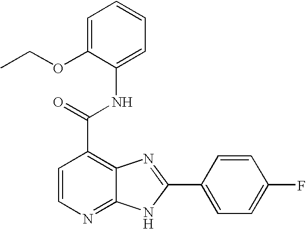 | CD₃OD: 2.22 (t, J = 2.5 Hz, 3H), 4.34 (q, J = 2.5 Hz, 2H), 6.99 (d, J = 3.4 Hz, 2H), 7.01- 7.22 (m, 3H), 7.86 (d, J = 2.4 Hz, 1H), 8.20 (d, J = 2.4 Hz, 2H), 8.39-8.40 (m, 1H), 8.46 (d, J = 2.4 Hz, 1H) | 376.4 |
| 280 | 2-(4-fluoro-phenyl)-3H- imidazo[4,5-b]pyridine- 7-carboxylic acid(2- hydroxy-cyclopentyl)- amide | 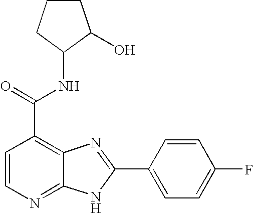 | CD₃OD: 1.62-2.02 (m, 6H), 3.40-3.51 (m, 1H), 4.19- 4.39 (m, 1H), 6.87 (d, J = 3.4 Hz, 2H), 7.76 (d, J = 2.4 Hz, 1H), 8.12 (d, J = 3.4 Hz, 2H), 8.26 (d, J = 2.4 Hz, 1H) | 340.4 |
| 281 | 2-(4-fluoro-phenyl)-3H- imidazo[4,5-b]pyridine- 7-carboxylic acid(1- hydroxymethyl- cyclopentyl)-amide | 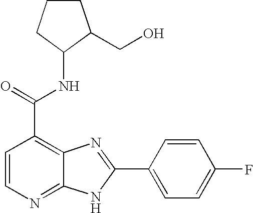 | CD₃OD: 1.72-2.42 (m, 8H), 3.20 (s, 2H), 3.40-3.51 (m, 1H), 6.66 (d, J = 3.6 Hz, 2H), 7.56 (d, J = 2.4 Hz, 1H), 8.02 (d, J = 3.6 Hz, 2H), 8.16 (d, J = 2.4 Hz, 1H) | 354.4 |
| 282 | 2-(4-fluoro-phenyl)-3H- imidazo[4,5-b]pyridine- 7-carboxylic acid(1,1- dimethyl-propyl)-amide | 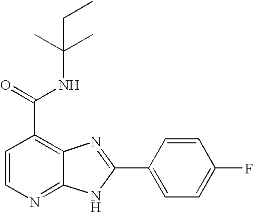 | CDCl₃: 1.13 (t, J = 2.8 Hz, 3H), 1.59 (s, 6H), 1.99 (q, J = 2.1 Hz, 2H), 7.39 (t, J = 3.4 Hz, 2H), 8.02 (d, J = 2.4 Hz, 1H), 8.25 (d, J = 3.4 Hz, 2H), 8.42 (d, J = 2.4 Hz, 1H) | 326.4 |
| 283 | 2-(4-fluoro-phenyl)-3H- imidazo[4,5-b]pyridine- 7-carboxylic acid(3,3,5- trimethyl-cyclohexyl)- amide | 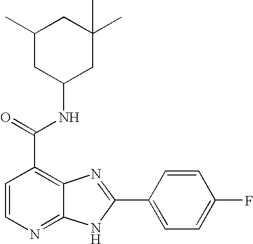 | DMSO: 0.83 (s, 3H), 1.00 (s, 3H), 1.08 (s, 3H), 1.18- 1.52 (m, 4H), 1.76-1.88 (m, 2H), 7.49 (t, J = 3.4 Hz, 2H), 7.72 (d, J = 2.4 Hz, 1H), 8.25 (t, J = 3.4 Hz, 2H), 8.42 (d, J = 2.4 Hz, 1H) | 380.5 |
| 284 | 2-(4-fluoro-phenyl)-3H- imidazo[4,5-b]pyridine- 7-carboxylic acid naphthalen-2-ylamide | 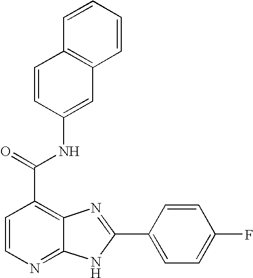 | 11.8 (s, 1H), 8.63 (s, 1H), 8.58 (d, 1H), 8.48 (m, 2H), 8.02 (m, 2H), 7.81 (m, 3H), 7.44 (m, 3H), 7.35 (d, 1H) | 382.4 |
| 285 | 2-(4-fluoro-phenyl)-3H- imidazo[4,5-b]pyridine- 7-carboxylic acid quinolin-6-ylamide | 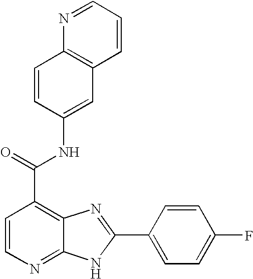 | 12.2 (s, 1H), 8.78 (m, 3H), 8.62 (d, 1H), 8.43 (m, 1H), 8.18 (d, 2H), 7.80 (m2H), 7.58 (d, 1H), 7.44 (m, 1H), 7.08 (s, 1H) | 383.4 |
| 286 | 2-(4-fluoro-phenyl)-3H- imidazo[4,5-b]pyridine- 7-carboxylic acid quinolin-3-ylamide | 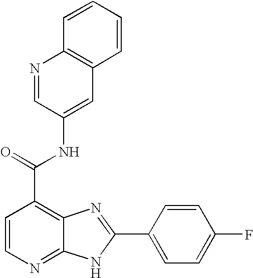 | 11.98 (s, 1H), 9.40 (s, 1H), 9.07 (s, 1H), 8.56 (m, 3H), 8.16 (m, 2H), 8.02 (m, 1H), 7.80 (m, 2H), 7.73 (m, 1H), 7.42 (m, 2H) | 383.4 |
| 287 | 2-(4-fluoro-phenyl)-3H- imidazo[4,5-b]pyridine- 7-carboxylic acid(4- ethoxy-pyridin-3-yl)- amide | 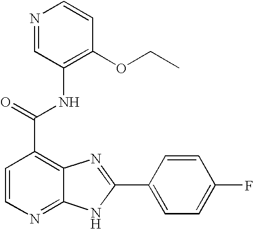 | 12.0 (s, 1H), 9.75 (s, 1H), 8.73 (d, 1H), 8.40 (d, 1H), 8.39 (m, 2H), 7.88 (m, 2H), 7.55 (m, 2H), 4.70 (q, 2H), 1.42 (t, 3H) | 377.4 |
| 288 | 2-(4-fluoro-phenyl)-3H- imidazo[4,5-b]pyridine- 7-carboxylic acid(6- methoxy-2-methyl- pyridin-3-yl)-amide | 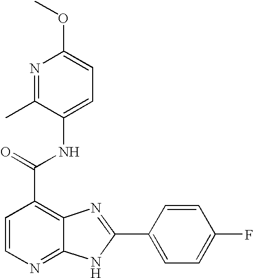 | 11.23 (s, 1H), 8.58 (d, 1H), 8.39 (m, 3H), 7.83 (d, 1H), 7.46 (m, 2H), 6.80 (d, 1H), 3.88 (s, 3H), 2.65 (s, 3H) | 377.4 |
| 289 | 2-(4-fluoro-phenyl)-3H- imidazo[4,5-b]pyridine- 7-carboxylic acid(1,3,3- trimethyl-butyl)-amide | 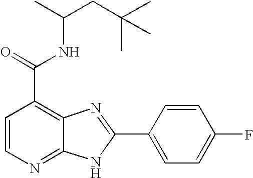 | CD₃OD: 1.12 (s, 9H), 1.28 (d, J = 1.4 Hz, 3H), 1.30 (d, J = 2.4 Hz, 2H), 4.10 (q, J = 1.8 Hz, 2H), 6.75 (d, J = 3.4 Hz, 2H), 7.73 (d, J = 2.4 Hz, 1H), 8.01 (d, J = 3.4 Hz, 2H), 8.31 (d, J = 2.4 Hz, 1H) | 354.4 |
| 290 | 2-(4-fluoro-phenyl)-3H- imidazo[4,5-b]pyridine- 7-carboxylic acid(1,4- dimethyl-pentyl)-amide | 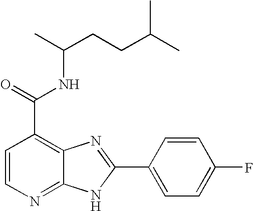 | CD₃OD: 0.96 (d, J = 4.4 Hz, 3H), 1.01 (d, J = 4.4 Hz, 3H), 1.26-1.29 (m, 2H), 1.29 (d, J = 3.1 Hz, 3H), 1.48-1.67 (m, 2H), 1.86-1.89 (m, 1H), 4.27 (q, J = 1.2 Hz, 2H), 6.74 (d, J = 3.4 Hz, 2H), 7.69 (d, J = 2.4 Hz, 1H), 7.99 (d, J = 3.4 Hz, 2H), 8.30 (d, J = 2.4 Hz, 1H) | 354.4 |
| 291 | 2-(4-fluoro-phenyl)-3H- imidazo[4,5-b]pyridine- 7-carboxylic acid(1- ethyl-propyl)-amide | 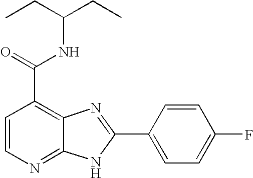 | CD₃OD: 1.23 (t, J = 3.4 Hz, 6H), 3.27 (q, J = 1.2 Hz, 4H), 4.21-4.28 (m, 1H), 6.99 (d, J = 3.1 Hz, 2H), 7.61 (d, J = 2.0 Hz, 1H), 7.89 (d, J = 3.1 Hz, 2H), 8.28 (d, J = 2.0 Hz, 1H) | 326.4 |
| 292 | 2-(4-fluoro-phenyl)-3H- imidazo[4,5-b]pyridine- 7-carboxylic acid (1,1,3,3-tetramethyl- butyl)-amide | 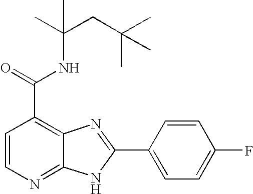 | 1.03 (s, 9H), 1.26-1.40 (m, 2H), 1.63 (s, 6H), 2.32 (s, 6H), 7.82 (d, 1H), 7.99 (d, 2H), 8.17 (d, 2H), 8.29 (d, 1H) | 368.5 |
| 293 | 2-(4-fluoro-phenyl)-3H- imidazo[4,5-b]pyridine- 7-carboxylic acid benzothiazo-2-ylamide | 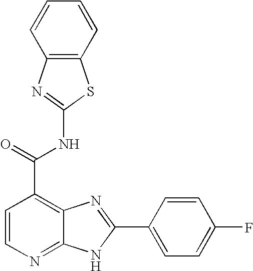 | 9.80 (s, 1H), 8.58 (d, 1H), 8.46 (m, 1H), 7.90 (d, 2H), 7.70 (d, 1H), 7.52 (m, 2H), 7.46 (m, 2H), 7.30 (m, 2H) | 389.4 |
| 294 | 2-(4-fluoro-phenyl)-3H- imidazo[4,5-b]pyridine- 7-carboxylic acid (1H- indazo-6-yl)-amide | 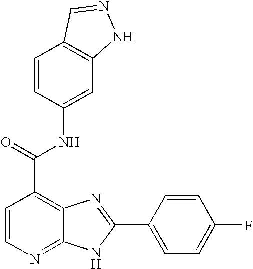 | 12.03 (s, 1H), 9.50 (s, 1H), 8.60 (m, 1H), 8.50 (m, 1H), 8.28 (m, 1H), 8.17 (s, 2H), 7.91 (d, 2H), 7.62 (s, 1H), 7.50 (m, 1H), 7.09 (d, 1H) | 372.4 |
| 295 | 2-(4-fluoro-phenyl)-3H- imidazo[4,5-b]pyridine- 7-carboxylic acid(2- methyl-1H-indol-5-yl)- amide | 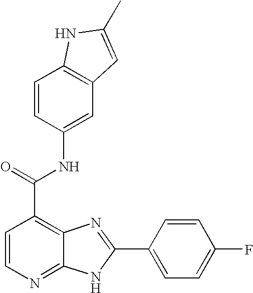 | 11.22 (s, 1H), 10.05 (s, 1H), 8.57 (d, 1H), 8.43 (m, 2H), 7.80 (m, 3H), 7.40 (m, 5H), 6.97 (d, 1H), 2.38 (s, 3H) | 385.4 |
| 296 | 2-(4-fluoro-phenyl)-3H- imidazo[4,5-b]pyridine- 7-carboxylic acid (1H- indol-5-yl)-amide | 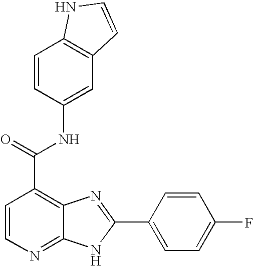 | 11.38 (s, 1H), 10.04 (s, 1H), 8.47 (m, 1H), 7.70 (m, 4H), 7.42 (m, 3H), 7.05 (d, 1H), 6.50 (s, 1H) | 371.4 |
| 297 | 2-(4-fluoro-phenyl)-3H- imidazo[4,5-b]pyridine- 7-carboxylic acid(3- trifluoromethoxy-phenyl)- amide | 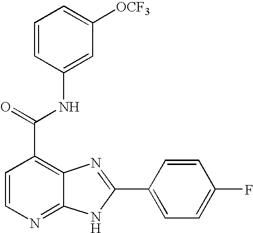 | 11.72 (s, 1H), 8.58 (d, 1H), 8.44 (m, 2H), 8.19 (s, 1H), 7.82 (d, 1H), 7.66 (d, 1H), 7.50 (m, 2H), 7.10 (m, 1H), 6.58 (m, 1H) | 416.3 |
| 298 | 2-(4-fluoro-phenyl)-3H- imidazo[4,5-b]pyridine- 7-carboxylic acid(3,5- dimethoxy-phenyl)- amide | 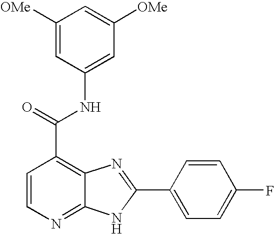 | 11.55 (s, 1H), 8.58 (d, 1H), 8.40 (m, 2H), 7.80 (d, 1H), 7.53 (m, 2H), 7.04 (s, 2H), 6.40 (s, 1H), 3.80 (s, 6H) | 392.4 |
| 299 | 2-(4-fluoro-phenyl)-3H- imidazo[4,5-b]pyridine- 7-carboxylic acid(3- acetylamino-phenyl)- amide | 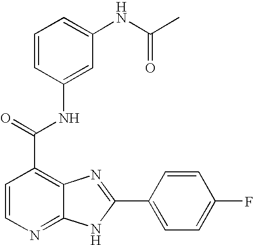 | 11.76 (s, 1H), 10.11 (s, 1H), 8.54 (d, 1H), 8.42 (m, 1H), 7.82 (m, 1H), 7.50 (m, 2H), 7.38 (m, 2H), 7.01 (d, 1H), 2.04 (s, 3H) | 389.4 |
| 300 | 2-(4-fluoro-phenyl)-3H- imidazo[4,5-b]pyridine- 7-carboxylic acid(3- cyano-phenyl)-amide | 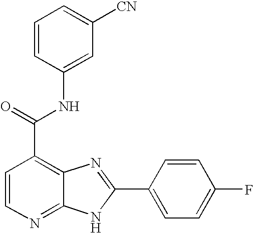 | 11.55 (s, 1H), 8.60 (d, 1H), 8.43 (m, 1H), 7.45 (m, 1H), 7.26 (m, 2H), 7.03 (m, 5H) | 357.3 |
| 301 | 2-(4-fluoro-phenyl)-3H- imidazo[4,5-b]pyridine- 7-carboxylic acid(3- benzyloxy-phenyl)-amide | 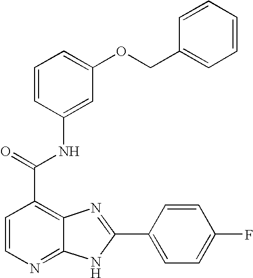 | 11.58 (s, 1H), 8.58 (d, 1H), 8.40 (m, 2H), 7.84 (d, 1H), 7.77 (s, 1H), 7.48 (m, 4H), 7.30 (m, 4H), 5.18 (s, 2H) | 438.5 |
| 302 | 2-(4-fluoro-phenyl)-3H- imidazo[4,5-b]pyridine- 7-carboxylic acid(4- phenyl-thiazo-2-yl)- amide | 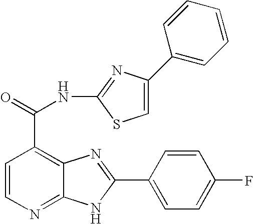 | 7.21-7.29 (m, 7H), 7.38 (d, 1H), 7.46 (d, 1H), 7.88-7.90 (d, 1H), 8.29-8.35 (m, 2H) | 415.5 |
| 303 | 2-(4-fluoro-phenyl)-3H- imidazo[4,5-b]pyridine- 7-carboxylic acid o- tolylamide | 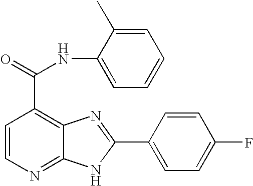 | 11.76 (s, 1H), 10.11 (s, 1H), 8.54 (d, 1H), 8.42 (m, 1H), 7.82 (m, 1H), 7.50 (m, 2H), 7.38 (m, 2H), 7.01 (d, 1H), 2.35 (s, 3H) | 346.4 |
| 304 | 2-(4-fluoro-phenyl)-3H- imidazo[4,5-b]pyridine- 7-carboxylic acid benzo[1,3]dioxol-5- ylamide | 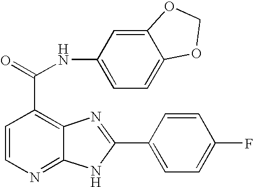 | 7.82 (m, 1H), 7.40~7.50 (m, 3H), 7.38 (m, 2H), 7.23 (m, 1H), 7.01 (m, 2H), 6.06 (s, 2H) | 376.4 |
| 305 | 2-(4-fluoro-phenyl)-3H- imidazo[4,5-b]pyridine- 7-carboxylic acid (2,6- dimethyl-pyridin-3-yl)- amide | 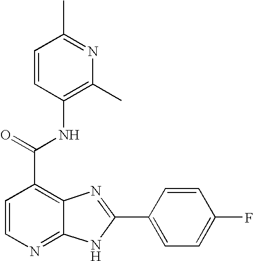 | 11.63 (s, 1H), 8.95 (s, 1H), 8.60 (d, 1H), 8.44 (m, 2H), 7.90 (d, 1H), 7.56 (m, 3H), 2.89 (s, 3H), 2.60 (s, 3H) | 361.4 |
| 306 | 2-(4-fluoro-phenyl)-3H- imidazo[4,5-b]pyridine- 7-carboxylic acid(2- chloro-6-methoxy- pyridin-3-yl)-amide | 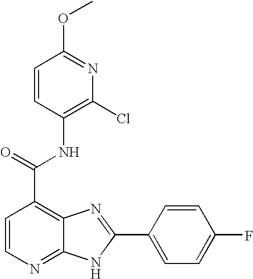 | 11.75 (s, 1H), 8.78 (d, 1H), 8.52 (d, 1H), 8.40 (m, 2H), 7.80 (d(1H), 7.47 (m, 2H), 7.00 (d, 1H), 3.90 (s, 3H) | 397.8 |
| 307 | 2-(4-fluoro-phenyl)-3H- imidazo[4,5-b]pyridine- 7-carboxylic acid(6- methoxy-5-methyl- pyridin-3-yl)-amide | 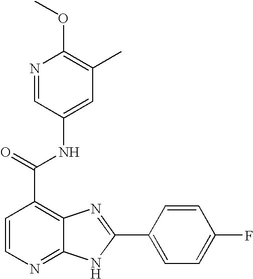 | 11.00 (s, 1H), 8.55 (d, 1H), 8.42 (m, 2H), 8.24 (d, 1H), 7.78 (d, 1H), 7.59 (s, 1H), 7.48 (m, 2H), 3.50 (s, 3H), 2.10 (s, 3H) | 377.4 |
| 308 | 2-(4-fluoro-phenyl)-3H- imidazo[4,5-b]pyridine- 7-carboxylic acid(6- methyl-pyridin-3-yl)-amide | 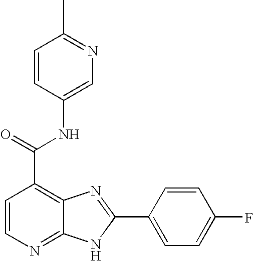 | 11.86 (s, 1H), 9.37 (s, 1H), 8.65 (m, 1H), 8.56 (d, 1H), 8.45 (m, 3H), 7.95 (d, 1H), 7.80 (d, 1H), 7.50 (m, 2H), 7.45 (m, 3H), 2.75 (s, 3H) | 347.4 |
| 309 | 2-(4-fluoro-phenyl)-3H- imidazo[4,5-b]pyridine- 7-carboxylic acid(2- methanesulfonyl- phenyl)-amide | 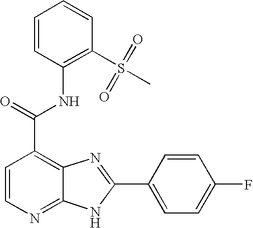 | 11.66 (s, 1H), 10.21 (s, 1H), 8.74 (d, 1H), 8.62 (m, 1H), 7.92 (m, 1H), 7.40 (m, 2H), 7.32 (m, 2H), 7.11 (d, 1H), 2.84 (s, 3H) | 410.4 |
| 310 | 2-(4-fluoro-phenyl)-3H- imidazo[4,5-b]pyridine- 7-carboxylic acid(6- chloro-4-methyl-pyridin- 3-yl)-amide | 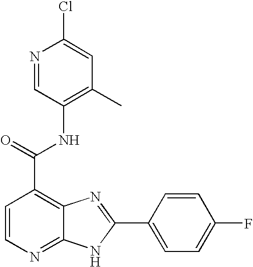 | 11.70 (s, 1H), 9.65 (s, 1H), 8.60 (m, 2H), 8.38 (m, 2H), 8.00 (m, 1H), 7.85 (m, 1H), 7.47 (m, 2H), 2.909 (s, 3H) | 381.8 |
| 311 | 2-(4-fluoro-phenyl)-3H- imidazo[4,5-b]pyridine- 7-carboxylic acid(4- isobutoxy-pyridin-3-yl)- amide | 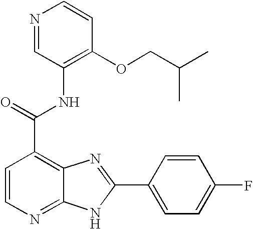 | 9.30 (br, 1H), 8.58 (t, 2H), 8.47 (s, 1H), 7.89 (d, 1H), 7.81 (d, 1H), 7.34 (t, 2H), 6.65 (d, 1H), 3.92 (d, 1H), 2.20 (m, 1H), 1.03 (d, 6H) | 405.4 |
| 312 | 2-(4-fluoro-phenyl)-3H- imidazo[4,5-b]pyridine- 7-carboxylic acid(4- cyclopropylmethoxy- pyridin-3-yl)-amide | 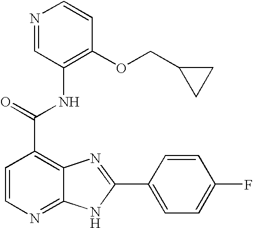 | 9.66 (br, 1H), 8.45 (m, 3H), 8.13 (d, 1H), 7.85 (d, 1H), 7.32 (t, 2H), 6.94 (d, 1H), 4.13 (d, 2H), 1.41 (m, 1H), 0.79 (m, 2H), 0.57 (m, 2H) | 403.4 |
| 313 | 2-(4-fluoro-phenyl)-3H- imidazo[4,5-b]pyridine- 7-carboxylic acid[4-(2- dimethylamino-ethoxy)- pyridin-3-yl]-amide | 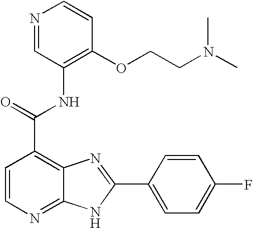 | 9.44 (br, 1H), 8.58~8.28 (m, 3H), 8.00 (d, 1H), 7.85 (d, 1H), 7.35 (m, 3H), 6.79 (d, 1H), 4.61 (t, 2H), 3.75 (t, 2H), 2.99 (s, 6H) | 420.5 |
| 314 | 2-(4-fluoro-phenyl)-3H- imidazo[4,5-b]pyridine- 7-carboxylic acid[4-(2- morpholin-4-yl-ethoxy)- pyridin-3-yl]-amide | 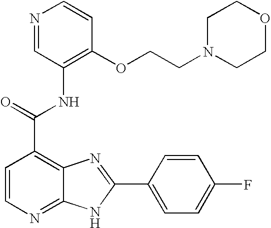 | 9.46 (br, 1H), 8.62~8.35 (m, 3H), 7.98 (d, 2H), 7.90 (d, 2H), 7.35 (m, 3H), 7.98 (d, 2H), 7.90 (d, 2H), 7.35 (m, 3H), 6.79 (d, 1H), 4.85 (t, 2H), 3.94 (br, 4H), 3.69 (t, 2H), 3.32 (br, 4H) | 462.5 |
| 315 | 2-(4-fluoro-phenyl)-3H- imidazo[4,5-b]pyridine- 7-carboxylic acid(2- chloro-4-methyl-pyridin- 3-yl)-amide | 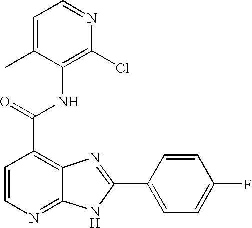 | 11.30 (s, 1H), 8.55 (d, 1H), 8.50 (m, 2H), 8.24 (d, 1H), 7.80 (d, 1H), 7.44 (m, 3H), 2.34 (s, 3H) | 381.8 |
| 316 | 2-(4-fluoro-phenyl)-3H- imidazo[4,5-b]pyridine- 7-carboxylic acid(6- chloro-5-methyl-pyridin- 3-yl)-amide | 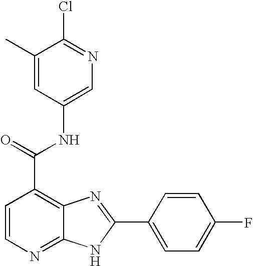 | 11.58 (s, 1H), 8.72 (s, 1H), 8.55 (d, 1H), 8.50 (m, 2H), 8.30 (s, 1H), 7.78 (m, 1H), 7.46 (m, 2H), 2.40 (s, 3H) | 381.8 |
| 317 | 2-(4-fluoro-phenyl)-3H- imidazo[4,5-b]pyridine- 7-carboxylic acid(2- chloro-5-methyl-pyridin- 3-yl)-amide | 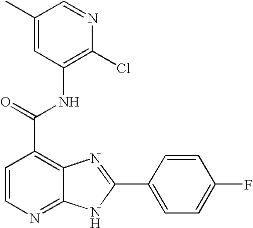 | 11.88 (s, 1H), 8.82 (s, 1H), 8.59 (d, 1H), 8.42 (m, 2H), 8.10 (s, 1H), 7.82 (d, 1H), 7.46 (m, 2H), 2.35 (s, 3H) | 381.8 |
| 318 | 2-(4-fluoro-phenyl)-3H- imidazo[4,5-b]pyridine- 7-carboxylic acid(2,5- dichloro-pyridin-3-yl)- amide | 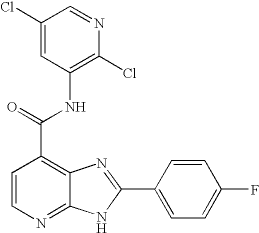 | 11.88 (s, 1H), 8.98 (s, 1H), 8.48 (s, 1H), 8.36 (m, 2H), 8.25 (s, 1H), 7.76 (s, 1H), 7.40 (m, 2H) | 402.2 |
| 319 | 2-(4-fluoro-phenyl)-3H- imidazo[4,5-b]pyridine- 7-carboxylic acid(4,6- dichloro-pyridin-5-yl)- amide | 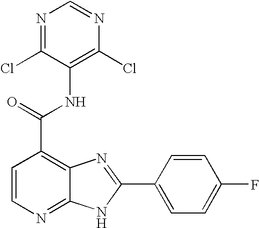 | 11.55 (s, 1H), 8.97 (s, 1H), 8.57 (d, 1H), 8.49 (m, 2H), 7.81 (d,1H), 7.43 (m, 2H) | 403.2 |
| 320 | 2-(4-fluoro-phenyl)-3H- imidazo[4,5-b]pyridine- 7-carboxylic acid(4- ethylsulfanyl-pyridin-3- yl)-amide | 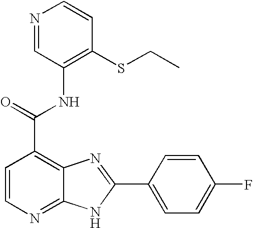 | 8.58 (d, 1H), 8.43 (m, 2H), 8.35 (m, 2H), 7.95 (d, 1H), 7.38 (m, 3H), 3.47 (q, 2H), 1.56 (t, 3H) | 393.4 |
| 321 | 2-(4-fluoro-phenyl)-3H- imidazo[4,5-b]pyridine- 7-carboxylic acid(6- dimethylamino-pyridin- 3-yl)-amide | 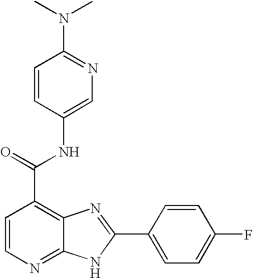 | 11.44 (s, 1H), 8.75 (s, 1H), 8.55 (d, 1H), 8.48 (m, 2H), 8.22 (m, 1H), 7.80 (d, 1H), 7.62 (m, 2H), 7.47 (m, 2H), 7.30 (d, 1H), 7.02 (d, 1H), 3.25 (s, 3H), 3.05 (s, 3H) | 376.4 |
| 322 | 2-(4-fluoro-phenyl)-3H- imidazo[4,5-b]pyridine- 7-carboxylic acid(6- acetylamino-pyridin-3- yl)-amide | 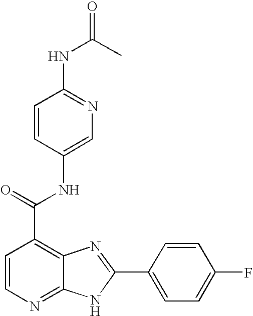 | 11.80 (s, 1H), 8.85 (s, 1H), 8.44 (d, 1H), 8.41 (m, 2H), 8.10 (m, 1H), 7.68 (d, 1H), 7.62 (m, 2H), 7.35 (m, 2H), 7.20 (d, 1H), 7.10 (d, 1H), 2.17 (s, 3H) | 390.4 |
| 323 | 2-(4-fluoro-phenyl)-3H- imidazo[4,5-b]pyridine- 7-carboxylic acid[6-(2- morpholin-4-yl-ethoxy)- pyridin-3-yl]-amide | 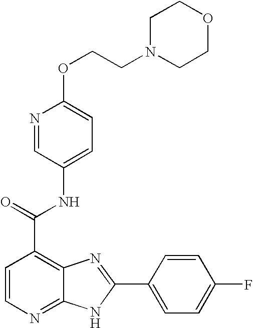 | 8.59 (br, 1H), 8.50 (d, 1H), 8.34 (m, 3H), 7.88~7.80 (m, 2H), 7.36 (t, 2H), 6.74 (d, 1H), 4.42 (t, 2H), 3.56 (t, 2H), 3.40~3.33 (m, 4H), 2.08 (m, 4H) | 462.5 |
| 324 | 2-(4-fluoro-phenyl)-3H- imidazo[4,5-b]pyridine- 7-carboxylic acid(4,6- dimethyl-pyridin-3-yl)- amide | 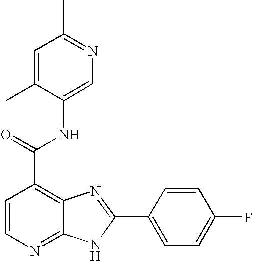 | 8.52 (d, 1H), 8.31 (d, 2H), 8.12 (s, 1H), 7.93 (d, 1H), 7.76 (m, 1H), 7.34 (m, 2H), 2.66 (s, 6H) | 361.4 |
| 325 | 2-(4-fluoro-phenyl)-3H- imidazo[4,5-b]pyridine- 7-carboxylic acid(4- ethoxy-6-methyl- pyridin-3-yl)-amide | 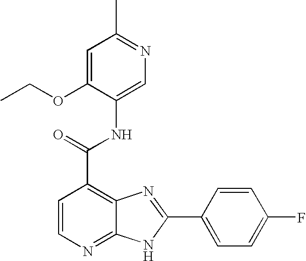 | 9.45 (br, 1H), 8.46 (d, 1H), 8.38 (m, 2H), 7.95 (d, 1H), 7.38 (t, 2H), 7.08 (s, 1H), 4.42 (q, 2H), 2.52 (s, 3H), 1.42 (t, 3H) | 391.4 |
| 326 | 2-(4-fluoro-phenyl)-3H- imidazo[4,5-b]pyridine- 7-carboxylic acid(4- ethylsulfanyl-6-methyl- pyridin-3-yl)-amide | 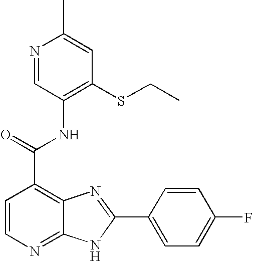 | 1.33 (t, 3H), 3.19 (s, 3H), 4.03 (s, 2H), 7.34 (s, 1H), 7.41 (t, 2H), 7.70 (s, 1H), 8.42 (s, 1H), 8.50 (t, 2H), 9.10 (s, 1H) | 407.5 |
| 327 | 2-[4-(4-methyl- piperazin-1-yl)-phenyl]- 3H-imidazo[4,5- b]pyridine-7-carboxylic acid pyridin-3-ylamide | 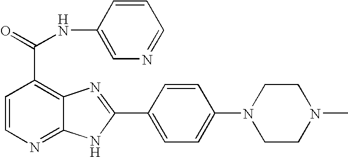 | 3.00 (s, 1H), 3.20~3.40 (m, 4H), 3.50~3.60 (m, 2H), 4.21-4.05 (m, 2H), 7.23 (d, 2H), 7.86 (d, 1H), 8.00 (m, 1H), 8.24 (d, 2H), 8.42 (d, 1H), 8.58 (br, 1H), 8.70 (d, 1H), 9.58 (br, 1H) | 413.5 |
| 328 | 2-(4- methanesulfinylmethyl- phenyl)-3H-imidazo[4,5- b]pyridine-7-carboxylic acid pyridin-3-ylamide | 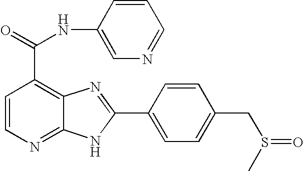 | 2.17 (s, 3H), 5.64 (s, 2H), 7.42 (t, 2H), 7.89 (br, 1H), 8.00 (d, 1H), 8.12~8.17 (m, 2H), 8.56~8.64 (m, 3H), 9.42 (br, 1H) | 391.5 |
| 329 | 2-(4-diethylamino- phenyl)-3H-imidazo[4,5- b]pyridine-7-carboxylic acid pyridin-3-ylamide | 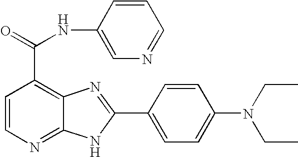 | 1.25 (t, 6H), 3.56 (q, 4H), 6.95 (d, 2H), 7.85 (d, 1H), 8.00 (br, 1H), 8.16 (d, 2H), 8.50 (m, 1H), 8.61 (br, 1H), 8.70 (d, 1H), 9.51 (br, 1H), | 386.5 |
| 330 | 2-(4-morpholin-4-yl- phenyl)-3H-imidazo[4,5- b]pyridine-7-carboxylic acid pyridin-3-ylamide | 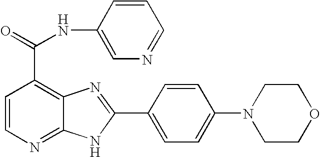 | 3.36 (m, 4H), 3.87 (m, 4H), 7.14 (d, 2H), 7.87 (d, 1H), 8.10 (m, 1H), 8.21 (d, 2H), 8.60~8.40 (m, 2H), 8.72 (d, 1H), 9.61 (br, 1H) | 400.4 |
| 331 | 2-(4-piperidin-1-yl- phenyl)-3H-imidazo[4,5- b]pyridine-7-carboxylic acid pyridin-3-ylamide | 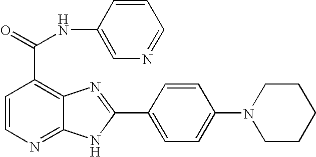 | 1.41~1.25 (m, 6H), 3.13 (m, 4H), 6.95 (d, 2H), 7.51 (d, 1H), 7.65 (m, 1H), 7.88 (d, 2H), 8.18 (d, 1H), 8.23 (m, 1H), 8.38 (d, 1H), 9.22 (s, 1H) | 398.5 |
| 332 | 2-(4-pyrrolidin-1-yl- phenyl)-3H-imidazo[4,5- b]pyridine-7-carboxylic acid(4-methyl-pyridin-3- yl)-amide | 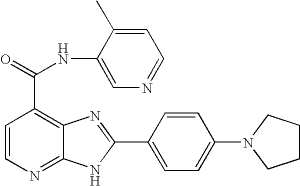 | 2.09 (m, 4H), 3.40 (m, 4H), 6.70 (d, 2H), 7.85 (d, 1H), 8.03 (m, 3H), 8.38 (m, 1H), 8.50 (m, 1H), 9.95 (br, 1H) | 398.5 |
| 333 | 2-(4-pyrrolidin-1-yl- phenyl)-3H-imidazo[4,5- b]pyridine-7-carboxylic acid(4-methyl-pyridin-3- yl)-amide | 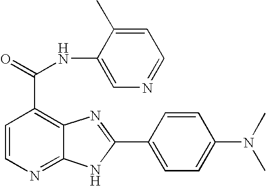 | 2.82 (s, 6H), 3.23 (s, 3H), 6.85 (d, 1H), 7.37 (t, 1H), 7.84~8.10 (m, 4H), 8.27 (m, 1H), 8.38 (br, 1H), 8.56 (m, 1H) | 372.4 |
| 334 | 2-(4-morpholin-4-yl- phenyl)-3H-imidazo[4,5- b]pyridine-7-carboxylic acid(4-methyl-pyridin-3- yl)-amide | 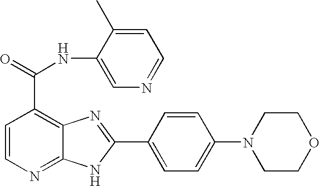 | 2.97 (s, 3H), 3.39 (m, 4H), 7.20 (d, 2H), 7.99 (d, 1H), 8.16 (d, 1H), 8.19 (d, 2H), 8.58 (m, 2H), 9.80 (s, 1H) | 414.5 |
| 335 | 2-(4-piperidin-1-yl- phenyl)-3H-imidazo[4,5- b]pyridine-7-carboxylic acid(4-methyl-pyridin-3- yl)-amide | 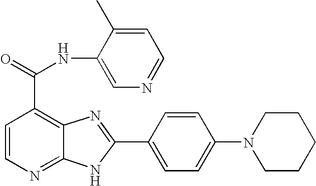 | 1.65~1.80 (m, 6H), 2.13 (m, 4H), 2.65 (s, 3H), 7.10 (d, 2H), 7.89 (d, 1H), 7.99 (m, 1H), 8.08 (d, 2H), 8.20~8.40 (m, 3H), 9.90 (br, 1H) | 412.5 |
| 336 | 2-(4-dimethylamino- phenyl)-3H-imidazo[4,5- b]pyridine-7-carboxylic acid pyridin-3-ylamide | 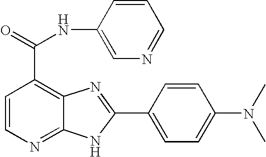 | 3.12 (s, 6H), 6.93 (d, 2H), 7.86 (d, 1H), 7.97 (m, 1H), 8.17 (d, 2H), 8.40~8.70 (m, 3H), 9.58 (br, 1H) | 358.4 |
| 337 | 2-[4-(4-methyl- piperazin-1-yl)-phenyl]- 3H-imidazo[4,5- b]pyridine-7-carboxylic acid(4-methyl-pyridin-3- yl)-amide | 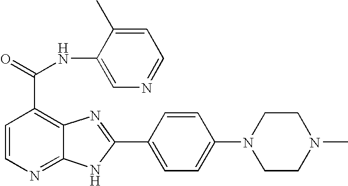 | 2.92 (s, 3H), 3.00 (s, 3H), 3.10~3.40 (bs, 4H), 3.64 (bs, 2H), 4.09 (bs, 2H), 7.17 (d, 2H), 7.84 (d, 1H), 8.00 (bs, 1H), 8.10 (d, 2H), 8.40 (s, 1H), 8.52 (bs, 1H), 9.92 (bs, 1H) | 427.5 |
| 338 | 2-[4-(2-morpholin-4-yl- ethylamino)-phenyl]-3H- imidazo[4,5-b]pyridine- 7-carboxylic acid(4- methyl-pyridin-3-yl)- amide | 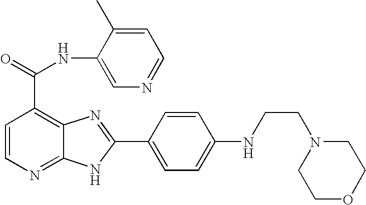 | 3.01 (s, 3H), 3.31~3.20 (m, 4H), 3.54 (t, 2H), 3.71 (t, 2H), 4.10~3.80 (m, 4H), 6.90 (d, 2H), 7.92 (d, 1H), 8.01 (d, 1H), 8.18 (d, 2H), 8.42 (d, 1H), 8.58 (m, 1H), 9.95 (s, 1H) | 457.5 |
| 339 | 2-[4-(3-morpholin-4-yl- propylamino)-phenyl]- 3H-imidazo[4,5- b]pyridine-7-carboxylic acid(4-methyl-pyridin-3- yl)-amide | 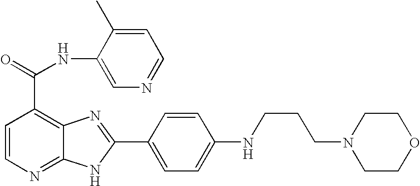 | 2.98 (s, 3H), 4.18~3.45 (m, 8H), 6.84 (d, 2H), 7.86 (d, 1H), 8.04 (m, 3H), 8.72~8.30 (m, 2H), 9.98 (br, 1H) | 471.6 |
| 340 | 2-[4-(3-pyrrolidin-1-yl- propylamino)-phenyl]- 3H-imidazo[4,5- b]pyridine-7-carboxylic acid(4-methyl-pyridin-3- yl)-amide | 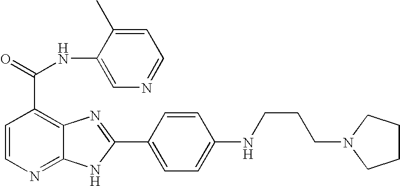 | 2.23~1.98 (m, 4H), 3.06 (s, 3H), 3.15 (m, 2H), 3.70 (m, 2H) 6.80 (d, 2H), 7.89 (d, 1H), 8.10~7.90 (m, 3H, 8.40 (d, 1H), 8.52 (d, 1H), 9.92 (s, 1H) | 455.6 |
| 341 | 2-[4-(2-dimethylamino- ethylamino)-phenyl]-3H- imidazo[4,5-b]pyridine- 7-carboxylic acid(4- methyl-pyridin-3-yl)- amide | 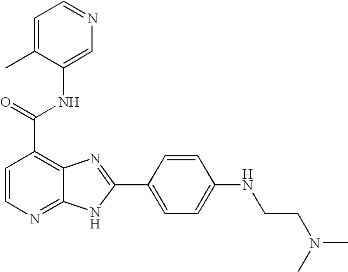 | 2.03 (s, 2H), 2.64 (s, 3H), 2.69 (t, 2H), 6.73 (d, 2H), 7.39 (bs, 1H), 7.80 (d, 1H), 7.97 (d, 2H), 8.24 (bs, 1H), 8.31 (d, 1H), 9.49 (s, 1H) | 415.5 |
| 342 | 2-[4-(3-dimethylamino- propylamino)-phenyl]- 3H-imidazo[4,5- b]pyridine-7-carboxylic acid(4-methyl-pyridin-3- yl)-amide | 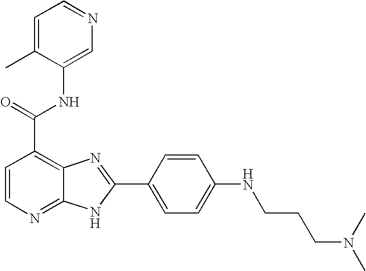 | 2.08 (m, 2H), 2.90-2.94 (m, 9H), 3.25-3.36 (m, 4H), 6.79 (d, 2H), 7.86 (d, 1H), 8.01-8.04 (m, 3H), 8.39 (bs, 1H), 8.6 (bs, 1H), 9.90 (bs, 1H) | 429.5 |
| 343 | 2-[4-(4-dimethylamino- butylamino)-phenyl]-3H- imidazo[4,5-b]pyridine- 7-carboxylic acid(4- methyl-pyridin-3-yl)- amide | 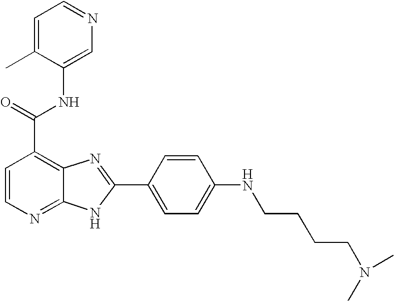 | 1.66 (bd, 2H), 2.01 (m, 2H), 2.36 (t, 2H), 3.21 (m, 2H), 6.70 (t, 2H), 7.41 (d, 1H), 7.78 (d, 1H), 8.02 (d, 1H), 8.24 (m, 2H), 8.30 (d, 1H), 9.49 (bd, 1H) | 443.6 |
| 344 | 2-{4-[(2-diethylamino- ethyl)-methyl-amino]- phenyl}-3H- imidazo[4,5-b]pyridine- 7-carboxylic acid(4- methyl-pyridin-3-yl)- amide | 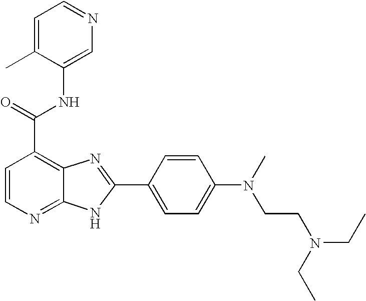 | 1.27 (t, 6H), 1.98-2.03 (m, 3H), 2.58 (bs, 3H), 2.90- 3.12 (m, 6H), 3.34 (bs, 2H), 3.70 (ds, 2H), 6.79 (bs, 2H), 7.37 (bs, 1H), 7.76 (bs, 1H), 7.92 (bs, 2H), 8.24 (bs, 2H), 9.44 (bs, 1H) | 457.6 |
| 345 | 2-[4-(4-methyl- piperazin-1-yl)-phenyl]- 3H-imidazo[4,5- b]pyridine-7-carboxylic acid(1,7,7-trimethyl- bicyclo[2.2.1]heptin-2- yl)-amide | 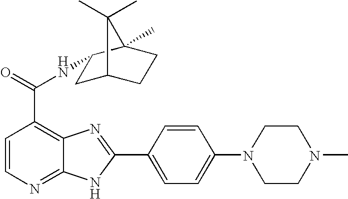 | CD₃OD: 0.95 (s, 3H), 0.96 (s, 3H), 1.23 (s, 3H), 1.15- 2.12 (m, 1H), 1.45-1.63 (m, 3H), 2.12-2.19 (m, 4H), 3.00 (s, 3H), 3.04-3.14 (m, 4H), 4.48-4.54 (m, 1H), 6.89 (d, J = 3.6 Hz, 2H), 7.69 (d, J = 2.4 Hz, 1H), 8.02 (d, J = 3.6 Hz, 2H), 8.30 (d, J = 2.4 Hz, 1H) | 472.6 |
| 346 | 2-(4-morpholin-4-yl- phenyl)-3H-imidazo[4,5- b]pyridine-7-carboxylic acid(1,7,7-trimethyl- bicyclo[2.2.1]heptin-2- yl)-amide | 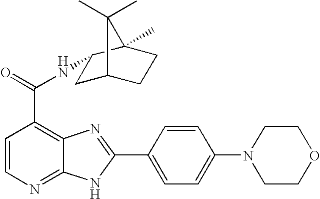 | CD₃OD: 0.99 (s, 3H), 1.01 (s, 3H), 1.23 (s, 3H), 1.17- 2.18 (m, 1H), 1.35-1.62 (m, 3H), 2.10-2.30 (b, 4H), 3.14-3.23 (b, 4H), 4.42- 4.51 (m, 1H), 6.99 (d, J = 3.6 Hz, 2H), 7.80 (d, J = 2.4 Hz, 1H), 8.02 (d, J = 3.6 Hz, 2H), 8.33 (d, J = 2.4 Hz, 1H) | 459.6 |
| 347 | 2-[4-(piperidin-4- ylamino)-phenyl]-3H- imidazo[4,5-b]pyridine- 7-carboxylic acid(1,7,7- trimethyl- bicyclo[2.2.1]heptin-2- yl)-amide | 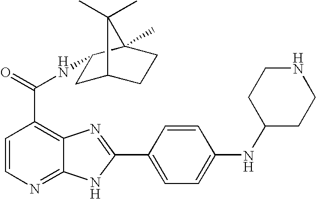 | 0.96 (s, 3H), 0.99 (s, 3H), 1.13 (s, 3H), 1.17- 2.02 (m, 9H), 1.41-1.63 (m, 3H), 1.78~2.02 (m, 3H), 4.48-4.54 (m, 1H), 6.82 (d, 2H), 7.79 (d, 1H), 8.12 (d, 2H), 8.40 (d, 1H) | 472.6 |
| 348 | 2-[4-(4-methyl- piperidin-1-yl)-phenyl]- 3H-imidazo[4,5- b]pyridine- 7-carboxylic acid adamantan-2- ylamide | 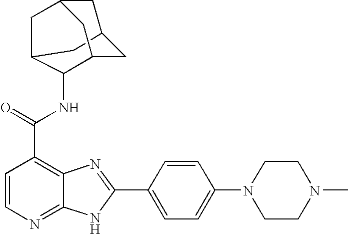 | CDCl₃: 1.25-2.01 (m, 15H), 2.24-2.43 (b, 4H), 2.88 (s, 3H), 3.43-3.67 (b, 4H), 6.83-7.78 (m, 4H), 8.15- 8.23 (m, 3H), 8.39-8.51 (b, 1H) | 470.6 |
| 349 | 2-(4-morpholin-4-yl- phenyl)-3H-imidazo[4,5- b]pyridine- 7-carboxylic acid adamantan-2- ylamide | 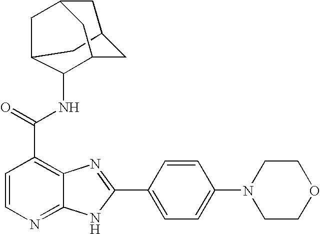 | CDCl₃: 1.22-1.90 (m, 15H), 2.45-2.56 (b, 4H), 3.51- 3.57 (b, 4H), 7.23-7.56 (m, 4H), 8.25-8.28 (m, 3H), 8.31-8.45 (b, 1H) | 457.6 |
| 350 | 2-[4-(4-methyl- piperidin-1-yl)-phenyl]- 3H-imidazo[4,5- b]pyridine- 7-carboxylic acid cycloheptylamide | 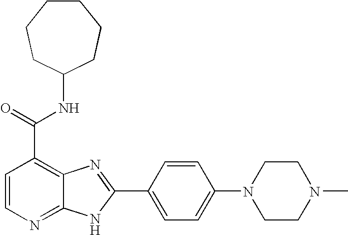 | CD₃OD: 1.34-1.99 (m, 12H), 2.34-2.43 (b, 4H), 2.98 (s, 3H), 3.32-3.56 (b, 4H), 4.24-4.28 (m, 1H), 6.82 (d, 3.6 Hz, 2H), 7.21 (d, 2.1 Hz, 1H), 7.82 (d, 3.6 Hz, 2H), 8.29 (d, 2.1 Hz, 1H) | 432.6 |
| 351 | 2-(4-morpholin-4-yl- phenyl)-3H-imidazo[4,5- b]pyridine- 7-carboxylic acid cycloheptylamide | 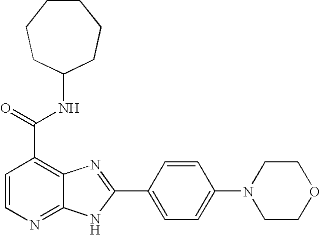 | CD₃OD:1.37-1.99 (m, 12H), 2.45-2.66 (b, 4H), 3.52- 3.63 (b, 4H), 4.21-4.28 (m, 1H), 6.92 (d, 3.4 Hz, 2H), 7.23 (d, 2.4 Hz, 1H), 7.79 (d, 3.4 Hz, 2H), 8.38 (d, 2.4 Hz, 1H) | 419.5 |
| 352 | 2-(4-thiomorpholin-4- yl-phenyl)-3H- imidazo[4,5-b]pyridine- 7-carboxylic acid cycloheptylamide | 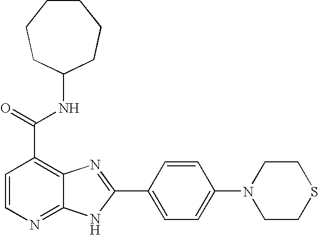 | CD₃OD: 1.37-1.99 (m, 12H), 2.45-2.66 (b, 4H), 3.52- 3.63 (b, 4H), 4.21-4.28 (m, 1H), 6.92 (d, 3.4 Hz, 2H), 7.23 (d, 2.4 Hz, 1H), 7.79 (d, 3.4 Hz, 2H), 8.38 (d, 2.4 Hz, 1H) | 435.6 |
| 353 | 2-[4-(4-methyl- piperidin-1-yl)-phenyl]- 3H-imidazo[4,5- b]pyridine- 7-carboxylic acid(1-aza- bicyclo[2.2.2]octyn-3- yl)-amide | 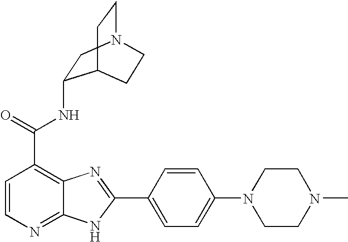 | CD₃OD: 2.18-2.24 (m, 3H), 2.40-2.53 (m, 2H), 3.00 (s, 3H), 3.20-3.64 (m, 11H), 3.92-3.99 (m, 1H), 4.02- 4.22 (m, 1H), 4.61-4.31 (m, 1H), 7.23 (d, J = 3.4 Hz, 2H), 7.78 (d, J = 2.4 Hz, 1H), 8.18 (d, J = 3.4 Hz, 2H), 8.41 (d, J = 2.4 Hz, 1H) | 445.6 |
| 354 | 2-[4-(piperidin-4- ylamino)-phenyl]-3H- imidazo[4,5-b]pyridine- 7-carboxylic acid cycloheptylamide | 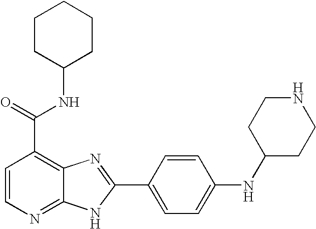 | CD₃OD: 1.36-1.66 (m, 5H), 1.62-1.79 (m, 5H), 2.02- 2.19 (m, 4H), 2.95-3.02 (m, 2H), 3.36-3.61 (m, 1H), 4.01-4.18 (m, 3H), 7.18 (d, J = 3.4 Hz, 2H), 7.76 (d, J = 2.4 Hz, 1H), 8.14 (d, J = 3.4 Hz, 2H), 8.41 (d, J = 2.4 Hz, 1H) | 418.5 |
| 355 | 2-[4-(2-piperazin-1-yl- ethylamino)-phenyl]-3H- imidazo[4,5-b]pyridine- 7-carboxylic acid cycloheptylamide | 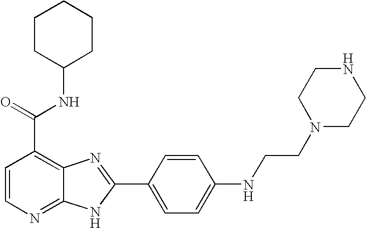 | CD₃OD: 1.46-1.60 (m, 6H), 1.82-1.95 (m, 4H), 1.98- 2.19 (m, 4H), 2.99-3.07 (m, 2H), 3.39 (t, 2.8 Hz, 2H), 3.59-3.61 (m, 1H), 3.64 (t, 2.8 Hz, 2H), 4.01- 4.18 (m, 1H), 6.81 (d, J = 3.4 Hz, 2H), 7.76 (d, J = 2.4 Hz, 1H), 8.13 (d, J = 3.4 Hz, 2H), 8.40 (d, J = 2.4 Hz, 1H) | 447.6 |
| 356 | 2-[4-(2-morpholin-4-yl- ethylamino)-phenyl]-3H- imidazo[4,5-b]pyridine- 7-carboxylic acid cycloheptylamide | 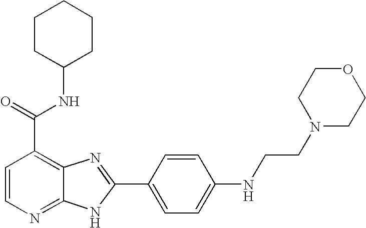 | CD₃OD: 1.46-1.60 (m, 6H), 1.80-1.95 (m, 2H), 2.00- 2.19 (m, 2H), 2.99-3.60 (m, 4H), 3.41 (t, 2.8 Hz, 2H), 3.64 (t, 2.8 Hz, 2H), 3.80- 4.15 (m, 5H), 6.91 (d, J = 3.4 Hz, 2H), 7.77 (d, J = 2.4 Hz, 1H), 8.10 (d, J = 3.4 Hz, 2H), 8.39 (d, J = 2.4 Hz, 1H) | 448.6 |
| 357 | 2-(4-morpholin-4-yl- phenyl)-3H-imidazo[4,5- b]pyridine-7-carboxylic acid cyclohexylamide | 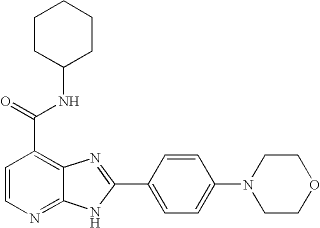 | CD₃OD: 1.34-1.62 (m, 5H), 1.60-1.73 (m, 5H), 2.12- 2.19 (m, 4H), 2.90-3.12 (m, 2H), 3.46-3.61 (m, 1H), 4.11-4.18 (m, 3H), 7.18 (d, J = 3.4 Hz, 2H), 7.76 (d, J = 2.4 Hz, 1H), 8.14 (d, J = 3.4 Hz, 2H), 8.41 (d, J = 2.4 Hz, 1H) | 405.5 |
| 358 | 2-[4-(4-methyl- piperazin-1-yl)-phenyl]- 3H-imidazo[4,5- b]pyridine-7-carboxylic acid cyclohexylamide | 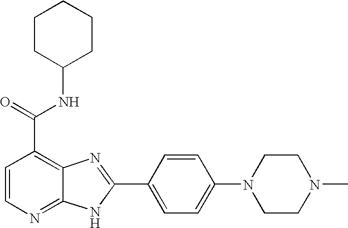 | CD₃OD: 1.39-1.65 (m, 6H), 1.80-2.19 (m, 4H), 3.00 (s, 3H), 3.19-3.44 (b, 4H), 3.52-3.69 (m, 2H), 4.05- 4.19 (b, 3H), 7.21 (d, J = 3.4 Hz, 2H), 7.77 (d, J = 2.4 Hz, 1H), 8.19 (d, J = 3.4 Hz, 2H), 8.42 (d, J = 2.4 Hz, 1H) | 418.5 |
| 359 | 2-[4-(2-dimethylamino- ethylamino)-phenyl]-3H- imidazo[4,5-b]pyridine- 7-carboxylic acid cyclohexylamide | 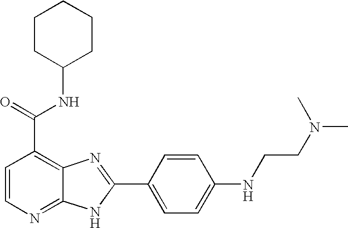 | CD₃OD: 1.47-1.590 (m, 6H), 1.81-1.92 (m, 2H), 2.01- 2.13 (m, 2H), 2.99 (s, 6H), 3.41 (t, 2.8 Hz, 2H), 3.63 (t, 2.8 Hz, 2H), 4.01-4.18 (m, 1H), 6.91 (d, J = 3.4 Hz, 2H), 7.78 (d, J = 2.4 Hz, 1H), 8.13 (d, J = 3.4 Hz, 2H), 8.41 (d, J = 2.4 Hz, 1H) | 406.5 |
| 360 | 2-{4-[(2-diethylamino- ethyl)-methyl-amino]- phenyl}-3H- imidazo[4,5-b]pyridine- 7-carboxylic acid cyclohexylamide | 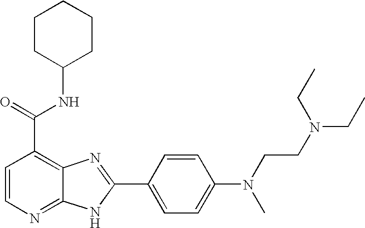 | CD₃OD: 1.39 (t, 2.8 Hz, 6H), 1.46-1.71 (m, 6H), 1.81- 1.92 (m, 2H), 2.01-2.13 (m, 2H), 3.19 (s, 3H), 3.31- 3.40 (m, 6H), 3.93 (t, 2.8 Hz, 2H), 4.05-4.17 (m, 1H), 7.01 (d, J = 3.4 Hz, 2H), 7.77 (d, J = 2.4 Hz, 1H), 8.16 (d, J = 3.4 Hz, 2H), 8.39 (d, J = 2.4 Hz, 1H) | 448.6 |
| 361 | 2-[4-(4-ethyl-piperazin- 1-yl)-phenyl]-3H- imidazo[4,5-b]pyridine- 7-carboxylic acid cyclohexylamide | 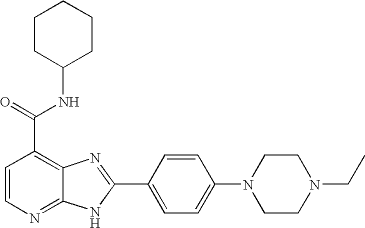 | CD₃OD: 1.39-1.69 (m, 9H), 1.80-2.19 (m, 4H), 3.33 (s, 3H), 3.19-3.39 (b, 4H), 3.62-3.80 (m, 2H), 4.02- 4.17 (b, 3H), 7.20 (d, J = 3.4 Hz, 2H), 7.79 (d, J = 2.4 Hz, 1H), 8.18 (d, J = 3.4 Hz, 2H), 8.41 (d, J = 2.4 Hz, 1H) | 432.6 |
| 362 | 2-(4-morpholin-4-yl- phenyl)-3H-imidazo[4,5- b]pyridine-7-carboxylic acid(1-aza- bicyclo[2.2.2]octyn-3- yl)-amide | 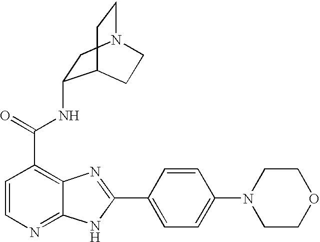 | CD₃OD: 2.11-2.25 (m, 3H), 2.42-2.51 (m, 2H), 3.19- 3.54 (m, 10H), 3.91-3.95 (m, 1H), 4.06-4.22 (m, 1H), 4.61-4.31 (m, 1H), 7.23 (d, J = 3.4 Hz, 2H), 7.78 (d, J = 2.4 Hz, 1H), 8.18 (d, J = 3.4 Hz, 2H), 8.41 (d, J = 2.4 Hz, 1H) | 432.5 |
| 363 | 2-[4-(2-morpholin-4-yl- ethylamino)-phenyl]-3H- imidazo[4,5-b]pyridine- 7-carboxylic acid(2,6,6- trimethyl- bicyclo[3.1.1]heptin-3- yl)-amide | 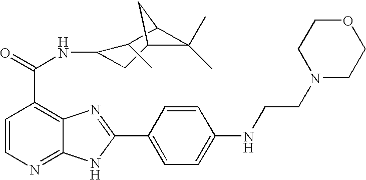 | CD₃OD: 1.17 (m, 3H), 1.23 (d, 2.6 Hz, 3H), 1.27- 1.38 (m, 1H), 1.37 (s, 3H), 1.79-1.83 (m, 1H), 1.92- 1.94 (m, 1H), 2.01-2.10 (m, 1H), 2.12-2.21 (m, 1H), 2.56-2.61 (m, 1H), 2.69- 2.81 (m, 1H), 3.24-3.60 (b, 4H), 3.23 (t, J = 2.5 Hz, 2H), 3.63 (t, J = 2.5 Hz, 2H), 3.90- 4.09 (b, 4H), 4.62-4.65 (m, 1H), 6.93 (d, J = 3.4 Hz, 2H), 7.79 (d, J = 2.4 Hz, 1H), 8.11 (d, J = 3.4 Hz, 2H), 8.40 (d, J = 2.4 Hz, 1H) | 502.7 |
| 364 | 2-[4-(2-morpholin-4-yl- ethylamino)-phenyl]-3H- imidazo[4,5-b]pyridine- 7-carboxylic acid bicyclo[2.2.1]heptin-2- ylamide | 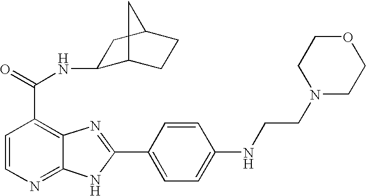 | CD₃OD: 1.03-1.15 (m, 2H), 1.47-1.52 (m, 4H), 1.64- 2.02 (m, 4H), 2.22-2.24 (m, 1H), 2.31-2.35 (m, 1H), 2.53-2.57 (m, 1H), 2.55- 2.59 (m, 1H), 3.22-3.25 (m, 2H), 3.29-3.60 (b, 4H), 3.43 (t, J = 3.5 Hz, 2H), 3.68 (t, J = 3.5 Hz, 2H), 3.80-4.00 (b, 4H), 4.42-4.45 (m, 1H), 6.91 (d, J =3.4 Hz, 2H), 7.77 (d, J = 2.4 Hz, 1H), 8.17 (d, J = 3.4 Hz, 2H), 8.39 (d, J = 2.4 Hz, 1H) | 460.6 |
| 365 | 2-[4-(2-morpholin-4-yl- ethylamino)-phenyl]-3H- imidazo[4,5-b]pyridine- 7-carboxylic acid(2,6- dimethyl-phenyl)-amide | 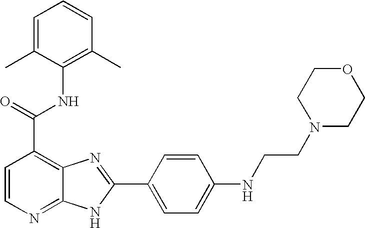 | CD₃OD: 2.59 (s, 6H), 3.42 (t, J = 3.6 Hz, 2H), 3.40-3.45 (m, 4H), 3.63 (t, J = 3.6 Hz, 2H), 3.71-4.02 (m, 4H), 6.92 (d, J = 3.6 Hz, 2H), 7.90 (d, J = 2.4 Hz, 1H), 8.18 (d, J = 3.6 Hz, 2H), 8.45 (d, J = 2.4 Hz, 1H) | 470.6 |
| 366 | 2-[4-(2-piperidin-1-yl- ethylamino)-phenyl]-3H- imidazo[4,5-b]pyridine- 7-carboxylic acid(1,7,7- trimethyl- bicyclo[2.2.1]heptin-2- yl)-amide | 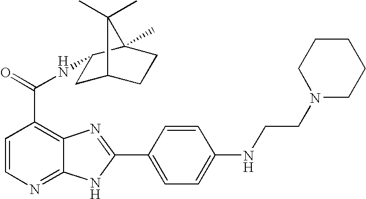 | CD₃OD: 0.96 (s, 3H), 0.99 (s, 3H), 1.13 (s, 3H), 1.17- 2.02 (m, 1H), 1.41-1.63 (m, 3H), 1 .78-2.02 (m, 7H), 2.12-2.19 (m, 1H), 2.43- 2.59 (m, 1H), 3.04-3.14 (m, 2H), 3.39 (t, J = 3.0 Hz, 2H), 3.59-3.62 (m, 2H), 4.48- 4.54 (m, 1H), 6.82 (d, J = 3.6 Hz, 2H), 7.79 (d, J = 2.4 Hz, 1H), 8.12 (d, J = 3.6 Hz, 2H), 8.40 (d, J = 2.4 Hz, 1H) | 500.7 |
| 367 | 2-[4-(2-morpholin-4-yl- ethylamino)-phenyl]-3H- imidazo[4,5-b]pyridine- 7-carboxylic acid(4- hydroxy-cyclohexyl)- amide | 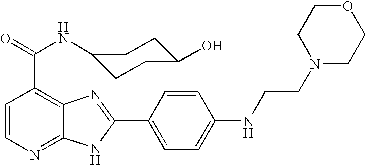 | CD₃OD: 1.53-1.59 (m, 4H), 2.09-2.23 (m, 4H), 3.42- 3.50 (m, 4H), 3.42 (t, J = 2.1 Hz, 2H), 3.71 (t, J = 2.1 Hz, 2H), 3.79-4.12 (m, 6H), 6.92 (d, J = 3.4 Hz, 2H), 7.77 (d, J = 2.4 Hz, 1H), 8.16 (d, J = 3.4 Hz, 2H), 8.41 (d, J = 2.4 Hz, 1H) | 464.6 |
| 368 | 2-[4-(2-morpholin-4-yl- ethylamino)-phenyl]-3H- imidazo[4,5-b]pyridine- 7-carboxylic acid(1,7,7- trimethyl- bicyclo[2.2.1]heptin-2- yl)-amide | 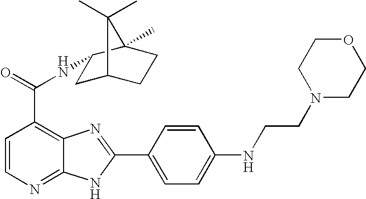 | CD₃OD: 0.93 (s, 3H), 1.01 (s, 3H), 1.10 (s, 3H), 1.15- 1.16 (m, 1H), 1.42-1.62 (m, 2H), 1.78-1.81 (m, 1H), 1.88-2.19 (m, 2H), 2.47- 2.59 (m, 1H), 3.24-3.44 (b, 4H), 3.44 (t, J = 3.2 Hz, 2H), 3.63 (t, J = 3.2 Hz, 2H), 4.48- 4.54 (m, 1H), 6.85 (d, J = 3.4 Hz, 2H), 7.77 (d, J = 2.4 Hz, 1H), 8.13 (d, J = 3.4 Hz, 2H), 8.42 (d, J = 2.4 Hz, 1H) | 502.7 |
| 369 | 2-[4-(2-piperazin-1-yl- ethylamino)-phenyl]-3H- imidazo[4,5-b]pyridine- 7-carboxylic acid(1,7,7- trimethyl- bicyclo[2.2.1]heptin-2- yl)-amide | 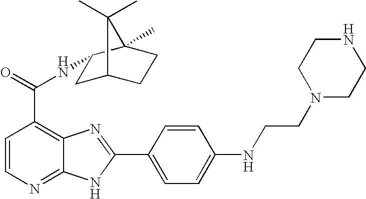 | CD₃OD: 0.93 (s, 3H), 1.01 (s, 3H), 1.10 (s, 3H), 1.19- 1.21 (m, 1H), 1.44-1.69 (m, 2H), 1.78-1.82 (m, 1H), 1.88-2.29 (m, 2H), 2.49- 2.59 (m, 1H), 3.24-3.44 (m, 6H), 3.24 (t, J = 3.2 Hz, 2H), 3.43-3.46 (b, 4H), 4.48- 4.54 (m, 1H), 7.19 (d, J = 3.1 Hz. 2H), 7.80 (d, J = 2.4 Hz, 1H), 8.19 (d, J = 3.4 Hz, 2H), 8.40 (d, J = 2.4 Hz, 1H) | 501.7 |
| 370 | 2-[4-(2-dimethylamino- ethylamino)-phenyl]-3H- imidazo[4,5-b]pyridine- 7-carboxylic acid(1,7,7- trimethyl- bicyclo[2.2.1]heptin-2- yl)-amide | 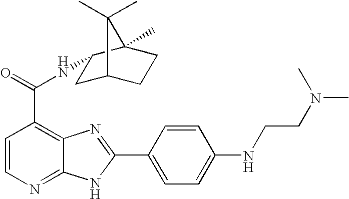 | CD₃OD: 0.92 (s, 3H), 1.00 (s, 3H), 1.09 (s, 3H), 1.19- 1.20 (m, 1H), 1.42-1.63 (m, 2H), 1.79-1.82 (m, 1H), 1.90-2.19 (m, 2H), 2.46- 2.59 (m, 1H), 2.99 (s, 6H), 3.41 (t, J = 3.6 Hz, 2H), 3.63 (t, J = 3.6 Hz, 2H), 4.48- 4.59 (m, 1H), 6.98 (d, J = 3.4 Hz, 2H), 7.79 (d, J = 2.4 Hz. 1H), 8.12 (d, J = 3.4 Hz, 2H), 8.40 (d, J = 2.4 Hz, 1H) | 460.6 |
| 371 | 2-[4-(4-methyl- piperazin-1-yl)-phenyl]- 3H-imidazo[4,5- b]pyridine-7-carboxylic acid bicyclo[2.2.1]heptin-2- ylamide | 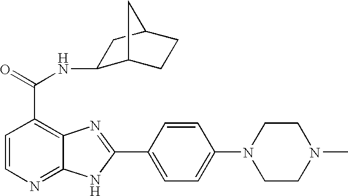 | CD₃OD: 1.18-1.24 (m, 1H), 1.44-1.61 (m, 4H), 1.69- 1.80 (m, 1H), 1.69-1.80 (m, 1H), 1.89-2.01 (m, 1H), 2.21-2.31 (m, 1H), 2.41- 2.43 (m, 1H), 2.59-2.60 (m, 1H), 3.00 (s, 3H) 3.14- 3.40 (m, 4H), 4.48-4.54 (m, 1H), 7.21 (d, J = 3.6 Hz, 2H), 7.79 (d, J = 2.4 Hz, 1H), 8.20 (d, J = 3.6 Hz, 2H), 8.42 (d, J = 2.4 Hz, 1H) | 430.6 |
| 372 | 2-[4-(2-dimethylamino- ethylamino)-phenyl]-3H- imidazo[4,5-b]pyridine- 7-carboxylic acid bicyclo[2.2.1]heptin-2- ylamide | 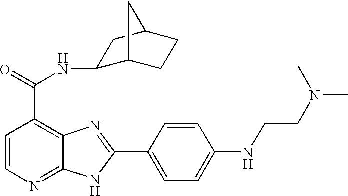 | CD₃OD: 1.25-1.27 (m, 1H), 1.43-1.45 (m, 2H), 1.60- 1.62 (m, 2H), 1.71-1.74 (m, 1H), 1.91-1.94 (m, 1H), 2.25-2.27 (m, 1H), 2.35- 2.38 (m, 1H), 2.59-2.60 (m, 1H), 2.99 (s, 6H), 3.41 (t, J = 2.4 Hz. 2H), 3.64 (t, J = 2.4 Hz, 2H), 6.92 (d, J = 3.4 Hz, 2H), 7.79 (d, J = 2.4 Hz, 1H), 8.11 (d, J = 3.4 Hz, 2H), 8.39 (d, J = 2.4 Hz, 1H) | 418.5 |
| 373 | 2-[4-(2-piperidin-1-yl- ethylamino)-phenyl]-3H- imidazo[4,5-b]pyridine- 7-carboxylic acid bicyclo[2.2.1]heptin-2- ylamide | 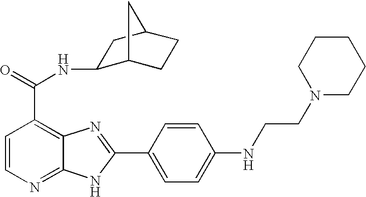 | CD₃OD: 1.23-1.25 (m, 2H), 1.49-1.52 (m, 4H), 1.74- 1.82 (m, 4H), 1.91-1.94 (m, 3H), 2.21-2.23 (m, 1H), 2.35-2.37 (m, 1H), 2.55-2.59 (m, 1H), 3.02-3.05 (m, 2H), 3.39 (t, J = 3.1 Hz, 2H), 3.72 (t, J = 3.1 Hz, 2H), 4.39-4.42 (m, 1H), 6.95 (d, J = 3.4 Hz, 2H), 7.79 (d, J = 2.4 Hz, 1H), 8.13 (d, J = 3.4 Hz, 2H), 8.35 (d, J = 2.4 Hz, 1H) | 458.6 |
| 374 | 2-[4-(2-dimethylamino- ethylamino)-phenyl]-3H- imidazo[4,5-b]pyridine- 7-carboxylic acid bicyclo[2.2.1]heptin-2- ylamide | 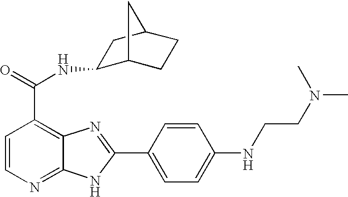 | CD₃OD + CDCl₃: 1.15- 1.17 (m, 3H), 1.38-1.71 (m, 4H), 1.98-2.03 (m, 1H), 2.42 (s, 2H), 3.14 (d, J = 3.1 Hz, 1H), 3.61 (d, J = 3.1 Hz, 1H), 3.99-4.01 (m, 1H), 6.94 (d, J = 3.4 Hz, 1H), 7.67 (d, J = 2.4 Hz, 1H) 8.16 (t, J = 3.4 Hz, 2H), 8.28 (d, J = 2.4 Hz, 1H) | 418.5 |
| 375 | 2-[4-(2-dimethylamino- ethylamino)-phenyl]-3H- imidazo[4,5-b]pyridine- 7-carboxylic acid(3- amino-cyclohexyl)-amide | 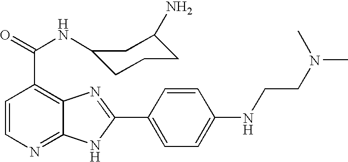 | CD₃OD: 1.60-2.28 (m, 8H), 3.39 (s, 6H), 3.43 (t, 1.2 Hz, 2H), 3.62 (t, 1.2 Hz, 2H), 4.43-4.44 (m, 1H), 6.86 (d, J = 3.4 Hz, 2H), 7.75 (d, J = 2.4 Hz, 1H), 8.09 (d, J = 3.4 Hz, 2H), 8.39 (d, J = 2.4 Hz, 1H) | 421.5 |
| 376 | 2-[4-(2-dimethylamino- ethylamino)-phenyl]-3H- imidazo[4,5-b]pyridine- 7-carboxylic acid(1,7,7- trimethyl- bicyclo[2.2.1]heptin-2- yl)-amide | 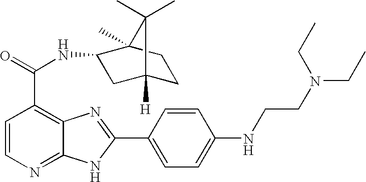 | CD₃OD: 0.93 (s, 3H), 0.97 (s, 3H), 1.12 (s, 9H), 1.05- 1.08 (m, 1H), 1.21-1.64 (m, 4H), 1.77-1.79 (m, 1H), 1.82-2.19 (m, 3H), 2.43- 2.63 (m, 6H), 4.42-4.47 (m, 1H), 6.72 (d, J = 3.4 Hz, 2H), 7.79 (d, J = 2.4 Hz, 1H), 8.01 (d, J = 3.4 Hz, 2H), 8.31 (d, J = 2.4 Hz, 1H) | 488.7 |
| 377 | 2-[4-(3-dimethylamino- propylamino)-phenyl]- 3H-imidazo[4,5- b]pyridine-7-carboxylic acid(1,7,7- trimethyl- bicyclo[2.2.1]heptin-2- yl)-amide | 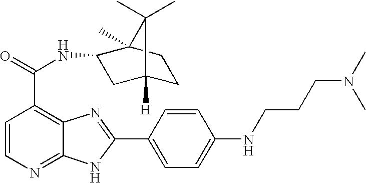 | CD₃OD: 0.93 (s, 3H), 0.99 (s, 3H), 1.08 (s, 3H), 1.15- 1.18 (m, 1H), 1.28-1.60 (m, 4H), 1.77-1.82 (m, 2H), 1.92-2.19 (m, 2H), 2.23- (s, 6H), 2.47-2.53 (m, 4H), 4.49-4.52 (m, 1H), 6.69 (d, J = 3.4 Hz, 2H), 7.71 (d, J = 2.4 Hz, 1H), 8.09 (d, J = 3.4 Hz, 2H), 8.36 (d, J = 2.4 Hz, 1H) | 474.7 |
| 378 | 2-[4-(2-pyrrolidin-1-yl- ethylamino)-phenyl]-3H- imidazo[4,5-b]pyridine- 7-carboxylic acid(1,7,7- trimethyl- bicyclo[2.2.1]heptin-2- yl)-amide | 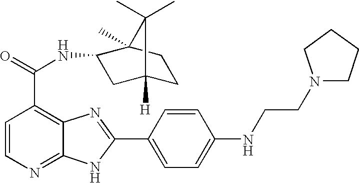 | CD₃OD: 0.93 (s, 3H), 0.98 (s, 3H), 1.06 (s, 3H), 1.12- 1.16 (m, 1H), 1.26-1.59 (m, 4H), 1.78-2.19 (m, 2H), 1.80-1.86 (m, 4H), 2.67- 2.71 (m, 4H), 2.83 (t, J = 2.2 Hz, 2H), 3.36 (t, J = 2.2 Hz, 2H), 4.43-4.54 (m, 1H), 6.75 (d, J = 3.4 Hz, 2H), 7.74 (d, J = 2.4 Hz, 1H), 8.03 (d, J = 3.4 Hz, 2H), 8.32 (d, J = 2.4 Hz, 1H) | 486.7 |
| 379 | 2-{4-[3-(4-methyl- piperazin-1-yl)- propylamino]-phenyl}- 3H-imidazo[4,5- b]pyridine-7-carboxylic acid(1,7,7- trimethyl- bicyclo[2.2.1]heptin-2- yl)-amide | 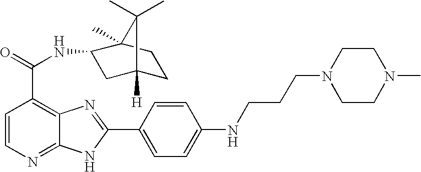 | CD₃OD: 0.93 (s, 3H), 0.98 (s, 3H), 1.06 (s, 3H), 1.12- 1.34 (m, 5H), 1.47-1.59 (m, 2H), 1.78-1.83 (m, 4H), 1.97-2.16 (m, 3H), 2.47- 2.53 (m, 10H), 3.22-3.28 (m, 2H), 4.43-4.51 (m, 1H), 6.72 (d, J = 3.6 Hz, 2H), 7.71 (d, J = 2.4 Hz, 1H), 8.01 (d, J = 3.6 Hz, 2H), 8.30 (d, J = 2.4 Hz, 1H) | 529.7 |
| 380 | 2-[4-(2-dimethylamino- ethylamino)-phenyl]-3H- imidazo[4,5-b]pyridine- 7-carboxylic acid(2- hydroxy-1,1-dimethyl- ethyl)-amide | 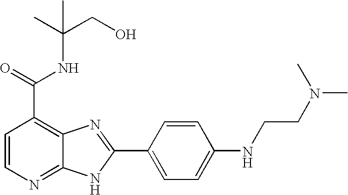 | CD₃OD: 1.58 (s, 6H), 2.99 (s, 6H), 3.41 (t, J = 2.6 Hz, 2H), 3.68 (d, J = 2.6 Hz, 1H), 3.81 (s, 2H), 6.93 (d, J = 4.4 Hz, 1H), 7.78 (d, J = 3.4 Hz, 2H), 8.15 (d, J = 4.4 Hz, 1H), 8.41 (d, J = 3.4 Hz, 2H) | 396.5 |
| 381 | 2-[4-(2-dimethylamino- ethylamino)-phenyl]-3H- imidazo[4,5-b]pyridine- 7-carboxylic acid(1,1- dimethyl-propyl)-amide | 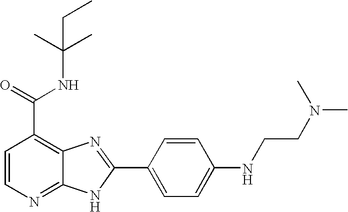 | CD₃OD: 1.13 (t, J = 2.8 Hz, 3H), 1.27 (d, J = 2.1 Hz, 1H), 1.59 (s, 6H), 1.27 (d, J = 2.1 Hz, 1H), 6.79 (d, J = 3.4 Hz, 2H), 7.62 (d, J = 2.4 Hz, 1H), 8.01 (d, J = 3.4 Hz, 2H), 8.34 (d, J = 2.4 Hz, 1H) | 394.5 |
| 382 | 2-[4-(2-dimethylamino- ethylamino)-phenyl]-3H- imidazo[4,5-b]pyridine- 7-carboxylic acid(3,3,5- trimethyl-cyclohexyl)- amide | 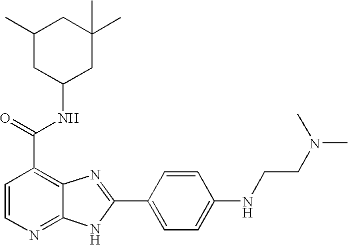 | CD₃OD: 0.72 (s, 3H), 1.45 (s, 6H), 1.24-1.60 (m, 6H), 2.39 (s, 6H), 2.68 (t, J = 2.4 Hz, 2H), 3.98-4.01 (m, 1H), 6.79 (d, J = 3.4 Hz, 2H), 7.78 (d, J = 2.4 Hz, 1H), 8.01 (d, J = 3.4 Hz, 2H), 8.34 (d, J = 2.4 Hz, 1H) | 448.6 |
| 383 | 2-[4-(2-dimethylamino- ethylamino)-phenyl]-3H- imidazo[4,5-b]pyridine- 7-carboxylic acid tert- butylamide | 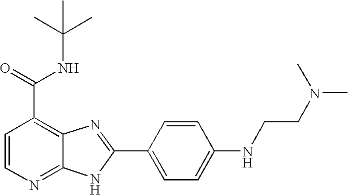 | CD₃OD: 1.59 (s, 9H), 2.99 (s, 6H), 3.42 (t, J = 2.1 Hz, 2H), 3.61 (t, J = 2.1 Hz, 2H), 6.91 (d, J = 3.4 Hz, 2H), 7.78 (d, J = 2.4 Hz, 1H), 8.10 (d, J = 3.4 Hz, 2H), 8.21 (d, J = 2.4 Hz, 1H) | 380.5 |
| 384 | 2-[4-(3-dimethylamino- pyrrolidin-1-yl)-phenyl]- 3H-imidazo[4,5- b]pyridine-7-carboxylic acid(1,7,7- trimethyl- bicyclo[2.2.1]heptin-2- yl)-amide | 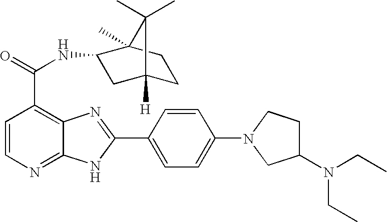 | CD₃OD: 0.92 (s, 3H), 0.98 (s, 3H), 1.05 (s, 6H), 1.04- 1.09 (m, 1H), 1.24-1.56 (m, 3H), 1.76-1.79 (m, 1H), 1.84-1.97 (m, 2H), 2.13- 2.23 (m, 2H), 2.49-2.52 (m, 1H), 269-2.74 (m, 4H), 3.05-3.21 (m, 1H), 3.33- 3.48 (m, 2H), 3.33-3.48 (m, 2H), 4.45-4.48 (m, 1H), 6.68 (d, J = 3.4 Hz, 2H), 6.79 (d, J = 2.4 Hz, 1H), 7.92 (d, J = 3.4 Hz, 2H), 8.21 (d, J = 2.4 Hz, 1H) | 514.7 |
| 385 | 2-[4-(2-dimethylamino- ethylamino)-phenyl]-3H- imidazo[4,5-b]pyridine- 7-carboxylic acid(2- dimethylamino-1-methyl- ethyl)-amide | 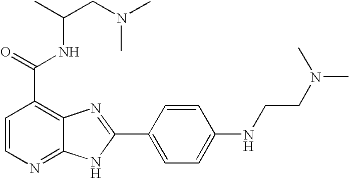 | CD₃OD: 1.42 (d, J = 3.6 Hz, 3H), 2.98 (s, 6H), 3.04- 3.69 (m, 10H), 6.88 (d, J = 3.4 Hz, 2H), 7.79 (d, J = 2.4 Hz, 1H), 8.14 (d, J = 3.4 Hz, 2H), 8.21 (d, J = 2.4 Hz, 1H) | 409.5 |
| 386 | 2-[4-(2-dimethylamino- ethylamino)-phenyl]-3H- imidazo[4,5-b]pyridine- 7-carboxylic acid(2- ethoxy-phenyl)-amide | 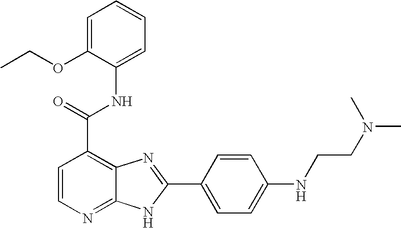 | CD₃OD: 2.21 (t, J = 2.7 Hz, 3H), 2.99 (s, 6H), 3.45 (t, J = 2.1 Hz, 2H), 3.69 (t, J = 2.1 Hz, 2H), 4.37 (q, J = 2.7 Hz, 2H), 6.97 (d, J = 3.4 Hz, 2H), 7.01-7.22 (m, 3H), 7.96 (d, J = 2.4 Hz, 1H), 8.22 (d, J = 3.4 Hz, 2H), 8.39- 8.40 (m, 1H), 8.46 (d, J = 2.4 Hz, 1H) | 444.5 |
| 387 | 2-[4-(2-dimethylamino- ethylamino)-phenyl]-3H- imidazo[4,5-b]pyridine- 7-carboxylic acid(2- hydroxy-cyclopentyl)- amide | 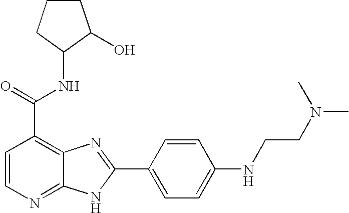 | CD₃OD: 1.62-2.02 (m, 6H), 2.99 (s, 6H), 3.40 (t, J = 2.1 Hz, 2H), 3.71 (t, J = 2.1 Hz, 2H), 4.19-4.39 (m, 2H), 6.87 (d, J = 3.4 Hz, 2H), 7.76 (d, J = 2.4 Hz, 1H), 8.12 (d, J = 3.4 Hz, 2H), 8.26 (d, J = 2.4 Hz, 1H) | 408.5 |
| 388 | 2-[4-(2-dimethylamino- ethylamino)-phenyl]-3H- imidazo[4,5-b]pyridine- 7-carboxylic acid(1- hydroxymethyl- cyclopentyl)-amide | 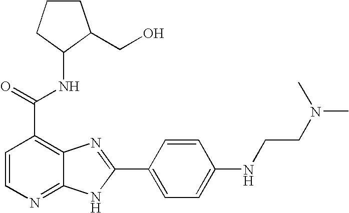 | 1.62-2.02 (m, 6H), 2.99 (s, 6H), 3.40 (t, 2H), 3.60_3.80 (m, 4H), 4.19- 4.39 (m, 2H), 6.87 (d, 2H), 7.76 (d, 1H), 8.12 (d, 2H), 8.26 (d, 1H) | 422.5 |
| 389 | 2-[4-(2-dimethylamino- ethylamino)-phenyl]-3H- imidazo[4,5-b]pyridine- 7-carboxylic acid(3- imidazo-1-yl-propyl)- amide | 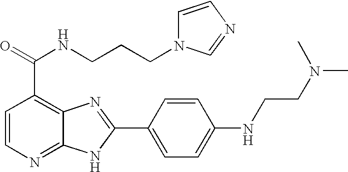 | CD₃OD: 2.06-2.15 (m, 2H), 3.00 (s, 6H), 2.31 (t, J = 1.4 Hz, 2H), 3.34 (t, J = 1.4 Hz, 2H), 3.46- 3.55 (m, 2H), 4.11 (t, J = 2.4 Hz, 2H), 6.99 (b, 1H), 7.21 (b, 1H), 7.23 (d, J = 3.4 Hz, 2H), 7.70 (d, J = 1.4 Hz, 1H), 8.15 (d, J = 3.4 Hz, 2H), 8.43 (d, J = 1.4 Hz, 1H), 9.42 (s, 1H) | 432.5 |
| 390 | 2-[4-(2-dimethylamino- ethylamino)-phenyl]-3H- imidazo[4,5-b]pyridine- 7-carboxylic acid[3- (4,5-dichloro-imidazo- 1-yl)-propyl]-amide | 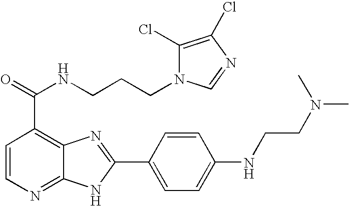 | CD₃OD: 2.02-2.13 (m, 2H), 2.99 (s, 6H), 2.30 (t, J = 2.4 Hz, 2H), 3.38 (t, J = 2.4 Hz, 2H), 3.46- 3.55 (m, 2H), 4.11 (t, J =2.4 Hz, 2H), 7.22 (d, J =3.4 Hz, 2H), 7.63 (d, J =1.4 Hz, 1H), 8.05 (d, J =3.4 Hz, 2H), 8.18 (d, J =1.4 Hz, 1H) | 501.4 |
| 391 | 2-[4-(3-pyrrolidin-1-yl- propylamino)-phenyl]- 3H-imidazo[4,5- b]pyridine-7-carboxylic acid(2-pyridin-3-yl- ethyl)-amide | 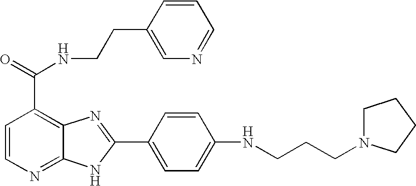 | 2.23~1.98 (m, 4H), 3.15 (m, 2H), 3.31 (t, 2H), 3.70 (m, 2H), 4.00 (t, 2H), 7.38 (t, 2H), 7.75 (d, 1H), 8.02 (m, 1H), 8.31 (m, 2H), 8.45 (d, 1H), 8.70 (m, 2H), 8.88 (s, 1H) | 469.6 |
| 392 | 2-[4-(2-dimethylamino- ethylamino)-phenyl]-3H- imidazo[4,5-b]pyridine- 7-carboxylic acid(1,3,3- trimethyl-butyl)-amide | 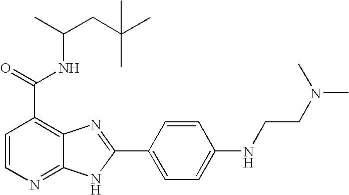 | CD₃OD: 1.12 (s, 9H), 1.28 (d, J = 1.4 Hz, 3H), 1.30 (d, J = 1.4 Hz, 3H), 2.34 (s, 6H), 2.62 (t, J = 2.4 Hz, 2H), 3.34 (t, J = 2.4 Hz, 2H), 4.10 (q, J = 1.8 Hz, 2H), 6.75 (d, J = 3.4 Hz, 2H), 7.73 (d, J = 2.4 Hz, 1H), 8.01 (d, J = 3.4 Hz, 2H), 8.31 (d, J = 2.4 Hz, 1H) | 422.6 |
| 393 | 2-[4-(2-dimethylamino- ethylamino)-phenyl]-3H- imidazo[4,5-b]pyridine- 7-carboxylic acid(1,4- dimethyl-pentyl)-amide | 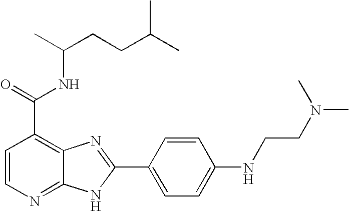 | CD₃OD: 0.96 (d, J = 4.4 Hz, 3H), 1.01 (d, J = 4.4 Hz, 3H), 1.26-1.29 (m, 2H), 1.29 (d, J = 3.1 Hz, 3H), 1.48-1.67 (m, 2H), 1.86-1.89 (m, 1H), 2.33 (s, 6H), 2.62 (t, J = 2.4 Hz, 2H), 3.33 (t, J = 2.4 Hz, 2H), 4.27 (q, J = 1.2 Hz, 2H), 6.74 (d, J = 3.4 Hz, 2H), 7.69 (d, J = 2.4 Hz, 1H), 7.99 (d, J = 3.4 Hz, 2H), 8.30 (d, J = 2.4 Hz, 1H) | 422.6 |
| 394 | 2-[4-(2-dimethylamino- ethylamino)-phenyl]-3H- imidazo[4,5-b]pyridine- 7-carboxylic acid(1- ethyl-propyl)-amide | 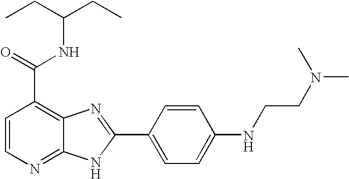 | CD₃OD: 1.23 (t, J = 3.4 Hz, 6H), 3.00 (s, 6H), 3.27 (q, J = 1.2 Hz, 4H), 3.32 (t, J = 2.4 Hz, 2H), 3.53 (t, J = 2.4 Hz, 2H), 4.21-4.28 (m, 1H), 6.99 (d, J = 3.1 Hz, 2H), 7.61 (d, J = 2.0 Hz, 1H), 7.89 (d, J = 3.1 Hz, 2H), 8.28 (d, J = 2.0 Hz, 1H) | 394.5 |
| 395 | 2-[4-(2-dimethylamino- ethylamino)-phenyl]-3H- imidazo[4,5-b]pyridine- 7-carboxylic acid(1,1,3,3-tetramethyl- butyl)-amide | 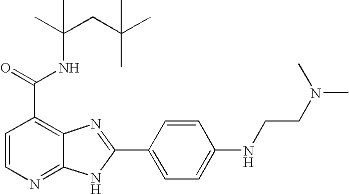 | CD₃OD: 1.03 (s, 9H), 1.01 (d, J = 4.4 Hz, 3H), 1.26-1.40 (m, 2H), 1.63 (s, 6H), 2.32 (s, 6H), 2.61 (d, J = 2.4 Hz, 3H), 3.33 (t, J = 2.4 Hz, 2H), 4.27 (q, J = 1.2 Hz, 2H), 6.79 (d, J = 3.1 Hz, 2H), 7.82 (d, J = 2.2 Hz, 1H), 7.99 (d, J = 3.1 Hz, 2H), 8.29 (d, J = 2.2 Hz, 1H) | 436.6 |
| 396 | 2-(4-methoxy-phenyl)- 3H-imidazo[4,5- b]pyridine-7-carboxylic acid (4-methyl-pyridin-3-yl)-amide | 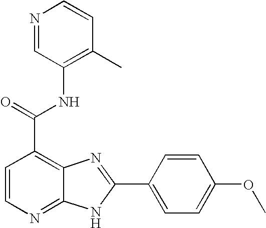 | 2.58 (s, 3H), 3.86 (s, 3H), 7.17 (d, 2H), 7.38 (d, 1H), 7.82 (d, 1H), 8.26 (s, 1H), 8.29 (t, 2H), 8.50 (s, 1H), 9.38 (s, 1H), 11.47 (s, 1H) | 359.4 |
| 397 | 2-(4-methoxy-phenyl)- 3H-imidazo[4,5- b]pyridine-7-carboxylic acid (4-ethoxy-pyridin-3-yl)-amide | 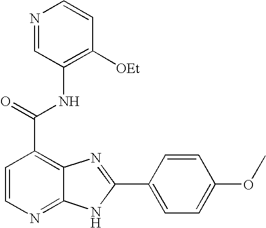 | 12.07 (s, 1H), 9.73 (s, 1H), 8.61 (d, 1H), 8.54 (d, 1H), 8.33 (d, 2H), 7.86 (d, 1H), 7.77 (d, 1H), 7.24 (d, 2H), 4.67 (q, 2H), 3.90 (s, 3H), 1.49 (t, 3H) | 389.4 |
| 398 | 2-(4-methoxy-phenyl)- 3H-imidazo[4,5- b]pyridine-7-carboxylic acid (6-methoxy-pyridin-3-yl)-amide | 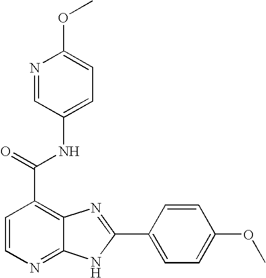 | 3.86 (d, 6H), 6.92 (d, 1H), 7.17 (d, 2H), 7.73 (d, 1H), 8.21 (dd, 1H), 8.34 (d, 2H), 8.45 (d, 1H), 8.64 (d, 2H), 11.68 (br s, 1H) | 375.4 |
| 399 | 2-(4-methoxy-phenyl)- 3H-imidazo[4,5- b]pyridine-7-carboxylic acid (6-methyl-pyridin-3-yl)-amide | 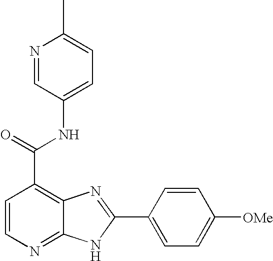 | 11.79 (s, 1H), 9.09 (s, 1H), 8.49 (d, 1H), 8.35 (m, 3H), 7.78 (, d, 1H), 7.58 (d, 1H), 7.20 (d, 2H), 3.88 (s, 3H), 2.60 (s, 3H) | 359.4 |
| 400 | 2-[4-(4-dimethylamino- butyl amino)-phenyl]- 3H-imidazo[4,5- b]pyridine-7-carboxylic acid(4-ethoxy-pyridin-3- yl)-amide | 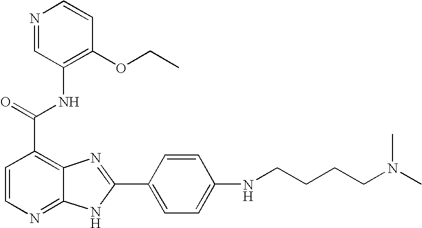 | 9.58 (br, 1H), 8.40~8.20 (m, 2H), 8.11 (d, 2H), 7.83 (d, 1H), 6.78 (d, 2H), 4.48 (m, 2H), 2.99 (m, 2H), 2.76 (s, 6H), 1.95-1.73 (m, 6H), 1.50 (t, 3H) | 473.6 |
| 401 | 2-(4-methoxy-phenyl)- 3H-imidazo[4,5- b]pyridine-7-carboxylic acid (6-methoxy-4- methyl-pyridin-3-yl)- amide | 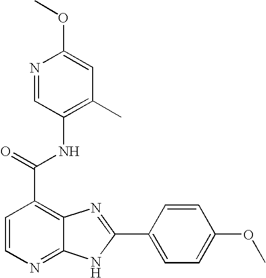 | 10.97 (s, 1H), 8.48 (d, 1H), 8.41 (s, 1H), 8.24 (d, 2H), 7.77 (d, 1H), 7.18 (d, 2H), 6.40 (s, 1H), 3.87 (s, 3H), 3.46 (s, 3H), 2.37 (s, 3H) | 389.4 |
| 402 | 2-[4-(4-methyl- piperazin-1-yl)-phenyl]- 3H-imidazo[4,5- b]pyridine-7-carboxylic acid(6-methyl-pyridin-3- yl)-amide | 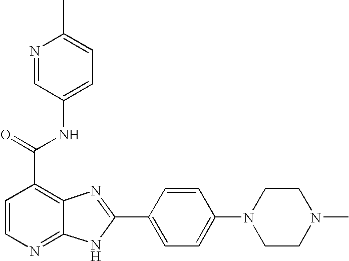 | 11.70 (s, 1H), 9.30 (s, 1H), 8.50 (d, 1H), 8.22 (m, 3H), 7.78 (d, 1H), 7.50 (d, 1H), 7.12 (d, 2H), 3.34 (m, 8H), 2.49 (s, 3H) | 427.5 |
| 403 | 2-(4-pyrrolidin-1-yl- phenyl)-3H-imidazo[4,5- b]pyridine-7-carboxylic acid(4-ethylsulfanyl- pyridin-3-yl)-amide | 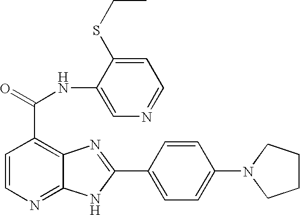 | 11.87 (br, 1H), 9.43 (s, 1H), 8.49 (d, 1H), 8.42 (d, 1H), 8.28 (d, 2H), 8.82 (d, 1H), 7.77 (d, 1H), 6.70 (d, 2H), 3.36 (m, 6H), 2.00 (m, 4H), 1.42 (t, 3H) | 444.6 |
| 404 | 2-(4-pyrrolidin-1-yl- phenyl)-3H-imidazo[4,5- b]pyridine-7-carboxylic acid(4-ethoxy-pyridin-3- yl)-amide | 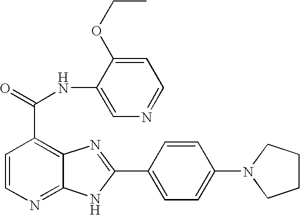 | 9.61 (br, 1H), 8.31 (d, 1H), 8.13 (m, 3H), 7.80 (d, 1H), 7.35 (m, 1H), 6.68 (d, 2H), 3.32 (m, 4H), 2.09 (m, 4H), 1.51 (t, 3H) | 428.5 |
| 405 | 2-(4-pyrrolidin-1-yl- phenyl)-3H-imidazo[4,5- b]pyridine-7-carboxylic acid(6-methyl-pyridin-3- yl)-amide | 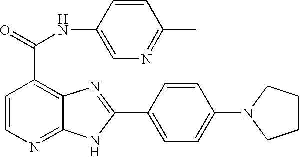 | 8.87 (br, 1H), 8.34 (d, 1H), 8.16 (d, 2H), 8.08 (br, 1H), 7.72 (d, 1H), 7.30 (d, 1H), 6.72 (d, 2H), 3.33 (m, 4H), 2.56 (s, 3H), 2.00 (m, 4H) | 398.5 |
| 406 | 2-[4-(3-diethylamino- pyrrolidin-1-yl)-phenyl]- 3H-imidazo[4,5- b]pyridine-7-carboxylic acid(4-ethylsulfanyl- pyridin-3-yl)-amide | 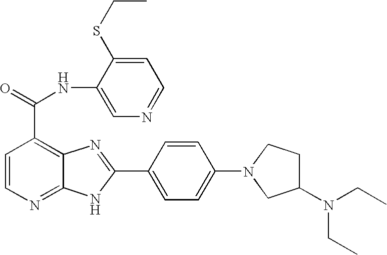 | 9.32 (br, 1H), 8.40-8.20 (m, 4H), 7.82 (d, 1H), 7.48 (d, 1H), 6.72 (d, 2H), 4.18 (t, 1H), 3.84 (t, 1H), 3.68 (t, 1H), 3.58~3.40 (m, 2H), 3.22 (m, 6H), 2.60 (m, 1H), 2.23 (m, 1H), 1.39 (m, 9H) | 515.7 |
| 407 | 2-[4-(4-methyl- piperazin-1-yl)-phenyl]- 3H-imidazo[4,5- b]pyridine-7-carboxylic acid(4-ethoxy-pyridin-3- yl)-amide | 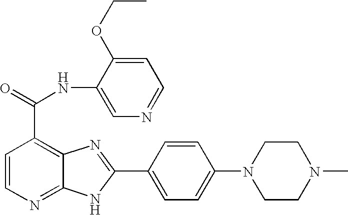 | 9.33 (br, 1H), 8.39 (m, 2H), 8.19 (d, 2H), 7.84 (d, 1H), 7.35 (m, 1H), 7.13 (m, 2H), 4.46 (q, 2H), 3.51 (m, 4H), 3.00 (m, 4H), 2.65 (s, 3H), 1.47 (t, 3H) | 457.5 |
| 408 | 2-(4-pyrrolidin-1-yl- phenyl)-3H-imidazo[4,5- b]pyridine-7-carboxylic acid(6-methoxy-4- methyl-pyridin-3-yl)- amide | 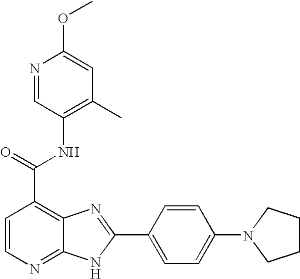 | 1.80 (t, 3H), 1.90 (t, 3H), 1.97 (s, 5H), 3.57 (t, 3H), 6.65 (d, 2H), 7.10 (d, 1H), 8.03 (d, 2H), 8.11 (d, 1H), 8.20 (d, 1H), 8.30 (d, 1H) | 428.5 |
| 409 | 2-[4-(3-diethylamino- pyrrolidin-1-yl)-phenyl]- 3H-imidazo[4,5- b]pyridine-7-carboxylic acid(6-methyl-pyridin-3- yl)-amide | 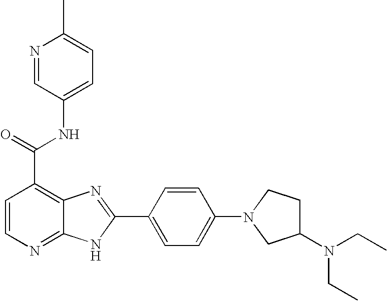 | 1.22-1.26 (m, 6H), 2.18 (t, 2H), 3.38 (s, 3H), 3.41- 3.53 (m, 2H), 3.64 (t, 2H), 3.82 (t, 2H), 4.15 (d, 2H), 6.85 (d, 2H), 7.35 (d, 1H), 7.73 (d, 1H), 8.25 (d, 2H), 8.41 (d, 1H), 8.88 (s, 1H), 9.42 (br s, 1H), 11.87 (br s, 1H) | 469.6 |
| 410 | 2-[4-(4-methyl- piperazin-1-yl)-phenyl]- 3H-imidazo[4,5- b]pyridine-7-carboxylic acid(6-methoxy-4- methyl-pyridin-3-yl)- amide | 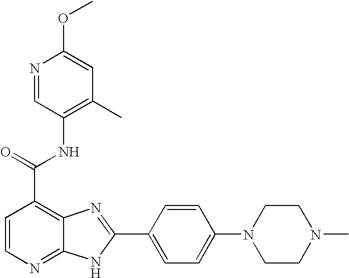 | 2.40 (s, 3H), 2.74 (s, 4H), 3.18 (s, 4H), 3.46 (s, 6H), 6.43 (s, 1H), 7.18 (d, 2H), 7.74 (d, 1H), 8.18 (d, 2H), 8.44 (d, 1H), 8.50 (s, 1H), 11.09 (s, 1H) | 457.5 |
| 411 | 2-(4-piperidin-1-yl- phenyl)-3H-imidazo[4,5- b]pyridine-7-carboxylic acid(4-ethoxy-pyridin-3- yl)-amide | 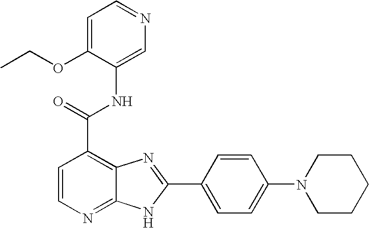 | 9.60 (br, 1H), 8.36 (d, 1H), 8.25 (d, 1H), 8.15 (d, 2H), 7.84 (d, 1H), 7.21 (d, 1H), 7.07 (d, 2H), 4.48 (q, 2H), 3.37 (m, 4H), 1.75 (m, 6H), 1.48 (t, 3H) | 442.5 |
| 412 | 2-(4-piperidin-1-yl- phenyl)-3H-imidazo[4,5- b]pyridine-7-carboxylic acid(6-methyl-pyridin-3- yl)-amide | 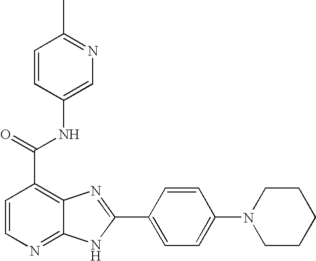 | 8.93 (br, 1H), 8.39 (d, 1H), 8.26 (d, 1H), 8.13 (d, 2H), 7.82 (d, 1H), 7.38 (d, 1H), 7.11 (d, 2H), 3.39 (m, 4H), 2.55 (s, 3H), 1.71 (m, 6H) | 412.5 |
| 413 | 2-(4-piperidin-1-yl- phenyl)-3H-imidazo[4,5- b]pyridine-7-carboxylic acid(6-methoxy-4- methyl-pyridin-3-yl)- amide | 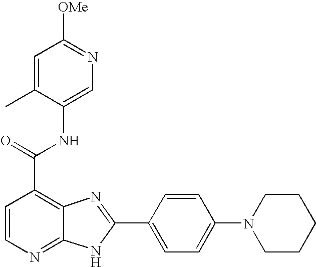 | 8.53 (d, 1H), 8.39 (s, 1H), 8.25 (d, 2H), 7.90 (d, 1H), 7.45 (d, 2H), 6.56 (s, 1H), 3.62 (s, 3H), 3.57 (m, 4H), 2.63 (s, 3H), 1.90~1.78 (m, 6H) | 442.5 |
| 414 | 2-(4-piperidin-1-yl- phenyl)-3H-imidazo[4,5- b]pyridine-7-carboxylic acid(4,6-dimethyl- pyridin-3-yl)-amide | 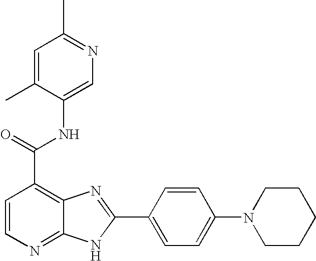 | 9.73 (s, 1H), 8.42 (d, 1H), 8.06 (q, 2H), 7.88 (m, 2H), 7.09 (d, 2H), 3.41 (m, 4H), 2.78 (s, 3H), 1.76 (m, 6H) | 426.5 |
| 415 | 2-(4-pyrrolidin-1-yl- phenyl)-3H-imidazo[4,5- b]pyridine-7-carboxylic acid(4,6-dimemethyl- pyridin-3-yl)-amide | 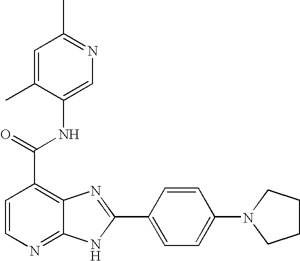 | 8.41 (m, 1H), 8.09 (d, 2H), 7.88 (d, 1H), 7.42 (s, 1H), 7.08 (d, 2H), 6.73 (d, 1H), 3.44 (m, 4H), 2.70 (m, 6H), 1.93 (br, 4H) | 412.5 |
| 416 | 2-[4-(4-methyl- piperazin-1-yl)-phenyl]- 3H-imidazo[4,5- b]pyridine-7-carboxylic acid(4,6-dimethyl- pyridin-3-yl)-amide | 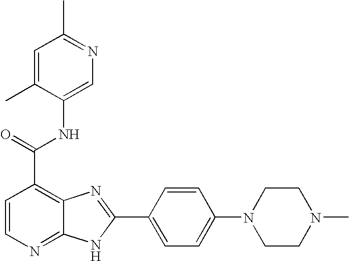 | 8.63 (d, 1H), 8.25 (d, 2H), 8.07 (m, 2H), 7.96 (d, 1H), 7.27 (d, 2H), 3.00 (s, 3H), 2.76 (s, 3H), 2.55 (s, 3H) | 441.5 |
| 417 | 2-(4-piperidin-1-yl- phenyl)-3H-imidazo[4,5- b]pyridine-7-carboxylic acid(4-ethylsulfanyl- pyridin-3-yl)-amide | 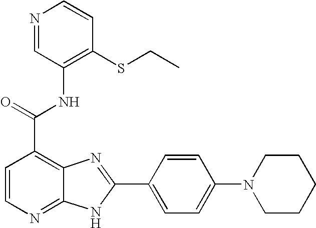 | 11.69 (s, 1H), 9.27 (s, 1H), 8.43 (d, 1H), 8.30 (m, 3H), 7.77 (d, 1H), 7.51 (d, 1H), 7.07 (2H), 3.37 (m, 4H), 3.21 (q, 2H), 1.61 (m, 6H), 1.36 (t, 3H) | 458.6 |
| 418 | 2-[4-(4-methyl- piperazin-1-yl)-phenyl]- 3H-imidazo[4,5- b]pyridine-7-carboxylic acid(4-ethylsulfanyl- pyridin-3-yl)-amide | 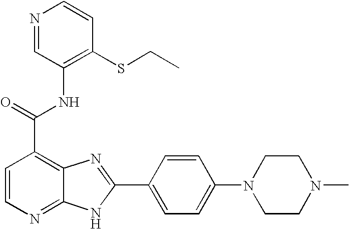 | 11.63 (s, 1H), 9.28 (s, 1H), 8.46 (d, 1H), 8.32 (m, 3H), 7.80 (d, 1H), 7.51 (d, 1H), 7.10 (d, 2H), 3.34 (m, 8H), 3.21 (q, 2H), 2.24 (s, 3H), 1.38 (t, 3H) | 473.6 |
| 419 | 2-{4-[(2- dimethylamino-ethyl)- methyl-amino]-phenyl}- 3H-imidazo[4,5- b]pyridine-7-carboxylic acid(4,6-dimethyl- pyridin-3-yl)-amide | 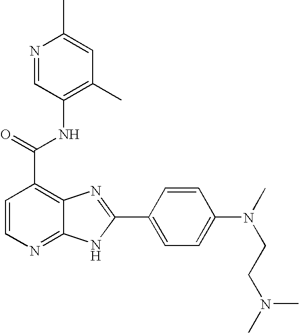 | 2.46 (s, 4H), 2.56 (s, 3H), 2.85 (s, 5H), 3.00 (s, 3H), 3.39 (s, 3H), 6.97 (d, 2H), 7.23 (s, 1H), 7.78 (s, 1H), 8.17 (d, 2H), 8.43 (d, 1H), 9.26 (s, 1H), 9.37 (br s, 1H), 11.45 (s, 1H) | 443.6 |
| 420 | 2-{4-[(2- dimethylamino-ethyl)- methyl-amino]-phenyl}- 3H-imidazo[4,5- b]pyridine-7-carboxylic acid(4-ethoxy-pyridin-3- yl)-amide | 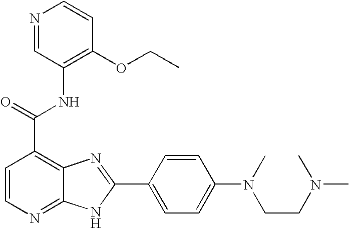 | 9.57 (br, 1H), 8.42~8.22 (m, 2H), 8.12 (d, 2H), 7.78 (d, 1H), 6.90 (d, 2H), 4.45 (q, 2H), 3.82 (t, 2H), 3.10 (s, 3H), 2.89 (m, 8H), 1.46 (t, 3H) | 459.6 |
| 421 | 2-{4-[(2- dimethylamino-ethyl)- methyl-amino]-phenyl}- 3H-imidazo[4,5- b]pyridine-7-carboxylic acid(6-methyl-pyridin-3- yl)-amide | 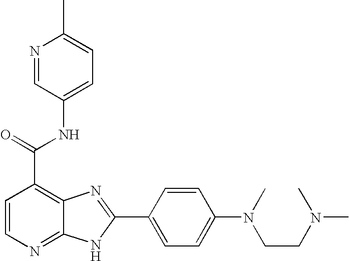 | 8.83 (br, 1H), 8.38 (d, 1H), 8.21 (de, 1H), 8.09 (d, 2H), 7.76 (d, 1H), 7.34 (d, 1H), 6.93 (d, 2H), 3.78 (t, 2H), 3.20 (t, 2H), 3.10 (s, 10H), 2.55 (s, 3H) | 429.5 |
| 422 | 2-{4-[(2- dimethylamino-ethyl)- methyl-amino]-phenyl}- 3H-imidazo[4,5- b]pyridine-7-carboxylic acid(4-ethylsulfanyl- pyridin-3-yl)-amide | 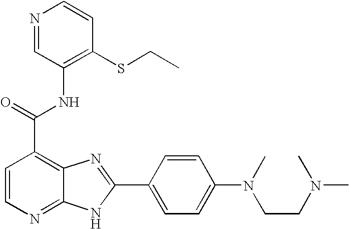 | 9.32 (br, 1H), 8.48~8.38 (m, 2H), 8.24 (d, 2H), 7.83 (d, 1H), 7.50 (d, 1H), 6.90 (d, 2H), 3.35 (t, 2H), 3.30~2.89 (m, 7H), 2.61 (s, 6H), 1.43 (t, 3H) | 475.6 |
| 423 | 2-[4-(4-methyl- piperazin-1-yl)-phenyl]- 3H-imidazo[4,5- b]pyridine-7-carboxylic acid(4-ethoxy-6-methyl- pyridin-3-yl)-amide | 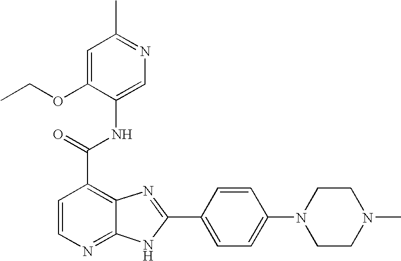 | 9.45 (br, 1H), 8.38 (dm 1H), 8.14 (d, 2H), 7.78 (d, 1H), 7.38 (m, 1H), 7.12 (d, 1H), 4.40 (q, 2H), 3.55 (br, 4H), 3.15 (br, 4H), 2.76 (s, 3H), 2.55 (s, 1H), 1.45 (t, 3H) | 471.6 |
| 424 | 2-(4-methoxy-phenyl)- 3H-imidazo[4,5- b]pyridine-7-carboxylic acid(4-methyl-pyridin- 3-yl)-amide | 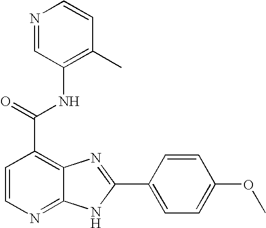 | 2.58 (s, 3H), 3.86 (s, 3H), 7.17 (d, 2H), 7.38 (d, 1H), 7.82 (d, 1H), 8.26 (s, 1H), 8.29 (t, 2H), 8.50 (s, 1H), 9.38 (s, 1H), 11.47 (s, 1H) | 359.4 |
| 425 | 2-(4-methoxy-phenyl)- 3H-imidazo[4,5- b]pyridine-7-carboxylic acid(4-ethoxy-pyridin- 3-yl)-amide | 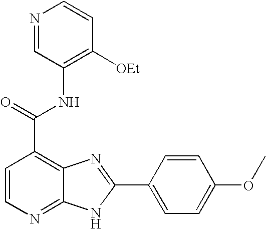 | 12.07 (s, 1H), 9.73 (s, 1H), 8.61 (d, 1H), 8.54 (d, 1H), 8.33 (d, 2H), 7.86 (d, 1H), 7.77 (d, 1H), 7.24 (d, 2H), 4.67 (q, 2H), 3.90 (s, 3H), 1.49 (t, 3H) | 389.4 |
| 426 | 2-(4-methoxy-phenyl)- 3H-imidazo[4,5- b]pyridine-7-carboxylic acid(4-methoxy-pyridin- 3-yl)-amide | 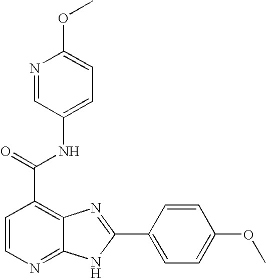 | 3.86 (d, 6H), 6.92 (d, 1H), 7.17 (d, 2H), 7.73 (d, 1H), 8.21 (dd, 1H), 8.34 (d, 2H), 8.45 (d, 1H), 8.64 (d, 2H), 11.68 (br s, 1H) | 375.4 |
| 427 | 2-(4-methoxy-phenyl)- 3H-imidazo[4,5- b]pyridine-7-carboxylic acid(4-methyl-pyridin- 3-yl)-amide | 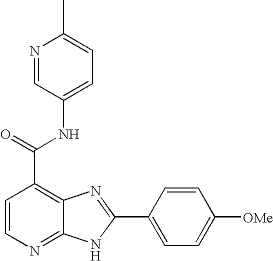 | 11.79 (s, 1H), 9.09 (s, 1H), 8.49 (d, 1H), 8.35 (m, 3H), 7.78 (, d, 1H), 7.58 (d, 1H), 7.20 (d, 2H), 3.88 (s, 3H), 2.60 (s, 3H) | 359.4 |
| 428 | 2-(4-methoxy-phenyl)- 3H-imidazo[4,5- b]pyridine-7-carboxylic acid(6-methoxy-4- methyl-pyridin-3-yl)- amide | 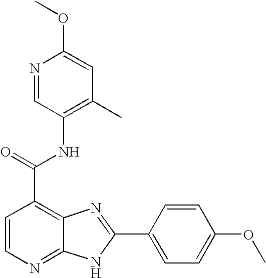 | 10.97 (s, 1H), 8.48 (d, 1H), 8.41 (s, 1H), 8.24 (d, 2H), 7.77 (d, 1H), 7.18 (d, 2H), 6.40 (s, 1H), 3.87 (s, 3H), 3.46 (s, 3H), 2.37 (s, 3H) | 389.4 |
| 429 | 6-bromo-2-(3-methoxy- phenyl)-3H-imidazo[4,5- b]pyridine-7-carboxylic acid(4-methyl-pyridin-3- yl)-amide | 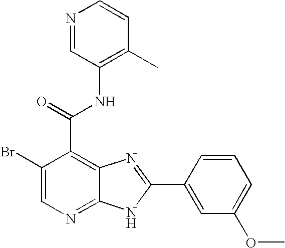 | 9.31 (br, 1H), 9.12 (br, 1H), 8.62 (d, 1H), 8.35~8.29 (m, 2H), 7.99 (s, 1H), 7.95 (d, 1H), 7.68~7.59 (m, 2H), 7.47 (t, 1H), 4.46 (s, 3H), 2.29 (s, 3H) | 438.3 |
| 430 | 2-(3-methoxy-phenyl)- 3H-imidazo[4,5- b]pyridine-7-carboxylic acid(4-methyl-pyridin- 3-yl)-amide | 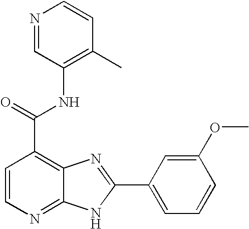 | 11.73 (s, 1H), 9.66 (s, 1H), 8.60 (d, 2H), 7.95 (m, 4H), 7.50 (m, 1H), 7.20 (d, 1H), 3.90 (s, 3H), 2.86 (s, 3H) | 359.4 |
| 431 | 2-(3-methoxy-phenyl)- 3H-imidazo[4,5- b]pyridine-7-carboxylic acid(6-methoxy-pyridin- 3-yl)-amide | 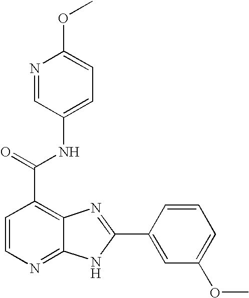 | 3.86 (s, 3H), 3.88 (s, 3H), 6.92 (d, 1H), 7.16 (q, 1H), 7.54 (t, 1H), 7.77 (d, 1H), 7.92 (s, 1H), 7.99 (d, 1H), 8.19 (q, 1H), 8.51 (d, 1H), 8.63 (d, 1H), 11, 58 (br s, 1H) | 375.4 |
| 432 | 2-(3-methoxy-phenyl)- 3H-imidazo[4,5- b]pyridine-7-carboxylic acid(4-ethoxy-pyridin- 3-yl)-amide | 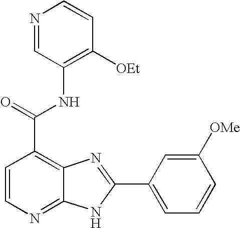 | 12.08 (s, 1H), 9.75 (s, 1H), 8.72 (d, 1H), 8.58 (d, 1H), 8.00 (m, 2H), 7.87 (m, 2H), 7.61 (m, 1H), 7.25 (d, 1H), 4.75 (q, 2H), 3.90 (s, 3H), 1.48 (t, 3H) | 389.4 |
| 433 | 2-(4-methoxy-phenyl)- 3H-imidazo[4,5- b]pyridine-7-carboxylic acid(6-methyl-pyridin- 3-yl)-amide | 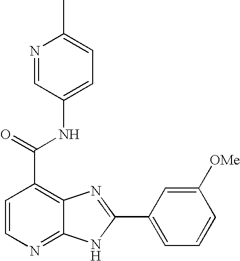 | 11.91 (s, 1H), 9.30 (s, 1H), 8.67 (d, 1H), 8.58 (d, 1H), 8.05 (m, 2H), 7.91 (d, 1H), 7.82 (d, 1H), 7.57 (m, 1H), 7.20 (m, 1H), 3.91 (s, 3H), 2.74 (s, 3H) | 359.4 |
| 434 | 2-pyridin-2-yl-3H- imidazo[4,5-b]pyridine- 7-carboxylic acid(4- methyl-pyridin-3-yl)- amide | 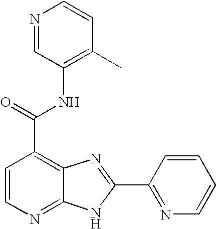 | 11.42 (s, 1H), 9.35 (s, 1H), 8.83 (s, 1H), 8.60 (d, 1H) 8.49 (d, 1H), 8.32 (m, 2H), 8.08 (m, 1H), 7.89 (m, 1H), 7.63 (m, 1H), 7.41 (d, 1H), 2.61 (s, 3H) | 330.4 |
| 435 | 2-pyridin-2-yl-3H- imidazo[4,5-b]pyridine- 7-carboxylic acid(4- ethoxy-pyridin-3-yl)- amide | 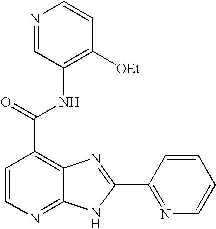 | 11.80 (s, 1H), 9.59 (s, 1H), 8.65 (d, 1H), 8.50 (d, 1H), 8.23 (m, 2H), 7.87 (m, 1H), 7.43 (d, 1H), 7.30 (m, 1H), 7.18 (d, 1H), 4.42 (q, 2H), 1.46 (t, 3H) | 360.4 |
| 436 | 2-pyridin-2-yl-3H- imidazo[4,5-b]pyridine- 7-carboxylic acid(2- chloro-pyridin-3-yl)- amide | 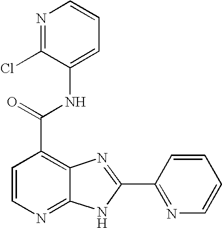 | 11.95 (s, 1H), 8.88 (d, 1H), 8.81 (s, 1H), 8.60 (m, 1H), 8.50 (m, 1H), 8.22 (d, 1H), 8.07 (m, 1H), 7.85 (d, 1H), 7.62 (m, 1H), 7.57 (m, 1H) | 350.8 |
| 437 | 2-pyridin-2-yl-3H- imidazo[4,5-b]pyridine- 7-carboxylic acid(6- methyl-pyridin-3-yl)- amide | 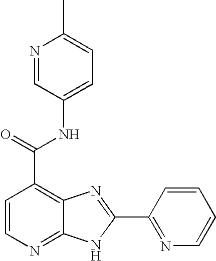 | 11.60 (s, 1H), 9.05 (s, 1H), 8.83 (d, 1H), 8.62 (m, 2H), 8.39 (d, 1H), 8.08 (m, 1H), 7.85 (d, 1H), 7.66 (m, 1H), 7.54 (d, 1H), 2.58 (s, 3H) | 330.4 |
| 438 | 2-pyridin-3-yl-3H- imidazo[4,5-b]pyridine- 7-carboxylic acid(4- ethoxy-pyridin-3-yl)- amide | 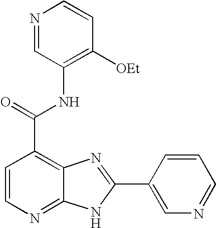 | 12.00 (s, 1H), 9.73 (s, 1H), 9.56 (d, 1H), 8.82 (dd, 1H), 8.68 (m, 2H), 8.62 (d, 1H), 7.90 (d, 1H), 7.83 (d, 1H), 7.72 (m, 1H), 4.73 (q, 2H), 1.45 (t, 3H) | 360.4 |
| 439 | 2-pyridin-3-yl-3H- imidazo[4,5-b]pyridine- 7-carboxylic acid(2- chloro-pyridin-3-yl)- amide | 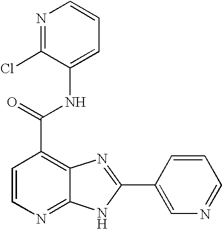 | 11.92 (s, 1H), 9.52 (s, 1H), 8.92 (d, 1H), 8.76 (d, 1H), 8.61 (m, 2H), 8.23 (d, 1H), 7.90 (d, 1H), 7.71 (m, 1H), 7.57 (m, 1H) | 350.8 |
| 440 | 2-pyridin-3-yl-3H- imidazo[4,5-b]pyridine- 7-carboxylic acid(6- chloro-pyridin-3-yl)- amide | 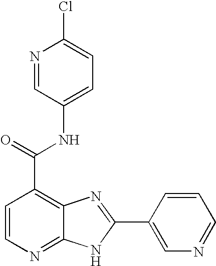 | 11.60 (s, 1H), 9.55 (s, 1H), 8.93 (d, 1H), 8.75 (m, 2H), 8.59 (d, 1H), 8.38 (dd, 1H), 7.82 (d, 1H), 7.70 (m, 1H), 7.60 (d, 1H) | 350.8 |
| 441 | 2-pyridin-3-yl-3H- imidazo[4,5-b]pyridine- 7-carboxylic acid(4- methyl-pyridin-3-yl)- amide | 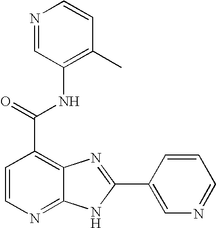 | 11.61 (s, 1H), 9.56 (s, 1H), 9.52 (s, 1H), 8.81 (d, 1H), 8.68 (m, 2H), 8.54 (d, 1H), 7.93 (d, 1H), 7.81 (d, 1H), 7.70 (m, 1H), 2.76 (s, 3H) | 330.4 |
| 442 | 2-pyridin-3-yl-3H- imidazo[4,5-b]pyridine- 7-carboxylic acid(6- methyl-pyridin-3-yl)- amide | 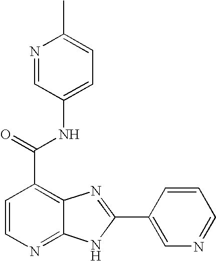 | 11.66 (s, 1H), 9.59 (s, 1H), 9.19 (s, 1H), 8.79 (m, 2H), 8.60 (d, 1H), 8.55 (m, 1H), 7.86 (d, 1H), 7.70 (m, 2H), 2.64 (s, 3H) | 330.4 |
| 443 | 2-pyridin-3-yl-3H- imidazo[4,5-b]pyridine- 7-carboxylic acid(6- methoxy-4-methyl- pyridin-3-yl)-amide | 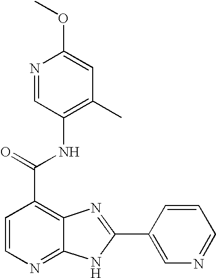 | 10.87 (s, 1H), 9.50 (s, 1H), 8.79 (d, 1H), 8.68 (d, 1H), 8.59 (d, 1H), 8.38 (s, 1H), 7.84 (d, 1H), 7.65 (m, 1H), 6.41 (s, 1H), 3.46 (s, 3H), 2.36 (s, 3H) | 360.4 |
| 444 | 2-pyridin-4-yl-3H- imidazo[4,5-b]pyridine- 7-carboxylic acid(4- methyl-pyridin-3-yl)- amide | 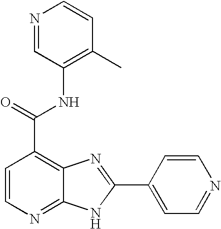 | 2.57 (s, 3H), 7.39 (d, 1H), 7.91 (s, 1H), 8.23 (s, 2H), 8.30 (d, 1H), 8.34 (d, 2H), 8.64 (d, 1H), 9.32 (s, 1H), 11.28 (s, 1H) | 330.4 |
| 445 | 2-pyridin-4-yl-3H- imidazo[4,5-b]pyridine- 7-carboxylic acid(4- ethoxy-pyridin-3-yl)- amide | 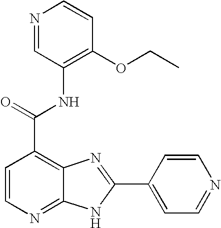 | 1.37 (t, 3H), 4.42 (q, 2H), 7.26 (d, 1H), 7.89 (d, 1H), 8.25 (d, 2H), 8.28 (d, 1H), 8.62 (d, 1H), 8.86 (d, 2H), 8.55 (d, 1H), 11.62 (d, 1H) | 360.4 |
| 446 | 2-pyridin-4-yl-3H- imidazo[4,5-b]pyridine- 7-carboxylic acid(6- methyl-pyridin-3-yl)- amide | 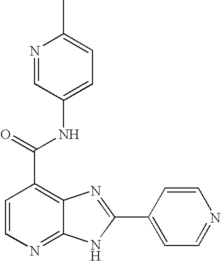 | 3.29 (s, 3H), 7.30 (d, 1H), 7.83 (d, 1H), 8.21 (d, 1H), 8.29 (d, 2H), 8.61 (d, 1H), 8.84 (d, 2H), 8.91 (d, 1H), 11.50 (br s, 1H) | 330.4 |
| 447 | 2-(3-chloro-phenyl)- 3H-imidazo[4,5- b]pyridine-7-carboxylic acid(4-ethoxy-pyridin- 3-yl)-amide | 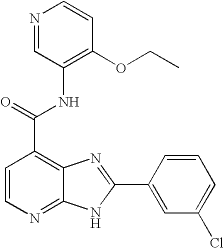 | 1.37 (t, 3H), 4.45 (q, 2H), 7.26 (d, 1H), 7.68 (d, 2H), 7.87 (d, 1H), 8.31 (t, 2H), 8.40 (s, 1H), 8.57 (d, 1H), 9.56 (s, 1H), 11.62 (s, 1H) | 393.6 |
| 448 | 2-(3-chloro-phenyl)- 3H-imidazo[4,5- b]pyridine-7-carboxylic acid(4-methyl-pyridin- 3-yl)-amide | 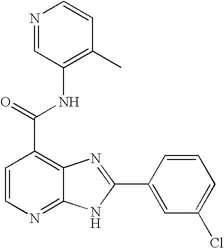 | 2.58 (s, 3H), 7.39 (d, 1H), 7.66 (s, 2H), 7.87 (d, 1H), 8.28 (d, 2H), 8.39 (s, 1H), 8.56 (q, 1H), 9.37 (s, 1H), 11.31 (s, 1H) | 363.8 |
| 449 | 2-(3-chloro-phenyl)- 3H-imidazo[4,5- b]pyridine-7-carboxylic acid(1,7,7-trimethyl- bicyclo[2.2.1]heptin-2- yl)-amide | 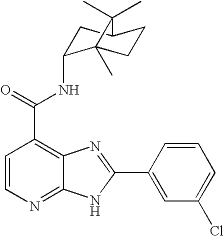 | 0.96 (s, 3H), 0.99 (s, 3H), 1.13 (s, 3H), 1.17-2.02 (m, 1H), 1.41-1.63 (m, 3H), 1.78-2.02 (m, 3H), 4.48- 4.54 (m, 1H), 7.67 (m, 3H), 7.83 (d, 1H), 8.40 (m, 3H), 8.59 (d, 1H), 9.11 (s, 1H), 11.60 (s, 1H) | 408.9 |
| 450 | 2-(3-chloro-phenyl)- 3H-imidazo[4,5- b]pyridine-7-carboxylic acid(6-methyl-pyridin- 3-yl)-amide | 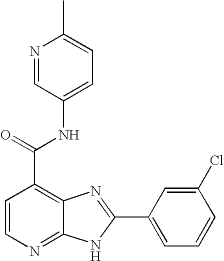 | 11.60 (s, 1H), 9.11 (s, 1H), 8.59 (d, 1H), 8.40 (m, 3H), 7.83 (d, 1H), 7.67 (m, 3H), 2.62 (s, 3H) | 363.8 |
| 451 | 2-(3-fluoro-phenyl)-3H- imidazo[4,5- b]pyridine- 7-carboxylic acid(4- methyl-pyridin-3-yl)- amide | 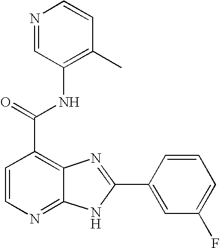 | 11.44 (br, 1H), 9.43 (br, 1H), 8.62 (d, 1H), 8.41 (br, 1H), 8.20 (m, 2H), 7.90 (m, 1H), 7.80~7.38 (m, 4H), 2.66 (s, 1H) | 347.4 |
| 452 | 6-bromo-2-(3-fluoro- phenyl)-3H-imidazo[4,5- b]pyridine-7-carboxylic acid(4-methyl-pyridin-3- yl)-amide | 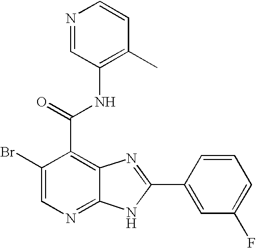 | 10.54 (br, 1H), 8.69~8.59 (m, 1H), 8.36 (d, 1H), 8.14~8.03 (m, 3H), 7.67 (m, 1H), 7.47~7.37 (m, 3H) | 426.3 |
| 453 | 2-(3-fluoro-phenyl)-3H- imidazo[4,5- b]pyridine- 7-carboxylic acid (1,7,7- trimethyl- bicyclo[2.2.1]heptin-2- yl)-amide | 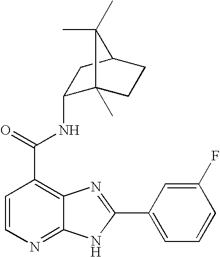 | 0.96 (s, 3H), 0.99 (s, 3H), 1.13 (s, 3H), 1.17-2.02 (m, 1H), 1.41-1.63 (m, 3H), 1.78-2.02 (m, 3H), 4.48- 4.54 (m, 1H), 7.767 (m, 3H), 7.83 (d, 1H), 8.50 (m, 3H), 8.39 (d, 1H), 9.71 (s, 1H), 11.20 (s, 1H) | 392.5 |
| 454 | 2-(3-fluoro-phenyl)-3H- imidazo[4,5- b]pyridine- 7-carboxylic acid(6- methyl-pyridin-3-yl)- amide | 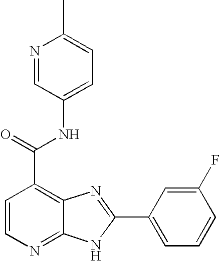 | 11.63 (s, 1H), 9.15 (s, 1H), 8.60 (d, 1H), 8.51 (d, 1H), 8.29 (m, 2H), 7.84 (d, 1H), 7.70 (m, 2H), 7.47 (m, 1H), 2.63 (s, 3H) | 347.4 |
| 455 | 2-(3-fluoro-phenyl)-3H- imidazo[4,5- b]pyridine- 7-carboxylic acid(4- ethoxy-pyridin-3-yl)- amide | 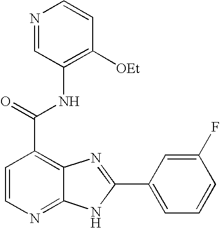 | 11.93 (s, 1H), 9.71 (s, 1H), 8.64 (m, 2H), 8.20 (m, 2H), 7.90 (d, 1H), 7.75 (m, 2H), 7.51 (m, 1H), 4.68 (q, 2H), 1.47 (t, 3H) | 377.4 |
| 456 | 2-(3-fluoro-phenyl)-3H- imidazo[4,5- b]pyridine- 7-carboxylic acid(4- ethylsulfanyl-pyridin-3- yl)-amide | 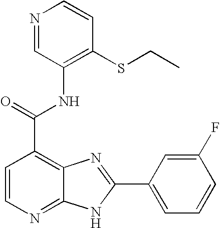 | 11.49 (s, 1H), 9.33 (S, 1H), 8.59 (d, 1H), 8.38 (d, 1H), 8.32 (d, 2H), 7.88 (d, 1H), 7.68 (m, 2H), 7.45 (m, 1H), 3.30 (q, 2H), 1.40 (t, 3H) | 393.4 |
| 457 | 6-bromo-2-(3-fluoro- phenyl)-3H-imidazo[4,5- b]pyridine-7-carboxylic acid(4-ethoxy-6-methyl- pyridin-3-yl)-amide | 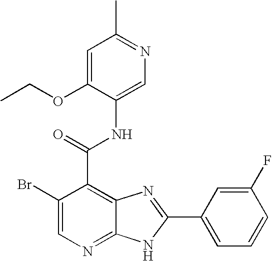 | 9.23 (s, 1H), 8.58 (s, 1H) 8.06!7.97 (m, 2H), 7.60 (m, 1H), 7.33 (m, 1H), 7.06 (s, 1H), 4.30 (m, 2H), 2.53 (s, 3H), 1.43 (t, 3H) | 470.3 |
| 458 | 2-(3-fluoro-phenyl)-3H- imidazo[4,5-b]pyridine- 7-carboxylic acid(4- ethoxy-6-methyl- pyridin-3-yl)-amide | 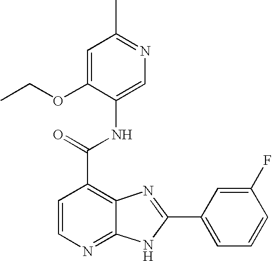 | 9.49 (br, 1H), 8.52 (s, 1H), 8.10 (m, 2H), 7.92 (m, 1H), 7.63 (m, 1H), 7.34 (m, 1H), 7.14 (br, 1H), 4.48 (m, 2H), 2.64 (s, 3H), 1.29 (t, 3H) | 391.4 |

The compound of formula 1 of the present invention may be in the form of a pharmaceutically acceptable salt derived from an inorganic or organic acid, or a base, and representative examples of the pharmaceutically acceptable salt derived from an inorganic or organic include salts obtained by adding an inorganic acid such as hydrochloric acid, hydrobromic acid, phosphoric acid or sulfonic acid, or organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid or malic acid, methanesulfonic acid, or para toluenesulfonic acid, which do not limit its scope, to the compound of formula 1. Such acid salts may be prepared by the conventional processes, and other acids, which themselves are not pharmaceutically acceptable, including oxalic acid may be employed in the preparation of the bases.

Further, the compound of formula 1 may be used in the form of a prodrug derivative thereof, wherein the derivative or prodrug thereof may be a physiologically hydrolysable ester or amide compound, e.g., indanyl, phthalidil, methoxymethyl, pivaloyloxymethyl, glycyloxymethyl, phenylglycyloxymethyl and 5-methyl-2-oxo-1,3-dioxolene-4-ylmethyl.

In accordance with another aspect of the present invention, there is provided a method for preparing the compound of formula 1.

A compound of formula 1 may be prepared by a method comprising the steps of:

1) hydrogenating a compound of formula 2 in the presence of a catalyst to obtain a compound of formula 3;

2) refluxing a mixture of the compound of formula 3 and R₁—(CO₂H) or R₁—(CHO) in the presence of an organic acid or heating the mixture in nitrobenzene by microwave irradiation to obtain a compound of formula 4; 3) reacting the compound of formula 4 with an oxidizing agent in an alkali hydroxide solution or an organic solvent, cooling the resulting mixture in an ice bath, adding SOCl₂ or H₂SO₄ thereto, and refluxing the resulting mixture to obtain a compound of formula 5;

4) refluxing the compound of formula 5 together with LiOH.H₂O in a solvent and adding an acid thereto to obtain a compound of formula 6; and

5) reacting the compound of formula 6 with a compound of formula R₂R₃NH in an organic solvent in the presence of a coupling agent to obtain the compound of formula 1:

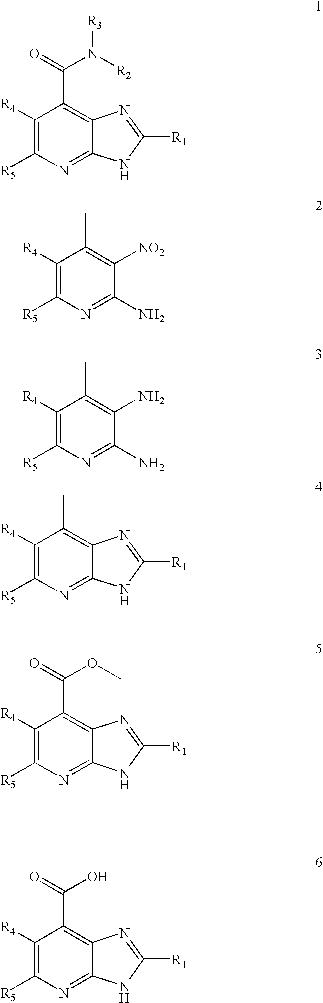

wherein, R₁ to R₅ have the same meanings as defined above.

The inventive method for preparing the compound of formula 1 is shown in Reaction Scheme 1.

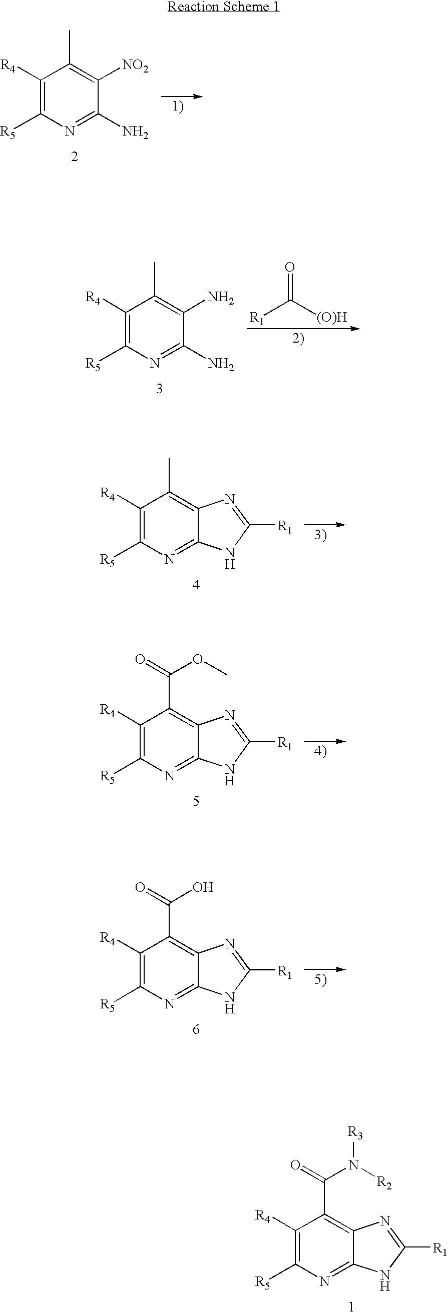

wherein,

R₁, R₂, R₃, R₄ and R₅ have the same meanings as defined above.

As shown in Reaction Scheme 1, the compound of formula 2 may be first hydrogenated in the presence of a catalyst such as 5% to 10% Pd/C or PtO₂ in an organic solvent in a hydrogenation reactor, the resulting mixture is filtered and concentrated under a reduced pressure to obtain a compound of formula 3. The compound of formula 2 used as a starting material may be prepared by a conventional method (see TANGA, M. J et al., J Heterocycl Chem 2003, 40 (4), 569-573) or commercially available. The organic solvent may be methanol, ethanol or methylene chloride, and the reaction may be carried out at room temperature.

In step 2, the compound 3 may be refluxed in the presence of an organic acid at 180 to 200° C. for 4 to 6 hours, or heated in a nitrobenzene by a microwave irradiation with a power of 200 to 300 W at a temperature of 180 to 200° C. for 20 to 40 minutes, with R₁—(CO₂H) or R₁—(CHO) in an amount preferably ranging from 1 to 2 equivalents based on the compound 3. The resulting mixture may be neutralized with aqueous NaOH, extracted, filtered to remove the solvent, and the resulting residue is subjected to flash column chromatography to obtain a compound 4. The organic acid may be POCl₃ or phosphoric acid (PPA).

In step 3, the compound 4 may be reacted with an oxidizing agent in an alkali hydroxide solution or an organic solvent, cooling the resulting mixture in an ice bath, adding SOCl₂ or H₂SO₄ thereto and refluxing the mixture in methanol to obtain a compound of formula 5. The alkali hydroxide may be NaOH, NaHCO₃ or Na₂CO₃, and the organic solvent may be pyridine or t-BuOH. The oxidizing agent may be KMnO₄, MnO₂ or SeO₂, and it is used in an amount ranging from 2 to 4 equivalents based on the compound of formula 4. SOCl₂ or H₂SO₄ may be employed in an amount ranging from 0.1 to 4 equivalents based on the compound 4.

In step 4, the compound 5 may be refluxed together with LiOH.H₂O in an amount preferably ranging from 2 to 3 equivalents based on the compound 5 in a mixture of water, MeOH and THF at 80° C., and the resulting mixture may be treated with HCl in an amount preferably ranging from 1 to 3 equivalents based on the compound 5 to obtain a compound of formula 6. The weight ratio of the water:MeOH:THF may range from 1:0.5˜2:1˜5, preferably about 1:1:3.

In step 5, the compound 6 may be reacted with a compound of formula R₂R₃NH in the presence of a coupling agent in an organic solvent to obtain a compound of formula 1. The organic solvent may be dimethylformamide (DMF), dimethyl sulfoxide (DMSO) or methylenechloride (MC). The coupling agent may be 1-hydroxybenzotriazole (HOBT)/1-(3-dimethylaminopropyl)-3-ethylcarbdiimide HCl salt (EDC)/triethylamine (Et₃N), and pyBop ((benzotriazole-1-yl-oxy)tripyrrolidinophosphonium hexafluorophosphate), HBTU (O-benzotriazole-N,N,N′,N′-tetramethyluronium hexafluorophosphate) or TBTU (O-(benzotriazole-1-yl)-N,N,N′,N′-tetramethyluronium tetrafluoroborate). Further, the coupling agent and R₂R₃NH may be each employed in an amount ranging from 2 to 3 equivalents based on the compound 5.

The compound of formula 2 used as the stating material is commercially available.

In accordance with further aspect of the present invention, there is provided a composition for inhibiting the activity of the protein kinase comprising said imidazopyridine derivatives, or a pharmaceutically acceptable salt, hydrate, solvate or isomer thereof as an active ingredient.

The protein kinases may be selected from the group consisting of glycogen synthase kinase-3 (GSK-3), aurora kinase, extracellular signal-regulated kinase (ERK), protein kinase B (AKT), cyclin-dependent kinase (CDK), p38 (protein 38) mitogen-activated protein kinase (MAPK), kinase insert domain protein receptor (KDR) or vascular endothelial growth factor receptor-2 (VEGFR-2), c-Jun N-terminal kinase (JNK) and pyruvate dehydrogenase kinase (PDK). The inventive compound has an IC₅₀ value of 3 nM to 50,000 nM for said protein kinases.

In addition, the inventive imidazopyridine derivative of formula 1, or a pharmaceutically acceptable salt, hydrate, solvate or isomer thereof as an active ingredient may be used in an pharmaceutical composition for preventing or treating diseases selected from the group consisting of diabetes, obesity, dementia, cancer, and inflammation since it can efficiently inhibit the activities of several protein kinases including aurora kinase and control signal transductions thereof. Accordingly, in the present invention, there is provided a pharmaceutical composition comprising said imidazopyridine derivative, or a pharmaceutically acceptable salt, hydrate, solvate or isomer thereof as an active ingredient.

The salt, hydrate, solvate or isomer of the compound of formula 1 may be prepared from the compound of formula 1 in accordance with the conventional method.

The pharmaceutically acceptable composition may be formulated for oral or parenteral administration. The composition for oral administration may take various forms such as tablets, powder, rigid or soft gelatin capsules, solution, dispersion, emulsions, syrups and granules, such formulations may comprise the active ingredient together with diluting agents (e.g., lactose, dextrose, sucrose, mannitol, sorbitol, cellulose and/or glycine), and lubricants (e.g., silica, talc, stearic acid and a magnesium or calsium salt thereof and/or polyethyleneglycol). Further, these tablets may comprise binding agents such as magnesium aluminium silicate, starch paste, gelatin, tragacanth, methylcellulose, sodium carboxymethylcellulose and/or polyvinylpyrrolidine, and may further comprise disintegrants such as starch, agarose, alginate or a sodium salt thereof or an effervescent mixture and/or an absorbing, colouring, flavouring, and sweetening agents.

Further, the inventive pharmaceutical composition may take forms of preferably injections further comprising saline solution or suspensions when formulated for parenteral administration.

The pharmaceutical composition may be sterilized and/or may further comprise preservatives, stabilizing agents, hydrating agents or emulsifiers, salts for controlling osmotic pressure and/or supplementary agents including buffer agents and other therapeutically available materials, and may be prepared by the conventional mixing, granulating or coating methods.

A proposed daily dose of the compound of formula 1 used as an active ingredient in the inventive composition for administration to a mammal including human is about from 2.5 mg/kg weight to 100 mg/kg weight, more preferably about from 5 mg/kg weight to 60 mg/kg weight. It should be understood that the daily dose should be determined in light of various relevant factors including the condition to be treated, the severity of the patient's symptoms, the route of administration, or the physiological form of the anticancer agent; and, therefore, the dosage suggested above should not be construed to limit the scope of the invention in anyway.

The following Examples are intended to further illustrate the present invention without limiting its scope.

### EXAMPLE 1

### Preparation of 2-phenyl-3H-imidazo[4,5-b]-pyridine-7-carboxylic acid [2-(4-acetylamino-phenyl)-ethyl]-amide

### Step 1) Preparation of 4-methylpyridine-2,3-diamine

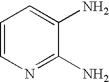

4-Methyl-3-nitropyridine-2-amine (25 g, 163.4 mmol) was dissolved in 100 ml of MeOH, and a catalytic amount of 5% Pd/C was added thereto, and the mixture was stirred for 2 hrs. The reaction solution was filtered through a celite pad, the pad was thoroughly washed with MeOH, and the combined mixture was concentrated under a reduced pressure to remove the solvent. The resulting residue was vacuum dried to obtain the title compound (18.49 g, 150.33 mmol; yield: 92%).

¹H-NMR (CDCl₃): 2.17 (s, 3H), 3.27 (br, 2H), 4.14 (br, 2H), 6.54 (d, 2H), 7.55 (d, 2H)

M.W.: 124

### Step 2) Preparation of 7-methyl-2-phenyl-3H-imidazo[4,5-b]pyridine

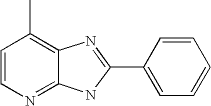

The compound obtained in Step 1 (5 g, 40.65 mmol), benzoic acid (4.96 g, 40.65 mmol) and 20 ml of POCl₃ were mixed, and the mixture was refluxed at 170-180° C. for 4 hours. The reaction mixture was concentrated under a reduced pressure to remove POCl₃, neutralized with aqueous NaOH and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (5.09 g, 24.39 mmol; yield: 60%).

¹H-NMR (CDCl₃): 2.78 (s, 3H), 7.09 (d, 1H), 7.54 (m, 3H), 8.30 (m, 3H)

M.W.: 210

### Step 3) Preparation of 2-phenyl-3H-imidazo[4,5-b]pyridine-7-carboxylic acid methyl ester

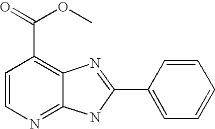

The compound obtained in Step 2 (200 mg, 0.96 mmol) and NaOH (76.80 mg, 1.92 mmol) were mixed, 20 ml of water was added thereto, and the resulting mixture was heated to 60° C. Aqueous KMnO₄ (311 mg, 1.92 mmol) obtained by heating KMnO₄ dissolved in water with heating was added to the mixture, and the mixture was stirred at 100° C. for 6 hours. The reaction mixture was filtered through a celite pad while keeping it hot, the pad was thoroughly washed with hot water, and the combined aqueous solution was concentrated under a reduced pressure to remove the solvent, followed by vacuum drying. The resulting residue was dissolved in 20 ml of MeOH, and the mixture was cooled to 0° C. in an ice bath. SOCl₂ was slowly added thereto in an amount of 7-10 equivalents and the mixture was refluxed for 4 hours. The resulting mixture was neutralized with aqueous NaOH, concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (109 mg, 0.43 mmol; yield: 45%).

¹H-NMR (MeOH-d₄): 4.10 (s, 3H), 7.64 (m, 3H), 7.89 (d, 1H), 8.25 (m, 2H), 8.61 (d, 2H)

M.W.: 254

### Step 4) Preparation of 2-phenyl-3H-imidazo[4,5-b]pyridine-7-carboxylic acid

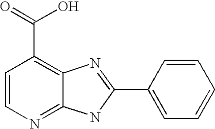

The compound obtained in Step 3 (30 mg, 0.12 mmol) was dissolved in a mixture of 10 ml of THF, 3 ml of water and 3 ml of MeOH. LiOH.H₂O (14 mg, 0.33 mmol) was added thereto and the mixture was refluxed for 8 hours. After the reaction was terminated by the addition of 4M HCl (0.66 mmol, 165 e) at room temperature, the reaction mixture was concentrated under a reduced pressure to remove the solvent. The resulting residue was vacuum dried to obtain the title compound (25.8 mg, 0.10 mmol; yield: 85%).

¹H-NMR (MeOH-d₄): 7.79˜7.67 (m, 3H), 8.14 (d, 1H), 8.28 (m, 2H), 8.78 (d, 1H)

M.W.: 254

### Step 5) Preparation of 2-phenyl-3H-imidazo[4,5-b]pyridine-7-carboxylic acid [(2-(4-acetylamino-phenyl)-ethyl]-amide

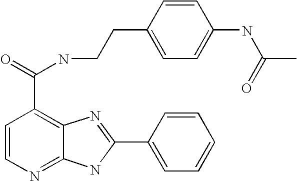

The compound obtained in Step 4 was dissolved in 3 ml of DMF, and 2 equivalents each of EDC and HOBt were further dissolved in the solution while stirring. 4-Acetylpentylamine was added to the mixture in an amount of 1.2 equivalents, and the mixture was stirred at room temperature for 12-24 hours. The reaction mixture was vacuum dried, and the resulting residue was dissolved in a small quantity of MeOH and filtered. The filtrate thus obtained was subjected to Prep. HPLC to obtain the title compound (yield: 60%).

### EXAMPLES 2 TO 31

The procedure of Example 1 was repeated except for using each of the corresponding amine compounds instead of 4-acetylpentylamine in Step 5, to obtain the respective title compounds.

### EXAMPLES 32 TO 42

The procedure of Example 1 was repeated except for using 2,4-dichloro-benzoic acid (7.76 g, 40.65 mmol) and corresponding amine compounds instead of benzoic acid and 4-acetylpentylamine, respectively, in Steps 2 and 5, to obtain the respective title compounds.

### EXAMPLE 43

### Preparation of 2-(4-chloro-phenyl)-3H-imidazo[4,5-b]pyridine-7-carboxylic acid [2-(4-ethanesulfonylamino-phenyl)-ethyl]-amide

### Step 1) Preparation of 2-(4-chlorophenyl)-7-methyl-3H-imidazo[4,5-b]pyridine

The compound obtained in Step 1 of Example 1 (0.75 g, 6.14 mmol), 4-chlorobenzoic acid (1.153 g, 7.37 mmol) and PPA (10 g) were mixed, and the mixture was stirred at 150-160° C. for 24 hours. The reaction mixture was cooled to room temperature and 20 ml of water was slowly added thereto, followed by the neutralization with aqueous and saturated NaOH in an ice bath. The formed precipitates were filtered and vacuum dried to obtain the crude title compound (1.24 g, 5.10 mmol; yield: 83%).

¹H-NMR (CDCl₃): 2.69 (s, 3H), 7.12 (d, 1H), 7.57 (d, 2H), 8.16 (d, 2H), 8.21 (m, 1H)

M.W.: 244

### Step 2) Preparation of methyl 2-(4-chlorophenyl)-3H-imidazo[4,5-b]pyridine-7-carboxylate

The compound obtained in Step 1 (0.138 g, 0.67 mmol) was dissolved in 3 ml of t-BuOH, and the mixture was stirred. Aqueous and hot KMnO₄ (450 mg, 2.85 mmol) dissolved in 4 ml of water with heating was added thereto with three portions, and the mixture was stirred at 60-80° C. for 24 hours. The reaction mixture was filtered through a celite pad while keeping it hot, the pad was thoroughly washed with hot water, and the combined mixture was concentrated under a reduced pressure to remove the solvent, followed by vacuum drying. The resulting residue was dissolved in 5 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (45 mg, 0.16 mmol; yield: 23%).

¹H-NMR (CDCl₃): 4.09 (s, 3H), 7.41 (d, 1H), 7.52 (d, 2H), 7.97 (d, 2H), 8.12 (d, 1H), 10.43 (br, 1H)

M.W.: 289

### Step 3) Preparation of 2-(4-chlorophenyl)-3H-imidazo[4,5-b]pyridine-7-carboxylic acid

The compound obtained in Step 2 (45 mg, 0.16 mmol) was dissolved in 2 ml of THF. LiOH.H₂O (20 mg, 0.48 mmol) dissolved in 1 ml of water was added thereto and the mixture was stirred at room temperature for 8 hours. The reaction mixture was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (45 mg, 0.16 mmol; yield: 102%).

¹H-NMR (MeOH-d₄): 7.32 (d, 2H), 7.56 (d, 1H), 7.90 (d, 2H), 8.39 (d, 1H)

M.W.: 275

### Step 4) Preparation of 2-(4-chlorophenyl)-3H-imidazo[4,5-b]pyridine-7-carboxylic acid [2-(4-ethanesulfonylamino-phenyl)-ethyl]-amide

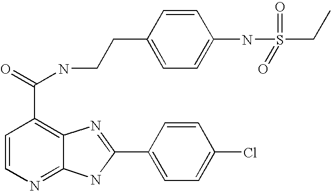

The compound obtained in Step 3 was dissolved in DMF, and 1.2 equivalents of benzotriazol-1-yl-oxytripyrrolidinophsphonium (PyBOP), 3 equivalents of TEA and 1.2 equivalents of ethanesulfonic acid [4-(2-aminoethyl)-phenyl]-amide were added thereto, and the mixture was stirred at room temperature for 12 hours. The reaction mixture was vacuum dried, and the resulting residue was dissolved in a small quantity of MeOH and filtered. The filtrate thus obtained was subjected to Prep. HPLC to obtain the title compound (yield: 65%).

### EXAMPLES 44 TO 61

The procedure of Example 43 was repeated except for using each of the corresponding amine compounds instead of ethanesulfonic acid [4-(2-aminoethyl)-phenyl]-amide in Step 4, to obtain the respective title compounds.

### EXAMPLE 62

### Preparation of 2-furan-2-yl-3H-imidazo[4,5-b]pyridine-7-carboxylic acid [3-(4,5-dichloro-3H-imidazol-1-yl)-propyl]-amide

### Step 1) Preparation of 2-furan-2-yl-7-methyl-3H-imidazo[4,5-b]pyridine

The compound obtained in Step 1 of Example 1 (1 g, 8.15 mmol) and furan 2-carboxyaldehyde (783 mg, 8.15 mmol) were dissolved in 3 ml of nitrobenzene, and the mixture was kept under a 300 W power at 180° C. for 15 mins. The reaction solution was subjected to flash column chromatography (eluent: n-hexane/ethyl acetate=1:1) to obtain the title compound (730 mg, 3.67 mmol; yield: 45%).

¹H-NMR (CDCl₃): 2.69 (s, 3H), 7.12 (d, 1H), 7.57 (d, 2H), 8.16 (d, 2H), 8.21 (m, 1H)

M.W.: 200

### Step 2) Preparation of methyl 2-furan-2-yl-3H-imidazo[4,5-b]pyridine-7-carboxylate

The compound obtained in Step 1 (0.469 g, 2.35 mmol) was dissolved in 5 ml of pyridine, and the mixture was stirred. SeO₂ (1.303 g, 11.75 mmol) was added thereto, and the mixture was refluxed at 120° C. for 24 hours. The reaction solution was filtered through a celite pad while keeping it hot, the pad was thoroughly washed with hot water and MeOH, and the combined mixture was concentrated under a reduced pressure to remove the solvent, followed by vacuum drying. The resulting residue was dissolved in 10 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (188 mg, 0.776 mmol; yield: 33%).

¹H-NMR (CDCl₃): 4.08 (s, 3H), 6.74 (d, 1H), 7.52 (d, 1H), 7.78 (d, 1H), 7.87 (d, 1H), 8.51 (d, 1H)

M.W.: 244

### Step 3) Preparation of 2-furan-2-yl-3H-imidazo[4,5-b]pyridine-7-carboxylic acid

The compound obtained in Step 2 (140 mg, 0.58 mmol) was dissolved in 2 ml of THF, and the mixture was stirred. LiOH.H₂O (73 mg, 1.74 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (109 mg, 0.48 mmol; yield: 83%).

¹H-NMR (MeOH-d₄): 6.71 (d, 1H), 7.40 (d, 1H), 7.67 (d, 1H), 7.83 (d, 1H), 8.39 (d, 1H)

M.W.: 230

### Step 4) Preparation of 2-furan-2-yl-3H-imidazo[4,5-b]pyridine-7-carboxylic acid [3-(4,5-dichloro-3H-imidazol-1-yl)-propyl]-amide

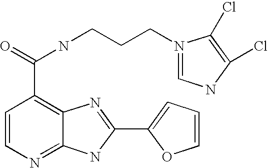

The compound obtained in Step 3 was dissolved in DMF, and 1.2 equivalents of PyBOP, 3 equivalents of TEA and 1.2 equivalents of 3-(2,4-dichloroimidazolyl)propylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 45%).

### EXAMPLES 63 TO 85

The procedure of Example 62 was repeated except for using each of the corresponding amine compounds instead of 3-(2,4-dichloroimidazolyl)propylamine in Step 4, to obtain the respective title compounds.

### EXAMPLE 86

### Preparation of 2-thiophen-2-yl-3H-imidazo[4,5-b]pyridine-7-carboxylic acid [2-(4-ethanesulfonylamino-phenyl)-ethyl]-amide

### Step 1) Preparation of 7-methyl-2-thiophen-2-yl-3H-imidazo[4,5-b]pyridine

The compound obtained in Step 1 of Example 1 (1 g, 8.15 mmol) and thiophen-2-carboxyaldehyde (912 mg, 8.15 mmol) were dissolved in 3 ml of nitrobenzene, and the mixture was kept under a 300 W power at 180° C. for 15 mins. The reaction solution was subjected to flash column chromatography (eluent: n-hexane/ethyl acetate=1:1) to obtain the title compound (877 mg, 4.08 mmol; yield: 50%).

¹H-NMR (CDCl₃): 2.17 (s, 3H), 7.36 (t, 1H), 8.00 (m, 2H), 8.48 (d, 1H), 8.82 (d, 1H)

M.W.: 216

### Step 2) Preparation of 2-thiophen-2-yl-3H-imidazo[4,5-b]pyridine-7-carboxylic acid methyl ester

The compound obtained in Step 1 (500 mg, 2.33 mmol) was dissolved in 5 ml of pyridine, and the mixture was stirred. SeO₂ (1.04 g, 9.32 mmol) was added thereto and the mixture was refluxed at 120° C. for 24 hours. The reaction solution was filtered through a celite pad while keeping it hot, the pad was thoroughly washed with hot water and MeOH, the combined mixture was concentrated under a reduced pressure to remove the solvent, followed by vacuum drying. The resulting residue was dissolved in 10 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (163 mg, 0.63 mmol; yield: 27%).

¹H-NMR (CDCl₃): 4.08 (s, 3H), 6.74 (d, 1H), 7.52 (d, 1H), 7.78 (d, 1H), 7.87 (d, 1H), 8.41 (d, 1H)

M.W.: 260

### Step 3) Preparation of 2-thiophen-2-yl-3H-imidazo[4,5-b]pyridine-7-carboxylic acid

The compound obtained in Step 2 (163 mg, 0.63 mmol) was dissolved in 2 ml of THF, and the mixture was stirred. LiOH.H₂O (126 mg, 2.52 mmol) dissolved in 1 ml of water was added thereto, and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (103 mg, 0.42 mmol; yield: 67%).

¹H-NMR (MeOH-d₄): 6.71 (d, 1H), 7.40 (d, 1H), 7.67 (d, 1H), 7.83 (d, 1H), 8.39 (d, 1H)

M.W.: 246

### Step 4) Preparation of 2-thiophen-2-yl-3H-imidazo[4,5-b]pyridine-7-carboxylic acid [2-(4-ethanesulfonylamino-phenyl)-ethyl]-amide

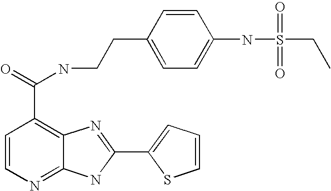

The compound obtained in Step 3 was dissolved in DMF, and 1.2 equivalents of PyBOP, 3 equivalents of TEA and 1.2 equivalents of 4-ethanesulfonylaminophenethylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 55%).

### EXAMPLES 87 TO 113

The procedure of Example 86 was repeated except for using each of the corresponding amine compounds instead of 4-ethanesulfonylaminophenethylamine in Step 4, to obtain the respective title compounds.

### EXAMPLE 114

### Preparation of 2-furan-3-yl-3H-imidazo[4,5-b]pyridine-7-carboxylic acid [2-(4-ethanesulfonylamino-phenyl)-ethyl]-amide

### Step 1) Preparation of 2-furan-3-yl-7-methyl-3H-imidazo[4,5-b]pyridine

The compound obtained in Step 1 of Example 1 (500 g, 4.07 mmol) and furan 3-carboxyaldehyde (391 mg, 4.07 mmol) were dissolved in 3 ml of nitrobenzene, and the mixture was kept under a 300 W power at 180° C. for 15 mins. The reaction solution was subjected to flash column chromatography (eluent: n-hexane/ethyl acetate=1:1) to obtain the title compound (243 mg, 1.22 mmol; yield: 30%).

¹H-NMR (CDCl₃): 2.88 (s, 3H), 7.09 (s, 1H), 7.17 (d, 2H), 8.38 (s, 1H), 8.44 (d, 1H)

M.W.: 200

### Step 2) Preparation of 2-furan-3-yl-3H-imidazo[4,5-b]pyridine-7-carboxylic acid methyl ester

The compound obtained in Step 1 (243 mg, 1.22 mmol) was dissolved in 5 ml of pyridine, and the mixture was stirred. SeO₂ (542 mg, 4.88 mmol) was added thereto and refluxed at 120° C. for 24 hours. The reaction solution was filtered through a celite pad while keeping it hot, the pad was thoroughly washed with hot water and MeOH, and the combined mixture was concentrated under a reduced pressure to remove the solvent, followed by vacuum drying. The resulting residue was dissolved in 10 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (154 mg, 0.63 mmol; yield: 52%).

¹H-NMR (CDCl₃): 4.13 (s, 3H), 7.09 (s, 1H), 7.17 (d, 2H), 8.38 (s, 1H), 8.44 (d, 1H)

M.W.: 244

### Step 3) Preparation of 2-furan-3-yl-3H-imidazo[4,5-b]pyridine-7-carboxylic acid

The compound obtained in Step 2 (154 mg, 0.63 mmol) was dissolved in 2 ml of THF, and the mixture was stirred. LiOH.H₂O (106 mg, 2.52 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (109 mg, 0.48 mmol; yield: 76%).

¹H-NMR (MeOH-d₄): 6.71 (d, 1H), 7.40 (d, 1H), 7.67 (d, 1H), 7.83 (d, 1H), 8.39 (d, 1H)

M.W.: 230

### Step 4) Preparation of 2-furan-3-yl-3H-imidazo[4,5-b]pyridine-7-carboxylic acid [2-(4-methanesulfonylamino-phenyl)-ethyl]-amide

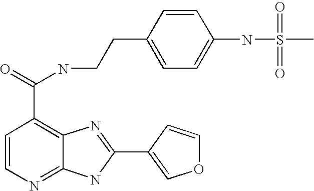

The compound obtained in Step 3 was dissolved in DMF, and 1.2 equivalents of PyBOP, 3 equivalents of TEA and 1.2 equivalents of 4-methanesulfonylaminophenethylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 45%).

### EXAMPLES 115 TO 141

The procedure of Example 114 was repeated except for using each of the corresponding amine compounds instead of 4-methanesulfonylaminophenethylamine in Step 4, to obtain the respective title compounds.

### EXAMPLE 142

### Step 1) Preparation of 2-(2,4-difluorophenyl)-7-methyl-3H-imidazo[4,5-b]pyridine

The compound obtained in Step 1 of Example 1 (0.75 g, 6.14 mmol), 2,4-difluorobezoic acid (1.456 g, 9.21 mmol) and PPA (10 g) were mixed, and the mixture was stirred at 150-160° C. for 24 hours. The reaction solution was cooled to room temperature and 20 ml of water was slowly added thereto, followed by the neutralization with aqueous and saturated NaOH in an ice bath. The formed precipitates were filtered and vacuum dried to obtain the crude title compound (200 mg, 0.816 mmol; yield: 13%).

¹H-NMR (CDCl₃): 2.68 (s, 3H), 7.14 (m, 3H), 8.16 (m, 1H), 8.25 (d, 1H)

M.W.: 246

### Step 2) Preparation of methyl 2-(4-difluorophenyl)-3H-imidazo[4,5-b]pyridine-7-carboxylate

The compound obtained in Step 1 (0.20 g, 0.82 mmol) was dissolved in ml of t-BuOH, and the mixture was stirred. Aqueous and hot KMnO₄ (648 mg, 4.1 mmol) dissolved in 4 ml of water with heating was added thereto with three portions, and stirred at 60-80° C. for 24 hours. The reaction solution was filtered through a celite pad while keeping it hot, the pad was thoroughly washed with hot water, and the combined mixture was concentrated under a reduced pressure to remove the solvent, followed by vacuum drying. The resulting residue was dissolved in 5 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (50 mg, 0.17 mmol; yield: 21%).

¹H-NMR (CDCl₃): 4.09 (s, 3H), 7.02 (m, 1H), 7.14 (m, 1H), 7.72 (d, 1H), 8.65 (m, 1H), 8.69 (d, 1H), 10.43 (br, 1H)

M.W.: 290

### Step 3) Preparation of 2-(2,4-difluorophenyl)-3H-imidazo[4,5-b]pyridine-7-carboxylic acid

The compound obtained in Step 2 (50 mg, 0.17 mmol) was dissolved in 2 ml of THF. LiOH.H₂O (21 mg, 0.51 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (70 mg, 0.256 mmol; yield: 150%).

¹H-NMR (MeOH-d₄): 7.23 (d, 2H), 7.79 (d, 1H), 8.38 (m, 2H), 8.48 (d, 1H)

M.W.: 275

### Step 4) Preparation of 2-(2,4-difluoro-phenyl)-3H-imidazo[4,5-b]pyridine-7-carboxylic acid [2-(4-methanesulfonylamino-phenyl)-ethyl]-amide

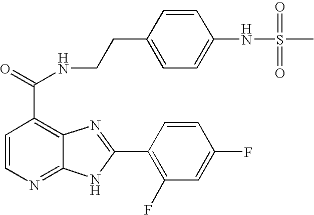

The compound obtained in Step 3 was dissolved in DMF, and 1.2 equivalents of PyBOP, 3 equivalents of TEA and 1.2 equivalents of methanesulfonylaminophic acid [4-(2-aminoethyl)-phenyl]-amide were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 45%).

### EXAMPLES 143 TO 144

The procedure of Example 142 was repeated except for using each of the corresponding amine compounds instead of 4-methanesulfonylaminophenethylamine in Step 4, to obtain the respective title compounds.

### EXAMPLE 145

### Step 1) Preparation of 2-cyclopropyl-7-methyl-3H-imidazo[4,5-b]pyridine

The compound obtained in Step 1 of Example 1 (500 mg, 4.08 mmol), cyclopropan carboxylic acid (351 mg, 4.08 mmol) and 25 ml of POCl₃ were mixed, and the mixture was refluxed at 170-180° C. for 4 hours. The reaction solution was concentrated under a reduced pressure to remove POCl₃, neutralized with aqueous NaOH and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=2:1) to obtain the title compound (423 mg, 2.45 mmol; yield: 60%).

¹H-NMR (CDCl₃): 1.25 (m, 2H), 1.31 (m, 2H), 2.31 (m, 1H), 2.91 (t, 2H), 7.70 (d, 1H), 8.41 (d, 1H)

M.W.: 174

### Step 2) Preparation of 2-cyclopropyl-3H-imidazo[4,5-b]pyridine-7-carboxylic acid methyl ester

The compound obtained in Step 1 (423 mg, 2.45 mmol) and NaOH (196 mg, 4.9 mmol) were mixed, 20 ml of water was added thereto, and the mixture was heated to 60° C. Aqueous KMnO₄ (774 mg, 4.9 mmol) dissolved in water (3 ml) with heating was added to the mixture, and the mixture was stirred at 100° C. for 6 hours. The reaction solution was filtered through a celite pad while keeping it hot, the pad was thoroughly washed with hot water, and the combined mixture was concentrated under a reduced pressure to remove the solvent, followed by vacuum drying. The resulting residue was dissolved in 20 ml of MeOH, and the mixture was cooled to 0° C. in an ice bath. SOCl₂ was slowly added thereto in an amount of 7-10 equivalents and the mixture was refluxed for 4 hours. The resulting mixture was neutralized with aqueous NaOH, concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=2:1) to obtain the title compound (170 mg, 0.78 mmol; yield: 32%).

¹H-NMR (CDCl₃): 1.25 (m, 2H), 1.31 (m, 2H), 2.31 (m, 1H), 2.91 (t, 2H), 3.80 (s, 3H), 7.70 (d, 1H), 8.41 (d, 1H)

M.W.: 218

### Step 3) Preparation of 2-chloropropyl-3H-imidazo[4,5-b]pyridine-7-carboxylic acid

The compound obtained in Step 2 (170 mg, 0.78 mmol) was dissolved in 2 ml of THF. LiOH.H₂O (66 mg, 1.56 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (140 mg, 0.69 mmol; yield: 88%).

¹H-NMR (MeOH-d₄): 1.25 (m, 2H), 1.31 (m, 2H), 2.31 (m, 1H), 7.70 (d, 1H), 8.41 (d, 1H)

M.W.: 204

### Step 4) Preparation of 2-cyclopropyl-3H-imidazo[4,5-b]pyridine-7-carboxylic acid [2-(4-methanesulfonylamino-phenyl)-ethyl]-amide

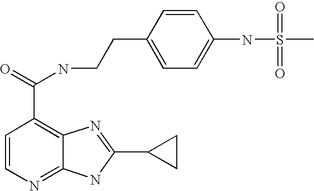

The compound obtained in Step 3 was dissolved in DMF, and 1.2 equivalents of PyBOP, 3 equivalents of TEA and 1.2 equivalents of 4-methanesulfonylaminophenethylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 65%).

### EXAMPLES 146 TO 150

The procedure of Example 145 was repeated except for using each of the corresponding amine compounds instead of 4-methanesulfonylaminophenethylamine in Step 4, to obtain the respective title compounds.

### EXAMPLE 151

### Step 1) Preparation of 7-methyl-2-thiophen-3-yl-3H-imidazo[4,5-b]pyridine

The compound obtained in Step 1 of Example 1 (500 mg, 4.08 mmol) and thiophen-2-carboxyaldehyde (456 mg, 4.08 mmol) were dissolved in 2 ml of nitrobenzene, and the mixture was kept under a 300 W power at 180° C. for 15 mins. The reaction solution was subjected to flash column chromatography (eluent: n-hexane/ethyl acetate=1:1) to obtain the title compound (526 mg, 2.45 mmol; yield: 45%).

¹H-NMR (CDCl₃): 2.66 (s, 3H), 7.11 (t, 1H), 7.64 (d, 1H), 7.86 (d, 1H), 8.19 (s, 1H), 8.29 (d, 1H)

M.W.: 216

### Step 2) Preparation of 2-thiophen-3-yl-3H-imidazo[4,5-b]pyridine-7-carboxylic acid methyl ester

The compound obtained in Step 1 (526 mg, 2.45 mmol) was dissolved in 5 ml of pyridine, and the mixture was stirred. SeO₂ (1.09 mg, 9.80 mmol) was added thereto, and the mixture was refluxed at 120° C. for 24 hours. The reaction solution was filtered through a celite pad while keeping it hot, the pad was thoroughly washed with hot water and MeOH, and the combined mixture was concentrated under a reduced pressure to remove the solvent, followed by vacuum drying. The resulting residue was dissolved in 10 ml of MeOH while stirring. SOCl₂ was slowly added thereto in an amount of 7-10 equivalents and the mixture was refluxed at 80° C. for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (260 mg, 1.01 mmol; yield: 41%).

¹H-NMR (CDCl₃): 4.18 (s, 3H), 7.11 (t, 1H), 7.64 (d, 1H), 7.86 (d, 1H), 8.19 (s, 1H), 8.29 (d, 1H)

M.W.: 260

### Step 3) Preparation of 2-thiophen-3-yl-3H-imidazo[4,5-b]pyridine-7-carboxylic acid

The compound obtained in Step 2 (260 mg, 1.01 mmol) was dissolved in 2 ml of THF, and the mixture was stirred. LiOH H₂O (196 mg, 4.04 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (200 mg, 0.82 mmol; yield: 81%).

¹H-NMR (CDCl₃): 7.13 (t, 1H), 7.68 (d, 1H), 7.86 (d, 1H), 8.19 (s, 1H), 8.29 (d, 1H)

M.W.: 246

### Step 4) Preparation of 2-thiophen-3-yl-3H-imidazo[4,5-b]pyridine-7-carboxylic acid [2-(3-hydroxy-phenyl)-ethyl]-amide

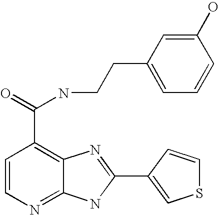

The compound obtained in Step 3 was dissolved in DMF, and 1.2 equivalents of PyBOP, 3 equivalents of TEA and 1.2 equivalents of 2′-hydroxypentylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 35%).

### EXAMPLES 152 TO 178 AND 181 TO 195

The procedure of Example 151 was repeated except for using each of the corresponding amine compounds instead of 2′-hydroxypentylamine in Step 4, to obtain the respective title compounds.

### EXAMPLES 179 TO 180

The procedure of Example 151 was repeated except for using the compound (89.65 mg, 0.246 mmol) obtained in Example 151 and 2 equivalents of mCPBA (metachloro perbenzoic acid) (85 mg) to obtain the respective title compounds.

### EXAMPLE 196

### Step 1) Preparation of 2-(2,4-fluorophenyl)-7-methyl-3H-imidazo[4,5-b]pyridine

The compound obtained in Step 1 of Example 1 (1.25 g, 10.24 mmol), 4-fluorobezoic acid (1.721 g, 12.29 mmol) and PPA (50 g) were mixed, and the mixture was stirred at 150-160° C. for 24 hours. The reaction solution was cooled to room temperature and 20 ml of water was slowly added thereto, followed by the neutralization with aqueous and saturated NaOH in an ice bath. The formed precipitates were filtered and vacuum dried to obtain the crude title compound (1.21 g, 5.32 mmol; yield: 52%).

¹H-NMR (CDCl₃): 2.67 (s, 3H), 7.11 (d, 1H), 7.28 (t, 2H), 8.21 (m, 3H)

M.W.: 228

### Step 2) Preparation of methyl 2-(4-fluorophenyl)-3H-imidazo[4,5-b]pyridine-7-carboxylate

The compound obtained in Step 1 (0.1 g, 0.44 mmol) was dissolved in 3 ml of t-BuOH, and the mixture was stirred. Aqueous and hot KMnO₄ (139 mg, 0.88 mmol) dissolved in 3 ml of water with heating was added thereto with three portions, and stirred at 60-80° C. for 24 hours. The reaction solution was filtered through a celite pad while keeping it hot, the pad was thoroughly washed with hot water, and the combined mixture was concentrated under a reduced pressure to remove the solvent, followed by vacuum drying. The resulting residue was dissolved in 6 ml of MeOH while stirring. SOCl₂ was slowly added thereto in an amount of 7-10 equivalents and the mixture was refluxed for 4 hours. The resulting mixture was concentrated under a reduced pressure to remove the solvent, and extracted with ethyl acetate. The resulting extract was washed with saline, dried over MgSO₄, filtered and concentrated under a reduced pressure to remove the solvent. The resulting residue was subjected to flash column chromatography (eluent: n-hexane/ethyl acetate=1:1) to obtain the title compound (50 mg, 0.19 mmol; yield: 42%).

¹H-NMR (CDCl₃): 4.09 (s, 3H), 7.23 (m, 2H), 7.67 (d, 1H), 8.20 (m, 2H), 8.66 (d, 2H), 10.43 (br, 1H)

M.W.: 272

### Step 3) Preparation of 2-(4-fluorophenyl)-3H-imidazo[4,5-b]pyridine-7-carboxylic acid

The compound obtained in Step 2 (30 mg, 0.11 mmol) was dissolved in 2 ml of THF. LiOH.H₂O (13.86 mg, 0.33 mmol) dissolved in 1 ml of water was added thereto and the mixture was stirred at room temperature for 8 hours. The reaction solution was extracted with ethyl acetate and the aqueous layer was washed with ethyl acetate, which was adjusted to pH 2 with 1N aqueous HCl. The resulting solution was concentrated under a reduced pressure to remove the solvent. The resulting residue was vacuum dried to obtain the crude title compound (60 mg, 0.12 mmol; yield: 106%).

¹H-NMR (MeOH-d₄): 7.32 (dd, 2H), 7.68 (d, 1H), 8.30 (dd, 2H), 8.41 (d, 1H)

M.W.: 258

### Step 4) Preparation of 2-(4-fluoro-phenyl)-3H-imidazo[4,5-b]pyridine-7-carboxylic acid [2-(4-acetylamino-phenyl)-ethyl]-amide

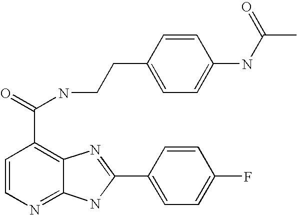

The compound obtained in Step 3 was dissolved in DMF, and 1.2 equivalents of PyBOP, 3 equivalents of TEA and 1.2 equivalents of 4-acetylpentylamine were added thereto, and the mixture was stirred at room temperature for 12 hours. The reaction solution was vacuum dried, and the resulting residue was dissolved in a small quantity of MeOH and filtered. The filtrate thus obtained was subjected to Prep. HPLC to obtain the title compound (yield: 50%).

### EXAMPLES 197 TO 326

The procedure of Example 196 was repeated except for using each of the corresponding amine compounds instead of 4-acetylpentylamine in Step 4, to obtain the respective title compounds.

### EXAMPLE 327

The compound obtained in Example 196 (10 mg, 0.03 mmol) was dissolved in 2 ml of a mixture of DMSO and N-methylpiperazine (1:1). The resulting solution was kept under a 200 W power and 100 psi, at 150° C. for 1 hour, and was subjected to Prep. HPLC to obtain the title compound (3.72 mg, 0.009 mmol; yield: 30%).

### EXAMPLES 328 TO 458

The procedure of Example 327 was repeated except for using each of the corresponding compounds instead of the compound obtained in Example 196 and N-methylpiperazine, to obtain the respective title compounds.

### TEST EXAMPLE 1

### Analysis of Inhibitory Capacity of Protein Kinase for Enzyme Activity

### <Glycogen Synthase Kinase-3β (GSK-3β)>

The activities of the compounds prepared in the Examples for inhibiting GSK-3β enzyme activity were assessed by a modified method of U.S. Pat. No. 6,153,618 (Shultz et al.). GSK-3β was prepared by a gene recombination method.

First, primers corresponding to 5′-end and 3′-end of polynucleotide encoding human GSK-3β were designed and synthesized from nucleotide sequence of human GSK-3β (GenBank Reg. No. L33801). Then, the primers were amplified by PCR (polymerase chain reaction) in which a human DNA sequence was employed as a template and treated with restriction enzyme BamH1/XhoI. The resulting gene fragments were inserted into the corresponding identical restriction sites of pGex vector (GE Healthcare Life Science) to prepare an expression vector for transformation of E. coli BL21 (DE3) strain (Invitrogen). The transformed E. coli strain was inoculated to LB medium (1% Bacto tryptone, 0.5% yeast extract, 1% sodium chloride) and cultured until the optical density of the bacterial cells was about 0.5 at 600 nm and 37° C. Then, IPTG (isopropyl-β-D-thiogalactoside) was added thereto to a final concentration to 0.5 mM at 18° C. 16 hours after IPTG addition, the cells were subjected to centrifugation at 10,000×g for 10 mins and cell precipitates were collected. The cell precipitates were suspended in a buffer solution (30 mM tris-HCl (pH 7.5), 100 mM NaCl, 5% glycerol, 2 mM DTT) and the cells were smashed in an ice bath using a Sonic Dismembrator (Fisher, USA). The resulting solution was centrifuged at 16,000 rpm for 30 mins.

The supernatant obtained above was introduced to a pre-equilibrated GST column (Pharmacia, USA) and eluted by 5 mM glutachione. The effuent was subjected to SDS-PAGE and GSK-3β protein was collected. GST protein was cutt using thrombin. The GSK-3β protein thus obtained was diluted with a buffer solution (20 mM HEPES (pH 7.5), 5% glycerol, 2 mM DTT) until the concentration of NaCl reached 50 mM. The diluted solution was introduced to Mono S column (Pharmacia, USA) equilibrated with the above buffer solution and eluted with a aqueous NaCl while changing the concentration from 0 to 1 M NaCl, and GSK-3β protein was collected by a electrophoresis. The purified protein was used in the analysis for the activity for enzyme activity.

Meanwhile, each of the compounds prepared in the Examples was dissolved in dimethylsulfoxide (DMSO) to a concentration of 12.5 mM to prepare a test solution. The enzyme reaction was conducted in a buffer solution (50 mM of tris-HCl (pH 7.5), 10 mM of MgCl₂, 1 mM of EGTA, 1 mM of EDTA and 1 mM of DTT). 100 μM of phosho-CREB peptide (NEB, USA), 100 μM of ATP and 1 μCi of ³²P-ATP were added to the buffer solution as substrates. And then 100 nM of recombinant GSK-3β was added thereto and the mixture was reacted at 30° C. for 1 hour. The reaction was terminated by the addition of 5 μl of 5% phosphoric acid solution to 25 μl of the reaction mixture. The resulting solution was subjected to centrifugation at 15,000 for 10 mins and 20 μl of the supernatant thus obtained was dropped on whatman p81 filter paper. The filter paper was washed in 0.5% phosphoric acid solution for 10 mins. After repeating the washing 3 times, the filter paper was dried and its cpm (counter per mins) was assessed.

The test solution prepared above by dissolving a test compound in DMSO was added to the reaction solution in an amount of less than 5% based on the total reaction solution to analyze the capacity for enzyme inhibitory activity. The cpm value obtained when the test compounds was present relative to the cpm value in the absence of the test compound was represented by percentage, and IC₅₀ (μM) was determined as the concentration of the test compound required to inhibit the enzyme activity by 50% relatively to the control solution.

### <Aurora Kinase A>

A test solution was prepared by dissolving one of the compounds of the Examples in DMSO at a concentration of 12.5 mM. Enzyme reaction was conducted in a buffer solution containing 20 mM of HEPES (pH 7.5), 5 mM MgCl₂, 0.5 mM ethylene glycol bis(b-aminoethylether) tetraacetic acid (EGTA), 200 mM of KCl, 1 mM of DTT and 0.05% triton X-100. 100 μM of Kemptide peptide (Upstate) and 1 μM of ATP were added to the buffer solution as substrates. Recombinant aurora kinase (Upstate) was added the resulting mixture at a concentration of 10 nM and reaction was carried out at 30° C. for 1 hour. 25 μl of the resulting solution was mixed with 25 μl of Kinase glo (promega), thereby inducing the second reaction by luciferase. The amount of remained ATP was measured by fusion a-FP (Packard, USA). Inhibitory capacities of the test compounds for the enzyme activity were assessed according to the same method as in GSK-3β analysis, and IC₅₀ value was calculated.

### <Extracellular Signal-Regulated Kinase-1 (ERK-1)>

The compounds prepared in the Examples were dissolved in dimethylsulfoxide (DMSO) at a concentration of 12.5 mM to prepare test compounds and enzyme reaction was conducted in a buffer solution containing 50 mM of tris-HCl (pH 7.5), 10 mM of MgCl₂, 1 mM of EGTA, 1 mM of EDTA and 1 mM of DTT. 0.33 mg/ml of MBP (Upstate), 100 μM of ATP and 0.25 μCi of ³²P-ATP were added to the buffer solution as substrates. 5 nM of recombinant Erk-1 (Upstate) was added the resulting mixture, and the mixture was reacted at 30° C. for 1 hour. The reaction was terminated by adding 5 μl of 5% phosphoric acid solution to 25 μl of the reaction mixture. 15 μl of the resulting solution was dropped on whatman p81 filter paper, which was then washed in 0.5% phosphoric acid solution for 10 mins. After repeating the washing 3 times, the filter paper was dried and cpm thereof was measured by a liquid scintillation counter (Packard, USA). Inhibitory capacities of the test compounds for the enzyme activity were assessed according to the same method as in GSK-3β analysis, and IC₅₀ value was calculated.

### <Cyclin-Dependent Kinase-2 (CDK-2)>

The procedure of ERK-1 analysis was repeated to assess inhibitory capacities of the test compounds for the enzyme activity, except for using 2.5 μg of histone H1 (Upstate) and 100 nM of recombinant CDK-2/cycline A (Upstate).

### <p38 Mitogen Activated Protein Kinase (MAPK)>

The procedure of ERK-1 analysis was repeated to assess inhibitory capacities of the test compounds for the enzyme activity, except for using 2.5 μg of histone H1 (Upstate) and 5 nM of recombinant p38a (Upstate).

<c-Jun N-Terminal Kinase-1 (JNK-1)>

The procedure of ERK-1 analysis was repeated to assess inhibitory capacities of the test compounds for the enzyme activity, except for using 272 nM of GST-ATF2 (Upstate) and 7 nM of recombinant JNK1 (Upstate).

### <Pyruvate Dehydrogenase Kinase-1 (PDK-1)>

The procedure of aurora kinase A analysis was repeated to assess inhibitory capacities of the test compounds for the enzyme activity, except for using a buffer solution containing 50 mM of tris-HCl (pH 7.5), 10 mM of MgCl₂, 1 mM of EGTA and 1 mM of DTA; 2.5 μM of PDKtide (peptide, Upstate) (Upstate); and 31.5 nM of recombinant PDK1 (Upstate).

### <Kinase Insert Domain Protein Receptor (KDR)>

The compounds prepared in the Examples were dissolved in dimethylsulfoxide (DMSO) at a concentration of 12.5 mM to prepare test solution, and enzyme reaction was conducted in a buffer solution containing 50 mM tris-HCl (pH 7.5), 5 mM MgCl₂, 1 mM MnCl₂, 0.01% tween-20 and 2 mM of DTT. 1 nM of Biotin-polyE4Y (Packard) and 0.1 μM of ATP were added to the buffer solution as substrates. 2 nM of recombinant KDR (Upstate) was added the resulting mixture and reaction was carried out at 30° C. for 1 hour. 10 μl of a diluted solution prepared by diluting alphascreen phosphotyrosine (T-Tyr-100, Packard) beads with a solution containing 6.25 mM of HEPES (pH 7.4), 250 mM of NaCl, 100 Mm EDTA, and 0.25% BSA was added to 15 μl of the KDR reaction solution. After a reaction at a room temperature for 1 hour, alphascreen signal was measured by a fusion a-FP (Packard). Inhibitory capacities of the test compounds for the enzyme activity were assessed according to the same method as in GSK-3β analysis, and IC₅₀ value was calculated.

Inhibitory capacities of the test compounds for the GSK-3 β are shown in Table 2 in comparison with that of a comparative compound, 99021 derivative (Chiron) (Diabetes, 52, 588-595 (2003)).

TABLE 2

| Examples | IC₅₀ (μM) |
|---|---|
| 1 | 0.020 |
| 44 | 0.038 |
| 61 | 0.003 |
| 109 | 0.052 |
| 154 | 0.003 |
| 332 | 0.012 |
| 308 | 0.008 |
| 320 | 0.005 |
| 325 | 0.002 |
| 327 | 0.050 |
| 332 | 0.012 |
| 396 | 0.002 |
| 404 | 0.003 |
| Comparative compound | 0.030 |
| (99021 derivative) | |

As can be seen from table 2, the compounds of formula 1 according to the inventive Examples exhibit more superior inhibitory capacity for GSK-3β than the Comparative compound.

Further, the inhibitory capacities for aurora kinase A, ERK-1, CDK-2, JNK-1, PDK-1, KDR and p38 Mitogen-activated protein kinase (MAPK) are shown in Table 3.

TABLE 3
Aurora Kinase A

| Examples | IC₅₀ (μM) |
|---|---|
| 264 | 0.070 |
| 343 | 0.198 |
| 368 | 0.040 |
| 371 | 0.113 |
| 381 | 0.033 |
| 313 | 1.100 |
| 409 | 0.250 |
| 418 | 0.200 |
| 423 | 0.105 |
| 425 | >6 |

TABLE 4
ERK-1

| Examples | IC₅₀ (μM) |
|---|---|
| 241 | 16.5 |
| 320 | 25.5 |
| 452 | 19.0 |

TABLE 5
CDK-2

| Examples | IC₅₀ (μM) |
|---|---|
| 198 | 2.4 |
| 246 | 0.225 |
| 340 | 0.4 |
| 343 | 0.3 |
| 287 | 0.12 |
| 308 | 0.95 |
| 320 | 0.17 |
| 451 | 0.36 |
| 452 | 0.35 |

TABLE 6
JNK-1

| Examples | IC₅₀ (μM) |
|---|---|
| 246 | 0.18 |
| 340 | 0.10 |
| 287 | 0.46 |
| 308 | 0.95 |
| 320 | 14.5 |
| 451 | 10.5 |

TABLE 7
PDK-1

| Examples | IC₅₀ (μM) |
|---|---|
| 343 | 3.1 |
| 241 | 28.5 |

TABLE 8
KDR

| Examples | IC₅₀ (μM) |
|---|---|
| 198 | 12.5 |
| 246 | 0.52 |
| 343 | 0.17 |
| 287 | 1.5 |
| 320 | 0.29 |
| 451 | 1.65 |

TABLE 9
p38 Mitogen-activated protein kinase (MAPK)

| Examples | IC₅₀ (μM) |
|---|---|
| 308 | 50 |
| 320 | 50 |
| 451 | 3.25 |

As can be seen from Table 3 to 9, the compounds of formula 1 according to the present invention exhibit inhibitory capacities for various protein kinases.

While the invention has been described with respect to the above specific embodiments, it should be recognized that various modifications and changes may be made to the invention by those skilled in the art which also fall within the scope of the invention as defined by the appended claims.

What is claimed is:

1. A compound of formula 1, or a pharmaceutically acceptable salt, hydrate, solvate or isomer thereof: 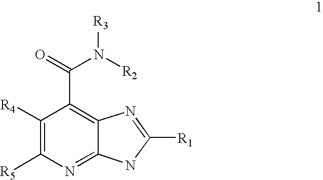
wherein,
R₁ is hydroxy, halogen, C₁₋₆alkyloxy, C₁₋₆alkyl, amino, C₁₋₆alkylamino, carboxyl, nitro, sulfonylamide, C₁₋₆alkylsulfonyl, amide, aryl or heteroaryl optionally substituted with halogen, —CN, NO₂, C₁₋₆alkyl, C₁₋₆alkylpiperazinyl, C₁₋₆alkylsulfinyl C₁₋₆alkyl, piperidinyl, morpholinyl, pyrrolidinyl, morpholinyl C₁₋₆ alkylamino, pyrrolidinyl C₁₋₆ alkylamino, —OR′, —C(O)OR′, —OC(O)R′, —NR′R″, —NHC(O)R′, —C(O)NR′R″, —NHC(S)R′, —C(S)NR′R″, —SR′, —S(O)R′, —SO₂R′, —NHSO₂R′, —SO₂NR′R″, —OSO₂R′, —SO₂OR′, aryl, heteroaryl, aryl-C₁₋₄alkyl, formyl or trifluoromethyl, R′ or R″ being each independently hydrogen; or C₁₋₄alkyl, C₃₋₇cycloalkyl, aryl or heteroaryl optionally substituted with C₁₋₄alkyl, C₁₋₄ alkoxy, CN, NO₂, NH₂, (C₁₋₄alkyl)amino, OH, COOH, COO(C₁₋₄alkyl), —CONH₂, formyl or trifluoromethyl; the aryl being phenyl, indanyl or naphthyl; and heteroaryl being 5-10 membered-ring aryl, or mono- or bicyclic heterocycle comprising one or more nitrogen, sulfur or oxygen atom in its ring structure;
R₂ is hydrogen; unsubstituted or substituted C₁₋₈alkyl; or unsubstituted or substituted C₁₋₇alkyl comprising nitrogen, sulfur or oxygen in its chain structure, the substituent of the alkyl being hydroxy, halogen, C₁₋₆alkyloxy, alkyl, amino, C₁₋₆alkylamino, carboxyl, nitro, sulfonylamide, alkylsulfonyl or amide; aryl or heteroaryl optionally substituted with C₁₋₄alkyl, hydroxy, halogen, C₁₋₆alkyloxy, amino, C₁₋₆alkylamino, aminoC₁₋₆alkyl, acetylamino, carboxyl, amide, dioxoindole, —CN, NO₂, —OR′, —C(O)OR′, —OC(O)R′, —NR′R″, —NHC(O)R′, —NHC(O)OR′, —C(O)NR′R″, —NHC(S)R′, —C(S)NR′R″, —SR′, —S(O)R′, —SO₂R′, —NHSO₂R′, —SO₂NR′R″, —OSO₂R′, —SO₂OR′, aryl, heteroaryl, aryl-C₁₋₄alkyl, formyl or trifluoromethyl, R′ or R″ being each independently hydrogen; or C₁₋₄alkyl, C₃₋₇cycloalkyl, aryl or heteroaryl optionally substituted with halogen, C₁₋₄alkyl, C₁₋₄alkoxy, CN, NO₂, NH₂, C₁₋₄alkylamino, aminoC₁₋₄alkyl, OH, COOH, —COOC₁₋₄alkyl, —CONH₂, formyl, C₁₋₆alkylpiperazinyl, morpholinyl or trifluoromethyl; the aryl being phenyl, indanyl or naphthyl; and heteroaryl being 5-10 membered-ring aryl, pyridone or mono or bicyclic heterocycle comprising one to four nitrogen, sulfur or oxygen atom in its ring structure; or unsubstituted or substituted aryl; or unsubstituted or substituted aryl comprising one or more nitrogen, sulfur or oxygen in its ring structure, the substituent of the aryl being hydroxy; halogen; C₁₋₆alkyloxy; C₁₋₆alkyl; amino; C₁₋₆alkylamino; carboxyl; nitro; sulfonylamide; C₁₋₆alkylsulfonyl; amide; unsubstituted or substituted C₁₋₆alkyl; or cyclicC₁₋₆alkyl comprising one or more nitrogen, sulfur or oxygen atome in its ring structure, the substituent of the alkyl being hydroxy; halogen; C₁₋₆alkyloxy; C₁₋₆alkyl; amino; C₁₋₆alkylamino; carboxyl; nitro; sulfonylamide; C₁₋₆alkylsulfonyl; amide; aryl optionally substituted with hydroxy, halogen, C₁₋₆alkyloxy, C₁₋₆alkyl, amino, C₁₋₆alkylamino, carboxyl, nitro, amide or dioxoisoindole; sulfonylaminoaryl having an aryl group substituted with hydroxy, halogen, C₁₋₆alkyloxy, C₁₋₆alkyl, amino, C₁₋₆alkylamino, carboxyl, nitro, sulfonylamide, C₁₋₆alkylsulfonyl or amide; aryl comprising one or more nitrogen, sulfur or oxygen atoms in its ring structure which is represented by pyrrole, pyrazole, imidazole, 1,2,3-triazole, 1,2,4-triazole, isooxazole, oxazole, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, oxypyridien, pyrimidine or triazine optionally substituted with hydroxy, halogen, C₁₋₆alkyloxy, C₁₋₆alkyl, amino, C₁₋₆alkylamino, carboxyl, nitro, sulfonylamide, C₁₋₆alkylsulfonyl or amide; or C₃₋₈cycloalkyl optionally substituted with hydroxy, halogen, C₁₋₆alkyloxy, C₁₋₆alkyl, amino, C₁₋₆alkylamino, carboxyl, nitro or amide;
R₃ is hydrogen; or C₁₋₄alkyl or C₃₋₇cycloalkyl optionally substituted with one or more substituent selected from the group consisting of halogen, C₁₋₄alkyl, C₁₋₄alkoxy, CN, NO₂, NH₂, (C₁₋₄alkyl)-amino, amino-(C₁₋₄alkyl), OH, COOH, —COO(C₁₋₄alkyl), and —CONH₂, having an optional substituent selected from the group consisting of hydroxy; halogen; alkyloxy; alkyl; amino; alkylamino; carboxyl; nitro; sulfonylamide; alkylsulfonyl; or amide; or
R₂ and R₃ are fused together with the nitrogen to which they are attached to form a ring, and
R₄ and R₅ are each independently hydrogen; or C₁₋₄alkyl or C₃₋₇cycloalkyl substituted with an optional substituent selected from the group consisting of halogen, C₁₋₄alkyl, C₁₋₄alkoxy, CN, NO₂, NH₂, C₁₋₄alkylamino, aminoC₁₋₄alkyl, OH, COOH, COOC₁₋₄alkyl and —CONH₂, each of which having an optional substituent, be selected from the group consisting of hydroxy, halogen, alkyloxy, alkyl, amino, alkylamino, carboxyl, nitro, sulfonylamide, alkylsulfonyl and amide.

2. The compound of claim 1, wherein R₁ is phenyl, pyrrolidinylphenyl, dichlorophenyl, chlorophenyl, fluorophenyl, difluorophenyl, furanyl, thiophene, cyclopropyl, C₁₋₂alkylpiperazinylphenyl, C₁₋₂alkylpiperazinylC₁₋₃alkylphenyl, C₁₋₂alkylpiperazinylC₁₋₃alkylaminophenyl, methanesulfinylphenyl, diC₁₋₂alkylaminophenyl, morpholinylphenyl, piperidinylphenyl, morpholinylC₁₋₃alkylaminophenyl, pyrrolidinylC₁₋₃alkylaminophenyl, dimethylaminoC₁₋₄alkylaminophenyl, diC₁₋₂alkylaminoethylmethylaminophenyl, piperazinylaminophenyl, piperazinylC₁₋₂alkylaminophenyl, thiomorpholinylphenyl, piperidinylaminophenyl, piperidinylC₁₋₂alkylaminophenyl, methoxyphenyl, diC₁₋₃alkylaminopyrrolidinylphenyl or pyridinyl; R₂ is C₁₋₅alkyl optionally substituted with sulfonylphenyl, C₁₋₂alkylpyridinyl, diC₁₋₂alkyl, triC₁₋₂alkyl, tetraC₁₋₂alkyl, pyridinyl, oxypyridinyl, chloropyridinyl, morpholinyl, aminoC₁₋₂alkylpyridinyl, acetylaminophenyl, imidazole, dichloroimidazole, C₁₋₂alkylimidazole, diC₁₋₂alkylaminosulfonylaminophenyl, trifluoroC₁₋₂alkylphenyl, benzyloxyoxopyridinyl, hydroxyoxopyridinyl, C₁₋₂alkanesulfonylaminophenyl, diC₁₋₂alkylaminoacetylaminophenyl, trifluoromethanesulfonylaminophenyl, fluoropyridinyl, fluorohydroxyphenyl, C₁₋₂alkylpiperazinylacetylaminophenyl, chlorooxypyridinyl, thiophenyl, C₁₋₂alkyloxypyridinyl, aminophenyl, hydroxyphenyl, C₁₋₂alkylpiperazincarbonylaminophenyl, morpholinylC₁₋₃alkoxyphenyl, benzyl, hydroxyl diC₁₋₂alkyl or diC₁₋₂alkylaminoC₁₋₂alkyl; cyclo C₃₋₇alkyl optionally substituted with triC₁₋₂alkyl, amino or hydroxy; pyridinyl optionally substituted with C₁₋₂alkyl, diC₁₋₂alkyl, chloroC₁₋₂alkoxy, C₁₋₂alkylamino, aminoC₁₋₂alkyl, C₁₋₂alkoxy, C₁₋₂alkoxyC₁₋₂alkyl, C₁₋₂alkylsulfanyl, chloroC₁₋₂alkyl, isobutoxy, cyclopropylmethoxy, diC₁₋₂alkylaminoC₁₋₂alkoxy, morpholinylC₁₋₂alkoxy, halogen, acetylamino or C₁₋₂alkylsulfanylC₁₋₂alkyl; phenyl substituted with benzoylamino, piperidinyl, hydroxy, C₁₋₂alkoxy, C₁₋₂alkyl, diC₁₋₂alkyl, diisopropyl, isopropyl, diC₁₋₂alkylaminoacetylamino, fluoro C₁₋₂alkyl, fluorohydroxy, trifluoroC₁₋₂alkoxy, diC₁₋₂alkoxy, acetylamino, cyano, benzyloxy, trifluoromethanesulfonylamino or C₁₋₂alkanesulfonyl; benzothiazoyl, indazolyl, C₁₋₂alkylindolyl, indolyl, naphthalenyl, quinolinyl, C₁₋₂alkylpyrazolyl, phenylthiazolyl, tolyl, benzodioxolyl, C₁₋₂alkylphenylacetamide, C₁₋₂alkylphenoxyacetyl, ethanesulfonylC₁₋₂alkylphenylamide, C₁₋₂alkylphenoxyacetic acid tert-butylester, C₁₋₂alkylphenylmethanesulfonamide, C₁₋₂alkylpiperazinyl, C₁₋₂alkoxyphenylamide, piperidinyl, benzyl piperidinyl, C₁₋₂alkylphenoxyacetyl, triC₁₋₂alkylbicycloheptinyl, adamantanyl, aminobicycloheptanecarboxyl, azabicyclooctyl, bicycloheptinyl, tert-butylamide or C₁₋₂alkylpyridinyl C₁₋₂alkylcarbamic acid tert-butylester; and R₃ is H, or R₂ and R₃ are fused together with the nitrogen to which they are attached to form a ring; R₄ is H or halogen; and R₅ is H.

3. The compound of claim 1, which is selected from the group consisting of:
1) 2-phenyl-3H-imidazo[4,5-b]pyridine-7-carboxylic acid [2-(4-acetylamino-phenyl)-ethyl]-amide;
2) (4-{2-[(2-phenyl-3H-imidazo[4,5-b]pyridine-7-carbonyl)-amino]-ethyl}-phenoxy)-acetic acid;
3) 2-phenyl-3H-imidazo[4,5-b]pyridine-7-carboxylic acid[2-(2-hydroxy-phenyl)-ethyl]-amide;
4) (3-{2-[(2-phenyl-3H-imidazo[4,5-b]pyridine-7-carbonyl)-amino]-ethyl}-phenoxy)-acetic acid;
5) 2-phenyl-3H-imidazo[4,5-b]pyridine-7-carboxylic acid[2-(4-ethanesulfonylamino-phenyl)-ethyl]-amide;
6) (3-{2-[(2-phenyl-3H-imidazo[4,5-b]pyridine-7-carbonyl)-amino]-ethyl}-phenoxy)-acetic acid tert-butylester;
7) (4-{2-[(2-phenyl-3H-imidazo[4,5-b]pyridine-7-carbonyl)-amino]-ethyl}-phenoxy)-acetic acid tert-butylester;
8) 2-phenyl-3H-imidazo[4,5-b]pyridine-7-carboxylic acid[2-(4-fluoro-3-hydroxy-phenyl)-ethyl]-amide;
9) 2-phenyl-3H-imidazo[4,5-b]pyridine-7-carboxylic acid[2-(3-hydroxy-phenyl)-ethyl]-amide;
10) 2-phenyl-3H-imidazo[4,5-b]pyridine-7-carboxylic acid[3-(4,5-dichloro-3H-imidazo-1-yl)-propyl]-amide;
11) 2-phenyl-3H-imidazo[4,5-b]pyridine-7-carboxylic acid[2-(4-methanesulfonylamino-phenyl)-ethyl]-amide;
12) 2-phenyl-3H-imidazo[4,5-b]pyridine-7-carboxylic acid(2-pyridin-2-yl-ethyl)-amide;
13) 2-phenyl-3H-imidazo[4,5-b]pyridine-7-carboxylic acid(2-pyridin-3-yl-ethyl)-amide;
14) 2-phenyl-3H-imidazo[4,5-b]pyridine-7-carboxylic acid(2-pyridin-4-yl-ethyl)-amide;
15) 2-phenyl-3H-imidazo[4,5-b]pyridine-7-carboxylic acid(3-morpholin-4-yl-propyl)-amide;
16) 2-phenyl-3H-imidazo[4,5-b]pyridine-7-carboxylic acid[2-(1-oxy-pyridin-2-yl)-ethyl]-amide;
17) 2-phenyl-3H-imidazo[4,5-b]pyridine-7-carboxylic acid[2-(1-oxy-pyridin-3-yl)-ethyl]-amide;
18) 2-phenyl-3H-imidazo[4,5-b]pyridine-7-carboxylic acid[2-(1-oxy-pyridin-4-yl)-ethyl]-amide;
19) 2-phenyl-3H-imidazo[4,5-b]pyridine-7-carboxylic acid[2-(2-chloro-pyridin-3-yl)-ethyl]-amide;
20) 2-phenyl-3H-imidazo[4,5-b]pyridine-7-carboxylic acid[2-(2-chloro-pyridin-4-yl)-ethyl]-amide;
21) 2-phenyl-3H-imidazo[4,5-b]pyridine-7-carboxylic acid[2-(6-chloro-pyridin-3-yl)-ethyl]-amide;
22) 2-phenyl-3H-imidazo[4,5-b]pyridine-7-carboxylic acid[2-(2-aminomethyl-pyridin-3-yl)-ethyl]-amide;
23) 2-phenyl-3H-imidazo[4,5-b]pyridine-7-carboxylic acid[2-(2-aminomethyl-pyridin-4-yl)-ethyl]-amide;
24) 2-phenyl-3H-imidazo[4,5-b]pyridine-7-carboxylic acid(4-benzoylamino-phenyl)-amide;
25) 2-phenyl-3H-imidazo[4,5-b]pyridine-7-carboxylic acid(4-piperidin-1-yl-phenyl)-amide;
26) 6-bromo-2-phenyl-3H-imidazo[4,5-b]pyridine-7-carboxylic acid(4-benzoylamino-phenyl)-amide;
27) 6-bromo-2-phenyl-3H-imidazo[4,5-b]pyridine-7-carboxylic acid[2-(2-chloro-pyridin-4-yl)-ethyl]-amide;
28) 6-bromo-2-phenyl-3H-imidazo[4,5-b]pyridine-7-carboxylic acid(2-thiophen-2-yl-ethyl)-amide;
29) 6-chloro-2-phenyl-3H-imidazo[4,5-b]pyridine-7-carboxylic acid[3-(4,5-dichloro-imidazo-1-yl)-propyl]-amide;
30) 2-phenyl-3H-imidazo[4,5-b]pyridine-7-carboxylic acid phenylamide;
31) 2-phenyl-3H-imidazo[4,5-b]pyridine-7-carboxylic acid cyclohexylamide;
32) 2-(2,4-dichloro-phenyl)-3H-imidazo[4,5-b]pyridine-7-carboxylic acid[2-(4-acetylamino-phenyl)-ethyl]-amide;
33) 2-(2,4-dichloro-phenyl)-3H-imidazo[4,5-b]pyridine-7-carboxylic acid[2-(3-hydroxy-phenyl)-ethyl]-amide;
34) 2-(2,4-dichloro-phenyl)-3H-imidazo[4,5-b]pyridine-7-carboxylic acid[2-(2-hydroxy-phenyl)-ethyl]-amide;
35) [4-(2-{[2-(2,4-dichloro-phenyl)-3H-imidazo[4,5-b]pyridine-7-carbonyl]-amino}-ethyl)-phenoxy]-acetic acid;
36) [3-(2-{[2-(2,4-dichloro-phenyl)-3H-imidazo[4,5-b]pyridine-7-carbonyl]-amino}-ethyl)-phenoxy]-acetic acid;
37) [4-(2-{[2-(2,4-dichloro-phenyl)-3H-imidazo[4,5-b]pyridine-7-carbonyl]-amino}-ethyl)-phenoxy]-acetic acid tert-butylester;
38) [3-(2-{[2-(2,4-dichloro-phenyl)-3H-imidazo[4,5-b]pyridine-7-carbonyl]-amino}-ethyl)-phenoxy]-acetic acid tert-butylester;
39) 2-(2,4-dichloro-phenyl)-3H-imidazo[4,5-b]pyridine-7-carboxylic acid[3-(3H-imidazo-1-yl)-propyl]-amide;
40) 2-(2,4-dichloro-phenyl)-3H-imidazo[4,5-b]pyridine-7-carboxylic acid {2-[4-(2-dimethylamino-acetylamino)-phenyl]-ethyl}-amide;
41) 2-(2,4-dichloro-phenyl)-3H-imidazo[4,5-b]pyridine-7-carboxylic acid[2-(4-sulfonylphenyl)-ethyl]-amide;
42) [2-(2-{[2-(2,4-dichloro-phenyl)-3H-imidazo[4,5-b]pyridine-7-carbonyl]-amino}-ethyl)-phenoxy]-acetic acid;
43) 2-(4-chloro-phenyl)-3H-imidazo[4,5-b]pyridine-7-carboxylic acid [2-(4-ethanesulfonylamino-phenyl)-ethyl]-amide;
44) 2-(4-chloro-phenyl)-3H-imidazo[4,5-b]pyridine-7-carboxylic acid[2-(4-acetylamino-phenyl)-ethyl]-amide;
45) 2-(4-chloro-phenyl)-3H-imidazo[4,5-b]pyridine-7-carboxylic acid[2-(3-hydroxy-phenyl)-ethyl]-amide;
46) 2-(4-chloro-phenyl)-3H-imidazo[4,5-b]pyridine-7-carboxylic acid{2-[4-(2-dimethylamino-acetylamino)-phenyl]-ethyl}-amide;
47) 2-(4-chloro-phenyl)-3H-imidazo[4,5-b]pyridine-7-carboxylic acid(3-imidazo-1-yl-propyl)-amide;
48) 2-(4-chloro-phenyl)-3H-imidazo[4,5-b]pyridine-7-carboxylic acid[3-(4,5-dichloro-imidazo-1-yl)-propyl]-amide;
49) 2-(4-chloro-phenyl)-3H-imidazo[4,5-b]pyridine-7-carboxylic acid[3-(4-methyl-imidazo-1-yl)-propyl]-amide;
50) 2-(4-chloro-phenyl)-3H-imidazo[4,5-b]pyridine-7-carboxylic acid[2-(4-methanesulfonylamino-phenyl)-ethyl]-amide;
51) 2-(4-chloro-phenyl)-3H-imidazo[4,5-b]pyridine-7-carboxylic acid[2-(4-dimethylaminosulfonylamino-phenyl)-ethyl]-amide;
52) 2-(4-chloro-phenyl)-3H-imidazo[4,5-b]pyridine-7-carboxylic acid[2-(2-hydroxy-phenyl)-ethyl]-amide;
53) 2-(4-chloro-phenyl)-3H-imidazo[4,5-b]pyridine-7-carboxylic acid[2-(3-trifluoromethyl-phenyl)-ethyl]-amide;
54) 2-(4-chloro-phenyl)-3H-imidazo[4,5-b]pyridine-7-carboxylic acid[3-(4-benzyloxy-2-oxo-2H-pyridin-1-yl)-propyl]-amide;
55) 2-(4-chloro-phenyl)-3H-imidazo[4,5-b]pyridine-7-carboxylic acid[2-(4-benzyloxy-2-oxo-2H-pyridin-1-yl)-ethyl]-amide;
56) 2-(4-chloro-phenyl)-3H-imidazo[4,5-b]pyridine-7-carboxylic acid[2-(4-hydroxy-2-oxo-2H-pyridin-1-yl)-ethyl]-amide;
57) 2-(4-chloro-phenyl)-3H-imidazo[4,5-b]pyridine-7-carboxylic acid(2-pyridin-4-yl-ethyl)-amide;
58) 2-(4-chloro-phenyl)-3H-imidazo[4,5-b]pyridine-7-carboxylic acid (4-methyl-pyridin-3-yl)-amide;
59) 2-(4-chloro-phenyl)-3H-imidazo[4,5-b]pyridine-7-carboxylic acid (4-ethoxy-pyridin-3-yl)-amide;
60) 2-(4-chloro-phenyl)-3H-imidazo[4,5-b]pyridine-7-carboxylic acid (6-methyl-pyridin-3-yl)-amide;
61) 2-(4-chloro-phenyl)-3H-imidazo[4,5-b]pyridine-7-carboxylic acid (4-ethylsulfanyl-pyridin-3-yl)-amide;
62) 2-furan-2-yl-3H-imidazo[4,5-b]pyridine-7-carboxylic acid[3-(4,5-dichloro-3H-imidazo-1-yl)-propyl]-amide;
63) 2-furan-2-yl-3H-imidazo[4,5-b]pyridine-7-carboxylic acid[2-(4-methanesulfonylamino-phenyl)-ethyl]-amide;
64) 2-furan-2-yl-3H-imidazo[4,5-b]pyridine-7-carboxylic acid[2-(3-hydroxy-phenyl)-ethyl]-amide;
65) 2-furan-2-yl-3H-imidazo[4,5-b]pyridine-7-carboxylic acid[2-(4-ethanesulfonylamino-phenyl)-ethyl]-amide;
66) 2-furan-2-yl-3H-imidazo[4,5-b]pyridine-7-carboxylic acid{2-[4-(2-dimethylamino-acetylamino)-phenyl]-ethyl}-amide;
67) 2-furan-2-yl-3H-imidazo[4,5-b]pyridine-7-carboxylic acid[3-(4-methyl-imidazo-1-yl)-propyl]-amide;
68) 2-furan-2-yl-3H-imidazo[4,5-b]pyridine-7-carboxylic acid[2-(2-hydroxy-phenyl)-ethyl]-amide;
69) 2-furan-2-yl-3H-imidazo[4,5-b]pyridine-7-carboxylic acid[2-(4-dimethylaminosulfonylamino-phenyl)-ethyl]-amide;
70) 2-furan-2-yl-3H-imidazo[4,5-b]pyridine-7-carboxylic acid(2-pyridin-2-yl-ethyl)-amide;
71) 2-furan-2-yl-3H-imidazo[4,5-b]pyridine-7-carboxylic acid(2-pyridin-3-yl-ethyl)-amide;
72) 2-furan-2-yl-3H-imidazo[4,5-b]pyridine-7-carboxylic acid(2-pyridin-4-yl-ethyl)-amide;
73) 2-furan-2-yl-3H-imidazo[4,5-b]pyridine-7-carboxylic acid[2-(4-fluoro-3-hydroxy-phenyl)-ethyl]-amide;
74) 2-furan-2-yl-3H-imidazo[4,5-b]pyridine-7-carboxylic acid(3-imidazo-1-yl-propyl)-amide;
75) 2-furan-2-yl-3H-imidazo[4,5-b]pyridine-7-carboxylic acid[2-(1-oxy-pyridin-2-yl)-ethyl]-amide;
76) 2-furan-2-yl-3H-imidazo[4,5-b]pyridine-7-carboxylic acid[2-(1-oxy-pyridin-3-yl)-ethyl]-amide;
77) 2-furan-2-yl-3H-imidazo[4,5-b]pyridine-7-carboxylic acid[2-(1-oxy-pyridin-4-yl)-ethyl]-amide;
78) 2-furan-2-yl-3H-imidazo[4,5-b]pyridine-7-carboxylic acid[2-(2-chloro-pyridin-4-yl)-ethyl]-amide;
79) 2-furan-2-yl-3H-imidazo[4,5-b]pyridine-7-carboxylic acid[2-(6-chloro-pyridin-3-yl)-ethyl]-amide;
80) 2-furan-2-yl-3H-imidazo[4,5-b]pyridine-7-carboxylic acid[2-(2-aminomethyl-pyridin-3-yl)-ethyl]-amide;
81) 2-furan-2-yl-3H-imidazo[4,5-b]pyridine-7-carboxylic acid(3-pyridin-3-yl-propyl)-amide;
82) 2-furan-2-yl-3H-imidazo[4,5-b]pyridine-7-carboxylic acid[2-(4-trifluoromethane sulfonylamino-phenyl)-ethyl]-amide;
83) 2-furan-2-yl-3H-imidazo[4,5-b]pyridine-7-carboxylic acid[3-(6-chloro-pyridin-3-yl)-propyl]-amide;
84) 2-furan-2-yl-3H-imidazo[4,5-b]pyridine-7-carboxylic acid(3-pyridin-4-yl-propyl)-amide;
85) 2-furan-2-yl-3H-imidazo[4,5-b]pyridine-7-carboxylic acid[3-(2-fluoro-pyridin-3-yl)-propyl]-amide;
86) 2-thiophen-2-yl-3H-imidazo[4,5-b]pyridine-7-carboxylic acid[2-(4-ethanesulfonylamino-phenyl)-ethyl]-amide;
87) 2-thiophen-2-yl-3H-imidazo[4,5-b]pyridine-7-carboxylic acid[3-(5-methyl-3H-imidazo-1-yl)-propyl]-amide;
88) 2-thiophen-2-yl-3H-imidazo[4,5-b]pyridine-7-carboxylic acid(2-pyridin-2-yl-ethyl)-amide;
89) 2-thiophen-2-yl-3H-imidazo[4,5-b]pyridine-7-carboxylic acid(2-pyridin-3-yl-ethyl)-amide;
90) 2-thiophen-2-yl-3H-imidazo[4,5-b]pyridine-7-carboxylic acid(2-pyridin-4-yl-ethyl)-amide;
91) 2-thiophen-2-yl-3H-imidazo[4,5-b]pyridine-7-carboxylic acid[2-(2-hydroxy-phenyl)-ethyl]-amide;
92) 2-thiophen-2-yl-3H-imidazo[4,5-b]pyridine-7-carboxylic acid[2-(3-hydroxy-phenyl)-ethyl]-amide;
93) 2-thiophen-2-yl-3H-imidazo[4,5-b]pyridine-7-carboxylic acid{2-[4-(2-dimethylamino-acetylamino)-phenyl]-ethyl}-amide;
94) 2-thiophen-2-yl-3H-imidazo[4,5-b]pyridine-7-carboxylic acid[2-(4-dimethylaminosulfonylamino-phenyl)-ethyl]-amide;
95) 2-thiophen-2-yl-3H-imidazo[4,5-b]pyridine-7-carboxylic acid[2-(4-methanesulfonylamino-phenyl)-ethyl]-amide;
96) 2-thiophen-2-yl-3H-imidazo[4,5-b]pyridine-7-carboxylic acid[2-(4-fluoro-3-hydroxy-phenyl)-ethyl]-amide;
97) 2-thiophen-2-yl-3H-imidazo[4,5-b]pyridine-7-carboxylic acid(3-imidazo-1-yl-propyl)-amide;
98) 2-thiophen-2-yl-3H-imidazo[4,5-b]pyridine-7-carboxylic acid[2-(1-oxy-pyridin-2-yl)-ethyl]-amide;
99) 2-thiophen-2-yl-3H-imidazo[4,5-b]pyridine-7-carboxylic acid[2-(1-oxy-pyridin-3-yl)-ethyl]-amide;
100) 2-thiophen-2-yl-3H-imidazo[4,5-b]pyridine-7-carboxylic acid[2-(1-oxy-pyridin-4-yl)-ethyl]-amide;
101) 2-thiophen-2-yl-3H-imidazo[4,5-b]pyridine-7-carboxylic acid[2-(2-chloro-pyridin-4-yl)-ethyl]-amide;
102) 2-thiophen-2-yl-3H-imidazo[4,5-b]pyridine-7-carboxylic acid[2-(6-chloro-pyridin-3-yl)-ethyl]-amide;
103) 2-thiophen-2-yl-3H-imidazo[4,5-b]pyridine-7-carboxylic acid[2-(2-aminomethyl-pyridin-3-yl)-ethyl]-amide;
104) (3-{2-[(2-thiophen-2-yl-3H-imidazo[4,5-b]pyridine-7-carbonyl)-amino]-ethyl}-pyridin-2-ylmethyl)-carbamic acid tert-butylester;
105) 2-thiophen-2-yl-3H-imidazo[4,5-b]pyridine-7-carboxylic acid[2-(2-aminomethyl-pyridin-4-yl)-ethyl]-amide;
106) 2-thiophen-2-yl-3H-imidazo[4,5-b]pyridine-7-carboxylic acid(3-pyridin-3-yl-propyl)-amide;
107) 2-thiophen-2-yl-3H-imidazo[4,5-b]pyridine-7-carboxylic acid[2-(4-trifluoromethanesulfonylamino-phenyl)-ethyl]-amide;
108) 2-thiophen-2-yl-3H-imidazo[4,5-b]pyridine-7-carboxylic acid[3-(6-chloro-pyridin-3-yl)-propyl]-amide;
109) 2-thiophen-2-yl-3H-imidazo[4,5-b]pyridine-7-carboxylic acid(3-pyridin-4-yl-propyl)-amide;
110) 2-thiophen-2-yl-3H-imidazo[4,5-b]pyridine-7-carboxylic acid[3-(2-fluoro-pyridin-3-yl)-propyl]-amide;
111) 2-thiophen-2-yl-3H-imidazo[4,5-b]pyridine-7-carboxylic acid[3-(2-chloro-pyridin-4-yl)-propyl]-amide;
112) 2-thiophen-2-yl-3H-imidazo[4,5-b]pyridine-7-carboxylic acid[2-(2-methyl-pyridin-3-yl)-ethyl]-amide;
113) 2-thiophen-2-yl-3H-imidazo[4,5-b]pyridine-7-carboxylic acid[3-(6-fluoro-pyridin-3-yl)-propyl]-amide;
114) 2-furan-3-yl-3H-imidazo[4,5-b]pyridine-7-carboxylic acid[2-(4-methanesulfonylamino-phenyl)-ethyl]-amide;
115) 2-furan-3-yl-3H-imidazo[4,5-b]pyridine-7-carboxylic acid[2-(4-ethanesulfonylamino-phenyl)-ethyl]-amide;
116) 2-furan-3-yl-3H-imidazo[4,5-b]pyridine-7-carboxylic acid[2-(4-dimethylaminosulfonylamino-phenyl)-ethyl]-amide;
117) 2-furan-3-yl-3H-imidazo[4,5-b]pyridine-7-carboxylic acid[3-(4,5-dichloro-3H-imidazo-1-yl)-propyl]-amide;
118) 2-furan-3-yl-3H-imidazo[4,5-b]pyridine-7-carboxylic acid[3-(5-methyl-3H-imidazo-1-yl)-propyl]-amide;
119) 2-furan-3-yl-3H-imidazo[4,5-b]pyridine-7-carboxylic acid[2-(3-hydroxy-phenyl)-ethyl]-amide;
120) 2-furan-3-yl-3H-imidazo[4,5-b]pyridine-7-carboxylic acid[2-(2-hydroxy-phenyl)-ethyl]-amide;
121) 2-furan-3-yl-3H-imidazo[4,5-b]pyridine-7-carboxylic acid[2-(4-acetylamino-phenyl)-ethyl]-amide;
122) 2-furan-3-yl-3H-imidazo[4,5-b]pyridine-7-carboxylic acid(2-{4-[2-(4-ethyl-piperazin-1-yl)-acetylamino]-phenyl}-ethyl)-amide;
123) 2-furan-3-yl-3H-imidazo[4,5-b]pyridine-7-carboxylic acid(2-pyridin-2-yl-ethyl)-amide;
124) 2-furan-3-yl-3H-imidazo[4,5-b]pyridine-7-carboxylic acid(2-pyridin-3-yl-ethyl)-amide;
125) 2-furan-3-yl-3H-imidazo[4,5-b]pyridine-7-carboxylic acid(2-pyridin-4-yl-ethyl)-amide;
126) 2-furan-3-yl-3H-imidazo[4,5-b]pyridine-7-carboxylic acid[2-(4-fluoro-3-hydroxy-phenyl)-ethyl]-amide;
127) 2-furan-3-yl-3H-imidazo[4,5-b]pyridine-7-carboxylic acid(3-imidazo-1-yl-propyl)-amide;
128) 2-furan-3-yl-3H-imidazo[4,5-b]pyridine-7-carboxylic acid[2-(1-oxy-pyridin-2-yl)-ethyl]-amide;
129) 2-furan-3-yl-3H-imidazo[4,5-b]pyridine-7-carboxylic acid[2-(1-oxy-pyridin-3-yl)-ethyl]-amide;
130) 2-furan-3-yl-3H-imidazo[4,5-b]pyridine-7-carboxylic acid[2-(1-oxy-pyridin-4-yl)-ethyl]-amide;
131) 2-furan-3-yl-3H-imidazo[4,5-b]pyridine-7-carboxylic acid[2-(2-chloro-pyridin-3-yl)-ethyl]-amide;
132) 2-furan-3-yl-3H-imidazo[4,5-b]pyridine-7-carboxylic acid[2-(2-chloro-pyridin-4-yl)-ethyl]-amide;
133) 2-furan-3-yl-3H-imidazo[4,5-b]pyridine-7-carboxylic acid[2-(6-chloro-pyridin-3-yl)-ethyl]-amide;
134) 2-furan-3-yl-3H-imidazo[4,5-b]pyridine-7-carboxylic acid[2-(2-aminomethyl-pyridin-3-yl)-ethyl]-amide;
135) 2-furan-3-yl-3H-imidazo[4,5-b]pyridine-7-carboxylic acid[2-(2-aminomethyl-pyridin-4-yl)-ethyl]-amide;
136) 2-furan-3-yl-3H-imidazo[4,5-b]pyridine-7-carboxylic acid(3-pyridin-3-yl-propyl)-amide;
137) 2-furan-3-yl-3H-imidazo[4,5-b]pyridine-7-carboxylic acid[2-(4-trifluoromethane sulfonylamino-phenyl)-ethyl]-amide;
138) 2-furan-3-yl-3H-imidazo[4,5-b]pyridine-7-carboxylic acid[3-(6-chloro-pyridin-3-yl)-propyl]-amide;
139) 2-furan-3-yl-3H-imidazo[4,5-b]pyridine-7-carboxylic acid(3-pyridin-4-yl-propyl)-amide;
140) 6-bromo-2-furan-3-yl-3H-imidazo[4,5-b]pyridine-7-carboxylic acid [3-(4,5-dichloro-imidazo-1-yl)-propyl]-amide;
141) 2-furan-3-yl-3H-imidazo[4,5-b]pyridine-7-carboxylic acid(4-benzoylamino-phenyl)-amide;
142) 2-(2,4-difluoro-phenyl)-3H-imidazo[4,5-b]pyridine-7-carboxylic acid[2-(4-ethanesulfonylamino-phenyl)-ethyl]-amide;
143) 2-(2,4-difluoro-phenyl)-3H-imidazo[4,5-b]pyridine-7-carboxylic acid[2-(4-methanesulfonylamino-phenyl)-ethyl]-amide;
144) 2-(2,4-difluoro-phenyl)-3H-imidazo[4,5-b]pyridine-7-carboxylic acid[3-(4,5-dichloro-imidazo-1-yl)-propyl]-amide;
145) 2-cyclopropyl-3H-imidazo[4,5-b]pyridine-7-carboxylic acid[2-(4-methanesulfonylamino-phenyl)-ethyl]-amide;
146) 2-cyclopropyl-3H-imidazo[4,5-b]pyridine-7-carboxylic acid[2-(4-ethanesulfonylamino-phenyl)-ethyl]-amide;
147) 2-cyclopropyl-3H-imidazo[4,5-b]pyridine-7-carboxylic acid[2-(4-dimethylaminosulfonylamino-phenyl)-ethyl]-amide;
148) 2-cyclopropyl-3H-imidazo[4,5-b]pyridine-7-carboxylic acid[3-(4,5-dichloro-3H-imidazo-1-yl)-propyl]-amide;
149) 2-cyclopropyl-3H-imidazo[4,5-b]pyridine-7-carboxylic acid[3-(5-methyl-3H-imidazo-1-yl)-propyl]-amide;
150) 2-cyclopropyl-3H-imidazo[4,5-b]pyridine-7-carboxylic acid[2-(3-hydroxy-phenyl)-ethyl]-amide;
151) 2-thiophen-3-yl-3H-imidazo[4,5-b]pyridine-7-carboxylic acid[2-(3-hydroxy-phenyl)-ethyl]-amide;
152) 2-thiophen-3-yl-3H-imidazo[4,5-b]pyridine-7-carboxylic acid[2-(4-acetylamino-phenyl)-ethyl]-amide;
153) 2-thiophen-3-yl-3H-imidazo[4,5-b]pyridine-7-carboxylic acid[2-(2-hydroxy-phenyl)-ethyl]-amide;
154) 2-thiophen-3-yl-3H-imidazo[4,5-b]pyridine-7-carboxylic acid[2-(4-ethanesulfonylamino-phenyl)-ethyl]-amide;
155) 2-(1-oxo-thiophen-3-yl)-3H-imidazo[4,5-b]pyridine-7-carboxylic acid[2-(4-ethanesulfonylamino-phenyl)-ethyl]-amide;
156) 2-thiophen-3-yl-3H-imidazo[4,5-b]pyridine-7-carboxylic acid(2-pyridin-2-yl-ethyl)-amide;
157) 2-thiophen-3-yl-3H-imidazo[4,5-b]pyridine-7-carboxylic acid(2-pyridin-3-yl-ethyl)-amide;
158) 2-thiophen-3-yl-3H-imidazo[4,5-b]pyridine-7-carboxylic acid(2-pyridin-4-yl-ethyl)-amide;
159) 2-thiophen-3-yl-3H-imidazo[4,5-b]pyridine-7-carboxylic acid[2-(4-methanesulfonylamino-phenyl)-ethyl]-amide;
160) 2-thiophen-3-yl-3H-imidazo[4,5-b]pyridine-7-carboxylic acid[2-(4-dimethylaminosulfonylamino-phenyl)-ethyl]-amide;
161) 2-thiophen-3-yl-3H-imidazo[4,5-b]pyridine-7-carboxylic acid[2-(4-fluoro-3-hydroxy-phenyl)-ethyl]-amide;
162) 2-thiophen-3-yl-3H-imidazo[4,5-b]pyridine-7-carboxylic acid[3-(4-methyl-imidazo-1-yl)-propyl]-amide;
163) 2-thiophen-3-yl-3H-imidazo[4,5-b]pyridine-7-carboxylic acid(3-imidazo-1-yl-propyl)-amide;
164) 2-thiophen-3-yl-3H-imidazo[4,5-b]pyridine-7-carboxylic acid[2-(1-oxy-pyridin-2-yl)-ethyl]-amide;
165) 2-thiophen-3-yl-3H-imidazo[4,5-b]pyridine-7-carboxylic acid[2-(1-oxy-pyridin-3-yl)-ethyl]-amide;
166) 2-thiophen-3-yl-3H-imidazo[4,5-b]pyridine-7-carboxylic acid[2-(1-oxy-pyridin-4-yl)-ethyl]-amide;
167) 2-thiophen-3-yl-3H-imidazo[4,5-b]pyridine-7-carboxylic acid[2-(2-chloro-pyridin-4-yl)-ethyl]-amide;
168) 2-thiophen-3-yl-3H-imidazo[4,5-b]pyridine-7-carboxylic acid[2-(2-aminomethyl-pyridin-3-yl)-ethyl]-amide;
169) 2-thiophen-3-yl-3H-imidazo[4,5-b]pyridine-7-carboxylic acid[2-(6-chloro-1-oxy-pyridin-3-yl)-ethyl]-amide;
170) 2-thiophen-3-yl-3H-imidazo[4,5-b]pyridine-7-carboxylic acid[2-(2-fluoro-pyridin-4-yl)-ethyl]-amide;
171) 2-thiophen-3-yl-3H-imidazo[4,5-b]pyridine-7-carboxylic acid[2-(2-methyl amino-pyridin-4-yl)-ethyl]-amide;
172) 2-thiophen-3-yl-3H-imidazo[4,5-b]pyridine-7-carboxylic acid(3-pyridin-3-yl-propyl)-amide;
173) 2-thiophen-3-yl-3H-imidazo[4,5-b]pyridine-7-carboxylic acid[2-(4-trifluoromethanesulfonylamino-phenyl)-ethyl]-amide;
174) 2-thiophen-3-yl-3H-imidazo[4,5-b]pyridine-7-carboxylic acid[3-(6-chloro-pyridin-3-yl)-propyl]-amide;
175) 2-thiophen-3-yl-3H-imidazo[4,5-b]pyridine-7-carboxylic acid(3-pyridin-4-yl-propyl)-amide;
176) 2-thiophen-3-yl-3H-imidazo[4,5-b]pyridine-7-carboxylic acid[3-(2-chloro-pyridin-4-yl)-propyl]-amide;
177) 2-thiophen-3-yl-3H-imidazo[4,5-b]pyridine-7-carboxylic acid[2-(2-methyl-pyridin-3-yl)-ethyl]-amide;
178) 2-thiophen-3-yl-3H-imidazo[4,5-b]pyridine-7-carboxylic acid[3-(6-fluoro-pyridin-3-yl)-propyl]-amide;
179) 2-(1-oxo-thiophen-3-yl)-3H-imidazo[4,5-b]pyridine-7-carboxylic acid[3-(1-oxy-pyridin-3-yl)-propyl]-amide;
180) 2-(1-oxo-1H-thiophen-3-yl)-3H-imidazo[4,5-b]pyridine-7-carboxylic acid(3-pyridin-3-yl-propyl)-amide;
181) 2-thiophen-3-yl-3H-imidazo[4,5-b]pyridine-7-carboxylic acid pyridin-3-yl-amide;
182) 2-thiophen-3-yl-3H-imidazo[4,5-b]pyridine-7-carboxylic acid pyridin-4-yl-amide;
183) 2-thiophen-3-yl-3H-imidazo[4,5-b]pyridine-7-carboxylic acid(6-methyloxy-pyridin-3-yl)-amide;
184) 2-thiophen-3-yl-3H-imidazo[4,5-b]pyridine-7-carboxylic acid[2-(4-amino-phenyl)-ethyl]-amide;
185) 2-thiophen-3-yl-3H-imidazo[4,5-b]pyridine-7-carboxylic acid(6-methyl-pyridin-3-yl)-amide;
186) 2-thiophen-3-yl-3H-imidazo[4,5-b]pyridine-7-carboxylic acid cycloheptyl amide;
187) 2-thiophen-3-yl-3H-imidazo[4,5-b]pyridine-7-carboxylic acid(5-methyl-2H-pyrazol-3-yl)-amide;
188) 2-thiophen-3-yl-3H-imidazo[4,5-b]pyridine-7-carboxylic acid(6-methyl amino-pyridin-3-yl)-amide;
189) 2-thiophen-3-yl-1H-pyrrolo[2,3-b]pyridine-4-carboxylic acid(4-benzoylamino-phenyl)-amide;
190) 2-thiophen-3-yl-3H-imidazo[4,5-b]pyridine-7-carboxylic acid[3-(6-methanesulfonylamino-pyridin-3-yl)-propyl]-amide;
191) 6-bromo-2-thiophen-3-yl-3H-imidazo[4,5-b]pyridine-7-carboxylic acid[2-(2-chloro-pyridin-4-yl)-ethyl]-amide;
192) 2-thiophen-3-yl-3H-imidazo[4,5-b]pyridine-7-carboxylic acid(6-methyl-pyridin-3-yl)-amide;
193) 2-thiophen-3-yl-3H-imidazo[4,5-b]pyridine-7-carboxylic acid(4-ethoxy-pyridin-3-yl)-amide;
194) 2-thiophen-3-yl-3H-imidazo[4,5-b]pyridine-7-carboxylic acid(4-ethylsulfanyl-pyridin-3-yl)-amide;
195) 2-thiophen-3-yl-3H-imidazo[4,5-b]pyridine-7-carboxylic acid(4-ethoxy-6-methyl-pyridin-3-yl)-amide;
196) 2-(4-fluoro-phenyl)-3H-imidazo[4,5-b]pyridine-7-carboxylic acid[2-(4-acetylamino-phenyl)-ethyl]-amide;
197) 2-(4-fluoro-phenyl)-3H-imidazo[4,5-b]pyridine-7-carboxylic acid[2-(2-hydroxy-phenyl)-ethyl]-amide;
198) 2-(4-fluoro-phenyl)-3H-imidazo[4,5-b]pyridine-7-carboxylic acid[2-(4-ethanesulfonylamino-phenyl)-ethyl]-amide;
199) 2-(4-fluoro-phenyl)-3H-imidazo[4,5-b]pyridine-7-carboxylic acid[3-(3H-imidazo-1-yl)-propyl]-amide;
200) 2-(4-fluoro-phenyl)-3H-imidazo[4,5-b]pyridine-7-carboxylic acid{2-[4-(2-dimethylamino-acetylamino)-phenyl]-ethyl}-amide;
201) 2-(4-fluoro-phenyl)-3H-imidazo[4,5-b]pyridine-7-carboxylic acid[2-(3-hydroxy-phenyl)-ethyl]-amide;
202) 2-(4-fluoro-phenyl)-3H-imidazo[4,5-b]pyridine-7-carboxylic acid[3-(4,5-dichloro-imidazo-1-yl)-propyl]-amide;
203) 2-(4-fluoro-phenyl)-3H-imidazo[4,5-b]pyridine-7-carboxylic acid[2-(3-chloro-phenyl)-ethyl]-amide;
204) 2-(4-fluoro-phenyl)-3H-imidazo[4,5-b]pyridine-7-carboxylic acid(2-{4-[(4-methyl-piperazine-1-carbonyl)-amino]-phenyl}-ethyl)-amide;
205) 2-(4-fluoro-phenyl)-3H-imidazo[4,5-b]pyridine-7-carboxylic acid[3-(4-methyl-imidazo-1-yl)-propyl]-amide;
206) 2-(4-fluoro-phenyl)-3H-imidazo[4,5-b]pyridine-7-carboxylic acid[2-(4-methanesulfonylamino-phenyl)-ethyl]-amide;
207) 2-(4-fluoro-phenyl)-3H-imidazo[4,5-b]pyridine-7-carboxylic acid[2-(4-dimethylamino-sulfonylamino-phenyl)-ethyl]-amide;
208) 2-(4-fluoro-phenyl)-3H-imidazo[4,5-b]pyridine-7-carboxylic acid[2-(3-trifluoro methyl-phenyl)-ethyl]-amide;
209) 2-(4-fluoro-phenyl)-3H-imidazo[4,5-b]pyridine-7-carboxylic acid{2-[3-(2-morpholin-4-yl-ethoxy)-phenyl]-ethyl}-amide;
210) 2-(4-fluoro-phenyl)-3H-imidazo[4,5-b]pyridine-7-carboxylic acid{2-[3-(3-morpholin-4-yl-propoxy)-phenyl]-ethyl}-amide;
211) 2-(4-fluoro-phenyl)-3H-imidazo[4,5-b]pyridine-7-carboxylic acid{2-[3-(2-dimethylamino-ethoxy)-phenyl]-ethyl}-amide;
212) 2-(4-fluoro-phenyl)-3H-imidazo[4,5-b]pyridine-7-carboxylic acid(2-pyridin-2-yl-ethyl)-amide;
213) 2-(4-fluoro-phenyl)-3H-imidazo[4,5-b]pyridine-7-carboxylic acid(2-pyridin-3-yl-ethyl)-amide;
214) 2-(4-fluoro-phenyl)-3H-imidazo[4,5-b]pyridine-7-carboxylic acid(2-pyridin-4-yl-ethyl)-amide;
215) 2-(4-fluoro-phenyl)-3H-imidazo[4,5-b]pyridine-7-carboxylic acid[2-(4-fluoro-3-hydroxy-phenyl)-ethyl]-amide;
216) 2-(4-fluoro-phenyl)-3H-imidazo[4,5-b]pyridine-7-carboxylic acid[2-(1-oxy-pyridin-4-yl)-ethyl]-amide;
217) 2-(4-fluoro-phenyl)-3H-imidazo[4,5-b]pyridine-7-carboxylic acid[2-(2-chloro-pyridin-3-yl)-ethyl]-amide;
218) 2-(4-fluoro-phenyl)-3H-imidazo[4,5-b]pyridine-7-carboxylic acid[2-(2-chloro-pyridin-4-yl)-ethyl]-amide;
219) 2-(4-fluoro-phenyl)-3H-imidazo[4,5-b]pyridine-7-carboxylic acid[2-(6-chloro-pyridin-3-yl)-ethyl]-amide;
220) 2-(4-fluoro-phenyl)-3H-imidazo[4,5-b]pyridine-7-carboxylic acid[2-(2-aminomethyl-pyridin-3-yl)-ethyl]-amide;
221) 2-(4-fluoro-phenyl)-3H-imidazo[4,5-b]pyridine-7-carboxylic acid[2-(2-aminomethyl-pyridin-4-yl)-ethyl]-amide;
222) 2-(4-fluoro-phenyl)-3H-imidazo[4,5-b]pyridine-7-carboxylic acid[2-(6-aminomethyl-pyridin-3-yl)-ethyl]-amide;
223) 2-(4-fluoro-phenyl)-3H-imidazo[4,5-b]pyridine-7-carboxylic acid[2-(2-fluoro-pyridin-4-yl)-ethyl]-amide;
224) 2-(4-fluoro-phenyl)-3H-imidazo[4,5-b]pyridine-7-carboxylic acid[2-(2-methylamino-pyridin-4-yl)-ethyl]-amide;
225) 2-(4-fluoro-phenyl)-3H-imidazo[4,5-b]pyridine-7-carboxylic acid[2-(4-trifluoromethane sulfonylamino-phenyl)-ethyl]-amide;
226) 2-(4-fluoro-phenyl)-3H-imidazo[4,5-b]pyridine-7-carboxylic acid(3-pyridin-3-yl-propyl)-amide;
227) 2-(4-fluoro-phenyl)-3H-imidazo[4,5-b]pyridine-7-carboxylic acid(3-pyridin-4-yl-propyl)-amide;
228) 2-(4-fluoro-phenyl)-3H-imidazo[4,5-b]pyridine-7-carboxylic acid[3-(2-chloro-pyridin-4-yl)-propyl]-amide;
229) 2-(4-fluoro-phenyl)-3H-imidazo[4,5-b]pyridine-7-carboxylic acid[2-(2-methyl-pyridin-3-yl)-ethyl]-amide;
230) 2-(4-fluoro-phenyl)-3H-imidazo[4,5-b]pyridine-7-carboxylic acid[3-(6-fluoro-pyridin-3-yl)-propyl]-amide;
231) 2-(4-fluoro-phenyl)-3H-imidazo[4,5-b]pyridine-7-carboxylic acid pyridin-2-ylamide;
232) 2-(4-fluoro-phenyl)-3H-imidazo[4,5-b]pyridine-7-carboxylic acid pyridin-3-ylamide;
233) 2-(4-fluoro-phenyl)-3H-imidazo[4,5-b]pyridine-7-carboxylic acid pyridin-4-ylamide;
234) [2-(4-fluoro-phenyl)-3H-imidazo[4,5-b]pyridin-7-yl]-piperidin-1-yl-methanone;
235) [2-(4-fluoro-phenyl)-3H-imidazo[4,5-b]pyridin-7-yl]-(4-methyl-piperazin-1-yl)-methanone;
236) 2-(4-fluoro-phenyl)-3H-imidazo[4,5-b]pyridine-7-carboxylic acid[2-(4-amino-phenyl)-ethyl]-amide;
237) 2-(4-fluoro-phenyl)-3H-imidazo[4,5-b]pyridine-7-carboxylic acid(6-methyloxy-pyridin-3-yl)-amide;
238) 2-(4-fluoro-phenyl)-3H-imidazo[4,5-b]pyridine-7-carboxylic acid[3-(6-chloro-pyridin-3-yl)-propyl]-amide;
239) 2-(4-fluoro-phenyl)-3H-imidazo[4,5-b]pyridine-7-carboxylic acid(2-methyloxy-pyridin-3-yl)-amide;
240) 2-(4-fluoro-phenyl)-3H-imidazo[4,5-b]pyridine-7-carboxylic acid cycloheptylamide;
241) 2-(4-fluoro-phenyl)-3H-imidazo[4,5-b]pyridine-7-carboxylic acid(4-methyl-pyridin-3-yl)-amide;
242) 2-(4-fluoro-phenyl)-3H-imidazo[4,5-b]pyridine-7-carboxylic acid(6-chloro-pyridin-3-yl)-amide;
243) 2-(4-fluoro-phenyl)-3H-imidazo[4,5-b]pyridine-7-carboxylic acid(5-methyl-2H-pyrazol-3-yl)-amide;
244) 2-(4-fluoro-phenyl)-3H-imidazo[4,5-b]pyridine-7-carboxylic acid(5-methyl-pyridin-2-yl)-amide;
245) 2-(4-fluoro-phenyl)-3H-imidazo[4,5-b]pyridine-7-carboxylic acid(2-chloro-pyridin-3-yl)-amide;
246) 2-(4-fluoro-phenyl)-3H-imidazo[4,5-b]pyridine-7-carboxylic acid(4-methyloxy-pyridin-3-yl)-amide;
247) 2-(4-fluoro-phenyl)-3H-imidazo[4,5-b]pyridine-7-carboxylic acid(4-chloro-pyridin-3-yl)-amide;
248) 2-(4-fluoro-phenyl)-3-methyl-3H-imidazo[4,5-b]pyridine-7-carboxylic acid [2-(4-ethanesulfonylamino-phenyl)-ethyl]-amide;
249) 2-(4-fluoro-phenyl)-3H-imidazo[4,5-b]pyridine-7-carboxylic acid(4-piperidin-1-yl-phenyl)-amide;
250) 2-(4-fluoro-phenyl)-3H-imidazo[4,5-b]pyridine-7-carboxylic acid(4-morpholin-4-yl-phenyl)-amide;
251) 6-bromo-2-(4-fluoro-phenyl)-3H-imidazo[4,5-b]pyridine-7-carboxylic acid[2-(2-chloro-pyridin-4-yl)-ethyl]-amide;
252) 6-chloro-2-(4-fluoro-phenyl)-3H-imidazo[4,5-b]pyridine-7-carboxylic acid[2-(2-chloro-pyridin-4-yl)-ethyl]-amide;
253) 2-(4-fluoro-phenyl)-3H-imidazo[4,5-b]pyridine-7-carboxylic acid phenylamide;
254) 2-(4-fluoro-phenyl)-3H-imidazo[4,5-b]pyridine-7-carboxylic acid(4-hydroxy-cyclohexyl)-amide;
255) 2-(4-fluoro-phenyl)-3H-imidazo[4,5-b]pyridine-7-carboxylic acid(1-benzyl-piperidin-4-yl)-amide;
256) 2-(4-fluoro-phenyl)-3H-imidazo[4,5-b]pyridine-7-carboxylic acid piperidin-4-ylamide;
257) 2-(4-fluoro-phenyl)-3H-imidazo[4,5-b]pyridine-7-carboxylic acid (2,6-diethyl-phenyl)-amide;
258) 2-(4-fluoro-phenyl)-3H-imidazo[4,5-b]pyridine-7-carboxylic acid (2,6-diisopropyl-phenyl)-amide;
259) 2-(4-fluoro-phenyl)-3H-imidazo[4,5-b]pyridine-7-carboxylic acid benzyl-ethyl-amide;
260) 2-(4-fluoro-phenyl)-3H-imidazo[4,5-b]pyridine-7-carboxylic acid(1-benzyl-pyrrolidin-3-yl)-amide;
261) 2-(4-fluoro-phenyl)-3H-imidazo[4,5-b]pyridine-7-carboxylic acid isopropyl-phenyl-amide;
262) 2-(4-fluoro-phenyl)-3H-imidazo[4,5-b]pyridine-7-carboxylic acid (1,7,7-trimethyl-bicyclo[2.2.1]heptin-2-yl)-amide;
263) 2-(4-fluoro-phenyl)-3H-imidazo[4,5-b]pyridine-7-carboxylic acid adamantan-1-ylamide;
264) 2-(4-fluoro-phenyl)-3H-imidazo[4,5-b]pyridine-7-carboxylic acid adamantan-2-ylamide;
265) 2-{[2-(4-fluoro-phenyl)-3H-imidazo[4,5-b]pyridine-7-carbonyl]-amino}-bicyclo[2.2.1]heptane-2-carboxylic acid;
266) 2-(4-fluoro-phenyl)-3H-imidazo[4,5-b]pyridine-7-carboxylic acid(1-azabicyclo[2.2.2]octyn-3-yl)-amide;
267) 2-(4-fluoro-phenyl)-3H-imidazo[4,5-b]pyridine-7-carboxylic acid cyclohexylamide;
268) 2-(4-fluoro-phenyl)-3H-imidazo[4,5-b]pyridine-7-carboxylic acid bicyclo[2.2.1]heptin-2-ylamide;
269) 2-(4-fluoro-phenyl)-3H-imidazo[4,5-b]pyridine-7-carboxylic acid (2,6,6-trimethyl-bicyclo[3.1.1]heptin-3-yl)-amide;
270) 2-(4-fluoro-phenyl)-3H-imidazo[4,5-b]pyridine-7-carboxylic acid (2,6,6-trimethyl-bicyclo[3.1.1]heptin-3-yl)-amide;
271) 2-(4-fluoro-phenyl)-3H-imidazo[4,5-b]pyridine-7-carboxylic acid (2,6-dimethyl-phenyl)-amide;
272) 2-(4-fluoro-phenyl)-3H-imidazo[4,5-b]pyridine-7-carboxylic acid(3-amino-cyclohexyl)-amide;
273) 2-(4-fluoro-phenyl)-3H-imidazo[4,5-b]pyridine-7-carboxylic acid cyclopentylamide;
274) 2-(4-fluoro-phenyl)-3H-imidazo[4,5-b]pyridine-7-carboxylic acid bicyclo[2.2.1]heptin-2-ylamide;
275) 2-(4-fluoro-phenyl)-3H-imidazo[4,5-b]pyridine-7-carboxylic acid(2-hydroxy-1,1-dimethyl-ethyl)-amide;
276) 2-(4-fluoro-phenyl)-3H-imidazo[4,5-b]pyridine-7-carboxylic acid(6-methoxy-4-methyl-pyridin-3-yl)-amide;
277) 2-(4-fluoro-phenyl)-3H-imidazo[4,5-b]pyridine-7-carboxylic acid tert-butylamide;
278) 2-(4-fluoro-phenyl)-3H-imidazo[4,5-b]pyridine-7-carboxylic acid(2-dimethylamino-1-methyl-ethyl)-amide;
279) 2-(4-fluoro-phenyl)-3H-imidazo[4,5-b]pyridine-7-carboxylic acid(2-ethoxy-phenyl)-amide;
280) 2-(4-fluoro-phenyl)-3H-imidazo[4,5-b]pyridine-7-carboxylic acid(2-hydroxy-cyclopentyl)-amide;
281) 2-(4-fluoro-phenyl)-3H-imidazo[4,5-b]pyridine-7-carboxylic acid(1-hydroxymethyl-cyclopentyl)-amide;
282) 2-(4-fluoro-phenyl)-3H-imidazo[4,5-b]pyridine-7-carboxylic acid (1,1-dimethyl-propyl)-amide;
283) 2-(4-fluoro-phenyl)-3H-imidazo[4,5-b]pyridine-7-carboxylic acid (3,3,5-trimethyl-cyclohexyl)-amide;
284) 2-(4-fluoro-phenyl)-3H-imidazo[4,5-b]pyridine-7-carboxylic acid naphthalen-2-ylamide;
285) 2-(4-fluoro-phenyl)-3H-imidazo[4,5-b]pyridine-7-carboxylic acid quinolin-6-ylamide;
286) 2-(4-fluoro-phenyl)-3H-imidazo[4,5-b]pyridine-7-carboxylic acid quinolin-3-ylamide;
287) 2-(4-fluoro-phenyl)-3H-imidazo[4,5-b]pyridine-7-carboxylic acid(4-ethoxy-pyridin-3-yl)-amide;
288) 2-(4-fluoro-phenyl)-3H-imidazo[4,5-b]pyridine-7-carboxylic acid(6-methoxy-2-methyl-pyridin-3-yl)-amide;
289) 2-(4-fluoro-phenyl)-3H-imidazo[4,5-b]pyridine-7-carboxylic acid (1,3,3-trimethyl-butyl)-amide;
290) 2-(4-fluoro-phenyl)-3H-imidazo[4,5-b]pyridine-7-carboxylic acid (1,4-dimethyl-pentyl)-amide;
291) 2-(4-fluoro-phenyl)-3H-imidazo[4,5-b]pyridine-7-carboxylic acid(1-ethyl-propyl)-amide;
292) 2-(4-fluoro-phenyl)-3H-imidazo[4,5-b]pyridine-7-carboxylic acid (1,1,3,3-tetramethyl-butyl)-amide;
293) 2-(4-fluoro-phenyl)-3H-imidazo[4,5-b]pyridine-7-carboxylic acid benzothiazo-2-ylamide;
294) 2-(4-fluoro-phenyl)-3H-imidazo[4,5-b]pyridine-7-carboxylic acid (1H-indazo-6-yl)-amide;
295) 2-(4-fluoro-phenyl)-3H-imidazo[4,5-b]pyridine-7-carboxylic acid(2-methyl-1H-indol-5-yl)-amide;
296) 2-(4-fluoro-phenyl)-3H-imidazo[4,5-b]pyridine-7-carboxylic acid (1H-indol-5-yl)-amide;
297) 2-(4-fluoro-phenyl)-3H-imidazo[4,5-b]pyridine-7-carboxylic acid(3-trifluoromethoxy-phenyl)-amide;
298) 2-(4-fluoro-phenyl)-3H-imidazo[4,5-b]pyridine-7-carboxylic acid (3,5-dimethoxy-phenyl)-amide;
299) 2-(4-fluoro-phenyl)-3H-imidazo[4,5-b]pyridine-7-carboxylic acid(3-acetylamino-phenyl)-amide;
300) 2-(4-fluoro-phenyl)-3H-imidazo[4,5-b]pyridine-7-carboxylic acid(3-cyano-phenyl)-amide;
301) 2-(4-fluoro-phenyl)-3H-imidazo[4,5-b]pyridine-7-carboxylic acid(3-benzyloxy-phenyl)-amide;
302) 2-(4-fluoro-phenyl)-3H-imidazo[4,5-b]pyridine-7-carboxylic acid(4-phenyl-thiazo-2-yl)-amide;
303) 2-(4-fluoro-phenyl)-3H-imidazo[4,5-b]pyridine-7-carboxylic acid o-tolylamide;
304) 2-(4-fluoro-phenyl)-3H-imidazo[4,5-b]pyridine-7-carboxylic acid benzo[1,3]dioxol-5-ylamide;
305) 2-(4-fluoro-phenyl)-3H-imidazo[4,5-b]pyridine-7-carboxylic acid (2,6-dimethyl-pyridin-3-yl)-amide;
306) 2-(4-fluoro-phenyl)-3H-imidazo[4,5-b]pyridine-7-carboxylic acid(2-chloro-6-methoxy-pyridin-3-yl)-amide;
307) 2-(4-fluoro-phenyl)-3H-imidazo[4,5-b]pyridine-7-carboxylic acid(6-methoxy-5-methyl-pyridin-3-yl)-amide;
308) 2-(4-fluoro-phenyl)-3H-imidazo[4,5-b]pyridine-7-carboxylic acid(6-methyl-pyridin-3-yl)-amide;
309) 2-(4-fluoro-phenyl)-3H-imidazo[4,5-b]pyridine-7-carboxylic acid(2-methanesulfonyl-phenyl)-amide;
310) 2-(4-fluoro-phenyl)-3H-imidazo[4,5-b]pyridine-7-carboxylic acid(6-chloro-4-methyl-pyridin-3-yl)-amide;
311) 2-(4-fluoro-phenyl)-3H-imidazo[4,5-b]pyridine-7-carboxylic acid(4-isobutoxy-pyridin-3-yl)-amide;
312) 2-(4-fluoro-phenyl)-3H-imidazo[4,5-b]pyridine-7-carboxylic acid(4-cyclopropylmethoxy-pyridin-3-yl)-amide;
313) 2-(4-fluoro-phenyl)-3H-imidazo[4,5-b]pyridine-7-carboxylic acid[4-(2-dimethylamino-ethoxy)-pyridin-3-yl]-amide;
314) 2-(4-fluoro-phenyl)-3H-imidazo[4,5-b]pyridine-7-carboxylic acid[4-(2-morpholin-4-yl-ethoxy)-pyridin-3-yl]-amide;
315) 2-(4-fluoro-phenyl)-3H-imidazo[4,5-b]pyridine-7-carboxylic acid(2-chloro-4-methyl-pyridin-3-yl)-amide;
316) 2-(4-fluoro-phenyl)-3H-imidazo[4,5-b]pyridine-7-carboxylic acid(6-chloro-5-methyl-pyridin-3-yl)-amide;
317) 2-(4-fluoro-phenyl)-3H-imidazo[4,5-b]pyridine-7-carboxylic acid(2-chloro-5-methyl-pyridin-3-yl)-amide;
318) 2-(4-fluoro-phenyl)-3H-imidazo[4,5-b]pyridine-7-carboxylic acid (2,5-dichloro-pyridin-3-yl)-amide;
319) 2-(4-fluoro-phenyl)-3H-imidazo[4,5-b]pyridine-7-carboxylic acid (4,6-dichloro-pyrimidin-5-yl)-amide;
320) 2-(4-fluoro-phenyl)-3H-imidazo[4,5-b]pyridine-7-carboxylic acid(4-ethylsulfanyl-pyridin-3-yl)-amide;
321) 2-(4-fluoro-phenyl)-3H-imidazo[4,5-b]pyridine-7-carboxylic acid(6-dimethylamino-pyridin-3-yl)-amide;
322) 2-(4-fluoro-phenyl)-3H-imidazo[4,5-b]pyridine-7-carboxylic acid(6-acetylamino-pyridin-3-yl)-amide;
323) 2-(4-fluoro-phenyl)-3H-imidazo[4,5-b]pyridine-7-carboxylic acid[6-(2-morpholin-4-yl-ethoxy)-pyridin-3-yl]-amide;
324) 2-(4-fluoro-phenyl)-3H-imidazo[4,5-b]pyridine-7-carboxylic acid (4,6-dimethyl-pyridin-3-yl)-amide;
325) 2-(4-fluoro-phenyl)-3H-imidazo[4,5-b]pyridine-7-carboxylic acid(4-ethoxy-6-methyl-pyridin-3-yl)-amide;
326) 2-(4-fluoro-phenyl)-3H-imidazo[4,5-b]pyridine-7-carboxylic acid(4-ethylsulfanyl-6-methyl-pyridin-3-yl)-amide;
327) 2-[4-(4-methyl-piperazin-1-yl)-phenyl]-3H-imidazo[4,5-b]pyridine-7-carboxylic acid pyridin-3-ylamide;
328) 2-(4-methanesulfinylmethyl-phenyl)-3H-imidazo[4,5-b]pyridine-7-carboxylic acid pyridin-3-ylamide;
329) 2-(4-diethylamino-phenyl)-3H-imidazo[4,5-b]pyridine-7-carboxylic acid pyridin-3-ylamide;
330) 2-(4-morpholin-4-yl-phenyl)-3H-imidazo[4,5-b]pyridine-7-carboxylic acid pyridin-3-ylamide;
331) 2-(4-piperidin-1-yl-phenyl)-3H-imidazo[4,5-b]pyridine-7-carboxylic acid pyridin-3-ylamide;
332) 2-(4-pyrrolidin-1-yl-phenyl)-3H-imidazo[4,5-b]pyridine-7-carboxylic acid(4-methyl-pyridin-3-yl)-amide;
333) 2-(4-pyrrolidin-1-yl-phenyl)-3H-imidazo[4,5-b]pyridine-7-carboxylic acid(4-methyl-pyridin-3-yl)-amide;
334) 2-(4-morpholin-4-yl-phenyl)-3H-imidazo[4,5-b]pyridine-7-carboxylic acid(4-methyl-pyridin-3-yl)-amide;
335) 2-(4-piperidin-1-yl-phenyl)-3H-imidazo[4,5-b]pyridine-7-carboxylic acid(4-methyl-pyridin-3-yl)-amide;
336) 2-(4-dimethylamino-phenyl)-3H-imidazo[4,5-b]pyridine-7-carboxylic acid pyridin-3-ylamide;
337) 2-[4-(4-methyl-piperazin-1-yl)-phenyl]-3H-imidazo[4,5-b]pyridine-7-carboxylic acid(4-methyl-pyridin-3-yl)-amide;
338) 2-[4-(2-morpholin-4-yl-ethylamino)-phenyl]-3H-imidazo[4,5-b]pyridine-7-carboxylic acid(4-methyl-pyridin-3-yl)-amide;
339) 2-[4-(3-morpholin-4-yl-propylamino)-phenyl]-3H-imidazo[4,5-b]pyridine-7-carboxylic acid(4-methyl-pyridin-3-yl)-amide;
340) 2-[4-(3-pyrrolidin-1-yl-propylamino)-phenyl]-3H-imidazo[4,5-b]pyridine-7-carboxylic acid(4-methyl-pyridin-3-yl)-amide;
341) 2-[4-(2-dimethylamino-ethylamino)-phenyl]-3H-imidazo[4,5-b]pyridine-7-carboxylic acid(4-methyl-pyridin-3-yl)-amide;
342) 2-[4-(3-dimethylamino-propylamino)-phenyl]-3H-imidazo[4,5-b]pyridine-7-carboxylic acid(4-methyl-pyridin-3-yl)-amide;
343) 2-[4-(4-dimethylamino-butylamino)-phenyl]-3H-imidazo[4,5-b]pyridine-7-carboxylic acid(4-methyl-pyridin-3-yl)-amide;
344) 2-{4-[(2-diethylamino-ethyl)-methyl-amino]-phenyl}-3H-imidazo[4,5-b]pyridine-7-carboxylic acid(4-methyl-pyridin-3-yl)-amide;
345) 2-[4-(4-methyl-piperazin-1-yl)-phenyl]-3H-imidazo[4,5-b]pyridine-7-carboxylic acid(1,7,7-trimethyl-bicyclo[2.2.1]heptin-2-yl)-amide;
346) 2-(4-morpholin-4-yl-phenyl)-3H-imidazo[4,5-b]pyridine-7-carboxylic acid(1,7,7-trimethyl-bicyclo[2.2.1]heptin-2-yl)-amide;
347) 2-[4-(piperidin-4-ylamino)-phenyl]-3H-imidazo[4,5-b]pyridine-7-carboxylic acid(1,7,7-trimethyl-bicyclo[2.2.1]heptin-2-yl)-amide;
348) 2-[4-(4-methyl-piperazin-1-yl)-phenyl]-3H-imidazo[4,5-b]pyridine-7-carboxylic acid adamantan-2-ylamide;
349) 2-(4-morpholin-4-yl-phenyl)-3H-imidazo[4,5-b]pyridine-7-carboxylic acid adamantan-2-ylamide;
350) 2-[4-(4-methyl-piperazin-1-yl)-phenyl]-3H-imidazo[4,5-b]pyridine-7-carboxylic acid cycloheptylamide;
351) 2-(4-morpholin-4-yl-phenyl)-3H-imidazo[4,5-b]pyridine-7-carboxylic acid cycloheptylamide;
352) 2-(4-thiomorpholin-4-yl-phenyl)-3H-imidazo[4,5-b]pyridine-7-carboxylic acid cycloheptylamide;
353) 2-[4-(4-methyl-piperazin-1-yl)-phenyl]-3H-imidazo[4,5-b]pyridine-7-carboxylic acid(1-aza-bicyclo[2.2.2]octyn-3-yl)-amide;
354) 2-[4-(piperidin-4-ylamino)-phenyl]-3H-imidazo[4,5-b]pyridine-7-carboxylic acid cyclohexylamide;
355) 2-[4-(2-piperazin-1-yl-ethylamino)-phenyl]-3H-imidazo[4,5-b]pyridine-7-carboxylic acid cyclohexylamide;
356) 2-[4-(2-morpholin-4-yl-ethylamino)-phenyl]-3H-imidazo[4,5-b]pyridine-7-carboxylic acid cyclohexylamide;
357) 2-(4-morpholin-4-yl-phenyl)-3H-imidazo[4,5-b]pyridine-7-carboxylic acid cyclohexylamide;
358) 2-[4-(4-methyl-piperazin-1-yl)-phenyl]-3H-imidazo[4,5-b]pyridine-7-carboxylic acid cyclohexylamide;
359) 2-[4-(2-dimethylamino-ethylamino)-phenyl]-3H-imidazo[4,5-b]pyridine-7-carboxylic acid cyclohexylamide;
360) 2-{4-[(2-diethylamino-ethyl)-methyl-amino]-phenyl}-3H-imidazo[4,5-b]pyridine-7-carboxylic acid cyclohexylamide;
361) 2-[4-(4-ethyl-piperazin-1-yl)-phenyl]-3H-imidazo[4,5-b]pyridine-7-carboxylic acid cyclohexylamide;
362) 2-(4-morpholin-4-yl-phenyl)-3H-imidazo[4,5-b]pyridine-7-carboxylic acid(1-aza-bicyclo[2.2.2]octyn-3-yl)-amide;
363) 2-[4-(2-morpholin-4-yl-ethylamino)-phenyl]-3H-imidazo[4,5-b]pyridine-7-carboxylic acid(2,6,6-trimethyl-bicyclo[3.1.1]heptin-3-yl)-amide;
364) 2-[4-(2-morpholin-4-yl-ethylamino)-phenyl]-3H-imidazo[4,5-b]pyridine-7-carboxylic acid bicyclo[2.2.1]heptin-2-ylamide;
365) 2-[4-(2-morpholin-4-yl-ethylamino)-phenyl]-3H-imidazo[4,5-b]pyridine-7-carboxylic acid(2,6-dimethyl-phenyl)-amide;
366) 2-[4-(2-piperidin-1-yl-ethylamino)-phenyl]-3H-imidazo[4,5-b]pyridine-7-carboxylic acid(1,7,7-trimethyl-bicyclo[2.2.1]heptin-2-yl)-amide;
367) 2-[4-(2-morpholin-4-yl-ethylamino)-phenyl]-3H-imidazo[4,5-b]pyridine-7-carboxylic acid(4-hydroxy-cyclohexyl)-amide;
368) 2-[4-(2-morpholin-4-yl-ethylamino)-phenyl]-3H-imidazo[4,5-b]pyridine-7-carboxylic acid(1,7,7-trimethyl-bicyclo[2.2.1]heptin-2-yl)-amide;
369) 2-[4-(2-piperazin-1-yl-ethylamino)-phenyl]-3H-imidazo[4,5-b]pyridine-7-carboxylic acid(1,7,7-trimethyl-bicyclo[2.2.1]heptin-2-yl)-amide;
370) 2-[4-(2-dimethylamino-ethylamino)-phenyl]-3H-imidazo[4,5-b]pyridine-7-carboxylic acid(1,7,7-trimethyl-bicyclo[2.2.1]heptin-2-yl)-amide;
371) 2-[4-(4-methyl-piperazin-1-yl)-phenyl]-3H-imidazo[4,5-b]pyridine-7-carboxylic acid bicyclo[2.2.1]heptin-2-ylamide;
372) 2-[4-(2-dimethylamino-ethylamino)-phenyl]-3H-imidazo[4,5-b]pyridine-7-carboxylic acid bicyclo[2.2.1]heptin-2-ylamide;
373) 2-[4-(2-piperidin-1-yl-ethylamino)-phenyl]-3H-imidazo[4,5-b]pyridine-7-carboxylic acid bicyclo[2.2.1]heptin-2-ylamide;
374) 2-[4-(2-dimethylamino-ethylamino)-phenyl]-3H-imidazo[4,5-b]pyridine-7-carboxylic acid bicyclo[2.2.1]heptin-2-ylamide;
375) 2-[4-(2-dimethylamino-ethylamino)-phenyl]-3H-imidazo[4,5-b]pyridine-7-carboxylic acid(3-amino-cyclohexyl)-amide;
376) 2-[4-(2-diethylamino-ethylamino)-phenyl]-3H-imidazo[4,5-b]pyridine-7-carboxylic acid(1,7,7-trimethyl-bicyclo[2.2.1]heptin-2-yl)-amide;
377) 2-[4-(3-dimethylamino-propylamino)-phenyl]-3H-imidazo[4,5-b]pyridine-7-carboxylic acid(1,7,7-trimethyl-bicyclo[2.2.1]heptin-2-yl)-amide;
378) 2-[4-(2-pyrrolidin-1-yl-ethylamino)-phenyl]-3H-imidazo[4,5-b]pyridine-7-carboxylic acid(1,7,7-trimethyl-bicyclo[2.2.1]heptin-2-yl)-amide;
379) 2-{4-[3-(4-methyl-piperazin-1-yl)-propylamino]-phenyl}-3H-imidazo[
4,5-b]pyridine-7-carboxylic acid(1,7,7-trimethyl-bicyclo[2.2.1]heptin-2-yl)-amide;
380) 2-[4-(2-dimethylamino-ethylamino)-phenyl]-3H-imidazo[4,5-b]pyridine-7-carboxylic acid(2-hydroxy-1,1-dimethyl-ethyl)-amide;
381) 2-[4-(2-dimethylamino-ethylamino)-phenyl]-3H-imidazo[4,5-b]pyridine-7-carboxylic acid(1,1-dimethyl-propyl)-amide;
382) 2-[4-(2-dimethylamino-ethylamino)-phenyl]-3H-imidazo[4,5-b]pyridine-7-carboxylic acid(3,3,5-trimethyl-cyclohexyl)-amide;
383) 2-[4-(2-dimethylamino-ethylamino)-phenyl]-3H-imidazo[4,5-b]pyridine-7-carboxylic acid tert-butylamide;
384) 2-[4-(3-diethylamino-pyrrolidin-1-yl)-phenyl]-3H-imidazo[4,5-b]pyridine-7-carboxylic acid(1,7,7-trimethyl-bicyclo[2.2.1]heptin-2-yl)-amide;
385) 2-[4-(2-dimethylamino-ethylamino)-phenyl]-3H-imidazo[4,5-b]pyridine-7-carboxylic acid(2-dimethylamino-1-methyl-ethyl)-amide;
386) 2-[4-(2-dimethylamino-ethylamino)-phenyl]-3H-imidazo[4,5-b]pyridine-7-carboxylic acid(2-ethoxy-phenyl)-amide;
387) 2-[4-(2-dimethylamino-ethylamino)-phenyl]-3H-imidazo[4,5-b]pyridine-7-carboxylic acid(2-hydroxy-cyclopentyl)-amide;
388) 2-[4-(2-dimethylamino-ethylamino)-phenyl]-3H-imidazo[4,5-b]pyridine-7-carboxylic acid(1-hydroxymethyl-cyclopentyl)-amide;
389) 2-[4-(2-dimethylamino-ethylamino)-phenyl]-3H-imidazo[4,5-b]pyridine-7-carboxylic acid(3-imidazo-1-yl-propyl)-amide;
390) 2-[4-(2-dimethylamino-ethylamino)-phenyl]-3H-imidazo[4,5-b]pyridine-7-carboxylic acid[3-(4,5-dichloro-imidazo-1-yl)-propyl]-amide;
391) 2-[4-(3-pyrrolidin-1-yl-propylamino)-phenyl]-3H-imidazo[4,5-b]pyridine-7-carboxylic acid(2-pyridin-3-yl-ethyl)-amide;
392) 2-[4-(2-dimethylamino-ethylamino)-phenyl]-3H-imidazo[4,5-b]pyridine-7-carboxylic acid(1,3,3-trimethyl-butyl)-amide;
393) 2-[4-(2-dimethylamino-ethylamino)-phenyl]-3H-imidazo[4,5-b]pyridine-7-carboxylic acid(1,4-dimethyl-pentyl)-amide;
394) 2-[4-(2-dimethylamino-ethylamino)-phenyl]-3H-imidazo[4,5-b]pyridine-7-carboxylic acid(1-ethyl-propyl)-amide;
395) 2-[4-(2-dimethylamino-ethylamino)-phenyl]-3H-imidazo[4,5-b]pyridine-7-carboxylic acid(1,1,3,3-tetramethyl-butyl)-amide;
396) 2-(4-methoxy-phenyl)-3H-imidazo[4,5-b]pyridine-7-carboxylic acid (4-methyl-pyridin-3-yl)-amide;
397) 2-(4-methoxy-phenyl)-3H-imidazo[4,5-b]pyridine-7-carboxylic acid (4-ethoxy-pyridin-3-yl)-amide;
398) 2-(4-methoxy-phenyl)-3H-imidazo[4,5-b]pyridine-7-carboxylic acid (6-methoxy-pyridin-3-yl)-amide;
399) 2-(4-methoxy-phenyl)-3H-imidazo[4,5-b]pyridine-7-carboxylic acid (6-methyl-pyridin-3-yl)-amide;
400) 2-[4-(4-dimethylamino-butyl amino)-phenyl]-3H-imidazo[4,5-b]pyridine-7-carboxylic acid(4-ethoxy-pyridin-3-yl)-amide;
401) 2-(4-methoxy-phenyl)-3H-imidazo[4,5-b]pyridine-7-carboxylic acid (6-methoxy-4-methyl-pyridin-3-yl)-amide;
402) 2-[4-(4-methyl-piperazin-1-yl)-phenyl]-3H-imidazo[4,5-b]pyridine-7-carboxylic acid(6-methyl-pyridin-3-yl)-amide;
403) 2-(4-pyrrolidin-1-yl-phenyl)-3H-imidazo[4,5-b]pyridine-7-carboxylic acid(4-ethylsulfanyl-pyridin-3-yl)-amide;
404) 2-(4-pyrrolidin-1-yl-phenyl)-3H-imidazo[4,5-b]pyridine-7-carboxylic acid(4-ethoxy-pyridin-3-yl)-amide;
405) 2-(4-pyrrolidin-1-yl-phenyl)-3H-imidazo[4,5-b]pyridine-7-carboxylic acid(6-methyl-pyridin-3-yl)-amide;
406) 2-[4-(3-diethylamino-pyrrolidin-1-yl)-phenyl]-3H-imidazo[4,5-b]pyridine-7-carboxylic acid(4-ethylsulfanyl-pyridin-3-yl)-amide;
407) 2-[4-(4-methyl-piperazin-1-yl)-phenyl]-3H-imidazo[4,5-b]pyridine-7-carboxylic acid(4-ethoxy-pyridin-3-yl)-amide;
408) 2-(4-pyrrolidin-1-yl-phenyl)-3H-imidazo[4,5-b]pyridine-7-carboxylic acid(6-methoxy-4-methyl-pyridin-3-yl)-amide;
409) 2-[4-(3-diethylamino-pyrrolidin-1-yl)-phenyl]-3H-imidazo[4,5-b]pyridine-7-carboxylic acid(6-methyl-pyridin-3-yl)-amide;
410) 2-[4-(4-methyl-piperazin-1-yl)-phenyl]-3H-imidazo[4,5-b]pyridine-7-carboxylic acid(6-methoxy-4-methyl-pyridin-3-yl)-amide;
411) 2-(4-piperidin-1-yl-phenyl)-3H-imidazo[4,5-b]pyridine-7-carboxylic acid(4-ethoxy-pyridin-3-yl)-amide;
412) 2-(4-piperidin-1-yl-phenyl)-3H-imidazo[4,5-b]pyridine-7-carboxylic acid(6-methyl-pyridin-3-yl)-amide;
413) 2-(4-piperidin-1-yl-phenyl)-3H-imidazo[4,5-b]pyridine-7-carboxylic acid(6-methoxy-4-methyl-pyridin-3-yl)-amide;
414) 2-(4-piperidin-1-yl-phenyl)-3H-imidazo[4,5-b]pyridine-7-carboxylic acid(4,6-dimethyl-pyridin-3-yl)-amide;
415) 2-(4-pyrrolidin-1-yl-phenyl)-3H-imidazo[4,5-b]pyridine-7-carboxylic acid(4,6-dimethyl-pyridin-3-yl)-amide;
416) 2-[4-(4-methyl-piperazin-1-yl)-phenyl]-3H-imidazo[4,5-b]pyridine-7-carboxylic acid(4,6-dimethyl-pyridin-3-yl)-amide;
417) 2-(4-piperidin-1-yl-phenyl)-3H-imidazo[4,5-b]pyridine-7-carboxylic acid(4-ethylsulfanyl-pyridin-3-yl)-amide;
418) 2-[4-(4-methyl-piperazin-1-yl)-phenyl]-3H-imidazo[4,5-b]pyridine-7-carboxylic acid(4-ethylsulfanyl-pyridin-3-yl)-amide;
419) 2-{4-[(2-dimethylamino-ethyl)-methyl-amino]-phenyl}-3H-imidazo[
4,5-b]pyridine-7-carboxylic acid(4,6-dimethyl-pyridin-3-yl)-amide;
420) 2-{4-[(2-dimethylamino-ethyl)-methyl-amino]-phenyl}-3H-imidazo[
4,5-b]pyridine-7-carboxylic acid(4-ethoxy-pyridin-3-yl)-amide;
421) 2-{4-[(2-dimethylamino-ethyl)-methyl-amino]-phenyl}-3H-imidazo[
4,5-b]pyridine-7-carboxylic acid(6-methyl-pyridin-3-yl)-amide;
422) 2-{4-[(2-dimethylamino-ethyl)-methyl-amino]-phenyl}-3H-imidazo[
4,5-b]pyridine-7-carboxylic acid(4-ethylsulfanyl-pyridin-3-yl)-amide;
423) 2-[4-(4-methyl-piperazin-1-yl)-phenyl]-3H-imidazo[4,5-b]pyridine-7-carboxylic acid(4-ethoxy-6-methyl-pyridin-3-yl)-amide;
424) 2-(4-methoxy-phenyl)-3H-imidazo[4,5-b]pyridine-7-carboxylic acid (4-methyl-pyridin-3-yl)-amide;
425) 2-(4-methoxy-phenyl)-3H-imidazo[4,5-b]pyridine-7-carboxylic acid (4-ethoxy-pyridin-3-yl)-amide;
426) 2-(4-methoxy-phenyl)-3H-imidazo[4,5-b]pyridine-7-carboxylic acid (6-methoxy-pyridin-3-yl)-amide;
427) 2-(4-methoxy-phenyl)-3H-imidazo[4,5-b]pyridine-7-carboxylic acid (6-methyl-pyridin-3-yl)-amide;
428) 2-(4-methoxy-phenyl)-3H-imidazo[4,5-b]pyridine-7-carboxylic acid (6-methoxy-4-methyl-pyridin-3-yl)-amide;
429) 6-bromo-2-(3-methoxy-phenyl)-3H-imidazo[4,5-b]pyridine-7-carboxylic acid(4-methyl-pyridin-3-yl)-amide;
430) 2-(3-methoxy-phenyl)-3H-imidazo[4,5-b]pyridine-7-carboxylic acid (4-methyl-pyridin-3-yl)-amide;
431) 2-(3-methoxy-phenyl)-3H-imidazo[4,5-b]pyridine-7-carboxylic acid (6-methoxy-pyridin-3-yl)-amide;
432) 2-(3-methoxy-phenyl)-3H-imidazo[4,5-b]pyridine-7-carboxylic acid (4-ethoxy-pyridin-3-yl)-amide;
433) 2-(3-methoxy-phenyl)-3H-imidazo[4,5-b]pyridine-7-carboxylic acid (6-methyl-pyridin-3-yl)-amide;
434) 2-pyridin-2-yl-3H-imidazo[4,5-b]pyridine-7-carboxylic acid(4-methyl-pyridin-3-yl)-amide;
435) 2-pyridin-2-yl-3H-imidazo[4,5-b]pyridine-7-carboxylic acid(4-ethoxy-pyridin-3-yl)-amide;
436) 2-pyridin-2-yl-3H-imidazo[4,5-b]pyridine-7-carboxylic acid(2-chloro-pyridin-3-yl)-amide;
437) 2-pyridin-2-yl-3H-imidazo[4,5-b]pyridine-7-carboxylic acid(6-methyl-pyridin-3-yl)-amide;
438) 2-pyridin-3-yl-3H-imidazo[4,5-b]pyridine-7-carboxylic acid(4-ethoxy-pyridin-3-yl)-amide;
439) 2-pyridin-3-yl-3H-imidazo[4,5-b]pyridine-7-carboxylic acid(2-chloro-pyridin-3-yl)-amide;
440) 2-pyridin-3-yl-3H-imidazo[4,5-b]pyridine-7-carboxylic acid(6-chloro-pyridin-3-yl)-amide;
441) 2-pyridin-3-yl-3H-imidazo[4,5-b]pyridine-7-carboxylic acid(4-methyl-pyridin-3-yl)-amide;
442) 2-pyridin-3-yl-3H-imidazo[4,5-b]pyridine-7-carboxylic acid(6-methyl-pyridin-3-yl)-amide;
443) 2-pyridin-3-yl-3H-imidazo[4,5-b]pyridine-7-carboxylic acid(6-methoxy-4-methyl-pyridin-3-yl)-amide;
444) 2-pyridin-4-yl-3H-imidazo[4,5-b]pyridine-7-carboxylic acid(4-methyl-pyridin-3-yl)-amide;
445) 2-pyridin-4-yl-3H-imidazo[4,5-b]pyridine-7-carboxylic acid(4-ethoxy-pyridin-3-yl)-amide;
446) 2-pyridin-4-yl-3H-imidazo[4,5-b]pyridine-7-carboxylic acid(6-methyl-pyridin-3-yl)-amide;
447) 2-(3-chloro-phenyl)-3H-imidazo[4,5-b]pyridine-7-carboxylic acid (4-ethoxy-pyridin-3-yl)-amide;
448) 2-(3-chloro-phenyl)-3H-imidazo[4,5-b]pyridine-7-carboxylic acid (4-methyl-pyridin-3-yl)-amide;
449) 2-(3-chloro-phenyl)-3H-imidazo[4,5-b]pyridine-7-carboxylic acid (1,7,7-trimethyl-bicyclo[2.2.1]heptin-2-yl)-amide;
450) 2-(3-chloro-phenyl)-3H-imidazo[4,5-b]pyridine-7-carboxylic acid (6-methyl-pyridin-3-yl)-amide;
451) 2-(3-fluoro-phenyl)-3H-imidazo[4,5-b]pyridine-7-carboxylic acid(4-methyl-pyridin-3-yl)-amide;
452) 6-bromo-2-(3-fluoro-phenyl)-3H-imidazo[4,5-b]pyridine-7-carboxylic acid(4-methyl-pyridin-3-yl)-amide;
453) 2-(3-fluoro-phenyl)-3H-imidazo[4,5-b]pyridine-7-carboxylic acid (1,7,7-trimethyl-bicyclo[2.2.1]heptin-2-yl)-amide;
454) 2-(3-fluoro-phenyl)-3H-imidazo[4,5-b]pyridine-7-carboxylic acid(6-methyl-pyridin-3-yl)-amide;
455) 2-(3-fluoro-phenyl)-3H-imidazo[4,5-b]pyridine-7-carboxylic acid(4-ethoxy-pyridin-3-yl)-amide;
456) 2-(3-fluoro-phenyl)-3H-imidazo[4,5-b]pyridine-7-carboxylic acid(4-ethylsulfanyl-pyridin-3-yl)-amide;
457) 6-bromo-2-(3-fluoro-phenyl)-3H-imidazo[4,5-b]pyridine-7-carboxylic acid(4-ethoxy-6-methyl-pyridin-3-yl)-amide; and
458) 2-(3-fluoro-phenyl)-3H-imidazo[4,5-b]pyridine-7-carboxylic acid(4-ethoxy-6-methyl-pyridin-3-yl)-amide.

4. A method for preparing a compound of formula 1, which comprises the steps of:
1) hydrogenating a compound of formula 2 in the presence of a catalyst to obtain a compound of formula 3;
2) refluxing a mixture of the compound of formula 3 and R₁—(CO₂H) or R₁—(CHO) in the presence of an organic acid or heating the mixture in nitrobenzene by microwave irradiation to obtain a compound of formula 4;
3) reacting the compound of formula 4 with an oxidizing agent in an alkali hydroxide solution or an organic solvent, cooling the resulting mixture in an ice bath, adding SOCl₂ or H₂SO₄ thereto, and refluxing the resulting mixture to obtain a compound of formula 5;
4) refluxing the compound of formula 5 together with LiOH.H₂O in a solvent and adding an acid thereto to obtain a compound of formula 6; and
5) reacting the compound of formula 6 with a compound of formula R₂R₃NH in an organic solvent in the presence of a coupling agent to obtain the compound of formula 1: 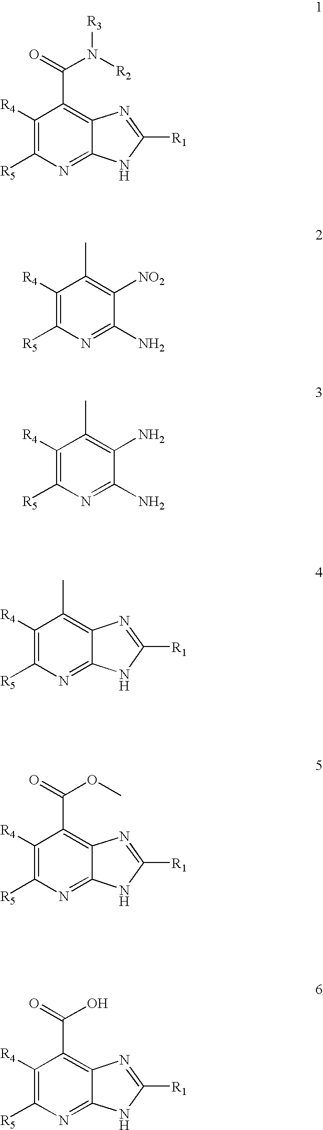
wherein, R₁ to R₅ have the same meanings as defined in claim 1.

5. The method of claim 4, wherein the catalyst used in step 1) is 5% to 10% Pd/C or PtO₂.

6. The method of claim 4, wherein the organic acid used in step 2) is POCl₃ or phosphoric acid (PPA).

7. The method of claim 4, wherein the alkali hydroxide solution used in step 3) is NaOH, NaHCO₃ or Na₂CO₃.

8. The method of claim 4, wherein the organic solvent used in step 3) is pyridine or t-BuOH.

9. The method of claim 4, wherein the oxidizing agent used in step 3) is KMnO₄, MnO₂ or SeO₂.

10. The method of claim 4, wherein the solvent used in step 4) is a mixture of water, MeOH and THF.

11. The method of claim 4, wherein the acid used in step 4) is HCl.

12. The method of claim 4, wherein the organic solvent used in step 5) is dimethylformamide (DMF), dimethyl sulfoxide (DMSO) or methylenechloride (MC).

13. The method of claim 4, wherein the coupling agent used in step 5) is 1-hydroxybenzotriazole (HOBT)/1-(3-dimethylaminopropyl)-3-ethylcarbdiimide HCl salt (EDC)/triethylamine (Et₃N), and pyBop ((benzotriazole-1-yl-oxy)tripyrrolidinophosphonium hexafluorophosphate), HBTU (O-benzotriazole-N,N,N′,N′-tetramethyluronium hexafluorophosphate) or TBTU (O-(benzotriazole-1-yl)-N,N,N′,N′-tetramethyluronium tetrafluoroborate).

14. A composition for inhibiting the activity of a protein kinase comprising the compound of formula 1, or a pharmaceutically acceptable salt, hydrate, solvate or isomer thereof defined in claim 1 as an active ingredient.

15. The composition of claim 14, wherein the protein kinase is selected from the group consisting of glycogen synthase kinase-3 (GSK-3), aurora kinase, extracellular signal-regulated kinase (ERK), protein kinase B (AKT), cyclin-dependent kinase (CDK), p38 (protein 38) mitogen-activated protein kinase (MAPK), kinase insert domain protein receptor (KDR) or vascular endothelial growth factor receptor-2 (VEGFR-2), c-Jun N-terminal kinase (JNK) and pyruvate dehydrogenase kinase (PDK).

16. A pharmaceutical composition for preventing or treating diseases selected from the group consisting of diabetes, obesity, dementia, cancer and inflammation comprising the compound of formula 1, or a pharmaceutically acceptable salt, hydrate, solvate or isomer thereof defined in claim 1 as an active ingredient.
